(19) United States
(12) Patent Application Publication
(10) Pub. No.: US 2026/0263504 A1
(43) Pub. Date: Sep. 10, 2026

(54) LIVER CELL REGENERATION

(71) Applicant: AGENCY FOR SCIENCE, TECHNOLOGY AND RESEARCH, Singapore (SG)

(72) Inventors: Torsten Wuestefeld, Singapore (SG); Shainan Hora, SG (SG); Amanpreet Kaur, SG (SG)

(21) Appl. No.: 19/489,241

(22) PCT Filed: May 31, 2024
(86) PCT No.: PCT/SG2024/050364

§ 371 Date: Dec. 1, 2025

(30) Foreign Application Priority Data
May 31, 2023 (SG) 10202301535X

(51) Int. Cl. A61K 31/713; A61K 31/7105; A61P 1/16; C12N 15/85

(52) U.S. Cl. CPC A61K 31/713; A61K 31/7105; A61P 1/16; C12N 15/85; C12N 2310/14; C12N 2800/107

(57) ABSTRACT

There is provided a method of regenerating a liver cell in a subject having a liver cell degeneration, comprising administering an agent that modulates one or more gene or marker comprising C1ORF131 (2810004N23Rik), SLC45A4, NFKBIB, ARL6IP5, DBNL, GOLGA7B, FAM117B, MED28, TSC22D4, EIF4EBP1, PFN1, PNRC2, ZNF672 (Zfp672), IER5, ADAMTSL5, COL4A5, MYH15, and ILKAP, wherein the agent counteracts the liver cell degeneration and/or enhances a liver cell regeneration. Also disclosed are nucleic acid encoding an agent for inhibiting the one or more gene or marker, composition or a vector or a plasmid comprising the nucleic acids, a kit comprising the nucleic acids, and a composition or vector or plasmid for use in therapy.

### TECHNICAL FIELD

The present disclosure relates broadly to methods and compositions for regenerating a liver cell.

### BACKGROUND

Non-Alcoholic Fatty Liver Disease (NAFLD) is now the number one chronic liver disease worldwide. In its progressive form of non-alcoholic steatohepatitis (NASH), NASH leads to liver fibrosis, cirrhosis, and liver cancer. However, despite its seemingly unstoppable rise, the mechanisms behind disease manifestation and progression remain unclear. Furthermore, other than liver transplantation, only one FDA-approved drug Resmitrom, for treatment of patients with moderate to advanced fibrosis but with a limited response rate of 30%, is available for NASH patients. Compounding to these difficulties, correct diagnosis and staging relies on invasive liver biopsy.

With a worldwide prevalence of 25% for NAFLD and 3 to 5% for NASH and no pharmacological therapy approved so far, the socio-economic impact of this disease as well as the burden for the health system cannot be overestimated. In Singapore it is even estimated that more than 40% of the population is affected by NAFLD.

In view of the above, there is a need to understand the underlying pathogenic mechanisms. There is also a need to provide alternative diagnostic methods and targets for therapy.

### SUMMARY

In one aspect, there is provided a method of regenerating a liver cell in a subject having a liver cell degeneration, comprising administering an agent that modulates one or more gene or marker comprising C1ORF131 (2810004N23Rik), SLC45A4, NFKBIB, ARL6IP5, DBNL, GOLGA7B, FAM117B, MED28, TSC22D4, EIF4EBP1, PFN1, PNRC2, ZNF672 (Zfp672), IER5, ADAMTSL5, COL4A5, MYH15, and ILKAP, wherein the agent counteracts the liver cell degeneration and/or enhances a liver cell regeneration.

In some examples, the one or more gene or marker further comprises ABCB10, LSM14A, ACTG1, YTHDF2, RPS6KA1, SEC13, IPO11, CTCF, UBAP2L, P2RY2, TRAF3IP2, FST, TUBG1, ZNF664, PVR, DYX1C1/DNAAF4, TEX264, NIF3L1, RPUSD1, TRIM6, GLRX3, BRD1, CKS1B, C6ORF120, WASF2, ZNF689, B4GALT7, LCORL, NR2F2, BAG6, and RNF10.

In some examples, the one or more gene or marker further comprises Uba6, Brwd3, Fyco1, ARID5B, C20ORF96 (6820408C15Rik), CD200R1 (Cd200r4), DALRD3, ELF3, ELFN1, FAM160A1, FAM50B, KIAA2026 (9930021J03Rik), KREMEN1, MACC1, NAA25, PROSC/PLPBP, RPS3, SHPK, SLAIN1, TCHP, ZNF503 (Zfp503), BUD13, CD93, PFKFB2, ZDBF2, FOXC2, GBGT1, RGS4, RNASEH1, TSNAXIP1, LYPD2, STIL, TMCO3, TMEM159, RIF1, IFIH1, EPN2, CLP1, RNF220, NOL12, APOL3, GTF3C6, CTPS2, IFITM2, IFITM1, KCNS3, CA2, EIF4H, MRM1, AKAP8, PAPOLA, SECTM1, TYK2, MLLT6, IL1RL2, IPO7, APBB1IP, RABEP2, PLSCR1, PYGO2, COIL, LRRC8E, TNRC6C, CHD3, USP21, SNX27, NOP14, ZC3H15, C15orf39, UTP20, TRAK1, RAB11FIP2, PHPT1, CUL3, and GNL3.

In some examples, the liver cell is a hepatocyte.

In some examples, the liver degeneration is non-alcoholic fatty liver disease (NAFLD) and/or non-alcoholic steatohepatitis (NASH).

In some examples, the agent reduces or inhibits the expression of the gene or marker.

In some examples, the agent is a nucleic acid capable of interfering with the expression of specific gene.

In another aspect, there is provided a nucleic acid encoding an agent for inhibiting the one or more gene or marker comprises C1ORF131 (2810004N23Rik), SLC45A4, NFKBIB, ARL6IP5, DBNL, GOLGA7B, FAM117B, MED28, TSC22D4, EIF4EBP1, PFN1, PNRC2, ZNF672 (Zfp672), IER5, ADAMTSL5, COL4A5, MYH15, and ILKAP.

In some examples, the one or more gene or marker further comprises ABCB10, LSM14A, ACTG1, YTHDF2, RPS6KA1, SEC13, IPO11, CTCF, UBAP2L, P2RY2, TRAF3IP2, FST, TUBG1, ZNF664, PVR, DYX1C1/DNAAF4, TEX264, NIF3L1, RPUSD1, TRIM6, GLRX3, BRD1, CKS1B, C6ORF120, WASF2, ZNF689, B4GALT7, LCORL, NR2F2, BAG6, and RNF10.

In some examples, the one or more gene or marker may further comprises UBA6, BRWD3, FYCO1, ARID5B, C20ORF96 (6820408C15Rik), CD200R1 (Cd200r4), DALRD3, ELF3, ELFN1, FAM160A1, FAM50B, KIAA2026 (9930021J03Rik), KREMEN1, MACC1, NAA25, PROSC/PLPBP, RPS3, SHPK, SLAIN1, TCHP, ZNF503 (Zfp503), BUD13, CD93, PFKFB2, ZDBF2, FOXC2, GBGT1, RGS4, RNASEH1, TSNAXIP1, LYPD2, STIL, TMCO3, TMEM159, RIF1, IFIH1, EPN2, CLP1, RNF220, NOL12, APOL3, GTF3C6, CTPS2, IFITM2, IFITM1, KCNS3, CA2, EIF4H, MRM1, AKAP8, PAPOLA, SECTM1, TYK2, MLLT6, IL1RL2, IPO7, APBB1IP, RABEP2, PLSCR1, PYGO2, COIL, LRRC8E, TNRC6C, CHD3, USP21, SNX27, NOP14, ZC3H15, C15ORF39, UTP20, TRAK1, RAB11FIP2, PHPT1, CUL3, and GNL3.

In some examples, the nucleic acid comprises one or more of an RNAi (RNA interference), a short hairpin RNA (shRNA), an antisense oligonucleotide (ASO), a gapmer, a short hairpin antisense oligonucleotide (shASO), a lipid nanoparticle, an adeno-associated virus vector, a gene editing agent, a ribozyme, or a nucleic acid-based nanoparticle.

In some examples, the nucleic acid is a shRNA.

In some examples, the nucleic acid is a shRNA and comprises the sequence selected from the group consisting of

| | | | SEQ |
|---|---|---|---|
| | Entrez | | ID |
| shRNA | ID | shRNA 97 mer sequence | NO: |
| C1orf131. | 128061 | TGCTGTTGACAGTGAGCGAAACCAAGAATCTAGTAATTTATA | 1 |
| 1320 | | GTGAAGCCACAGATGTATAAATTACTAGATTCTTGGTTCTGCC | |
| | | TACTGCCTCGGA | |
| C1orf131. | 128061 | TGCTGTTGACAGTGAGCGCACAGAAGAAGATCATAAAGAATA | 2 |
| 179 | | GTGAAGCCACAGATGTATTCTTTATGATCTTCTTCTGTTTGCC | |
| | | TACTGCCTCGGA | |
| C1orf131. | 128061 | TGCTGTTGACAGTGAGCGCCAGAAGAAGATCATAAAGAAATA | 3 |
| 180 | | GTGAAGCCACAGATGTATTTCTTTATGATCTTCTTCTGTTGCC | |
| | | TACTGCCTCGGA | |
| C1orf131. | 128061 | TGCTGTTGACAGTGAGCGACAGATCATAACAAGAACACAATA | 4 |
| 466 | | GTGAAGCCACAGATGTATTGTGTTCTTGTTATGATCTGGTGC | |
| | | CTACTGCCTCGGA | |
| C1orf131. | 128061 | TGCTGTTGACAGTGAGCGAAGAGATGTGGATACACAAGAATA | 5 |
| 513 | | GTGAAGCCACAGATGTATTCTTGTGTATCCACATCTCTCTGC | |
| | | CTACTGCCTCGGA | |
| C1orf131. | 128061 | TGCTGTTGACAGTGAGCGCAAAGAGTTATGTGAATTACAATA | 6 |
| 653 | | GTGAAGCCACAGATGTATTGTAATTCACATAACTCTTTTTGCC | |
| | | TACTGCCTCGGA | |
| SLC45A4. | 57210 | TGCTGTTGACAGTGAGCGCACAGAAGTACTTGGACAACTATA | 7 |
| 1893 | | GTGAAGCCACAGATGTATAGTTGTCCAAGTACTTCTGTATGC | |
| | | CTACTGCCTCGGA | |
| SLC45A4. | 57210 | TGCTGTTGACAGTGAGCGACACAATGTGGTTCTCTTAGAATA | 8 |
| 2558 | | GTGAAGCCACAGATGTATTCTAAGAGAACCACATTGTGGTGC | |
| | | CTACTGCCTCGGA | |
| SLC45A4. | 57210 | TGCTGTTGACAGTGAGCGCCGGGCAAGTACTGCAGCTATTTA | 9 |
| 3229 | | GTGAAGCCACAGATGTAAATAGCTGCAGTACTTGCCCGATGC | |
| | | CTACTGCCTCGGA | |
| SLC45A4. | 57210 | TGCTGTTGACAGTGAGCGCACCACAGTCTCTTTAGAACTATA | 10 |
| 3266 | | GTGAAGCCACAGATGTATAGTTCTAAAGAGACTGTGGTTTGC | |
| | | CTACTGCCTCGGA | |
| SLC45A4. | 57210 | TGCTGTTGACAGTGAGCGACCCGCACATCACACAGATAAATA | 11 |
| 3340 | | GTGAAGCCACAGATGTATTTATCTGTGTGATGTGCGGGGTGC | |
| | | CTACTGCCTCGGA | |
| SLC45A4. | 57210 | TGCTGTTGACAGTGAGCGCCACACAGATAAAGTCAGACAATA | 12 |
| 3349 | | GTGAAGCCACAGATGTATTGTCTGACTTTATCTGTGTGATGC | |
| | | CTACTGCCTOGGA | |

15. The nucleic acid of any one of claims 9 to 14, wherein the nucleic acid is a siRNA.

16. The nucleic acid of any one of claims 9 to 15, wherein the nucleic acid is a siRNA and comprises the sequence selected from the group consisting of
C1ORF131 (Variant 1) Isoform a

| | | | SEQ | | SEQ | | |
|---|---|---|---|---|---|---|---|
| siRNA_ | | | ID | | ID | | Corrected_ |
| id | Position | SS Sequence | NO: | AS Sequence | NO: | Score | Score |
| 1 | 1251 | GCUAGUAGUUUA | 13 | UUUAAUGUAAAC | 63 | 104.1 | 92.1 |
| | | CAUUAAAUU | | UACUAGCAU | | | |
| 2 | 1316 | CCAAGAAUCUAG | 14 | UAAAUUACUAGA | 64 | 102.8 | 89.8 |
| | | UAAUUUAUC | | UUCUUGGUU | | | |
| 3 | 649 | AGAGUUAUGUGA | 15 | UUGUAAUUCACA | 65 | 99.9 | 93.8 |
| | | AUUACAAGG | | UAACUCUUU | | | |
| 4 | 171 | GAACAGAAGAAG | 16 | UUAUGAUCUUCU | 66 | 99.1 | 98.1 |
| | | AUCAUAAAG | | UCUGUUCUG | | | |
| 5 | 562 | GGUUUGGUAUCA | 17 | UAACCCGUGAUA | 67 | 99 | 94 |
| | | CGGGUUAUG | | CCAAACCGG | | | |
| 6 | 986 | CUAGAUAGAUCG | 18 | UUAAGAACGAUC | 68 | 98.4 | 89.4 |
| | | UUCUUAACA | | UAUCUAGAG | | | |
| 7 | 143 | CGACUUUGGAGG | 19 | UUCUGUACCUCC | 69 | 98.2 | 97.2 |
| | | UACAGAAGG | | AAAGUCGUA | | | |
| 8 | 1201 | GGUGCAUUUGCU | 20 | UAUUACAAGCAA | 70 | 98.2 | 86.2 |
| | | UGUAAUACA | | AUGCACCAA | | | |
| 9 | 1250 | UGCUAGUAGUUU | 21 | UUAAUGUAAACU | 71 | 96.4 | 84.4 |
| | | ACAUUAAAU | | ACUAGCAUU | | | |
| 10 | 1244 | UGUAAAUGCUAG | 22 | UAAACUACUAGC | 72 | 96.3 | 84.3 |
| | | UAGUUUACA | | AUUUACAAA | | | |
| 11 | 503 | GGAGAGAGAUGU | 23 | UGUAUCCACAUC | 73 | 96.2 | 91.2 |
| | | GGAUACACA | | UCUCUCCAA | | | |
| 12 | 407 | GGAACAAGUAGA | 24 | UACCACUUCUAC | 74 | 96.1 | 92.1 |
| | | AGUGGUAGA | | UUGUUCCCU | | | |
| 13 | 976 | CUGUGCGACUCU | 25 | UCUAUCUAGAGU | 75 | 95.7 | 86.7 |
| | | AGAUAGAUC | | CGCACAGGG | | | |
| 14 | 1110 | AGUGCAGCAUGA | 26 | UUCACAUUCAUG | 76 | 95.5 | 84.5 |
| | | AUGUGAAGU | | CUGCACUGG | | | |
| 15 | 162 | GGUGAAACAGAA | 27 | UCUUCUGUUCUG | 77 | 95.5 | 94.5 |
| | | CAGAAGAAG | | UUUCACCUU | | | |
| 16 | 385 | CCUCUUCCCUAA | 28 | UUGUUCUUUAGG | 78 | 94.3 | 91.3 |
| | | AGAACAAUA | | GAAGAGGGA | | | |
| 17 | 150 | GGAGGUACAGAA | 29 | UUUCACCUUCUG | 79 | 93.7 | 92.7 |
| | | GGUGAAACA | | UACCUCCAA | | | |
| 18 | 1390 | CUUCAUAAGAAG | 30 | UAUAUAACUUCU | 80 | 93.6 | 80.6 |
| | | UUAUAUAAA | | UAUGAAGUA | | | |
| 19 | 170 | AGAACAGAAGAA | 31 | UAUGAUCUUCUU | 81 | 93.6 | 92.6 |
| | | GAUCAUAAA | | CUGUUCUGU | | | |
| 20 | 255 | CUACCUGGUUCU | 32 | UUAUGAGAGAAC | 82 | 93.5 | 91.5 |
| | | CUCAUAAGA | | CAGGUAGGG | | | |
| 21 | 254 | CCUACCUGGUUC | 33 | UAUGAGAGAACC | 83 | 93.1 | 91.1 |
| | | UCUCAUAAG | | AGGUAGGGG | | | |
| 22 | 398 | GAACAAUAGGGA | 34 | UACUUGUUCCCU | 84 | 93 | 90 |
| | | ACAAGUAGA | | AUUGUUCUU | | | |
| 23 | 1317 | CAAGAAUCUAGU | 35 | AUAAAUUACUAG | 85 | 92.8 | 79.8 |
| | | AAUUUAUCA | | AUUCUUGGU | | | |
| 24 | 423 | GUAGAAUUUCAC | 36 | UAUUGCUGUGAA | 86 | 92.5 | 88.5 |
| | | AGCAAUAAA | | AUUCUACCA | | | |
| 25 | 910 | AAUGAAGUACUU | 37 | UUUGACAAAGUA | 87 | 92.1 | 83.1 |
| | | UGUCAAAUA | | CUUCAUUUG | | | |
| 26 | 401 | CAAUAGGGAACA | 38 | UUCUACUUGUUC | 88 | 92.1 | 88.1 |
| | | AGUAGAAGU | | CCUAUUGUU | | | |
| 27 | 912 | UGAAGUACUUUG | 39 | UAUUUGACAAAG | 89 | 92 | 83 |
| | | UCAAAUAAA | | UACUUCAUU | | | |
| 28 | 909 | AAAUGAAGUACU | 40 | UUGACAAAGUAC | 90 | 91.8 | 82.8 |
| | | UUGUCAAAU | | UUCAUUUGG | | | |
| 29 | 1294 | AUGUUAUGAUAU | 41 | UUUCAUGAUAUC | 91 | 91.8 | 79.8 |
| | | CAUGAAAGA | | AUAACAUUA | | | |
| 30 | 1313 | GAACCAAGAAUC | 42 | AUUACUAGAUUC | 92 | 91.6 | 78.6 |
| | | UAGUAAUUU | | UUGGUUCUU | | | |
| 31 | 392 | CCUAAAGAACAA | 43 | UUCCCUAUUGUU | 93 | 91.3 | 88.3 |
| | | UAGGGAACA | | CUUUAGGGA | | | |
| 32 | 1180 | GUUUCAUGUUCA | 44 | AUACCAGUGAAC | 94 | 91.3 | 80.3 |
| | | CUGGUAUUU | | AUGAAACAG | | | |
| 33 | 289 | GCGCUUCGAGCU | 45 | UUGAAGAAGCUC | 95 | 91.3 | 89.3 |
| | | UCUUCAAGG | | GAAGCGCUC | | | |
| 34 | 934 | CGAGGAUGGGAA | 46 | UCAGUUGUUCCC | 96 | 91.2 | 82.2 |
| | | CAACUGAGU | | AUCCUCGUU | | | |
| 35 | 1286 | AUGAUUUAAUGU | 47 | UAUCAUAACAUU | 97 | 91 | 79 |
| | | UAUGAUAUC | | AAAUCAUUA | | | |
| 36 | 1393 | CAUAAGAAGUUA | 48 | AUUUAUAUAACU | 98 | 90.9 | 77.9 |
| | | UAUAAAUAA | | UCUUAUGAA | | | |
| 37 | 413 | AGUAGAAGUGGU | 49 | AAAUUCUACCAC | 99 | 90.9 | 86.9 |
| | | AGAAUUUCA | | UUCUACUUG | | | |
| 38 | 378 | GUUCCUCCCUCU | 50 | UUAGGGAAGAGG | 100 | 90.9 | 87.9 |
| | | UCCCUAAAG | | GAGGAACUG | | | |
| 39 | 764 | AGGACAGGAGGA | 51 | UUUCCUGUCCUC | 101 | 90.6 | 83.6 |
| | | CAGGAAAUC | | CUGUCCUUU | | | |
| 40 | 972 | GACCCUGUGCGA | 52 | UCUAGAGUCGCA | 102 | 90.5 | 81.5 |
| | | CUCUAGAUA | | CAGGGUCCA | | | |
| 41 | 422 | GGUAGAAUUUCA | 53 | AUUGCUGUGAAA | 103 | 90.4 | 86.4 |
| | | CAGCAAUAA | | UUCUACCAC | | | |
| 42 | 172 | AACAGAAGAAGA | 54 | UUUAUGAUCUUC | 104 | 90.2 | 89.2 |
| | | UCAUAAAGA | | UUCUGUUCU | | | |
| 43 | 1386 | UCUACUUCAUAA | 55 | UAACUUCUUAUG | 105 | 90.1 | 77.1 |
| | | GAAGUUAUA | | AAGUAGAUU | | | |
| 44 | 974 | CCCUGUGCGACU | 56 | UAUCUAGAGUCG | 106 | 89.9 | 80.9 |
| | | CUAGAUAGA | | CACAGGGUC | | | |
| 45 | 552 | GAAGUGCACCGG | 57 | UACCAAACCGGU | 107 | 89.7 | 84.7 |
| | | UUUGGUAUC | | GCACUUCUA | | | |
| 46 | 1394 | AUAAGAAGUUAU | 58 | UAUUUAUAUAAC | 108 | 89.7 | 76.7 |
| | | AUAAAUAAA | | UUCUUAUGA | | | |
| 47 | 648 | AAGAGUUAUGUG | 59 | UGUAAUUCACAU | 109 | 89.6 | 83.6 |
| | | AAUUACAAG | | AACUCUUUU | | | |
| 48 | 765 | GGACAGGAGGAC | 60 | AUUUCCUGUCCU | 110 | 89.5 | 82.5 |
| | | AGGAAAUCC | | CCUGUCCUU | | | |
| 49 | 359 | AGAGAUCCUUGC | 61 | UGCAGCAGCAAG | 111 | 89.4 | 86.4 |
| | | UGCUGCAGU | | GAUCUCUGG | | | |
| 50 | 1249 | AUGCUAGUAGUU | 62 | UAAUGUAAACUA | 112 | 89.4 | 77.4 |
| | | UACAUUAAA | | CUAGCAUUU | | | |

C1ORF131 (Variant 2) Isoform b

| | | | SEQ | | SEQ | | |
|---|---|---|---|---|---|---|---|
| | | | ID | | ID | | |
| SIRNA_id | Position | SS Sequence | NO: | AS Sequence | NO: | Score | Corrected_Score |
| 1 | 1248 | GCUAGUAGUUUA | 113 | UUUAAUGUAAAC | 163 | 104.1 | 92.1 |
| | | CAUUAAAUU | | UACUAGCAU | | | |
| 2 | 1313 | CCAAGAAUCUAG | 114 | UAAAUUACUAGA | 164 | 102.8 | 89.8 |
| | | UAAUUUAUC | | UUCUUGGUU | | | |
| 3 | 646 | AGAGUUAUGUGA | 115 | UUGUAAUUCACA | 165 | 99.8 | 93.8 |
| | | AUUACAAGG | | UAACUCUUU | | | |
| 4 | 171 | GAACAGAAGAAG | 116 | UUAUGAUCUUCU | 166 | 99.1 | 98.1 |
| | | AUCAUAAAG | | UCUGUUCUG | | | |
| 5 | 559 | GGUUUGGUAUCA | 117 | UAACCCGUGAUA | 167 | 99 | 94 |
| | | CGGGUUAUG | | CCAAACCGG | | | |
| 6 | 983 | CUAGAUAGAUCG | 118 | UUAAGAACGAUC | 168 | 98.4 | 89.4 |
| | | UUCUUAACA | | UAUCUAGAG | | | |
| 7 | 143 | CGACUUUGGAGG | 119 | UUCUGUACCUCC | 169 | 98.2 | 97.2 |
| | | UACAGAAGG | | AAAGUCGUA | | | |
| 8 | 1198 | GGUGCAUUUGCU | 120 | UAUUACAAGCAA | 170 | 98.2 | 87.2 |
| | | UGUAAUACA | | AUGCACCAA | | | |
| 9 | 1247 | UGCUAGUAGUUU | 121 | UUAAUGUAAACU | 171 | 96.4 | 84.4 |
| | | ACAUUAAAU | | ACUAGCAUU | | | |
| 10 | 1241 | UGUAAAUGCUAG | 122 | UAAACUACUAGC | 172 | 96.3 | 84.3 |
| | | UAGUUUACA | | AUUUACAAA | | | |
| 11 | 500 | GGAGAGAGAUGU | 123 | UGUAUCCACAUC | 173 | 96.2 | 91.2 |
| | | GGAUACACA | | UCUCUCCAA | | | |
| 12 | 407 | GGAACAAGUAGA | 124 | UACCACUUCUAC | 174 | 96.1 | 92.1 |
| | | AGUGGUAGA | | UUGUUCCCU | | | |
| 13 | 973 | CUGUGCGACUCU | 125 | UCUAUCUAGAGU | 175 | 95.7 | 86.7 |
| | | AGAUAGAUC | | CGCACAGGG | | | |
| 14 | 1107 | AGUGCAGCAUGA | 126 | UUCACAUUCAUG | 176 | 95.5 | 84.5 |
| | | AUGUGAAGU | | CUGCACUGG | | | |
| 15 | 162 | GGUGAAACAGAA | 127 | UCUUCUGUUCUG | 177 | 95.5 | 94.5 |
| | | CAGAAGAAG | | UUUCACCUU | | | |
| 16 | 385 | CCUCUUCCCUAA | 128 | UUGUUCUUUAGG | 178 | 94.3 | 91.3 |
| | | AGAACAAUA | | GAAGAGGGA | | | |
| 17 | 150 | GGAGGUACAGAA | 129 | UUUCACCUUCUG | 179 | 93.7 | 92.7 |
| | | GGUGAAACA | | UACCUCCAA | | | |
| 18 | 1387 | CUUCAUAAGAAG | 130 | UAUAUAACUUCU | 180 | 93.6 | 80.6 |
| | | UUAUAUAAA | | UAUGAAGUA | | | |
| 19 | 170 | AGAACAGAAGAA | 131 | UAUGAUCUUCUU | 181 | 93.6 | 92.6 |
| | | GAUCAUAAA | | CUGUUCUGU | | | |
| 20 | 255 | CUACCUGGUUCU | 132 | UUAUGAGAGAAC | 182 | 93.5 | 91.5 |
| | | CUCAUAAGA | | CAGGUAGGG | | | |
| 21 | 254 | CCUACCUGGUUC | 133 | UAUGAGAGAACC | 183 | 93.1 | 91.1 |
| | | UCUCAUAAG | | AGGUAGGGG | | | |
| 22 | 398 | GAACAAUAGGGA | 134 | UACUUGUUCCCU | 184 | 93 | 90 |
| | | ACAAGUAGA | | AUUGUUCUU | | | |
| 23 | 1314 | CAAGAAUCUAGU | 135 | AUAAAUUACUAG | 185 | 92.8 | 79.8 |
| | | AAUUUAUCA | | AUUCUUGGU | | | |
| 24 | 423 | GUAGAAUUUCAC | 136 | UAUUGCUGUGAA | 186 | 92.5 | 88.5 |
| | | AGCAAUAAA | | AUUCUACCA | | | |
| 25 | 907 | AAUGAAGUACUU | 137 | UUUGACAAAGUA | 187 | 92.1 | 83.1 |
| | | UGUCAAAUA | | CUUCAUUUG | | | |
| 26 | 40 | CAAUAGGGAACA | 138 | UUCUACUUGUUC | 188 | 92.1 | 88.1 |
| | | AGUAGAAGU | | CCUAUUGUU | | | |
| 27 | 909 | UGAAGUACUUUG | 139 | UAUUUGACAAAG | 189 | 92 | 83 |
| | | UCAAAUAAA | | UACUUCAUU | | | |
| 28 | 906 | AAAUGAAGUACU | 140 | UUGACAAAGUAC | 190 | 91.8 | 82.8 |
| | | UUGUCAAAU | | UUCAUUUGG | | | |
| 29 | 1291 | AUGUUAUGAUAU | 141 | UUUCAUGAUAUC | 191 | 91.8 | 79.8 |
| | | CAUGAAAGA | | AUAACAUUA | | | |
| 30 | 1310 | GAACCAAGAAUC | 142 | AUUACUAGAUUC | 192 | 91.6 | 78.6 |
| | | UAGUAAUUU | | UUGGUUCUU | | | |
| 31 | 392 | CCUAAAGAACAA | 143 | UUCCCUAUUGUU | 193 | 91.3 | 88.3 |
| | | UAGGGAACA | | CUUUAGGGA | | | |
| 32 | 1177 | GUUUCAUGUUCA | 144 | AUACCAGUGAAC | 194 | 91.3 | 80.3 |
| | | CUGGUAUUU | | AUGAAACAG | | | |
| 33 | 289 | GCGCUUCGAGCU | 145 | UUGAAGAAGCUC | 195 | 91.3 | 89.3 |
| | | UCUUCAAGG | | GAAGCGCUC | | | |
| 34 | 931 | CGAGGAUGGGAA | 146 | UCAGUUGUUCCC | 196 | 91.2 | 82.2 |
| | | CAACUGAGU | | AUCCUCGUU | | | |
| 35 | 1283 | AUGAUUUAAUGU | 147 | UAUCAUAACAUU | 197 | 91 | 79 |
| | | UAUGAUAUC | | AAAUCAUUA | | | |
| 36 | 1390 | CAUAAGAAGUUA | 148 | AUUUAUAUAACU | 198 | 90.9 | 77.9 |
| | | UAUAAAUAA | | UCUUAUGAA | | | |
| 37 | 413 | AGUAGAAGUGGU | 149 | AAAUUCUACCAC | 199 | 90.9 | 86.9 |
| | | AGAAUUUCA | | UUCUACUUG | | | |
| 38 | 378 | GUUCCUCCCUCU | 150 | UUAGGGAAGAGG | 200 | 90.9 | 87.9 |
| | | UCCCUAAAG | | GAGGAACUG | | | |
| 39 | 761 | AGGACAGGAGGA | 151 | UUUCCUGUCCUC | 201 | 90.6 | 83.6 |
| | | CAGGAAAUC | | CUGUCCUUU | | | |
| 40 | 969 | GACCCUGUGCGA | 152 | UCUAGAGUCGCA | 202 | 90.5 | 81.5 |
| | | CUCUAGAUA | | CAGGGUCCA | | | |
| 41 | 422 | GGUAGAAUUUCA | 153 | AUUGCUGUGAAA | 203 | 90.4 | 86.4 |
| | | CAGCAAUAA | | UUCUACCAC | | | |
| 42 | 172 | AACAGAAGAAGA | 154 | UUUAUGAUCUUC | 204 | 90.2 | 89.2 |
| | | UCAUAAAGA | | UUCUGUUCU | | | |
| 43 | 1383 | UCUACUUCAUAA | 155 | UAACUUCUUAUG | 205 | 90.1 | 77.1 |
| | | GAAGUUAUA | | AAGUAGAUU | | | |
| 44 | 971 | CCCUGUGCGACU | 156 | UAUCUAGAGUCG | 206 | 89.9 | 80.9 |
| | | CUAGAUAGA | | CACAGGGUC | | | |
| 45 | 549 | GAAGUGCACCGG | 157 | UACCAAACCGGU | 207 | 89.7 | 84.7 |
| | | UUUGGUAUC | | GCACUUCUA | | | |
| 46 | 1391 | AUAAGAAGUUAU | 158 | UAUUUAUAUAAC | 208 | 89.7 | 76.7 |
| | | AUAAAUAAA | | UUCUUAUGA | | | |
| 47 | 645 | AAGAGUUAUGUG | 159 | UGUAAUUCACAU | 209 | 89.6 | 83.6 |
| | | AAUUACAAG | | AACUCUUUU | | | |
| 48 | 762 | GGACAGGAGGAC | 160 | AUUUCCUGUCCU | 210 | 89.5 | 82.5 |
| | | AGGAAAUCC | | CCUGUCCUU | | | |
| 49 | 359 | AGAGAUCCUUGC | 161 | UGCAGCAGCAAG | 211 | 89.4 | 86.4 |
| | | UGCUGCAGU | | GAUCUCUGG | | | |
| 50 | 1246 | AUGCUAGUAGUU | 162 | UAAUGUAAACUA | 212 | 89.4 | 77.4 |
| | | UACAUUAAA | | CUAGCAUUU | | | |

SLC45A4 (Variant 1) Isoform 1

| | | | SEQ | | SEQ | | |
|---|---|---|---|---|---|---|---|
| | | | ID | | ID | | |
| SIRNA_id | Position | SS Sequence | NO: | AS Sequence | NO: | Score | Corrected_Score |
| 1 | 553 | GAUAGAAUAUAU | 213 | UUUAUCUAUAUA | 263 | 105.5 | 100.5 |
| | | AGAUAAAUA | | UUCUAUCUA | | | |
| 2 | 621 | GGAAUCUAUGCA | 214 | UUGAACUUGCAU | 264 | 102.8 | 96.8 |
| | | AGUUCAAGA | | AGAUUCCGG | | | |
| 3 | 3990 | GGAACGACUGUU | 215 | UACAUUAAACAG | 265 | 102.2 | 63.2 |
| | | UAAUGUACU | | UCGUUCCUU | | | |
| 4 | 3803 | GUCAGACAAUUG | 216 | UUAAUUACAAUU | 266 | 102 | 64 |
| | | UAAUUAAUA | | GUCUGACUU | | | |
| 5 | 4761 | CCCUCUGAAUAG | 217 | UAACACACUAUU | 267 | 100.9 | 53.9 |
| | | UGUGUUAAC | | CAGAGGGGG | | | |
| 6 | 6695 | AGUUCAGUUCCG | 218 | UUCAACUCGGAA | 268 | 100.7 | 34.7 |
| | | AGUUGAACA | | CUGAACUGA | | | |
| 7 | 620 | CGGAAUCUAUGC | 219 | UGAACUUGCAUA | 269 | 100.7 | 94.7 |
| | | AAGUUCAAG | | GAUUCCGGG | | | |
| 8 | 501 | GAGAAGACGUCU | 220 | UAAAUUAAGACG | 270 | 99.5 | 94.5 |
| | | UAAUUUAUC | | UCUUCUCUU | | | |
| 9 | 3002 | CAAUGUGGUUCU | 221 | UUCUAAGAGAAC | 271 | 99.4 | 69.4 |
| | | CUUAGAAGA | | CACAUUGUG | | | |
| 10 | 2330 | AGAAGUACUUGG | 222 | UAGUUGUCCAAG | 272 | 99.2 | 76.2 |
| | | ACAACUACG | | UACUUCUGU | | | |
| 11 | 552 | AGAUAGAAUAUA | 223 | UUAUCUAUAUAU | 273 | 98.7 | 93.7 |
| | | UAGAUAAAU | | UCUAUCUAU | | | |
| 12 | 5682 | GGAUAAUGUUGG | 224 | AAUGAGACCAAC | 274 | 98.7 | 42.7 |
| | | UCUCAUUUG | | AUUAUCCUA | | | |
| 13 | 6168 | GGUCUGUGACCA | 225 | UUCUAGGUGGUC | 275 | 98.4 | 37.4 |
| | | CCUAGAACC | | ACAGACCAA | | | |
| 14 | 4561 | GAAUCAAGAUCC | 226 | UUGGGUCGGAUC | 276 | 98.3 | 53.3 |
| | | GACCCAAAC | | UUGAUUCGG | | | |
| 15 | 4228 | GGCAAGUCCGAC | 227 | UUCAGUAGUCGG | 277 | 98.1 | 56.1 |
| | | UACUGAAAG | | ACUUGCCCU | | | |
| 16 | 4955 | CACUUUGUAGAC | 228 | UUGACGAGUCUA | 278 | 97.5 | 48.5 |
| | | UCGUCAAGC | | CAAAGUGUG | | | |
| 17 | 5627 | GGGUGUGUCGGU | 229 | UCGACCAACCGA | 279 | 97.4 | 41.4 |
| | | UGGUCGAGU | | CACACCCUG | | | |
| 18 | 1346 | GCAUCGACGAGG | 230 | UACUGCUCCUCG | 280 | 97 | 84 |
| | | AGCAGUACA | | UCGAUGCUG | | | |
| 19 | 5716 | CCCUAUACAAGU | 231 | UUAGCUGACUUG | 281 | 97 | 40 |
| | | CAGCUAAGU | | UAUAGGGAA | | | |
| 20 | 4981 | GCACUUCAGGUG | 232 | UUGCAAACACCU | 282 | 96.9 | 47.9 |
| | | UUUGCAAGC | | GAAGUGCUG | | | |
| 21 | 3884 | GCAAUAUGAUCA | 233 | UCUAGAAUGAUC | 283 | 96.6 | 58.6 |
| | | UUCUAGAGC | | AUAUUGCAG | | | |
| 22 | 3643 | GUUCUGGUUUCA | 234 | UAUUCGUUGAAA | 284 | 96.5 | 60.5 |
| | | ACGAAUAAG | | CCAGAACGA | | | |
| 23 | 1713 | GGAUAAUCACUU | 235 | UUCAUUCAAGUG | 285 | 96.1 | 79.1 |
| | | GAAUGAAGC | | AUUAUCCAG | | | |
| 24 | 3995 | GACUGUUUAAUG | 236 | UCAAGUACAUUA | 286 | 96.1 | 57.1 |
| | | UACUUGAUG | | AACAGUCGU | | | |
| 25 | 2999 | CCACAAUGUGGU | 237 | UAAGAGAACCAC | 287 | 95.9 | 66.9 |
| | | UCUCUUAGA | | AUUGUGGAA | | | |
| 26 | 801 | CGCUCUGGUCAC | 238 | UAUUGGUGUGAC | 288 | 95.7 | 87.7 |
| | | ACCAAUACU | | CAGAGCGGU | | | |
| 27 | 181 | GGAGGAGACAGA | 239 | UGGAUGAUCUGU | 289 | 95.5 | 94.5 |
| | | UCAUCCAGU | | CUCCUCCGG | | | |
| 28 | 4054 | CUUUGUAGAUUG | 240 | AAACACACAAUC | 290 | 95.5 | 55.5 |
| | | UGUGUUUGU | | UACAAAGUG | | | |
| 29 | 1265 | GCUGGUUCCGGA | 241 | UUCUGGGUCCG | 291 | 95.4 | 83.4 |
| | | CCCAGAACC | | GAACCAGCUG | | | |
| 30 | 557 | GAAUAUAUAGAU | 242 | UGUAUUUAUCUA | 292 | 95.4 | 90.4 |
| | | AAAUACAUA | | UAUAUUCUA | | | |
| 31 | 1952 | GCAUGAGCGACC | 243 | UCGUACAGGUCG | 293 | 95.1 | 76.1 |
| | | UGUACGACA | | CUCAUGCUG | | | |
| 32 | 284 | CUUCAGUAAUAA | 244 | UUCAUCAUUAUU | 294 | 95.1 | 93.1 |
| | | UGAUGAAAC | | ACUGAAGAG | | | |
| 33 | 526 | CAGAUAUAUGAA | 245 | UAAUGCUUUCAU | 295 | 95 | 90 |
| | | AGCAUUAUU | | AUAUCUGUA | | | |
| 34 | 1862 | CCAGCACCUUCU | 246 | UAGUAGGAGAAG | 296 | 95 | 77 |
| | | CCUACUACG | | GUGCUGGAG | | | |
| 35 | 5668 | CAGUUACCAAAC | 247 | UAUCCUAGUUUG | 297 | 95 | 39 |
| | | UAGGAUAAU | | GUAACUGGU | | | |
| 36 | 497 | GAAAGAGAAGAC | 248 | UUAAGACGUCUU | 298 | 95 | 91 |
| | | GUCUUAAUU | | CUCUUUCUG | | | |
| 37 | 627 | UAUGCAAGUUCA | 249 | UAACUCUUGAAC | 299 | 94.9 | 88.9 |
| | | AGAGUUAUC | | UUGCAUAGA | | | |
| 38 | 486 | CAGCAGAAGCAG | 250 | UCUCUUUCUGCU | 300 | 94.9 | 90.9 |
| | | AAAGAGAAG | | UCUGCUGUG | | | |
| 39 | 487 | AGCAGAAGCAGA | 251 | UUCUCUUUCUGC | 301 | 94.8 | 90.8 |
| | | AAGAGAAGA | | UUCUGCUGU | | | |
| 40 | 3710 | CACAGUCUCUUU | 252 | UAGUUCUAAAGA | 302 | 94.8 | 57.8 |
| | | AGAACUAAG | | GACUGUGGU | | | |
| 41 | 3673 | GGCAAGUACUGC | 253 | AAUAGCUGCAGU | 303 | 94.7 | 58.7 |
| | | AGCUAUUUG | | ACUUGCCCG | | | |
| 42 | 278 | GCACCUCUUCAG | 254 | AUUAUUACUGAA | 304 | 94.7 | 92.7 |
| | | UAAUAAUGA | | GAGGUGCUG | | | |
| 43 | 812 | CACCAAUACUGU | 255 | AUCUGCAACAGU | 305 | 94.6 | 86.6 |
| | | UGCAGAUUG | | AUUGGUGUG | | | |
| 44 | 5224 | AGAUUGCACUCA | 256 | UUUCAAGUGAGU | 306 | 94.5 | 42.5 |
| | | CUUGAAACC | | GCAAUCUGG | | | |
| 45 | 452 | AAGAGGAAUGUA | 257 | UAACACUUACAU | 307 | 94.5 | 90.5 |
| | | AGUGUUACC | | UCCUCUUUG | | | |
| 46 | 3284 | CGUCUGAAGGAC | 258 | UAGGAGAGUCCU | 308 | 94.4 | 62.4 |
| | | UCUCCUAGG | | UCAGACGGG | | | |
| 47 | 5663 | CAGACCAGUUAC | 259 | UAGUUUGGUAAC | 309 | 94.3 | 38.3 |
| | | CAAACUAGG | | UGGUCUGCG | | | |
| 48 | 4141 | GCAGAGGGCUGA | 260 | UUGAAAGUCAGC | 310 | 94.2 | 53.2 |
| | | CUUUCAAGA | | CCUCUGCGA | | | |
| 49 | 3461 | CGGGUGUGUGGA | 261 | UCGCACAUCCAC | 311 | 94 | 60 |
| | | UGUGCGAGG | | ACACCCGAG | | | |
| 50 | 4227 | GGGCAAGUCCGA | 262 | UCAGUAGUCGGA | 312 | 93.9 | 51.9 |
| | | CUACUGAAA | | CUUGCCCUG | | | |

| | | | SEQ | | SEQ | | |
|---|---|---|---|---|---|---|---|
| | | | ID | | ID | | |
| siRNA_id | Position | SS Sequence | NO: | AS Sequence | NO: | Score | Corrected_Score |
| 1 | 3545 | GGAACGACUGUU | 313 | UACAUUAAACAG | 363 | 102.2 | 67.2 |
| | | UAAUGUACU | | UCGUUCCUU | | | |
| 2 | 3358 | GUCAGACAAUUG | 314 | UUAAUUACAAUU | 364 | 102 | 69 |
| | | UAAUUAAUA | | GUCUGACUU | | | |
| 3 | 4316 | CCCUCUGAAUAG | 315 | UAACACACUAUU | 365 | 100.9 | 57.9 |
| | | UGUGUUAAC | | CAGAGGGGG | | | |
| 4 | 6250 | AGUUCAGUUCCG | 316 | UUCAACUCGGAA | 366 | 100.7 | 38.7 |
| | | AGUUGAACA | | CUGAACUGA | | | |
| 5 | 2557 | CAAUGUGGUUCU | 317 | UUCUAAGAGAAC | 367 | 994 | 74.4 |
| | | CUUAGAAGA | | CACAUUGUG | | | |
| 6 | 1892 | AGAAGUACUUGG | 318 | UAGUUGUCCAAG | 368 | 99.2 | 81.2 |
| | | ACAACUACG | | UACUUCUGU | | | |
| 7 | 5237 | GGAUAAUGUUGG | 319 | AAUGAGACCAAC | 339 | 98.7 | 46.7 |
| | | UCUCAUUUG | | AUUAUCCUA | | | |
| 8 | 5723 | GGUCUGUGACCA | 320 | UUCUAGGUGGUC | 340 | 98.4 | 41.4 |
| | | CCUAGAACC | | ACAGACCAA | | | |
| 9 | 4116 | GAAUCAAGAUCC | 321 | UUGGGUCGGAUC | 341 | 98.3 | 57.3 |
| | | GACCCAAAC | | UUGAUUCGG | | | |
| 10 | 3783 | GGCAAGUCCGAC | 322 | UUCAGUAGUCGG | 342 | 98.1 | 61.1 |
| | | UACUGAAAG | | ACUUGCCCU | | | |
| 11 | 4510 | CACUUUGUAGAC | 323 | UUGACGAGUCUA | 343 | 97.5 | 52.5 |
| | | UCGUCAAGC | | CAAAGUGUG | | | |
| 12 | 5182 | GGGUGUGUCGG | 324 | UCGACCAACCGA | 344 | 97.4 | 46.4 |
| | | UUGGUCGAGU | | CACACCCUG | | | |
| 13 | 908 | GCAUCGACGAGG | 325 | UACUGCUCCUCG | 345 | 97 | 88 |
| | | AGCAGUACA | | UCGAUGCUG | | | |
| 14 | 5271 | CCCUAUACAAGU | 326 | UUAGCUGACUUG | 346 | 97 | 45 |
| | | CAGCUAAGU | | UAUAGGGAA | | | |
| 15 | 4536 | GCACUUCAGGUG | 327 | UUGCAAACACCU | 347 | 96.9 | 51.9 |
| | | UUUGCAAGC | | GAAGUGCUG | | | |
| 16 | 3439 | GCAAUAUGAUCA | 328 | UCUAGAAUGAUC | 348 | 96.6 | 62.6 |
| | | UUCUAGAGC | | AUAUUGCAG | | | |
| 17 | 3198 | GUUCUGGUUUCA | 329 | UAUUCGUUGAAA | 349 | 96.5 | 65.5 |
| | | ACGAAUAAG | | CCAGAACGA | | | |
| 18 | 1275 | GGAUAAUCACUU | 330 | UUCAUUCAAGUG | 350 | 96.1 | 84.1 |
| | | GAAUGAAGC | | AUUAUCCAG | | | |
| 19 | 3550 | GACUGUUUAAUG | 331 | UCAAGUACAUUA | 351 | 96.1 | 61.1 |
| | | UACUUGAUG | | AACAGUCGU | | | |
| 20 | 2554 | CCACAAUGUGGU | 332 | UAAGAGAACCAC | 352 | 95.9 | 70.9 |
| | | UCUCUUAGA | | AUUGUGGAA | | | |
| 21 | 3609 | CUUUGUAGAUUG | 333 | AAACACACAAUCU | 353 | 95.5 | 59.5 |
| | | UGUGUUUGU | | ACAAAGUG | | | |
| 22 | 827 | GCUGGUUCCGGA | 334 | UUCUGGGUCCGG | 354 | 95.4 | 87.4 |
| | | CCCAGAACC | | AACCAGCUG | | | |
| 23 | 1514 | GCAUGAGCGACC | 335 | UCGUACAGGUCG | 355 | 95.1 | 80.1 |
| | | UGUACGACA | | CUCAUGCUG | | | |
| 24 | 1424 | CCAGCACCUUCU | 336 | UAGUAGGAGAAG | 356 | 95 | 81 |
| | | CCUACUACG | | GUGCUGGAG | | | |
| 25 | 5223 | CAGUUACCAAAC | 337 | UAUCCUAGUUUG | 357 | 95 | 43 |
| | | UAGGAUAAU | | GUAACUGGU | | | |
| 26 | 3265 | CACAGUCUCUUU | 338 | UAGUUCUAAAGA | 358 | 94.8 | 62.8 |
| | | AGAACUAAG | | GACUGUGGU | | | |
| 27 | 3228 | GGCAAGUACUGC | 339 | AAUAGCUGCAGU | 359 | 94.7 | 62.7 |
| | | AGCUAUUUG | | ACUUGCCCG | | | |
| 28 | 4779 | AGAUUGCACUCA | 340 | UUUCAAGUGAGU | 360 | 94.5 | 47.5 |
| | | CUUGAAACC | | GCAAUCUGG | | | |
| 29 | 2839 | CGUCUGAAGGAC | 341 | UAGGAGAGUCCU | 361 | 94.4 | 66.4 |
| | | UCUCCUAGG | | UCAGACGGG | | | |
| 30 | 5218 | CAGACCAGUUAC | 342 | UAGUUUGGUAAC | 362 | 94.3 | 42.3 |
| | | CAAACUAGG | | UGGUCUGCG | | | |
| 31 | 3696 | GCAGAGGGCUGA | 343 | UUGAAAGUCAGC | 363 | 94.2 | 58.2 |
| | | CUUUCAAGA | | CCUCUGCGA | | | |
| 32 | 226 | CCUUGUUGAAGA | 344 | UUUCUCCUCUUC | 364 | 94.2 | 92.2 |
| | | GGAGAAACA | | AACAAGGAG | | | |
| 33 | 3016 | CGGGUGUGUGGA | 345 | UCGCACAUCCAC | 365 | 94 | 64 |
| | | UGUGCGAGG | | ACACCCGAG | | | |
| 34 | 3782 | GGGCAAGUCCGA | 346 | UCAGUAGUCGGA | 366 | 93.9 | 56.9 |
| | | CUACUGAAA | | CUUGCCCUG | | | |
| 35 | 3456 | GAGCAAAUAUAU | 347 | UGUAUAUAUAUA | 367 | 93.9 | 59.9 |
| | | AUAUACACG | | UUUGCUCUA | | | |
| 36 | 4625 | GGGUCAGUAGAG | 348 | AAACCGACUCUA | 368 | 93.7 | 47.7 |
| | | UCGGUUUCU | | CUGACCCUG | | | |
| 37 | 4317 | CCUCUGAAUAGU | 349 | UUAACACACUAU | 369 | 93.7 | 50.7 |
| | | GUGUUAACC | | UCAGAGGGG | | | |
| 38 | 5872 | GAUACCAACUGU | 350 | UAAGGGAACAGU | 370 | 93.5 | 35.5 |
| | | UCCCUUAUA | | UGGUAUCUU | | | |
| 39 | 1430 | CCUUCUCCUACU | 351 | UUGCCGUAGUAG | 371 | 93.3 | 79.3 |
| | | ACGGCAAGC | | GAGAAGGUG | | | |
| 40 | 3706 | GACUUUCAAGAC | 352 | UCUUGAAGUCUU | 372 | 93.3 | 56.3 |
| | | UUCAAGAAC | | GAAAGUCAG | | | |
| 41 | 1877 | GUUCAGCCCUGU | 353 | UUCUGUAACAGG | 373 | 93.3 | 75.3 |
| | | UACAGAAGU | | GCUGAACAA | | | |
| 42 | 289 | GCAGGGACAGCA | 354 | UAGAUGAUGCUG | 374 | 93.1 | 91.1 |
| | | UCAUCUAUG | | UCCCUGCCG | | | |
| 43 | 3426 | UGAGAAUCAUGC | 355 | UAUUGCAGCAUG | 375 | 93 | 59 |
| | | UGCAAUAUG | | AUUCUCAUG | | | |
| 44 | 1709 | GGUUCUCUGUCA | 356 | UCGGCGAUGACA | 376 | 93 | 76 |
| | | UCGCCGAGG | | GAGAACCAG | | | |
| 45 | 3484 | AUUUCAAGAUGA | 357 | UUUAGUUUCAUC | 378 | 93 | 59 |
| | | AACUAAAGC | | UUGAAAUAU | | | |
| 46 | 2457 | GGUAUUGACGUC | 358 | UCUGACAGACGU | 379 | 93 | 69 |
| | | UGUCAGAUC | | CAAUACCUG | | | |
| 47 | 4127 | CGACCCAAACCA | 359 | UUUGGUGUGGUU | 380 | 92.9 | 51.9 |
| | | CACCAAAUG | | UGGGUCGGA | | | |
| 48 | 3446 | GAUCAUUCUAGA | 360 | UAUUUGCUCUAG | 381 | 92.8 | 58.8 |
| | | GCAAAUAUA | | AAUGAUCAU | | | |
| 49 | 1294 | GCUAAAGUCCCA | 361 | UUCCGUUUGGGA | 382 | 92.8 | 80.8 |
| | | AACGGAAGU | | CUUUAGCUU | | | |
| 50 | 4541 | UCAGGUGUUUGC | 362 | UUUGCUUGCAAA | 383 | 92.7 | 47.7 |
| | | AAGCAAAGC | | CACCUGAAG | | | |

and SLC45A4 (Variant 3) Isoform 3

| | | | SEQ | | SEQ | | |
|---|---|---|---|---|---|---|---|
| | | | ID | | ID | | |
| SIRNA_id | Position | SS Sequence | NO: | AS Sequence | NO: | Score | Corrected_Score |
| 1 | 2637 | GGUUUGAGCUAC | 384 | UUGCUUAGUAGC | 434 | 106.5 | 80.5 |
| | | UAAGCAAAU | | UCAAACCUA | | | |
| 2 | 1892 | AGAAGUACUUGG | 385 | UAGUUGUCCAAG | 435 | 99.2 | 81.2 |
| | | ACAACUACG | | UACUUCUGU | | | |
| 3 | 2633 | GGUAGGUUUGAG | 386 | UUAGUAGCUCAA | 436 | 98.3 | 72.3 |
| | | CUACUAAGC | | ACCUACCCU | | | |
| 4 | 2632 | GGGUAGGUUUGA | 387 | UAGUAGCUCAAA | 437 | 97.9 | 71.9 |
| | | GCUACUAAG | | CCUACCCUA | | | |
| 5 | 908 | GCAUCGACGAGG | 388 | UACUGCUCCUCG | 438 | 97 | 88 |
| | | AGCAGUACA | | UCGAUGCUG | | | |
| 6 | 1275 | GGAUAAUCACUU | 389 | UUCAUUCAAGUG | 439 | 96.1 | 84.1 |
| | | GAAUGAAGC | | AUUAUCCAG | | | |
| 7 | 827 | GCUGGUUCCGGA | 390 | UUCUGGGUCCGG | 440 | 95.4 | 87.4 |
| | | CCCAGAACC | | AACCAGCUG | | | |
| 8 | 1514 | GCAUGAGCGACC | 391 | UCGUACAGGUCG | 441 | 95.1 | 80.1 |
| | | UGUACGACA | | CUCAUGCUG | | | |
| 9 | 1424 | CCAGCACCUUCU | 392 | UAGUAGGAGAAG | 442 | 95 | 81 |
| | | CCUACUACG | | GUGCUGGAG | | | |
| 10 | 226 | CCUUGUUGAAGA | 393 | UUUCUCCUCUUC | 443 | 94.2 | 92.2 |
| | | GGAGAAACA | | AACAAGGAG | | | |
| 11 | 2638 | GUUUGAGCUACU | 394 | UUUGCUUAGUAG | 444 | 93.5 | 67.5 |
| | | AAGCAAAUA | | CUCAAACCU | | | |
| 12 | 1430 | CCUUCUCCUACU | 395 | UUGCCGUAGUAG | 445 | 93.3 | 79.3 |
| | | ACGGCAAGC | | GAGAAGGUG | | | |
| 13 | 1877 | GUUCAGCCCUGU | 396 | UUCUGUAACAGG | 446 | 93.3 | 75.3 |
| | | UACAGAAGU | | GCUGAACAA | | | |
| 14 | 289 | GCAGGGACAGCA | 397 | UAGAUGAUGCUG | 447 | 93.1 | 91.1 |
| | | UCAUCUAUG | | UCCCUGCCG | | | |
| 15 | 1709 | GGUUCUCUGUCA | 398 | UCGGCGAUGACA | 448 | 93 | 76 |
| | | UCGCCGAGG | | GAGAACCAG | | | |
| 16 | 1294 | GCUAAAGUCCCA | 399 | UUCCGUUUGGGA | 449 | 92.8 | 80.8 |
| | | AACGGAAGU | | CUUUAGCUU | | | |
| 17 | 1261 | GACGAGACCUUG | 400 | UAUCCAGCAAGG | 450 | 92.4 | 80.4 |
| | | CUGGAUAAU | | UCUCGUCCU | | | |
| 18 | 2648 | CUAAGCAAAUAC | 401 | UAGUGUGGUAUU | 451 | 92.3 | 66.3 |
| | | CACACUAAC | | UGCUUAGUA | | | |
| 19 | 1484 | ACGCCGUGGUGC | 402 | UUGAUCAGCACC | 452 | 91.6 | 77.6 |
| | | UGAUCAAGC | | ACGGCGUUG | | | |
| 20 | 1511 | GCAGCAUGAGCG | 403 | UACAGGUCGCUC | 453 | 91.5 | 76.5 |
| | | ACCUGUACG | | AUGCUGCGC | | | |
| 21 | 1757 | AGGUCAUCUUCG | 404 | UCGCCUUCGAAG | 454 | 91.1 | 74.1 |
| | | AAGGCGACC | | AUGACCUGG | | | |
| 22 | 1004 | CAGACGAGGUAC | 405 | UCCGACUGUACC | 455 | 91 | 81 |
| | | AGUCGGAGC | | UCGUCUGGG | | | |
| 23 | 1880 | CAGCCCUGUUAC | 406 | UACUUCUGUAAC | 456 | 90.9 | 72.9 |
| | | AGAAGUACU | | AGGGCUGAA | | | |
| 24 | 403 | GAGCAGUACUAC | 407 | UGAGGCUGUAGU | 457 | 90.9 | 86.9 |
| | | AGCCUCACC | | ACUGCUCCG | | | |
| 25 | 1272 | GCUGGAUAAUCA | 408 | AUUCAAGUGAUU | 458 | 90.4 | 78.4 |
| | | CUUGAAUGA | | AUCCAGCAA | | | |
| 26 | 1523 | ACCUGUACGACA | 409 | UUCUGCAUGUCG | 459 | 90.3 | 75.3 |
| | | UGCAGAAGC | | UACAGGUCG | | | |
| 27 | 1287 | GAAUGAAGCUAA | 410 | UGGGACUUUAGC | 460 | 90.2 | 78.2 |
| | | AGUCCCAAA | | UUCAUUCAA | | | |
| 28 | 355 | GUGAGUCCACUG | 411 | UUGACCUCAGUG | 461 | 89.7 | 86.7 |
| | | AGGUCAAAU | | GACUCACAA | | | |
| 29 | 1281 | UCACUUGAAUGA | 412 | UUUAGCUUCAUU | 462 | 89.6 | 77.6 |
| | | AGCUAAAGU | | CAAGUGAUU | | | |
| 30 | 1265 | AGACCUUGCUGG | 413 | UGAUUAUCCAGC | 463 | 88.8 | 76.8 |
| | | AUAAUCACU | | AAGGUCUCG | | | |
| 31 | 2629 | GUAGGGUAGGUU | 414 | UAGCUCAAACCU | 464 | 88.7 | 62.7 |
| | | UGAGCUACU | | ACCCUACGA | | | |
| 32 | 1882 | GCCCUGUUACAG | 415 | AGUACUUCUGUA | 465 | 88.5 | 70.5 |
| | | AAGUACUUG | | ACAGGGCUG | | | |
| 33 | 1883 | CCCUGUUACAGA | 416 | AAGUACUUCUGU | 466 | 88.4 | 70.4 |
| | | AGUACUUGG | | AACAGGGCU | | | |
| 34 | 1517 | UGAGCGACCUGU | 417 | AUGUCGUACAGG | 467 | 88.4 | 73.4 |
| | | ACGACAUGC | | UCGCUCAUG | | | |
| 35 | 1121 | AGCUGCUGUUCC | 418 | UCGUGCAGGAAC | 468 | 88.2 | 77.2 |
| | | UGCACGACA | | AGCAGCUCG | | | |
| 36 | 1346 | ACACCAGGGUGG | 419 | UUCGUGUCCACC | 469 | 88.2 | 75.2 |
| | | ACACGAAGC | | CUGGUGUAG | | | |
| 37 | 340 | CGUCCAAAGACC | 420 | UCACAAGGGUCU | 470 | 87.9 | 84.9 |
| | | CUUGUGAGU | | UUGGACGGG | | | |
| 38 | 331 | GGGCUGUCCCGU | 421 | UCUUUGGACGGG | 471 | 87.8 | 84.8 |
| | | CCAAAGACC | | ACAGCCCUG | | | |
| 39 | 1454 | GGUCCCACUGCU | 422 | UAGCGGUAGCAG | 472 | 87.7 | 73.7 |
| | | ACCGCUACC | | UGGGACCCA | | | |
| 40 | 1732 | GUGUUCUACACC | 423 | UGAAGUCGGUGU | 473 | 87.6 | 70.6 |
| | | GACUUCAUG | | AGAACACGG | | | |
| 41 | 902 | UGUUCAGCAUCG | 424 | UCCUCGUCGAUG | 474 | 87.6 | 78.6 |
| | | ACGAGGAGC | | CUGAACAGG | | | |
| 42 | 1733 | UGUUCUACACCG | 425 | AUGAAGUCGGUG | 475 | 87.4 | 70.4 |
| | | ACUUCAUGG | | UAGAACACG | | | |
| 43 | 375 | UCAAUGGAGUCU | 426 | UUUCUGAAGACU | 476 | 87 | 84 |
| | | UCAGAAAGG | | CCAUUGAUU | | | |
| 44 | 1448 | AGCUUGGGUCCC | 427 | UAGCAGUGGGAC | 477 | 86.7 | 72.7 |
| | | ACUGCUACC | | CCAAGCUUG | | | |
| 45 | 1727 | AGGCCGUGUUCU | 428 | UCGGUGUAGAAC | 478 | 86.5 | 69.5 |
| | | ACACCGACU | | ACGGCCUCG | | | |
| 46 | 1262 | ACGAGACCUUGC | 429 | UUAUCCAGCAAG | 479 | 86 | 74 |
| | | UGGAUAAUC | | GUCUCGUCC | | | |
| 47 | 1916 | UGAGCGUCAGGG | 430 | UAGAUCACCCUG | 480 | 86 | 67 |
| | | UGAUCUACG | | ACGCUCAGG | | | |
| 48 | 364 | CUGAGGUCAAAU | 431 | UCCAUUGAUUUG | 481 | 85.9 | 82.9 |
| | | CAAUGGAGU | | ACCUCAGUG | | | |
| 49 | 2535 | GGAGCAGAGACA | 432 | UCCGCGGUGUCU | 482 | 85.7 | 60.7 |
| | | CCGCGGAAC | | CUGCUCCCG | | | |
| 50 | 830 | GGUUCCGGACCC | 433 | UGGUUCUGGGUC | 483 | 85.5 | 77.5 |
| | | AGAACCAGG | | CGGAACCAG | | | |

In yet another aspect, there is provided a composition or a vector or a plasmid comprising one or more of the nucleic acids as disclosed herein.

In yet another aspect, there is provided a composition or a vector or a plasmid comprising one or more of the nucleic acids as disclosed herein for use in therapy.

In yet another aspect, there is provided a kit comprising the nucleic acids as disclosed herein.

### DESCRIPTION OF EMBODIMENTS

Exemplary, non-limiting embodiments of methods and compositions for regenerating a liver cell are disclosed hereinafter.

In one aspect, there is provided a method of regenerating a liver cell in a subject having a liver cell degeneration, comprising administering an agent that modulates one or more gene or marker comprising C1ORF131 (2810004N23Rik), SLC45A4, NFKBIB, ARL6IP5, DBNL, GOLGA7B, FAM117B, MED28, TSC22D4, EIF4EBP1, PFN1, PNRC2, ZNF672 (Zfp672), IER5, ADAMTSL5, COL4A5, MYH15, and ILKAP, wherein the agent counteracts the liver cell degeneration and/or enhances a liver cell regeneration.

Also disclosed is a method of regenerating a liver cell in a subject in need thereof, comprising administering an agent that modulates one or more gene or marker including, but is not limited to, C1ORF131 (2810004N23Rik), SLC45A4, NFKBIB, ARL6IP5, DBNL, GOLGA7B, FAM117B, MED28, TSC22D4, EIF4EBP1, PFN1, PNRC2, ZNF672 (Zfp672), IER5, ADAMTSL5, COL4A5, MYH15, ILKAP, and the like.

Also disclosed is a method of regenerating a liver cell, comprising contacting a liver cell to an agent that modulates one or more gene or marker including, but is not limited to, C1ORF131 (2810004N23Rik), SLC45A4, NFKBIB, ARL6IP5, DBNL, GOLGA7B, FAM117B, MED28, TSC22D4, EIF4EBP1, PFN1, PNRC2, ZNF672 (Zfp672), IER5, ADAMTSL5, COL4A5, MYH15, ILKAP, and the like.

Also disclosed is a method of treating or preventing a liver cell degeneration in a subject in need thereof, comprising administering an agent that modulates one or more gene or marker including, but is not limited to, C1ORF131 (2810004N23Rik), SLC45A4, NFKBIB, ARL6IP5, DBNL, GOLGA7B, FAM117B, MED28, TSC22D4, EIF4EBP1, PFN1, PNRC2, ZNF672 (Zfp672), IER5, ADAMTSL5, COL4A5, MYH15, ILKAP, and the like.

In various examples, the term “subject” as used herein includes patients and non-patients. The term “patient” refers to individuals suffering or are likely to suffer from a medical condition such as a liver degenerative disease/disorder, while “non-patients” refer to individuals not suffering and are likely to not suffer from the medical condition. “Non-patients” include healthy individuals, non-diseased individuals and/or an individual free from the medical condition. The term “subject” includes humans and animals. Animals may include, but is not limited to, mammals (for example non-human primates, canine, murine, rabbits, and the like), and the like. “Murine” refers to any mammal from the family Muridae, such as mouse, rat, and the like. “Rabbits” refers to Leporidae, such as rabbit, hare, and the like.

Also disclosed is the use of an agent for modulating one or more gene or marker in the manufacture of a medicament for treating or preventing a liver cell degeneration, wherein the one or more gene or marker includes, but is not limited to, C1ORF131 (2810004N23Rik), SLC45A4, NFKBIB, ARL6IP5, DBNL, GOLGA7B, FAM117B, MED28, TSC22D4, EIF4EBP1, PFN1, PNRC2, ZNF672 (Zfp672), IER5, ADAMTSL5, COL4A5, MYH15, ILKAP, and the like.

Also disclosed is a method of diagnosing or predicting the presence of or the likelihood of developing a liver cell degeneration in a subject in need thereof, comprising determining the level of one or more gene or marker in a sample obtained from the subject, wherein said gene or marker includes, but is not limited to, C1ORF131 (2810004N23Rik), SLC45A4, NFKBIB, ARL6IP5, DBNL, GOLGA7B, FAM117B, MED28, TSC22D4, EIF4EBP1, PFN1, PNRC2, ZNF672 (Zfp672), IER5, ADAMTSL5, COL4A5, MYH15, ILKAP, and the like.

In some examples, the level of one or more gene or marker in the sample is increased as compared to a control. In some examples, the increase is a statistically significant increase in expression level. In some examples, the increase is at least 2, 3, 4, 5, 6, 7, 8, 9, 10, 11, 12, 13, 14, 15, 16, 17, 18, 19, 20, 30, 100, 200, 300, 400, 500, 600, 700, 800, 900, 1000 or more increase in expression level.

In various examples, the term “sample” may include any biological sample, for example blood, plasma, and the like.

In some examples, the methods or uses as described herein may comprise two, three, four, five, six, seven, eight, nine, 10, 11, 12, 13, 14, 15, 16, 17, or 18 of the gene or marker in priority list #1 or as selected from the group consisting of C1ORF131 (2810004N23Rik), SLC45A4, NFKBIB, ARL6IP5, DBNL, GOLGA7B, FAM117B, MED28, TSC22D4, EIF4EBP1, PFN1, PNRC2, ZNF672 (Zfp672), IER5, ADAMTSL5, COL4A5, MYH15, and ILKAP.

In various examples, the term “treating”, “treat” and “therapy”, and synonyms thereof refer to both therapeutic treatment and prophylactic or preventative measures, wherein the object is to prevent or slow down (lessen) a medical condition, which includes but is not limited to, liver degeneration symptoms and disorders. A medical condition also includes a body's response to a disease or disorder, e.g. fibrosis. Those in need of such treatment include those already with a medical condition as well as those prone to getting the medical condition or those in whom a medical condition is to be prevented.

In some examples, the term “preventing” refers to the process of reducing the severity of symptoms or to process(es) of delaying the onset, reducing the severity of symptoms, reducing and/or preventing cell death, preventing death, inhibiting deterioration/degeneration, inhibiting further deterioration/degeneration, and/or ameliorating at least one sign or symptom of liver degeneration.

In some examples, the one or more gene or marker may further include, but is not limited to, ABCB10, LSM14A, ACTG1, YTHDF2, RPS6KA1, SEC13, IPO11, CTCF, UBAP2L, P2RY2, TRAF3IP2, FST, TUBG1, ZNF664, PVR, DYX1C1/DNAAF4, TEX264, NIF3L1, RPUSD1, TRIM6, GLRX3, BRD1, CKS1B, C6ORF120, WASF2, ZNF689, B4GALT7, LCORL, NR2F2, BAG6, RNF10, and the like.

In some examples, the methods or uses as described herein may comprise two, three, four, five, six, seven, eight, nine, 10, 11, 12, 13, 14, 15, 16, 17, 18, 19, 20, 21, 22, 23, 24, 25, 26, 27, 28, 29, 30, or 31 of the gene or marker in priority list #2 or as selected from the group consisting of ABCB10, LSM14A, ACTG1, YTHDF2, RPS6KA1, SEC13, IPO11, CTCF, UBAP2L, P2RY2, TRAF3IP2, FST, TUBG1, ZNF664, PVR, DYX1C1/DNAAF4, TEX264, NIF3L1, RPUSD1, TRIM6, GLRX3, BRD1, CKS1B, C6ORF120, WASF2, ZNF689, B4GALT7, LCORL, NR2F2, BAG6, and RNF10.

In some examples, the one or more gene or marker may further include, but is not limited to, Uba6, Brwd3, Fyco1, ARID5B, C20ORF96 (6820408C15Rik), CD200R1 (Cd200r4), DALRD3, ELF3, ELFN1, FAM160A1, FAM50B, KIAA2026 (9930021J03Rik), KREMEN1, MACC1, NAA25, PROSC/PLPBP, RPS3, SHPK, SLAIN1, TCHP, ZNF503 (Zfp503), BUD13, CD93, PFKFB2, ZDBF2, FOXC2, GBGT1, RGS4, RNASEH1, TSNAXIP1, LYPD2, STIL, TMCO3, TMEM159, RIF1, IFIH1, EPN2, CLP1, RNF220, NOL12, APOL3, GTF3C6, CTPS2, IFITM2, IFITM1, KCNS3, CA2, EIF4H, MRM1, AKAP8, PAPOLA, SECTM1, TYK2, MLLT6, IL1RL2, IPO7, APBB1IP, RABEP2, PLSCR1, PYGO2, COIL, LRRC8E, TNRC6C, CHD3, USP21, SNX27, NOP14, ZC3H15, C15orf39, UTP20, TRAK1, RAB11FIP2, PHPT1, CUL3, GNL3, and the like.

In some examples, the gene or marker may comprise two, three, four, five, six, seven, eight, nine, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, or 74 of the gene or marker in priority list #3 or as selected from the group consisting of Uba6, Brwd3, Fyco1, ARID5B, C20ORF96 (6820408C15Rik), CD200R1 (Cd200r4), DALRD3, ELF3, ELFN1, FAM160A1, FAM50B, KIAA2026 (9930021J03Rik), KREMEN1, MACC1, NAA25, PROSC/PLPBP, RPS3, SHPK, SLAIN1, TCHP, ZNF503 (Zfp503), BUD13, CD93, PFKFB2, ZDBF2, FOXC2, GBGT1, RGS4, RNASEH1, TSNAXIP1, LYPD2, STIL, TMCO3, TMEM159, RIF1, IFIH1, EPN2, CLP1, RNF220, NOL12, APOL3, GTF3C6, CTPS2, IFITM2, IFITM1, KCNS3, CA2, EIF4H, MRM1, AKAP8, PAPOLA, SECTM1, TYK2, MLLT6, IL1RL2, IPO7, APBB1IP, RABEP2, PLSCR1, PYGO2, COIL, LRRC8E, TNRC6C, CHD3, USP21, SNX27, NOP14, ZC3H15, C15orf39, UTP20, TRAK1, RAB11FIP2, PHPT1, CUL3, and GNL3.

In some examples, the gene or marker may comprise one, two, three, four, five, six, seven, eight, nine, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, or all of the gene or marker selected from the group consisting of
C1ORF131 (2810004N23Rik), SLC45A4, NFKBIB, ARL6IP5, DBNL, GOLGA7B, FAM117B, MED28, TSC22D4, EIF4EBP1, PFN1, PNRC2, ZNF672 (Zfp672), IER5, ADAMTSL5, COL4A5, MYH15, ILKAP,
ABCB10, LSM14A, ACTG1, YTHDF2, RPS6KA1, SEC13, IPO11, CTCF, UBAP2L, P2RY2, TRAF3IP2, FST, TUBG1, ZNF664, PVR, DYX1C1/DNAAF4, TEX264, NIF3L1, RPUSD1, TRIM6, GLRX3, BRD1, CKS1B, C6ORF120, WASF2, ZNF689, B4GALT7, LCORL, NR2F2, BAG6, RNF10,
Uba6, Brwd3, Fyco1, ARID5B, C20ORF96 (6820408C15Rik), CD200R1 (Cd200r4), DALRD3, ELF3, ELFN1, FAM160A1, FAM50B, KIAA2026 (9930021J03Rik), KREMEN1, MACC1, NAA25, PROSC/PLPBP, RPS3, SHPK, SLAIN1, TCHP, ZNF503 (Zfp503), BUD13, CD93, PFKFB2, ZDBF2, FOXC2, GBGT1, RGS4, RNASEH1, TSNAXIP1, LYPD2, STIL, TMCO3, TMEM159, RIF1, IFIH1, EPN2, CLP1, RNF220, NOL12, APOL3, GTF3C6, CTPS2, IFITM2, IFITM1, KCNS3, CA2, EIF4H, MRM1, AKAP8, PAPOLA, SECTM1, TYK2, MLLT6, IL1RL2, IPO7, APBB1IP, RABEP2, PLSCR1, PYGO2, COIL, LRRC8E, TNRC6C, CHD3, USP21, SNX27, NOP14, ZC3H15, C15orf39, UTP20, TRAK1, RAB11FIP2, PHPT1, CUL3, and GNL3.

In some examples, the gene or marker may include two or more, three or more, four or more, five or more, 10 or more, 15 or more, 20 or more, 25 or more, 30 or more, 40 or more, 50 or more, 60 or more, 70 or more, 80 or more, 90 or more, 100 or more, 110 or more, 120 or more, or 123 genes or markers as described herein.

In some examples, the gene or marker may include no more than two, no more than three, no more than four, no more than five, no more than 10, no more than 15, no more than 20, no more than 30, no more than 40, no more than 50, no more than 60, no more than 70, no more than 80, no more than 90, no more than 100, no more than 110, no more than 120, no more than 123 genes or markers as described herein.

In some examples, the gene or marker may include two to 20, or 20 to 30 or 30 to 40, or 40 to 50, or 50 to 60, or 60 to 70, or 70 to 80, or 80 to 90, or 90 to 100, or 100 to 110, or 110 to 120, or all genes or markers as described herein.

In some examples, the agent enhances a liver cell regeneration and/or counteracts a liver cell degeneration.

Without wishing to be bound by theory, it is believed (and shown in the experimental data) that targeting the markers/targets as described herein leads to the enhancement of a liver cell regeneration. In some examples, the modulation of the one or more gene or marker as described herein increases the proliferation rate of a liver cell. The enhancement of a liver cell regeneration thereby attenuate fibrosis and progressive disease.

As used herein, the term “a liver cell degeneration” and “liver degeneration” may be used interchangeably to refer to the gradual deterioration and/or damage of a liver cell, tissue, and/or organ with corresponding impairment or loss of function caused by the deterioration and/or damage. The liver degeneration in the present disclosure may or may not have underlying pathogenic mechanisms.

In some examples, the liver cell is a hepatocyte.

In some examples, the liver degeneration may be due to a liver disease or condition such as, but is not limited to, acute liver disease, chronic liver disease, metabolic liver disease, steatosis, liver fibrosis, primary sclerosing cholangitis (PSC), cirrhosis, mild liver fibrosis, advanced liver fibrosis, non-alcoholic fatty liver disease (NAFLD), non-alcoholic steatohepatitis (NASH), alcoholic fatty liver disease (ALFD), alcohol related liver disease (ARLD), hepatic ischemia reperfusion injury, primary biliary cirrhosis (PBC), hepatitis, liver damage, liver injury, liver failure, metabolic syndrome, obesity, diabetes mellitus, end-stage liver disease, inflammation of the liver, lobular inflammation, hepatocellular carcinoma (HCC), and the like.

In some examples, liver degeneration may include, but is not limited to the various stages of simple steatosis (non-alcoholic fatty liver disease (NAFLD)), non-alcoholic steatohepatitis (including steatosis inflammation ballooning fibrosis), cirrhosis, and hepatocellular carcinoma (HCC).

In some examples, the liver degeneration is non-alcoholic fatty liver disease (NAFLD) and/or non-alcoholic steatohepatitis (NASH).

In some examples, the subject is a candidate for liver transplantation. In some examples, the subject is not a candidate for liver transplantation.

In some examples, the agent that modulates the gene or marker may be an activator, an inhibitor, an antagonist, an agonist, and the like.

In some examples, the agent may be natural compounds such as a secondary metabolic product of plant and/or fungi that is known to inhibit the target gene or marker.

In some examples, the agent reduces or inhibits the expression of the gene or marker.

In some examples, the term “expression” refers to the transcription and/or translation of a particular nucleotide sequence driven by its promoter. In some embodiments, “expression” may refer to display of a polypeptide product of the transcription and/or translation of the nucleotide on the surface of a cell.

In some examples, the agent as described herein modulates gene expression to induce therapeutic effects. For example, the agent as described herein may directly inhibit gene expression in a cell by preventing the activation of the gene. In some examples, the agent as described herein may inhibit downstream translation of the gene as described herein. In some examples, the agent may reduce the activity of the gene or marker as described herein. Thus, in some examples, the agent as described herein may target protein and/or gene expression. In some examples, where the agent is a nucleic acid (such as shRNA or siRNA), the nucleic acid reduces the expression level of the gene or marker by interacting with the corresponding mRNA.

In some examples, the agent inhibits or reduces the expression of the one or more gene or marker by about 1 to 100%, or about 5%, about 10%, about 20%, about 30%, about 40%, about 50%, about 60%, about 70%, about 80%, about 90%, about 100%, or at least 5%, at least 10%, at least 20%, at least 30%, at least 40%, at least 50%, at least 60%, at least 70%, at least 80%, at least 90%, at least 100%, or no more than 5%, no more than 10%, no more than 20%, no more than 30%, no more than 40%, no more than 50%, no more than 60%, no more than 70%, no more than 80%, no more than 90%, or no more than 100%.

In some examples, the agent increases or activates the expression of the one or more gene or marker by about 1 to 100%, or about 5%, about 10%, about 20%, about 30%, about 40%, about 50%, about 60%, about 70%, about 80%, about 90%, about 100%, or at least 5%, at least 10%, at least 20%, at least 30%, at least 40%, at least 50%, at least 60%, at least 70%, at least 80%, at least 90%, at least 100%, or no more than 5%, no more than 10%, no more than 20%, no more than 30%, no more than 40%, no more than 50%, no more than 60%, no more than 70%, no more than 80%, no more than 90%, or no more than 100%.

In some examples, the agent that modulates the gene or marker is selected from a nucleic acid, a peptide, or a small molecule.

In some examples, wherein the agent is a peptide, the peptide may inhibit a marker comprising a sequence selected from the group consisting of:

>NP_689592.2 C1orf131 isoform a
(SEQ ID NO: 484)
MRVDSSADPTMSQEQGPGSSTPPSSPTLLDALLQNLYDFGGTEGETEQKKI
IKKRENKKRDVMASAALAAEPSPLPGSLIRGQRKSASSFFKELREERHCAP
SGTPTGPEILAAAVPPSSLKNNREQVEVVEFHSNKKRKLTPDHNKNTKQAN
PSVLERDVDTQEFNLEKARLEVHRFGITGYGKGKERILEQERAIMLGAKPP
KKSYVNYKVLQEQIKEKKAAKEEEKRLAQETDIFKKKKRKGQEDRKSKKKS
APSILSNGRIGQVGKFKNGTLILSPVDIKKINSSRVAK
>NP_001287759.1 C1orf131 isoform b
(SEQ ID NO: 485)
MRVDSSADPTMSQEQGPGSSTPPSSPTLLDALLQNLYDFGGTEGETEQKKI
IKKRENKKRDVMASAALAAEPSPLPGSLIRGQRKSASSFFKELREERHCAP
SGTPTGPEILAAAVPPSSLKNNREQVEVVEFHSNKKRKLTPDHNKNTKANP
SVLERDVDTQEFNLEKARLEVHRFGITGYGKGKERILEQERAIMLGAKPPK
KSYVNYKVLQEQIKEKKAAKEEEKRLAQETDIFKKKKRKGQEDRKSKKKSA
PSILSNGRIGQVGKFKNGTLILSPVDIKKINSSRVAK
>NP_001273575.1 SLC45a4 isoform 1
(SEQ ID NO: 486)
MKMAPQNADPESMQVQELSVPLPDPQKAGGAEAENCETISEGSIDRIPMRL
WVMHGAVMFGREFCYAMETALVTPILLQIGLPEQYYSLTWFLSPILGLIFT
PLIGSASDRCTLSWGRRRPFILALCVGVLFGVALFLNGSAIGLALGDVPNR
QPIGIVLTVLGVVVLDFSADATEGPIRAYLLDVVDSEEQDMALNIHAFSAG
LGGAIGYVLGGLDWTQTFLGSWFRTQNQVLFFFAAIIFTVSVALHLFSIDE
EQYSPQQERSAEEPGALDGGEPHGVPAFPDEVQSEHELALDYPDVDIMRSK
SDSALHVPDTALDLEPELLFLHDIEPSIFHDASYPATPRSTSQELAKTKLP
RLATFLKEAAKEDETLLDNHLNEAKVPNGSGSPTKDALGGYTRVDTKPSAT
SSSMRRRRHAFRRQASSTFSYYGKLGSHCYRYRRANAVVLIKPSRSMSDLY
DMQKRQRQHRHRNQSGATTSSGDTESEEGEGETTVRLLWLSMLKMPRELMR
LCLCHLLTWFSVIAEAVFYTDFMGQVIFEGDPKAPSNSTAWQAYNAGVKMG
CWGLVIYAATGAICSALLQKYLDNYDLSVRVIYVLGTLGFSVGTAVMAMFP
NVYVAMVTISTMGIVSMSISYCPYALLGQYHDIKQYIHHSPGNSKRGFGID
CAILSCQVYISQILVASALGGVVDAVGTVRVIPMVASVGSFLGFLTATFLV
IYPNVSEEAKEEQKGLSSPLAGEGRAGGNSEKPTVLKLTRKEGLQGPVETE
SVTPAGIDVCQISSHWLVPQLLESIFLYDYFRKKIFFSTMWFS
>NP_001073900.1 SLC45a4 isoform 2
(SEQ ID NO: 487)
MGKASPASGLSRPKTLVSPLRSNQWSLQKGLPEQYYSLTWFLSPILGLIFT
PLIGSASDRCTLSWGRRRPFILALCVGVLFGVALFLNGSAIGLALGDVPNR
QPIGIVLTVLGVVVLDFSADATEGPIRAYLLDVVDSEEQDMALNIHAFSAG
LGGAIGYVLGGLDWTQTFLGSWFRTQNQVLFFFAAIIFTVSVALHLFSIDE
EQYSPQQERSAEEPGALDGGEPHGVPAFPDEVQSEHELALDYPDVDIMRSK
SDSALHVPDTALDLEPELLFLHDIEPSIFHDASYPATPRSTSQELAKTKLP
RLATFLKEAAKEDETLLDNHLNEAKVPNGSGSPTKDALGGYTRVDTKPSAT
SSSMRRRRHAFRRQASSTFSYYGKLGSHCYRYRRANAVVLIKPSRSMSDLY
DMQKRQRQHRHRNQSGATTSSGDTESEEGEGETTVRLLWLSMLKMPRELMR
LCLCHLLTWFSVIAEAVFYTDFMGQVIFEGDPKAPSNSTAWQAYNAGVKMG
CWGLVIYAATGAICSALLQKYLDNYDLSVRVIYVLGTLGFSVGTAVMAMFP
NVYVAMVTISTMGIVSMSISYCPYALLGQYHDIKQYIHHSPGNSKRGFGID
CAILSCQVYISQILVASALGGVVDAVGTVRVIPMVASVGSFLGFLTATFLV
IYPNVSEEAKEEQKGLSSPLAGEGRAGGNSEKPTVLKLTRKEGLQGPVETE
RLQVLTSVRSRHIGWCRSCWRVFFFMIILEKKFSFPQCGSLRRMTYLLFLS
ELDTLCPGQPCPWAATAHQSWEEAGPGGLGRRQ
>NP_001273577.1 SLC45a4isoform 3
(SEQ ID NO: 487)
MGKASPASGLSRPKTLVSPLRSNQWSLQKGLPEQYYSLTWFLSPILGLIFT
PLIGSASDRCTLSWGRRRPFILALCVGVLFGVALFLNGSAIGLALGDVPNR
QPIGIVLTVLGVVVLDFSADATEGPIRAYLLDVVDSEEQDMALNIHAFSAG
LGGAIGYVLGGLDWTQTFLGSWFRTQNQVLFFFAAIIFTVSVALHLFSIDE
EQYSPQQERSAEEPGALDGGEPHGVPAFPDEVQSEHELALDYPDVDIMRSK
SDSALHVPDTALDLEPELLFLHDIEPSIFHDASYPATPRSTSQELAKTKLP
RLATFLKEAAKEDETLLDNHLNEAKVPNGSGSPTKDALGGYTRVDTKPSAT
SSSMRRRRHAFRRQASSTFSYYGKLGSHCYRYRRANAVVLIKPSRSMSDLY
DMQKRQRQHRHRNQSGATTSSGDTESEEGEGETTVRLLWLSMLKMPRELMR
LCLCHLLTWFSVIAEAVFYTDFMGQVIFEGDPKAPSNSTAWQAYNAGVKMG
CWGLVIYAATGAICSALLQKYLDNYDLSVRVIYVLGTLGFSVGTAVMAMFP
NVYVAMVTISTMGIVSMSISYCPYALLGQYHDIKQYIHHSPGNSKRGFGID
CAILSCQVYISQILVASALGGVVDAVGTVRVIPMVASVGSFLGFLTATFLV
IYPNVSEEAKEEQKGLSSPLAGEGRAGGNSEKPTVLKLTRKEGLQGPVETE
SVV

In some examples, where the agent is a nucleic acid, the agent is a nucleic acid molecule capable of interfering with the expression of specific gene.

In some examples, where the agent is a nucleic acid, the agent is an RNAi (RNA interference) (including, but is not limited to, short hairpin molecule (shRNA), an siRNA (small interfering RNA or silencing RNA), and the like), an antisense oligonucleotide (ASO) or nucleoside and nucleotide analogues thereof, a gapmer, a short hairpin antisense oligonucleotide (shASO), a lipid nanoparticle, an adeno-associated virus vector, a gene editing agent (such as a clustered regularly interspaced short palindromic repeats/CRISPR), a ribozyme, a nucleic acid-based nanoparticle, and the like.

As used herein, the term “RNAi” refers to a conserved pathway found in most eukaryotes where double-stranded RNA molecules (dsRNA), inhibit the expression of genes having sequences complementary to the dsRNA.

In some examples, when the agent is a nucleic acid, the agent may be modified by modifications known in the art, such as, but is not limited to, modifications to their phosphate backbone, sugar, or nucleobase, or with conjugations. That is, the agent as disclosed herein may contain alterations to their phosphate backbone (e.g. phosphorothioate instead of phosphate linkages). They may contain nucleotides with modified sugar moieties or sugar moiety analogs. Sugar moiety modifications include, but are not limited to, 2′-O-aminoethoxy, 2′-O-aminoethyl(2′-OAE), 2′-O-methoxy, 2-guanidoethyl(2′-OGE), 2′-O,4′-C-methylene (LNA), 2′-O—(N-(methyl) acetamido) (2′-OMA), 2′-O-methyl, 2′-fluoro, 2′-O-(methoxyethyl) (2′-OME), and the like. They can also contain nucleobase modifications, e.g. 5-methylcytosine or pseudouridine. Such modifications are introduced to improve stability and reduce immunogenicity of the agent. Methods of introducing such modification to nucleic acid agent are common general knowledge in the art.

In some examples, the agent may be GalNAc-nucleic acid conjugates such as GalNAc motif linked siRNAs, ASO, shRNAs, gRNAs, and the like. In some examples, the agent may be GalNac-siRNAs.

In some examples, the agent that modulates the expression of the marker may be a nucleic acid-based therapy, a protein-based therapy, or small molecule-based therapy.

In some examples, the protein-based therapy may include, but is not limited to, antibody, antigen-binding protein/molecule, bispecific antibodies, and the like.

In some examples, the small molecule-based therapy may include a small molecule inhibitor.

In some examples, the subject is genetically of one or more descent such as Southeast Asian, South American, Middle American of Spanish genetic descent, Arabian Peninsula descent, and the like.

In another aspect, there is provided a nucleic acid encoding an agent for inhibiting the one or more gene or marker selected from the group consisting of C1ORF131 (2810004N23Rik), SLC45A4, NFKBIB, ARL6IP5, DBNL, GOLGA7B, FAM117B, MED28, TSC22D4, EIF4EBP1, PFN1, PNRC2, ZNF672 (Zfp672), IER5, ADAMTSL5, COL4A5, MYH15, ILKAP, and the like.

In some examples, the one or more gene or marker may further include, but is not limited to, ABCB10, LSM14A, ACTG1, YTHDF2, RPS6KA1, SEC13, IPO11, CTCF, UBAP2L, P2RY2, TRAF3IP2, FST, TUBG1, ZNF664, PVR, DYX1C1/DNAAF4, TEX264, NIF3L1, RPUSD1, TRIM6, GLRX3, BRD1, CKS1B, C6ORF120, WASF2, ZNF689, B4GALT7, LCORL, NR2F2, BAG6, RNF10, and the like.

In some examples, the one or more gene or marker may further include, but is not limited to, UBA6, BRWD3, FYCO1, ARID5B, C20ORF96 (6820408C15Rik), CD200R1 (Cd200r4), DALRD3, ELF3, ELFN1, FAM160A1, FAM50B, KIAA2026 (9930021J03Rik), KREMEN1, MACC1, NAA25, PROSC/PLPBP, RPS3, SHPK, SLAIN1, TCHP, ZNF503 (Zfp503), BUD13, CD93, PFKFB2, ZDBF2, FOXC2, GBGT1, RGS4, RNASEH1, TSNAXIP1, LYPD2, STIL, TMCO3, TMEM159, RIF1, IFIH1, EPN2, CLP1, RNF220, NOL12, APOL3, GTF3C6, CTPS2, IFITM2, IFITM1, KCNS3, CA2, EIF4H, MRM1, AKAP8, PAPOLA, SECTM1, TYK2, MLLT6, IL1RL2, IPO7, APBB1IP, RABEP2, PLSCR1, PYGO2, COIL, LRRC8E, TNRC6C, CHD3, USP21, SNX27, NOP14, ZC3H15, C15ORF39, UTP20, TRAK1, RAB11FIP2, PHPT1, CUL3, GNL3, and the like.

In some examples, the one or more gene marker may include, but is not limited to, genes provided as disclosed herein. In some examples, the one or more gene marker may include, but is not limited to genes provided in the following tables:

Target marker list 1

C1ORF131 (2810004N23Rik)
SLC45A4
NFKBIB
ARL6IP5
DBNL
GOLGA7B
FAM117B
MED28
TSC22D4
EIF4EBP1
PFN1
PNRC2
ZNF672 (Zfp672)
IER5
ADAMTSL5
COL4A5
MYH15

and/or

Target marker list 2

ABCB10
LSM14A
ACTG1
YTHDF2
RPS6KA1
SEC13
IPO11
CTCF
UBAP2L
P2RY2
TRAF3IP2
FST
TUBG1
ZNF664
PVR
DYX1C1/DNAAF4
TEX264
NIF3L1
RPUSD1
TRIM6
GLRX3
BRD1
CKS1B
C6ORF120
WASF2
ZNF689
B4GALT7
LCORL
NR2F2
BAG6
RNF10

and/or

Target marker list 3

Uba6
Brwd3
Fyco1
ARID5B
C20ORF96 (6820408C15Rik)
CD200R1 (Cd200r4)
DALRD3
ELF3
ELFN1
FAM160A1
FAM50B
KIAA2026 (9930021J03Rik)
KREMEN1
MACC1
NAA25
PROSC/PLPBP
RPS3
SHPK
SLAIN1
TCHP
ZNF503 (Zfp503)
BUD13
CD93
PFKFB2
ZDBF2
FOXC2
GBGT1
RGS4
RNASEH1
TSNAXIP1
LYPD2
STIL
TMCO3
TMEM159
RIF1
IFIH1
EPN2
CLP1
RNF220
NOL12
APOL3
GTF3C6
CTPS2
IFITM2, IFITM1
KCNS3
CA2
EIF4H
MRM1
AKAP8
PAPOLA
SECTM1
TYK2
MLLT6
IL1RL2
IPO7
APBB1IP
RABEP2
PLSCR1
PYGO2
COIL
LRRC8E
TNRC6C
CHD3
USP21
SNX27
NOP14
ZC3H15
C15orf39
UTP20
TRAK1
RAB11FIP2
PHPT1
CUL3
GNL3
Uba6
Brwd3
Fyco1
ARID5B
C20ORF96 (6820408C15Rik)
CD200R1 (Cd200r4)
DALRD3
ELF3
ELFN1
FAM160A1
FAM50B
KIAA2026 (9930021J03Rik)
KREMEN1
MACC1
NAA25
PROSC/PLPBP
RPS3
SHPK
SLAIN1
TCHP
ZNF503 (Zfp503)
BUD13
CD93
PFKFB2
ZDBF2
FOXC2
GBGT1
RGS4
RNASEH1
TSNAXIP1
LYPD2
STIL
TMCO3
TMEM159
RIF1
IFIH1
EPN2
CLP1
RNF220
NOL12
APOL3
GTF3C6
CTPS2
IFITM2, IFITM1
KCNS3
CA2
EIF4H
MRM1
AKAP8
PAPOLA
SECTM1
TYK2
MLLT6
IL1RL2
IPO7
APBB1IP
RABEP2
PLSCR1
PYGO2
COIL
LRRC8E
TNRC6C
CHD3
USP21
SNX27
NOP14
ZC3H15
C15orf39
UTP20
TRAK1
RAB11FIP2
PHPT1
CUL3
GNL3

In some examples, the nucleic acid is an RNAi (RNA interference) (including, but is not limited to, short hairpin molecule (shRNA), an siRNA (small interfering RNA or silencing RNA), and the like), an antisense oligonucleotide (ASO) or nucleoside and nucleotide analogues thereof, a gapmer, a short hairpin antisense oligonucleotide (shASO), a lipid nanoparticle, an adeno-associated virus vector, a gene editing agent (such as a clustered regularly interspaced short palindromic repeats/CRISPR), a ribozyme, or a nucleic acid-based nanoparticle.

In some examples, the nucleic acid is a shRNA.

In some examples, the nucleic acid is a shRNA and comprises the sequence selected from the group consisting of

| | | | SEQ |
|---|---|---|---|
| | Entrez | | ID |
| shRNA | ID | shRNA 97mer sequence | NO: |
| C1orf132031.1 | 128061 | TGCTGTTGACAGTGAGCGAAACCAAGAATCTAGTAATTTATA | 1 |
| | | GTGAAGCCACAGATGTATAAATTACTAGATTCTTGGTTCTGCC | |
| | | TACTGCCTCGGA | |
| C1orf131.179 | 128061 | TGCTGTTGACAGTGAGCGCACAGAAGAAGATCATAAAGAATA | 2 |
| | | GTGAAGCCACAGATGTATTCTTTATGATCTTCTTCTGTTTGCC | |
| | | TACTGCCTCGGA | |
| C1orf131.180 | 128061 | TGCTGTTGACAGTGAGCGCCAGAAGAAGATCATAAAGAAATA | 3 |
| | | GTGAAGCCACAGATGTATTTCTTTATGATCTTCTTCTGTTGCC | |
| | | TACTGCCTCGGA | |
| C1orf131.466 | 128061 | TGCTGTTGACAGTGAGCGACAGATCATAACAAGAACACAATA | 4 |
| | | GTGAAGCCACAGATGTATTGTGTTCTTGTTATGATCTGGTGC | |
| | | CTACTGCCTCGGA | |
| C1orf131.513 | 128061 | TGCTGTTGACAGTGAGCGAAGAGATGTGGATACACAAGAATA | 5 |
| | | GTGAAGCCACAGATGTATTCTTGTGTATCCACATCTCTCTGC | |
| | | CTACTGCCTCGGA | |
| C1orf131.653 | 128061 | TGCTGTTGACAGTGAGCGCAAAGAGTTATGTGAATTACAATA | 6 |
| | | GTGAAGCCACAGATGTATTGTAATTCACATAACTCTTTTTGCC | |
| | | TACTGCCTCGGA | |
| SLC45A4.1893 | 57210 | TGCTGTTGACAGTGAGCGCACAGAAGTACTTGGACAACTATA | 7 |
| | | GTGAAGCCACAGATGTATAGTTGTCCAAGTACTTCTGTATGC | |
| | | CTACTGCCTCGGA | |
| SLC45A4.2558 | 57210 | TGCTGTTGACAGTGAGCGACACAATGTGGTTCTCTTAGAATA | 8 |
| | | GTGAAGCCACAGATGTATTCTAAGAGAACCACATTGTGGTGC | |
| | | CTACTGCCTCGGA | |
| SLC45A4.3229 | 57210 | TGCTGTTGACAGTGAGCGCCGGGCAAGTACTGCAGCTATTTA | 9 |
| | | GTGAAGCCACAGATGTAAATAGCTGCAGTACTTGCCCGATGC | |
| | | CTACTGCCTCGGA | |
| SLC45A4.3266 | 57210 | TGCTGTTGACAGTGAGCGCACCACAGTCTCTTTAGAACTATA | 10 |
| | | GTGAAGCCACAGATGTATAGTTCTAAAGAGACTGTGGTTTGC | |
| | | CTACTGCCTCGGA | |
| SLC45A4.3340 | 57210 | TGCTGTTGACAGTGAGCGACCCGCACATCACACAGATAAATA | 11 |
| | | GTGAAGCCACAGATGTATTTATCTGTGTGATGTGCGGGGTGC | |
| | | CTACTGCCTCGGA | |
| SLC45A4.3349 | 57210 | TGCTGTTGACAGTGAGCGCCACACAGATAAAGTCAGACAATA | 12 |
| | | GTGAAGCCACAGATGTATTGTCTGACTTTATCTGTGTGATGC | |
| | | CTACTGCCTCGGA | |

In some examples, the nucleic acid is a siRNA.

In some examples, the nucleic acid is a siRNA and comprises the sequence selected from the group consisting of
C1ORF131 (Variant 1) Isoform a

| | | | SEQ | | SEQ | | |
|---|---|---|---|---|---|---|---|
| | | | ID | | ID | | |
| SIRNA_id | Position | SS Sequence | NO: | AS Sequence | NO: | Score | Corrected_Score |
| 1 | 1251 | GCUAGUAGUUUA | 13 | UUUAAUGUAAAC | 63 | 104.1 | 92.1 |
| | | CAUUAAAUU | | UACUAGCAU | | | |
| 2 | 1316 | CCAAGAAUCUAG | 14 | UAAAUUACUAGA | 64 | 102.8 | 89.8 |
| | | UAAUUUAUC | | UUCUUGGUU | | | |
| 3 | 649 | AGAGUUAUGUGA | 15 | UUGUAAUUCACA | 65 | 99.8 | 93.8 |
| | | AUUACAAGG | | UAACUCUUU | | | |
| 4 | 171 | GAACAGAAGAAG | 16 | UUAUGAUCUUCU | 66 | 99.1 | 98.1 |
| | | AUCAUAAAG | | UCUGUUCUG | | | |
| 5 | 562 | GGUUUGGUAUCA | 17 | UAACCCGUGAUA | 67 | 99 | 94 |
| | | CGGGUUAUG | | CCAAACCGG | | | |
| 6 | 986 | CUAGAUAGAUCG | 18 | UUAAGAACGAUC | 68 | 98.4 | 89.4 |
| | | UUCUUAACA | | UAUCUAGAG | | | |
| 7 | 143 | CGACUUUGGAGG | 19 | UUCUGUACCUCC | 69 | 98.2 | 97.2 |
| | | UACAGAAGG | | AAAGUCGUA | | | |
| 8 | 1201 | GGUGCAUUUGCU | 20 | UAUUACAAGCAA | 70 | 98.2 | 86.2 |
| | | UGUAAUACA | | AUGCACCAA | | | |
| 9 | 1250 | UGCUAGUAGUUU | 21 | UUAAUGUAAACU | 71 | 96.4 | 84.4 |
| | | ACAUUAAAU | | ACUAGCAUU | | | |
| 10 | 1244 | UGUAAAUGCUAG | 22 | UAAACUACUAGC | 72 | 96.3 | 84.3 |
| | | UAGUUUACA | | AUUUACAAA | | | |
| 11 | 503 | GGAGAGAGAUGU | 23 | UGUAUCCACAUC | 73 | 96.2 | 91.2 |
| | | GGAUACACA | | UCUCUCCAA | | | |
| 12 | 407 | GGAACAAGUAGA | 24 | UACCACUUCUAC | 74 | 96.1 | 92.1 |
| | | AGUGGUAGA | | UUGUUCCCU | | | |
| 13 | 976 | CUGUGCGACUCU | 25 | UCUAUCUAGAGU | 75 | 95.7 | 86.7 |
| | | AGAUAGAUC | | CGCACAGGG | | | |
| 14 | 1110 | AGUGCAGCAUGA | 26 | UUCACAUUCAUG | 76 | 95.5 | 84.5 |
| | | AUGUGAAGU | | CUGCACUGG | | | |
| 15 | 162 | GGUGAAACAGAA | 27 | UCUUCUGUUCUG | 77 | 95.5 | 94.5 |
| | | CAGAAGAAG | | UUUCACCUU | | | |
| 16 | 385 | CCUCUUCCCUAA | 28 | UUGUUCUUUAGG | 78 | 94.3 | 91.3 |
| | | AGAACAAUA | | GAAGAGGGA | | | |
| 17 | 150 | GGAGGUACAGAA | 29 | UUUCACCUUCUG | 79 | 93.7 | 92.7 |
| | | GGUGAAACA | | UACCUCCAA | | | |
| 18 | 1390 | CUUCAUAAGAAG | 30 | UAUAUAACUUCU | 80 | 93.6 | 80.6 |
| | | UUAUAUAAA | | UAUGAAGUA | | | |
| 19 | 170 | AGAACAGAAGAA | 31 | UAUGAUCUUCUU | 81 | 93.6 | 92.6 |
| | | GAUCAUAAA | | CUGUUCUGU | | | |
| 20 | 255 | CUACCUGGUUCU | 32 | UUAUGAGAGAAC | 82 | 93.5 | 91.5 |
| | | CUCAUAAGA | | CAGGUAGGG | | | |
| 21 | 254 | CCUACCUGGUUC | 33 | UAUGAGAGAACC | 83 | 93.1 | 91.1 |
| | | UCUCAUAAG | | AGGUAGGGG | | | |
| 22 | 398 | GAACAAUAGGGA | 34 | UACUUGUUCCCU | 84 | 93 | 90 |
| | | ACAAGUAGA | | AUUGUUCUU | | | |
| 23 | 1317 | CAAGAAUCUAGU | 35 | AUAAAUUACUAG | 85 | 92.8 | 79.8 |
| | | AAUUUAUCA | | AUUCUUGGU | | | |
| 24 | 423 | GUAGAAUUUCAC | 36 | UAUUGCUGUGAA | 86 | 92.5 | 88.5 |
| | | AGCAAUAAA | | AUUCUACCA | | | |
| 25 | 910 | AAUGAAGUACUU | 37 | UUUGACAAAGUA | 87 | 92.1 | 83.1 |
| | | UGUCAAAUA | | CUUCAUUUG | | | |
| 26 | 401 | CAAUAGGGAACA | 38 | UUCUACUUGUUC | 88 | 92.1 | 88.1 |
| | | AGUAGAAGU | | CCUAUUGUU | | | |
| 27 | 912 | UGAAGUACUUUG | 39 | UAUUUGACAAAG | 89 | 92 | 83 |
| | | UCAAAUAAA | | UACUUCAUU | | | |
| 28 | 909 | AAAUGAAGUACU | 40 | UUGACAAAGUAC | 90 | 91.8 | 82.8 |
| | | UUGUCAAAU | | UUCAUUUGG | | | |
| 29 | 1294 | AUGUUAUGAUAU | 41 | UUUCAUGAUAUC | 91 | 91.8 | 79.8 |
| | | CAUGAAAGA | | AUAACAUUA | | | |
| 30 | 1313 | GAACCAAGAAUC | 42 | AUUACUAGAUUC | 92 | 91.6 | 78.6 |
| | | UAGUAAUUU | | UUGGUUCUU | | | |
| 31 | 392 | CCUAAAGAACAA | 43 | UUCCCUAUUGUU | 93 | 91.3 | 88.3 |
| | | UAGGGAACA | | CUUUAGGGA | | | |
| 32 | 1180 | GUUUCAUGUUCA | 44 | AUACCAGUGAAC | 94 | 91.3 | 80.3 |
| | | CUGGUAUUU | | AUGAAACAG | | | |
| 33 | 289 | GCGCUUCGAGCU | 45 | UUGAAGAAGCUC | 95 | 91.3 | 89.3 |
| | | UCUUCAAGG | | GAAGCGCUC | | | |
| 34 | 934 | CGAGGAUGGGAA | 46 | UCAGUUGUUCCC | 96 | 91.2 | 82.2 |
| | | CAACUGAGU | | AUCCUCGUU | | | |
| 35 | 1286 | AUGAUUUAAUGU | 47 | UAUCAUAACAUU | 97 | 91 | 79 |
| | | UAUGAUAUC | | AAAUCAUUA | | | |
| 36 | 1393 | CAUAAGAAGUUA | 48 | AUUUAUAUAACU | 98 | 90.9 | 77.9 |
| | | UAUAAAUAA | | UCUUAUGAA | | | |
| 37 | 413 | AGUAGAAGUGGU | 49 | AAAUUCUACCAC | 99 | 90.9 | 86.9 |
| | | AGAAUUUCA | | UUCUACUUG | | | |
| 38 | 378 | GUUCCUCCCUCU | 50 | UUAGGGAAGAGG | 100 | 90.9 | 87.9 |
| | | UCCCUAAAG | | GAGGAACUG | | | |
| 39 | 764 | AGGACAGGAGGA | 51 | UUUCCUGUCCUC | 101 | 90.6 | 83.6 |
| | | CAGGAAAUC | | CUGUCCUUU | | | |
| 40 | 972 | GACCCUGUGCGA | 52 | UCUAGAGUCGCA | 102 | 90.5 | 81.5 |
| | | CUCUAGAUA | | CAGGGUCCA | | | |
| 41 | 422 | GGUAGAAUUUCA | 53 | AUUGCUGUGAAA | 103 | 90.4 | 86.4 |
| | | CAGCAAUAA | | UUCUACCAC | | | |
| 42 | 172 | AACAGAAGAAGA | 54 | UUUAUGAUCUUC | 104 | 90.2 | 89.2 |
| | | UCAUAAAGA | | UUCUGUUCU | | | |
| 43 | 1386 | UCUACUUCAUAA | 55 | UAACUUCUUAUG | 105 | 90.1 | 77.1 |
| | | GAAGUUAUA | | AAGUAGAUU | | | |
| 44 | 974 | CCCUGUGCGACU | 56 | UAUCUAGAGUCG | 106 | 89.9 | 80.9 |
| | | CUAGAUAGA | | CACAGGGUC | | | |
| 45 | 552 | GAAGUGCACCGG | 57 | UACCAAACCGGU | 107 | 89.7 | 84.7 |
| | | UUUGGUAUC | | GCACUUCUA | | | |
| 46 | 1394 | AUAAGAAGUUAU | 58 | UAUUUAUAUAAC | 108 | 89.7 | 76.7 |
| | | AUAAAUAAA | | UUCUUAUGA | | | |
| 47 | 648 | AAGAGUUAUGUG | 59 | UGUAAUUCACAU | 109 | 89.6 | 83.6 |
| | | AAUUACAAG | | AACUCUUUU | | | |
| 48 | 765 | GGACAGGAGGAC | 60 | AUUUCCUGUCCU | 110 | 89.5 | 82.5 |
| | | AGGAAAUCC | | CCUGUCCUU | | | |
| 49 | 359 | AGAGAUCCUUGC | 61 | UGCAGCAGCAAG | 111 | 89.4 | 86.4 |
| | | UGCUGCAGU | | GAUCUCUGG | | | |
| 50 | 1249 | AUGCUAGUAGUU | 62 | UAAUGUAAACUA | 112 | 89.4 | 77.4 |
| | | UACAUUAAA | | CUAGCAUUU | | | |

C1ORF131 (Variant 2) Isoform b

| | | | SEQ | | SEQ | | |
|---|---|---|---|---|---|---|---|
| | | | ID | | ID | | |
| SIRNA_id | Position | SS Sequence | NO: | AS Sequence | NO: | Score | Corrected_Score |
| 1 | 1248 | GCUAGUAGUUUA | 113 | UUUAAUGUAAAC | 163 | 104.1 | 92.1 |
| | | CAUUAAAUU | | UACUAGCAU | | | |
| 2 | 1313 | CCAAGAAUCUAG | 114 | UAAAUUACUAGA | 164 | 102.8 | 89.8 |
| | | UAAUUUAUC | | UUCUUGGUU | | | |
| 3 | 646 | AGAGUUAUGUGA | 115 | UUGUAAUUCACA | 165 | 99.8 | 93.8 |
| | | AUUACAAGG | | UAACUCUUU | | | |
| 4 | 171 | GAACAGAAGAAG | 116 | UUAUGAUCUUCU | 166 | 99.1 | 98.1 |
| | | AUCAUAAAG | | UCUGUUCUG | | | |
| 5 | 559 | GGUUUGGUAUCA | 117 | UAACCCGUGAUA | 167 | 99 | 94 |
| | | CGGGUUAUG | | CCAAACCGG | | | |
| 6 | 983 | CUAGAUAGAUCG | 118 | UUAAGAACGAUC | 168 | 98.4 | 89.4 |
| | | UUCUUAACA | | UAUCUAGAG | | | |
| 7 | 143 | CGACUUUGGAGG | 119 | UUCUGUACCUCC | 169 | 98.2 | 97.2 |
| | | UACAGAAGG | | AAAGUCGUA | | | |
| 8 | 1198 | GGUGCAUUUGCU | 120 | UAUUACAAGCAA | 170 | 98.2 | 87.2 |
| | | UGUAAUACA | | AUGCACCAA | | | |
| 9 | 1247 | UGCUAGUAGUUU | 121 | UUAAUGUAAACU | 171 | 96.4 | 84.4 |
| | | ACAUUAAAU | | ACUAGCAUU | | | |
| 10 | 1241 | UGUAAAUGCUAG | 122 | UAAACUACUAGC | 172 | 96.3 | 84.3 |
| | | UAGUUUACA | | AUUUACAAA | | | |
| 11 | 500 | GGAGAGAGAUGU | 123 | UGUAUCCACAUC | 173 | 96.2 | 91.2 |
| | | GGAUACACA | | UCUCUCCAA | | | |
| 12 | 407 | GGAACAAGUAGA | 124 | UACCACUUCUAC | 174 | 96.1 | 92.1 |
| | | AGUGGUAGA | | UUGUUCCCU | | | |
| 13 | 973 | CUGUGCGACUCU | 125 | UCUAUCUAGAGU | 175 | 95.7 | 86.7 |
| | | AGAUAGAUC | | CGCACAGGG | | | |
| 14 | 1107 | AGUGCAGCAUGA | 126 | UUCACAUUCAUG | 176 | 95.5 | 84.5 |
| | | AUGUGAAGU | | CUGCACUGG | | | |
| 15 | 162 | GGUGAAACAGAA | 127 | UCUUCUGUUCUG | 177 | 95.5 | 94.5 |
| | | CAGAAGAAG | | UUUCACCUU | | | |
| 16 | 385 | CCUCUUCCCUAA | 128 | UUGUUCUUUAGG | 178 | 94.3 | 91.3 |
| | | AGAACAAUA | | GAAGAGGGA | | | |
| 17 | 150 | GGAGGUACAGAA | 129 | UUUCACCUUCUG | 179 | 93.7 | 92.7 |
| | | GGUGAAACA | | UACCUCCAA | | | |
| 18 | 138 | CUUCAUAAGAAG | 130 | UAUAUAACUUCU | 180 | 93.6 | 80.6 |
| | 7 | UUAUAUAAA | | UAUGAAGUA | | | |
| 19 | 170 | AGAACAGAAGAA | 131 | UAUGAUCUUCUU | 181 | 93.6 | 92.6 |
| | | GAUCAUAAA | | CUGUUCUGU | | | |
| 20 | 255 | CUACCUGGUUCU | 132 | UUAUGAGAGAAC | 182 | 93.5 | 91.5 |
| | | CUCAUAAGA | | CAGGUAGGG | | | |
| 21 | 254 | CCUACCUGGUUC | 133 | UAUGAGAGAACC | 183 | 93.1 | 91.1 |
| | | UCUCAUAAG | | AGGUAGGGG | | | |
| 22 | 398 | GAACAAUAGGGA | 134 | UACUUGUUCCCU | 184 | 93 | 90 |
| | | ACAAGUAGA | | AUUGUUCUU | | | |
| 23 | 1314 | CAAGAAUCUAGU | 135 | AUAAAUUACUAG | 185 | 92.8 | 79.8 |
| | | AAUUUAUCA | | AUUCUUGGU | | | |
| 24 | 423 | GUAGAAUUUCAC | 136 | UAUUGCUGUGAA | 186 | 92.5 | 88.5 |
| | | AGCAAUAAA | | AUUCUACCA | | | |
| 25 | 907 | AAUGAAGUACUU | 137 | UUUGACAAAGUA | 187 | 92.1 | 83.1 |
| | | UGUCAAAUA | | CUUCAUUUG | | | |
| 26 | 401 | CAAUAGGGAACA | 138 | UUCUACUUGUUC | 188 | 92.1 | 88.1 |
| | | AGUAGAAGU | | CCUAUUGUU | | | |
| 27 | 909 | UGAAGUACUUUG | 139 | UAUUUGACAAAG | 189 | 92 | 83 |
| | | UCAAAUAAA | | UACUUCAUU | | | |
| 28 | 906 | AAAUGAAGUACU | 140 | UUGACAAAGUAC | 190 | 91.8 | 82.8 |
| | | UUGUCAAAU | | UUCAUUUGG | | | |
| 29 | 1291 | AUGUUAUGAUAU | 141 | UUUCAUGAUAUC | 191 | 91.8 | 79.8 |
| | | CAUGAAAGA | | AUAACAUUA | | | |
| 30 | 1310 | GAACCAAGAAUC | 142 | AUUACUAGAUUC | 192 | 91.6 | 78.6 |
| | | UAGUAAUUU | | UUGGUUCUU | | | |
| 1 | 392 | CCUAAAGAACAA | 143 | UUCCCUAUUGUU | 193 | 91.3 | 88.3 |
| | | UAGGGAACA | | CUUUAGGGA | | | |
| 32 | 1177 | GUUUCAUGUUCA | 144 | AUACCAGUGAAC | 194 | 91.3 | 80.3 |
| | | CUGGUAUUU | | AUGAAACAG | | | |
| 33 | 289 | GCGCUUCGAGCU | 145 | UUGAAGAAGCUC | 195 | 91.3 | 89.3 |
| | | UCUUCAAGG | | GAAGCGCUC | | | |
| 34 | 931 | CGAGGAUGGGAA | 146 | UCAGUUGUUCCC | 196 | 91.2 | 82.2 |
| | | CAACUGAGU | | AUCCUCGUU | | | |
| 35 | 1283 | AUGAUUUAAUGU | 147 | UAUCAUAACAUU | 197 | 91 | 79 |
| | | UAUGAUAUC | | AAAUCAUUA | | | |
| 36 | 1390 | CAUAAGAAGUUA | 148 | AUUUAUAUAACU | 198 | 90.9 | 77.9 |
| | | UAUAAAUAA | | UCUUAUGAA | | | |
| 37 | 413 | AGUAGAAGUGGU | 149 | AAAUUCUACCAC | 199 | 90.9 | 86.9 |
| | | AGAAUUUCA | | UUCUACUUG | | | |
| 38 | 378 | GUUCCUCCCUCU | 150 | UUAGGGAAGAGG | 200 | 90.9 | 87.9 |
| | | UCCCUAAAG | | GAGGAACUG | | | |
| 39 | 761 | AGGACAGGAGGA | 151 | UUUCCUGUCCUC | 201 | 90.6 | 83.6 |
| | | CAGGAAAUC | | CUGUCCUUU | | | |
| 40 | 969 | GACCCUGUGCGA | 152 | UCUAGAGUCGCA | 202 | 90.5 | 81.5 |
| | | CUCUAGAUA | | CAGGGUCCA | | | |
| 41 | 422 | GGUAGAAUUUCA | 153 | AUUGCUGUGAAA | 203 | 90.4 | 86.4 |
| | | CAGCAAUAA | | UUCUACCAC | | | |
| 42 | 172 | AACAGAAGAAGA | 154 | UUUAUGAUCUUC | 204 | 90.2 | 89.2 |
| | | UCAUAAAGA | | UUCUGUUCU | | | |
| 43 | 1383 | UCUACUUCAUAA | 155 | UAACUUCUUAUG | 205 | 90.1 | 77.1 |
| | | GAAGUUAUA | | AAGUAGAUU | | | |
| 44 | 971 | CCCUGUGCGACU | 156 | UAUCUAGAGUCG | 206 | 89.9 | 80.9 |
| | | CUAGAUAGA | | CACAGGGUC | | | |
| 45 | 549 | GAAGUGCACCGG | 157 | UACCAAACCGGU | 207 | 89.7 | 84.7 |
| | | UUUGGUAUC | | GCACUUCUA | | | |
| 46 | 1391 | AUAAGAAGUUAU | 158 | UAUUUAUAUAAC | 208 | 89.7 | 76.7 |
| | | AUAAAUAAA | | UUCUUAUGA | | | |
| 47 | 645 | AAGAGUUAUGUG | 159 | UGUAAUUCACAU | 209 | 89.6 | 83.6 |
| | | AAUUACAAG | | AACUCUUUU | | | |
| 48 | 762 | GGACAGGAGGAC | 160 | AUUUCCUGUCCU | 210 | 89.5 | 82.5 |
| | | AGGAAAUCC | | CCUGUCCUU | | | |
| 49 | 359 | AGAGAUCCUUGC | 161 | UGCAGCAGCAAG | 211 | 89.4 | 86.4 |
| | | UGCUGCAGU | | GAUCUCUGG | | | |
| 50 | 1246 | AUGCUAGUAGUU | 162 | UAAUGUAAACUA | 212 | 894 | 77.4 |
| | | UACAUUAAA | | CUAGCAUUU | | | |

SLC45A4 (Variant 1) Isoform 1

| siRNA_ | | | SEQ | | SEQ | | Corrected_ |
|---|---|---|---|---|---|---|---|
| id | Position | SS Sequence | ID NO: | AS Sequence | ID NO: | Score | Score |
| 1 | 553 | GAUAGAAUAUAU | 213 | UUUAUCUAUAUA | 263 | 105.5 | 100.5 |
| | | AGAUAAAUA | | UUCUAUCUA | | | |
| 2 | 621 | GGAAUCUAUGCA | 214 | UUGAACUUGCAU | 264 | 102.8 | 96.8 |
| | | AGUUCAAGA | | AGAUUCCGG | | | |
| 3 | 3990 | GGAACGACUGUU | 215 | UACAUUAAACAG | 265 | 102.2 | 63.2 |
| | | UAAUGUACU | | UCGUUCCUU | | | |
| 4 | 3803 | GUCAGACAAUUG | 216 | UUAAUUACAAUU | 266 | 102 | 64 |
| | | UAAUUAAUA | | GUCUGACUU | | | |
| 5 | 4761 | CCCUCUGAAUAG | 217 | UAACACACUAUU | 267 | 100.9 | 53.9 |
| | | UGUGUUAAC | | CAGAGGGGG | | | |
| 6 | 6695 | AGUUCAGUUCCG | 218 | UUCAACUCGGAA | 268 | 100.7 | 34.7 |
| | | AGUUGAACA | | CUGAACUGA | | | |
| 7 | 620 | CGGAAUCUAUGC | 219 | UGAACUUGCAUA | 269 | 100.7 | 94.7 |
| | | AAGUUCAAG | | GAUUCCGGG | | | |
| 8 | 501 | GAGAAGACGUCU | 220 | UAAAUUAAGACG | 270 | 99.5 | 94.5 |
| | | UAAUUUAUC | | UCUUCUCUU | | | |
| 9 | 3002 | CAAUGUGGUUCU | 221 | UUCUAAGAGAAC | 271 | 99.4 | 69.4 |
| | | CUUAGAAGA | | CACAUUGUG | | | |
| 10 | 2330 | AGAAGUACUUGG | 222 | UAGUUGUCCAAG | 272 | 99.2 | 76.2 |
| | | ACAACUACG | | UACUUCUGU | | | |
| 11 | 552 | AGAUAGAAUAUA | 223 | UUAUCUAUAUAU | 273 | 98.7 | 93.7 |
| | | UAGAUAAAU | | UCUAUCUAU | | | |
| 12 | 5682 | GGAUAAUGUUGG | 224 | AAUGAGACCAAC | 274 | 98.7 | 42.7 |
| | | UCUCAUUUG | | AUUAUCCUA | | | |
| 13 | 6168 | GGUCUGUGACCA | 225 | UUCUAGGUGGUC | 275 | 98.4 | 37.4 |
| | | CCUAGAACC | | ACAGACCAA | | | |
| 14 | 4561 | GAAUCAAGAUCC | 226 | UUGGGUCGGAUC | 276 | 98.3 | 53.3 |
| | | GACCCAAAC | | UUGAUUCGG | | | |
| 15 | 4228 | GGCAAGUCCGAC | 227 | UUCAGUAGUCGG | 277 | 98.1 | 56.1 |
| | | UACUGAAAG | | ACUUGCCCU | | | |
| 16 | 4955 | CACUUUGUAGAC | 228 | UUGACGAGUCUA | 278 | 97.5 | 48.5 |
| | | UCGUCAAGC | | CAAAGUGUG | | | |
| 17 | 5627 | GGGUGUGUCGGU | 229 | UCGACCAACCGA | 279 | 97.4 | 41.4 |
| | | UGGUCGAGU | | CACACCCUG | | | |
| 18 | 1346 | GCAUCGACGAGG | 230 | UACUGCUCCUCG | 280 | 97 | 84 |
| | | AGCAGUACA | | UCGAUGCUG | | | |
| 19 | 5716 | CCCUAUACAAGU | 231 | UUAGCUGACUUG | 281 | 97 | 40 |
| | | CAGCUAAGU | | UAUAGGGAA | | | |
| 20 | 4981 | GCACUUCAGGUG | 232 | UUGCAAACACCU | 282 | 96.9 | 47.9 |
| | | UUUGCAAGC | | GAAGUGCUG | | | |
| 21 | 3884 | GCAAUAUGAUCA | 233 | UCUAGAAUGAUC | 283 | 96.6 | 58.6 |
| | | UUCUAGAGC | | AUAUUGCAG | | | |
| 22 | 3643 | GUUCUGGUUUCA | 234 | UAUUCGUUGAAA | 284 | 96.5 | 60.5 |
| | | ACGAAUAAG | | CCAGAACGA | | | |
| 23 | 1713 | GGAUAAUCACUU | 235 | UUCAUUCAAGUG | 285 | 96.1 | 79.1 |
| | | GAAUGAAGC | | AUUAUCCAG | | | |
| 24 | 3995 | GACUGUUUAAUG | 236 | UCAAGUACAUUA | 286 | 96.1 | 57.1 |
| | | UACUUGAUG | | AACAGUCGU | | | |
| 25 | 2999 | CCACAAUGUGGU | 237 | UAAGAGAACCAC | 287 | 95.9 | 66.9 |
| | | UCUCUUAGA | | AUUGUGGAA | | | |
| 26 | 801 | CGCUCUGGUCAC | 238 | UAUUGGUGUGAC | 288 | 95.7 | 87.7 |
| | | ACCAAUACU | | CAGAGCGGU | | | |
| 27 | 181 | GGAGGAGACAGA | 239 | UGGAUGAUCUGU | 289 | 95.5 | 94.5 |
| | | UCAUCCAGU | | CUCCUCCGG | | | |
| 28 | 4054 | CUUUGUAGAUUG | 240 | AAACACACAAUC | 290 | 95.5 | 55.5 |
| | | UGUGUUUGU | | UACAAAGUG | | | |
| 29 | 1265 | GCUGGUUCCGGA | 241 | UUCUGGGUCCG | 291 | 95.4 | 83.4 |
| | | CCCAGAACC | | GAACCAGCUG | | | |
| 30 | 557 | GAAUAUAUAGAU | 242 | UGUAUUUAUCUA | 292 | 95.4 | 90.4 |
| | | AAAUACAUA | | UAUAUUCUA | | | |
| 31 | 1952 | GCAUGAGCGACC | 243 | UCGUACAGGUCG | 293 | 95.1 | 76.1 |
| | | UGUACGACA | | CUCAUGCUG | | | |
| 32 | 284 | CUUCAGUAAUAA | 244 | UUCAUCAUUAUU | 294 | 95.1 | 93.1 |
| | | UGAUGAAAC | | ACUGAAGAG | | | |
| 33 | 526 | CAGAUAUAUGAA | 245 | UAAUGCUUUCAU | 295 | 95 | 90 |
| | | AGCAUUAUU | | AUAUCUGUA | | | |
| 34 | 1862 | CCAGCACCUUCU | 246 | UAGUAGGAGAAG | 296 | 95 | 77 |
| | | CCUACUACG | | GUGCUGGAG | | | |
| 35 | 5668 | CAGUUACCAAAC | 247 | UAUCCUAGUUUG | 297 | 95 | 39 |
| | | UAGGAUAAU | | GUAACUGGU | | | |
| 36 | 497 | GAAAGAGAAGAC | 248 | UUAAGACGUCUU | 298 | 95 | 91 |
| | | GUCUUAAUU | | CUCUUUCUG | | | |
| 37 | 627 | UAUGCAAGUUCA | 249 | UAACUCUUGAAC | 299 | 94.9 | 88.9 |
| | | AGAGUUAUC | | UUGCAUAGA | | | |
| 38 | 486 | CAGCAGAAGCAG | 250 | UCUCUUUCUGCU | 300 | 94.9 | 90.9 |
| | | AAAGAGAAG | | UCUGCUGUG | | | |
| 39 | 487 | AGCAGAAGCAGA | 251 | UUCUCUUUCUGC | 301 | 94.8 | 90.8 |
| | | AAGAGAAGA | | UUCUGCUGU | | | |
| 40 | 3710 | CACAGUCUCUUU | 252 | UAGUUCUAAAGA | 302 | 94.8 | 57.8 |
| | | AGAACUAAG | | GACUGUGGU | | | |
| 41 | 3673 | GGCAAGUACUGC | 253 | AAUAGCUGCAGU | 303 | 94.7 | 58.7 |
| | | AGCUAUUUG | | ACUUGCCCG | | | |
| 42 | 278 | GCACCUCUUCAG | 254 | AUUAUUACUGAA | 304 | 94.7 | 92.7 |
| | | UAAUAAUGA | | GAGGUGCUG | | | |
| 43 | 812 | CACCAAUACUGU | 255 | AUCUGCAACAGU | 305 | 94.6 | 86.6 |
| | | UGCAGAUUG | | AUUGGUGUG | | | |
| 44 | 5224 | AGAUUGCACUCA | 256 | UUUCAAGUGAGU | 306 | 94.5 | 42.5 |
| | | CUUGAAACC | | GCAAUCUGG | | | |
| 45 | 452 | AAGAGGAAUGUA | 257 | UAACACUUACAU | 307 | 94.5 | 90.5 |
| | | AGUGUUACC | | UCCUCUUUG | | | |
| 46 | 3284 | CGUCUGAAGGAC | 258 | UAGGAGAGUCCU | 308 | 94.4 | 62.4 |
| | | UCUCCUAGG | | UCAGACGGG | | | |
| 47 | 5663 | CAGACCAGUUAC | 259 | UAGUUUGGUAAC | 309 | 94.3 | 38.3 |
| | | CAAACUAGG | | UGGUCUGCG | | | |
| 48 | 4141 | GCAGAGGGCUGA | 260 | UUGAAAGUCAGC | 310 | 94.2 | 53.2 |
| | | CUUUCAAGA | | CCUCUGCGA | | | |
| 49 | 3461 | CGGGUGUGUGGA | 261 | UCGCACAUCCAC | 311 | 94 | 60 |
| | | UGUGCGAGG | | ACACCCGAG | | | |
| 50 | 4227 | GGGCAAGUCCGA | 262 | UCAGUAGUCGGA | 312 | 93.9 | 51.9 |
| | | CUACUGAAA | | CUUGCCCUG | | | |

| siRNA_ | | | SEQ | | SEQ | | Corrected_ |
|---|---|---|---|---|---|---|---|
| id | Position | SS Sequence | ID NO: | AS Sequence | ID NO: | Score | Score |
| 1 | 3545 | GGAACGACUGUU | 313 | UACAUUAAACAG | 363 | 102.2 | 67.2 |
| | | UAAUGUACU | | UCGUUCCUU | | | |
| 2 | 3358 | GUCAGACAAUUG | 314 | UUAAUUACAAUU | 364 | 102 | 69 |
| | | UAAUUAAUA | | GUCUGACUU | | | |
| 3 | 4316 | CCCUCUGAAUAG | 315 | UAACACACUAUU | 365 | 100.9 | 57.9 |
| | | UGUGUUAAC | | CAGAGGGGG | | | |
| 4 | 6250 | AGUUCAGUUCCG | 316 | UUCAACUCGGAA | 366 | 100.7 | 38.7 |
| | | AGUUGAACA | | CUGAACUGA | | | |
| 5 | 2557 | CAAUGUGGUUCU | 317 | UUCUAAGAGAAC | 367 | 99.4 | 74.4 |
| | | CUUAGAAGA | | CACAUUGUG | | | |
| 6 | 1892 | AGAAGUACUUGG | 318 | UAGUUGUCCAAG | 368 | 992 | 81.2 |
| | | ACAACUACG | | UACUUCUGU | | | |
| 7 | 5237 | GGAUAAUGUUGG | 319 | AAUGAGACCAAC | 339 | 98.7 | 46.7 |
| | | UCUCAUUUG | | AUUAUCCUA | | | |
| 8 | 5723 | GGUCUGUGACCA | 320 | UUCUAGGUGGUC | 340 | 98.4 | 41.4 |
| | | CCUAGAACC | | ACAGACCAA | | | |
| 9 | 4116 | GAAUCAAGAUCC | 321 | UUGGGUCGGAUC | 341 | 98.3 | 57.3 |
| | | GACCCAAAC | | UUGAUUCGG | | | |
| 10 | 3783 | GGCAAGUCCGAC | 322 | UUCAGUAGUCGG | 342 | 98.1 | 61.1 |
| | | UACUGAAAG | | ACUUGCCCU | | | |
| 11 | 4510 | CACUUUGUAGAC | 323 | UUGACGAGUCUA | 343 | 97.5 | 52.5 |
| | | UCGUCAAGC | | CAAAGUGUG | | | |
| 12 | 5182 | GGGUGUGUCGG | 324 | UCGACCAACCGA | 344 | 97.4 | 46.4 |
| | | UUGGUCGAGU | | CACACCCUG | | | |
| 13 | 908 | GCAUCGACGAGG | 325 | UACUGCUCCUCG | 345 | 97 | 88 |
| | | AGCAGUACA | | UCGAUGCUG | | | |
| 14 | 5271 | CCCUAUACAAGU | 326 | UUAGCUGACUUG | 346 | 97 | 45 |
| | | CAGCUAAGU | | UAUAGGGAA | | | |
| 15 | 4536 | GCACUUCAGGUG | 327 | UUGCAAACACCU | 347 | 96.9 | 51.9 |
| | | UUUGCAAGC | | GAAGUGCUG | | | |
| 16 | 3439 | GCAAUAUGAUCA | 328 | UCUAGAAUGAUC | 348 | 96.6 | 62.6 |
| | | UUCUAGAGC | | AUAUUGCAG | | | |
| 17 | 3198 | GUUCUGGUUUCA | 329 | UAUUCGUUGAAA | 349 | 96.5 | 65.5 |
| | | ACGAAUAAG | | CCAGAACGA | | | |
| 18 | 1275 | GGAUAAUCACUU | 330 | UUCAUUCAAGUG | 350 | 96.1 | 84.1 |
| | | GAAUGAAGC | | AUUAUCCAG | | | |
| 19 | 3550 | GACUGUUUAAUG | 331 | UCAAGUACAUUA | 351 | 96.1 | 61.1 |
| | | UACUUGAUG | | AACAGUCGU | | | |
| 20 | 2554 | CCACAAUGUGGU | 332 | UAAGAGAACCAC | 352 | 95.9 | 70.9 |
| | | UCUCUUAGA | | AUUGUGGAA | | | |
| 21 | 3609 | CUUUGUAGAUUG | 333 | AAACACACAAUCU | 353 | 95.5 | 59.5 |
| | | UGUGUUUGU | | ACAAAGUG | | | |
| 22 | 827 | GCUGGUUCCGGA | 334 | UUCUGGGUCCGG | 354 | 95.4 | 87.4 |
| | | CCCAGAACC | | AACCAGCUG | | | |
| 23 | 1514 | GCAUGAGCGACC | 335 | UCGUACAGGUCG | 355 | 95.1 | 80.1 |
| | | UGUACGACA | | CUCAUGCUG | | | |
| 24 | 1424 | CCAGCACCUUCU | 336 | UAGUAGGAGAAG | 356 | 95 | 81 |
| | | CCUACUACG | | GUGCUGGAG | | | |
| 25 | 5223 | CAGUUACCAAAC | 337 | UAUCCUAGUUUG | 357 | 95 | 43 |
| | | UAGGAUAAU | | GUAACUGGU | | | |
| 26 | 3265 | CACAGUCUCUUU | 338 | UAGUUCUAAAGA | 358 | 94.8 | 62.8 |
| | | AGAACUAAG | | GACUGUGGU | | | |
| 27 | 3228 | GGCAAGUACUGC | 339 | AAUAGCUGCAGU | 359 | 94.7 | 62.7 |
| | | AGCUAUUUG | | ACUUGCCCG | | | |
| 28 | 4779 | AGAUUGCACUCA | 340 | UUUCAAGUGAGU | 360 | 94.5 | 47.5 |
| | | CUUGAAACC | | GCAAUCUGG | | | |
| 29 | 2839 | CGUCUGAAGGAC | 341 | UAGGAGAGUCCU | 361 | 94.4 | 66.4 |
| | | UCUCCUAGG | | UCAGACGGG | | | |
| 30 | 5218 | CAGACCAGUUAC | 342 | UAGUUUGGUAAC | 362 | 94.3 | 42.3 |
| | | CAAACUAGG | | UGGUCUGCG | | | |
| 31 | 3696 | GCAGAGGGCUGA | 343 | UUGAAAGUCAGC | 363 | 94.2 | 58.2 |
| | | CUUUCAAGA | | CCUCUGCGA | | | |
| 32 | 226 | CCUUGUUGAAGA | 344 | UUUCUCCUCUUC | 364 | 94.2 | 92.2 |
| | | GGAGAAACA | | AACAAGGAG | | | |
| 33 | 3016 | CGGGUGUGUGGA | 345 | UCGCACAUCCAC | 365 | 94 | 64 |
| | | UGUGCGAGG | | ACACCCGAG | | | |
| 34 | 3782 | GGGCAAGUCCGA | 346 | UCAGUAGUCGGA | 366 | 93.9 | 56.9 |
| | | CUACUGAAA | | CUUGCCCUG | | | |
| 35 | 3456 | GAGCAAAUAUAU | 347 | UGUAUAUAUAUA | 367 | 93.9 | 59.9 |
| | | AUAUACACG | | UUUGCUCUA | | | |
| 36 | 4625 | GGGUCAGUAGAG | 348 | AAACCGACUCUA | 368 | 93.7 | 47.7 |
| | | UCGGUUUCU | | CUGACCCUG | | | |
| 37 | 4317 | CCUCUGAAUAGU | 349 | UUAACACACUAU | 369 | 93.7 | 50.7 |
| | | GUGUUAACC | | UCAGAGGGG | | | |
| 38 | 5872 | GAUACCAACUGU | 350 | UAAGGGAACAGU | 370 | 93.5 | 35.5 |
| | | UCCCUUAUA | | UGGUAUCUU | | | |
| 39 | 1430 | CCUUCUCCUACU | 351 | UUGCCGUAGUAG | 371 | 93.3 | 79.3 |
| | | ACGGCAAGC | | GAGAAGGUG | | | |
| 40 | 3706 | GACUUUCAAGAC | 352 | UCUUGAAGUCUU | 372 | 93.3 | 56.3 |
| | | UUCAAGAAC | | GAAAGUCAG | | | |
| 41 | 1877 | GUUCAGCCCUGU | 353 | UUCUGUAACAGG | 373 | 93.3 | 75.3 |
| | | UACAGAAGU | | GCUGAACAA | | | |
| 42 | 289 | GCAGGGACAGCA | 354 | UAGAUGAUGCUG | 374 | 93.1 | 91.1 |
| | | UCAUCUAUG | | UCCCUGCCG | | | |
| 43 | 3426 | UGAGAAUCAUGC | 355 | UAUUGCAGCAUG | 375 | 93 | 59 |
| | | UGCAAUAUG | | AUUCUCAUG | | | |
| 44 | 1709 | GGUUCUCUGUCA | 356 | UCGGCGAUGACA | 376 | 93 | 76 |
| | | UCGCCGAGG | | GAGAACCAG | | | |
| 45 | 3484 | AUUUCAAGAUGA | 357 | UUUAGUUUCAUC | 378 | 93 | 59 |
| | | AACUAAAGC | | UUGAAAUAU | | | |
| 46 | 2457 | GGUAUUGACGUC | 358 | UCUGACAGACGU | 379 | 93 | 69 |
| | | UGUCAGAUC | | CAAUACCUG | | | |
| 47 | 4127 | CGACCCAAACCA | 359 | UUUGGUGUGGUU | 380 | 92.9 | 51.9 |
| | | CACCAAAUG | | UGGGUCGGA | | | |
| 48 | 3446 | GAUCAUUCUAGA | 360 | UAUUUGCUCUAG | 381 | 92.8 | 58.8 |
| | | GCAAAUAUA | | AAUGAUCAU | | | |
| 49 | 1294 | GCUAAAGUCCCA | 361 | UUCCGUUUGGGA | 382 | 92.8 | 80.8 |
| | | AACGGAAGU | | CUUUAGCUU | | | |
| 50 | 4541 | UCAGGUGUUUGC | 362 | UUUGCUUGCAAA | 383 | 92.7 | 47.7 |
| | | AAGCAAAGC | | CACCUGAAG | | | |

SLC45A4 (Variant 3) Isoform 3

| siRNA_ | | | SEQ | | SEQ | | Corrected_ |
|---|---|---|---|---|---|---|---|
| id | Position | SS Sequence | ID NO: | AS Sequence | ID NO: | Score | Score |
| 1 | 2637 | GGUUUGAGCUAC | 384 | UUGCUUAGUAGC | 434 | 106.5 | 80.5 |
| | | UAAGCAAAU | | UCAAACCUA | | | |
| 2 | 1892 | AGAAGUACUUGG | 385 | UAGUUGUCCAAG | 435 | 99.2 | 81.2 |
| | | ACAACUACG | | UACUUCUGU | | | |
| 3 | 2633 | GGUAGGUUUGAG | 386 | UUAGUAGCUCAA | 436 | 98.3 | 72.3 |
| | | CUACUAAGC | | ACCUACCCU | | | |
| 4 | 2632 | GGGUAGGUUUGA | 387 | UAGUAGCUCAAA | 437 | 97.9 | 71.9 |
| | | GCUACUAAG | | CCUACCCUA | | | |
| 5 | 908 | GCAUCGACGAGG | 388 | UACUGCUCCUCG | 438 | 97 | 88 |
| | | AGCAGUACA | | UCGAUGCUG | | | |
| 6 | 1275 | GGAUAAUCACUU | 389 | UUCAUUCAAGUG | 439 | 96.1 | 84.1 |
| | | GAAUGAAGC | | AUUAUCCAG | | | |
| 7 | 827 | GCUGGUUCCGGA | 390 | UUCUGGGUCCGG | 440 | 95.4 | 87.4 |
| | | CCCAGAACC | | AACCAGCUG | | | |
| 8 | 1514 | GCAUGAGCGACC | 391 | UCGUACAGGUCG | 441 | 95.1 | 80.1 |
| | | UGUACGACA | | CUCAUGCUG | | | |
| 9 | 1424 | CCAGCACCUUCU | 392 | UAGUAGGAGAAG | 442 | 95 | 81 |
| | | CCUACUACG | | GUGCUGGAG | | | |
| 10 | 226 | CCUUGUUGAAGA | 393 | UUUCUCCUCUUC | 443 | 94.2 | 92.2 |
| | | GGAGAAACA | | AACAAGGAG | | | |
| 11 | 2638 | GUUUGAGCUACU | 394 | UUUGCUUAGUAG | 444 | 93.5 | 67.5 |
| | | AAGCAAAUA | | CUCAAACCU | | | |
| 12 | 1430 | CCUUCUCCUACU | 395 | UUGCCGUAGUAG | 445 | 93.3 | 79.3 |
| | | ACGGCAAGC | | GAGAAGGUG | | | |
| 13 | 1877 | GUUCAGCCCUGU | 396 | UUCUGUAACAGG | 446 | 93.3 | 75.3 |
| | | UACAGAAGU | | GCUGAACAA | | | |
| 14 | 289 | GCAGGGACAGCA | 397 | UAGAUGAUGCUG | 447 | 93.1 | 91.1 |
| | | UCAUCUAUG | | UCCCUGCCG | | | |
| 15 | 1709 | GGUUCUCUGUCA | 398 | UCGGCGAUGACA | 448 | 93 | 76 |
| | | UCGCCGAGG | | GAGAACCAG | | | |
| 16 | 1294 | GCUAAAGUCCCA | 399 | UUCCGUUUGGGA | 449 | 92.8 | 80.8 |
| | | AACGGAAGU | | CUUUAGCUU | | | |
| 17 | 1261 | GACGAGACCUUG | 400 | UAUCCAGCAAGG | 450 | 92.4 | 80.4 |
| | | CUGGAUAAU | | UCUCGUCCU | | | |
| 18 | 2648 | CUAAGCAAAUAC | 401 | UAGUGUGGUAUU | 451 | 92.3 | 66.3 |
| | | CACACUAAC | | UGCUUAGUA | | | |
| 19 | 1484 | ACGCCGUGGUGC | 402 | UUGAUCAGCACC | 452 | 91.6 | 77.6 |
| | | UGAUCAAGC | | ACGGCGUUG | | | |
| 20 | 1511 | GCAGCAUGAGCG | 403 | UACAGGUCGCUC | 453 | 91.5 | 76.5 |
| | | ACCUGUACG | | AUGCUGCGC | | | |
| 21 | 1757 | AGGUCAUCUUCG | 404 | UCGCCUUCGAAG | 454 | 91.1 | 74.1 |
| | | AAGGCGACC | | AUGACCUGG | | | |
| 22 | 1004 | CAGACGAGGUAC | 405 | UCCGACUGUACC | 455 | 91 | 81 |
| | | AGUCGGAGC | | UCGUCUGGG | | | |
| 23 | 1880 | CAGCCCUGUUAC | 406 | UACUUCUGUAAC | 456 | 90.9 | 72.9 |
| | | AGAAGUACU | | AGGGCUGAA | | | |
| 24 | 403 | GAGCAGUACUAC | 407 | UGAGGCUGUAGU | 457 | 90.9 | 86.9 |
| | | AGCCUCACC | | ACUGCUCCG | | | |
| 25 | 1272 | GCUGGAUAAUCA | 408 | AUUCAAGUGAUU | 458 | 90.4 | 78.4 |
| | | CUUGAAUGA | | AUCCAGCAA | | | |
| 26 | 1523 | ACCUGUACGACA | 409 | UUCUGCAUGUCG | 459 | 90.3 | 75.3 |
| | | UGCAGAAGC | | UACAGGUCG | | | |
| 27 | 1287 | GAAUGAAGCUAA | 410 | UGGGACUUUAGC | 460 | 90.2 | 78.2 |
| | | AGUCCCAAA | | UUCAUUCAA | | | |
| 28 | 355 | GUGAGUCCACUG | 411 | UUGACCUCAGUG | 461 | 89.7 | 86.7 |
| | | AGGUCAAAU | | GACUCACAA | | | |
| 29 | 1281 | UCACUUGAAUGA | 412 | UUUAGCUUCAUU | 462 | 89.6 | 77.6 |
| | | AGCUAAAGU | | CAAGUGAUU | | | |
| 30 | 1265 | AGACCUUGCUGG | 413 | UGAUUAUCCAGC | 463 | 88.8 | 76.8 |
| | | AUAAUCACU | | AAGGUCUCG | | | |
| 31 | 2629 | GUAGGGUAGGUU | 414 | UAGCUCAAACCU | 464 | 88.7 | 62.7 |
| | | UGAGCUACU | | ACCCUACGA | | | |
| 32 | 1882 | GCCCUGUUACAG | 415 | AGUACUUCUGUA | 465 | 88.5 | 70.5 |
| | | AAGUACUUG | | ACAGGGCUG | | | |
| 33 | 1883 | CCCUGUUACAGA | 416 | AAGUACUUCUGU | 466 | 88.4 | 70.4 |
| | | AGUACUUGG | | AACAGGGCU | | | |
| 34 | 1517 | UGAGCGACCUGU | 417 | AUGUCGUACAGG | 467 | 88.4 | 73.4 |
| | | ACGACAUGC | | UCGCUCAUG | | | |
| 35 | 1121 | AGCUGCUGUUCC | 418 | UCGUGCAGGAAC | 468 | 88.2 | 77.2 |
| | | UGCACGACA | | AGCAGCUCG | | | |
| 36 | 1346 | ACACCAGGGUGG | 419 | UUCGUGUCCACC | 469 | 88.2 | 75.2 |
| | | ACACGAAGC | | CUGGUGUAG | | | |
| 37 | 340 | CGUCCAAAGACC | 420 | UCACAAGGGUCU | 470 | 87.9 | 84.9 |
| | | CUUGUGAGU | | UUGGACGGG | | | |
| 38 | 331 | GGGCUGUCCCGU | 421 | UCUUUGGACGGG | 471 | 87.8 | 84.8 |
| | | CCAAAGACC | | ACAGCCCUG | | | |
| 39 | 1454 | GGUCCCACUGCU | 422 | UAGCGGUAGCAG | 472 | 87.7 | 73.7 |
| | | ACCGCUACC | | UGGGACCCA | | | |
| 40 | 1732 | GUGUUCUACACC | 423 | UGAAGUCGGUGU | 473 | 87.6 | 70.6 |
| | | GACUUCAUG | | AGAACACGG | | | |
| 41 | 902 | UGUUCAGCAUCG | 424 | UCCUCGUCGAUG | 474 | 87.6 | 78.6 |
| | | ACGAGGAGC | | CUGAACAGG | | | |
| 42 | 1733 | UGUUCUACACCG | 425 | AUGAAGUCGGUG | 475 | 87.4 | 70.4 |
| | | ACUUCAUGG | | UAGAACACG | | | |
| 43 | 375 | UCAAUGGAGUCU | 426 | UUUCUGAAGACU | 476 | 87 | 84 |
| | | UCAGAAAGG | | CCAUUGAUU | | | |
| 44 | 1448 | AGCUUGGGUCCC | 427 | UAGCAGUGGGAC | 477 | 86.7 | 72.7 |
| | | ACUGCUACC | | CCAAGCUUG | | | |
| 45 | 1727 | AGGCCGUGUUCU | 428 | UCGGUGUAGAAC | 478 | 86.5 | 69.5 |
| | | ACACCGACU | | ACGGCCUCG | | | |
| 46 | 1262 | ACGAGACCUUGC | 429 | UUAUCCAGCAAG | 479 | 86 | 74 |
| | | UGGAUAAUC | | GUCUCGUCC | | | |
| 47 | 1916 | UGAGCGUCAGGG | 430 | UAGAUCACCCUG | 480 | 86 | 67 |
| | | UGAUCUACG | | ACGCUCAGG | | | |
| 48 | 364 | CUGAGGUCAAAU | 431 | UCCAUUGAUUUG | 481 | 85.9 | 82.9 |
| | | CAAUGGAGU | | ACCUCAGUG | | | |
| 49 | 2535 | GGAGCAGAGACA | 432 | UCCGCGGUGUCU | 482 | 85.7 | 60.7 |
| | | CCGCGGAAC | | CUGCUCCCG | | | |
| 50 | 830 | GGUUCCGGACCC | 433 | UGGUUCUGGGUC | 483 | 85.5 | 77.5 |
| | | AGAACCAGG | | CGGAACCAG | | | |

In some examples, the nucleic acid is a mRNA.

In some examples, the nucleic acid is a mRNA and comprises the sequence selected from the group consisting of

>NM_152379.4 Clorf131, transcript variant 1, mRNA
(SEQ ID NO: 488)
GCGGAACCCGGAAGCGGGGGTGCAGCGCGGCAGAATGAGGGTTGATTCCTCGGCTGACCCCACAATGTCG
CAGGAGCAAGGGCCGGGGTCCTCCACGCCTCCCAGTTCTCCGACACTTCTTGACGCTCTGCTCCAGAACC
TTTACGACTTTGGAGGTACAGAAGGTGAAACAGAACAGAAGAAGATCATAAAGAAAAGGGAAAACAAGAA
GAGAGATGTGATGGCTTCAGCGGCCTTGGCAGCAGAGCCATCTCCCCTACCTGGTTCTCTCATAAGAGGC
CAGAGGAAGAGCGCTTCGAGCTTCTTCAAGGAACTTAGAGAAGAGCGGCATTGTGCTCCTTCTGGGACCC
CCACAGGACCAGAGATCCTTGCTGCTGCAGTTCCTCCCTCTTCCCTAAAGAACAATAGGGAACAAGTAGA
AGTGGTAGAATTTCACAGCAATAAAAAAAGAAAATTGACGCCAGATCATAACAAGAACACAAAGCAGGCT
AATCCTAGTGTTTTGGAGAGAGATGTGGATACACAAGAATTTAACCTAGAAAAAGCTCGTTTAGAAGTGC
ACCGGTTTGGTATCACGGGTTATGGAAAAGGAAAGGAGAGAATCCTGGAACAGGAACGTGCCATTATGCT
GGGCGCTAAGCCTCCTAAAAAGAGTTATGTGAATTACAAGGTTTTACAGGAGCAAATTAAAGAAAAAAAG
GCAGCAAAGGAAGAAGAAAAGAGACTGGCCCAAGAAACAGATATTTTCAAGAAAAAGAAGAGGAAAGGAC
AGGAGGACAGGAAATCCAAAAAGAAGTCCGCTCCCAGTATTTTGTCAAATGGACGGATTGGACAGGTTGG
AAAATTCAAAAATGGAACACTGATTCTGAGCCCAGTTGATATCAAGAAAATAAATTCTTCCAGAGTGGCC
AAATGAAGTACTTTGTCAAATAAAACGAGGATGGGAACAACTGAGTCAACTGCAGCGGCCCTGGACCCTG
TGCGACTCTAGATAGATCGTTCTTAACACGCATTTCACAGACTGCTTTTTCATTTTAGAAAAAAAGTCAA
ACAGAAGTAACATTTCTAGTTGCCGAAGTGTCTGCTTCATGGTGCTGAACGTGTCAGGCCCAGTGCAGCA
TGAATGTGAAGTGAACCCACTGAAAAGTGATTTTTAATGTTCTCCAGCAAACTTCACTCCTGTTTCATGT
TCACTGGTATTTGGTGCATTTGCTTGTAATACAGTTTTTTATAACTTTACTGTTTTGTAAATGCTAGTAG
TTTACATTAAATTGCCTTATATTTTTAATGATTTAATGTTATGATATCATGAAAGAACCAAGAATCTAGT
AATTTATCAGTTTTGTGGAAAAGCAGTTTTAATTTTTAGCCTACCATGTGTACAAAATCTACTTCATAAG
AAGTTATATAAATAAAATAGGTTTGTAGTA
>NM_001300830.2 Clorf131, transcript variant 2, mRNA
(SEQ ID NO: 489)
GCGGAACCCGGAAGCGGGGGTGCAGCGCGGCAGAATGAGGGTTGATTCCTCGGCTGACCCCACAATGTCG
CAGGAGCAAGGGCCGGGGTCCTCCACGCCTCCCAGTTCTCCGACACTTCTTGACGCTCTGCTCCAGAACC
TTTACGACTTTGGAGGTACAGAAGGTGAAACAGAACAGAAGAAGATCATAAAGAAAAGGGAAAACAAGAA
GAGAGATGTGATGGCTTCAGCGGCCTTGGCAGCAGAGCCATCTCCCCTACCTGGTTCTCTCATAAGAGGC
CAGAGGAAGAGCGCTTCGAGCTTCTTCAAGGAACTTAGAGAAGAGCGGCATTGTGCTCCTTCTGGGACCC
CCACAGGACCAGAGATCCTTGCTGCTGCAGTTCCTCCCTCTTCCCTAAAGAACAATAGGGAACAAGTAGA
AGTGGTAGAATTTCACAGCAATAAAAAAAGAAAATTGACGCCAGATCATAACAAGAACACAAAGGCTAAT
CCTAGTGTTTTGGAGAGAGATGTGGATACACAAGAATTTAACCTAGAAAAAGCTCGTTTAGAAGTGCACC
GGTTTGGTATCACGGGTTATGGAAAAGGAAAGGAGAGAATCCTGGAACAGGAACGTGCCATTATGCTGGG
CGCTAAGCCTCCTAAAAAGAGTTATGTGAATTACAAGGTTTTACAGGAGCAAATTAAAGAAAAAAAGGCA
GCAAAGGAAGAAGAAAAGAGACTGGCCCAAGAAACAGATATTTTCAAGAAAAAGAAGAGGAAAGGACAGG
AGGACAGGAAATCCAAAAAGAAGTCCGCTCCCAGTATTTTGTCAAATGGACGGATTGGACAGGTTGGAAA
ATTCAAAAATGGAACACTGATTCTGAGCCCAGTTGATATCAAGAAAATAAATTCTTCCAGAGTGGCCAAA
TGAAGTACTTTGTCAAATAAAACGAGGATGGGAACAACTGAGTCAACTGCAGCGGCCCTGGACCCTGTGC
GACTCTAGATAGATCGTTCTTAACACGCATTTCACAGACTGCTTTTTCATTTTAGAAAAAAAGTCAAACA
GAAGTAACATTTCTAGTTGCCGAAGTGTCTGCTTCATGGTGCTGAACGTGTCAGGCCCAGTGCAGCATGA
ATGTGAAGTGAACCCACTGAAAAGTGATTTTTAATGTTCTCCAGCAAACTTCACTCCTGTTTCATGTTCA
CTGGTATTTGGTGCATTTGCTTGTAATACAGTTTTTTATAACTTTACTGTTTTGTAAATGCTAGTAGTTT
ACATTAAATTGCCTTATATTTTTAATGATTTAATGTTATGATATCATGAAAGAACCAAGAATCTAGTAAT
TTATCAGTTTTGTGGAAAAGCAGTTTTAATTTTTAGCCTACCATGTGTACAAAATCTACTTCATAAGAAG
TTATATAAATAAAATAGGTTTGTAGTA
>NM_01286646.2 SLC45A4, transcript variant 1, mRNA
(SEQ ID NO: 490)
GCAGCCTCGCAGCGCTCTCTCCGCGCCCGCGTCGCTGACTGACCGCCCGGCCGGCCGGCCGAGGGAGCAG
CCCCCGCCCGGACCCGCAGCCGCCTCCCGACCCCGGTGCGCCCGGGGCTCCGCGCCCCCGTAGCCCCTGC
CCGGCCCGGCCCGGCCGCCCCGCCGCCCCCGCGGCCCCGGCCGGAGGAGACAGATCATCCAGTCACTCGA
TGGGGATTACCAGCGGACCACCTGTTTGTATCACCTTCCCACCTCTCTGTAGCAAGAAAAATCACTTCAG
CACCTCTTCAGTAATAATGATGAAACTGGTATTAACTGAATGGCAATTATGGATTTTTAACTCTAATTCA
CAGTAAACCAGCAAGCCATATGTTTAAAATCCAACCAAAAATCGTTCTGTGTTGCTGCATGTCTCATACC
GTTGTGAGAAGTIGTGAGATAGCCACTTGTGCAAAGAGGAATGTAAGTGTTACCTGCCCGAGGAACACAG
CAGAAGCAGAAAGAGAAGACGTCTTAATTTATCATTACAGATATATGAAAGCATTATTTATATAGATAGA
ATATATAGATAAATACATATATTTTTGGTGGTAATGAAAATGGCTCCGCAGAATGCCGACCCGGAATCTA
TGCAAGTTCAAGAGTTATCCGTGCCCCTGCCGGACCCGCAGAAAGCCGGAGGCGCAGAGGCCGAGAACTG
CGAGACCATCAGCGAGGGGTCCATAGACCGAATCCCCATGCGCCTGTGGGTGATGCACGGGGCGGTGATG
TTTGGCAGGGAGTTCTGTTACGCCATGGAAACCGCTCTGGTCACACCAATACTGTTGCAGATTGGCCTTC
CGGAGCAGTACTACAGCCTCACCTGGTTCCTGAGCCCCATCCTTGGCCTCATCTTCACACCTCTCATTGG
GTCTGCGAGTGACCGGTGCACCCTGAGCTGGGGCCGCCGGCGGCCCTTCATCCTCGCCCTCTGCGTTGGC
GTCCTCTTTGGCGTTGCACTTTTCCTTAACGGCTCTGCCATCGGTCTGGCCCTCGGCGATGTCCCCAACC
GGCAGCCCATTGGCATCGTGCTCACGGTGCTGGGAGTGGTGGTCCTGGACTTCAGCGCCGATGCCACCGA
GGGGCCCATCCGTGCCTATCTGCTGGACGTGGTGGACAGCGAGGAGCAGGACATGGCCCTCAACATCCAC
GCCTTCTCTGCCGGCCTCGGCGGAGCCATCGGCTACGTGCTGGGTGGGCTGGACTGGACCCAGACCTTCC
TGGGCAGCTGGTTCCGGACCCAGAACCAGGTGCTCTTCTTCTTTGCCGCCATCATCTTCACGGTGTCCGT
GGCCCTGCACCTGTTCAGCATCGACGAGGAGCAGTACAGCCCGCAGCAGGAGCGCAGCGCTGAGGAGCCC
GGCGCCCTGGATGGGGGCGAGCCGCACGGCGTCCCTGCCTTCCCAGACGAGGTACAGTCGGAGCACGAGC
TGGCCCTGGACTACCCGGACGTGGACATCATGCGCAGCAAAAGCGACTCGGCATTGCACGTGCCGGACAC
CGCGCTGGACCTGGAGCCCGAGCTGCTGTTCCTGCACGACATCGAGCCCTCCATCTTCCACGACGCCTCC
TACCCCGCCACCCCCCGCAGCACCAGCCAGGAGCTCGCCAAGACCAAGCTGCCCCGCCTGGCCACCTTCC
TCAAGGAAGCCGCCAAGGAGGACGAGACCTTGCTGGATAATCACTTGAATGAAGCTAAAGTCCCAAACGG
AAGTGGCTCCCCCACAAAAGACGCCCTCGGCGGCTACACCAGGGTGGACACGAAGCCCTCGGCCACGTCG
AGCTCCATGCGGCGGCGGCGGCACGCGTTCCGCAGGCAGGCCTCCAGCACCTTCTCCTACTACGGCAAGC
TTGGGTCCCACTGCTACCGCTACCGGCGCGCCAACGCCGTGGTGCTGATCAAGCCGTCGCGCAGCATGAG
CGACCTGTACGACATGCAGAAGCGGCAGCGGCAGCACCGGCACCGGAACCAGAGCGGGGCCACCACCTCC
AGCGGGGACACCGAGAGTGAGGAGGGGGAGGGCGAGACCACGGTGCGCCTGCTGTGGCTCTCCATGCTGA
AGATGCCCAGGGAGCTGATGCGGCTGTGCCTCTGCCACCTCCTCACCTGGTTCTCTGTCATCGCCGAGGC
CGTGTTCTACACCGACTTCATGGGCCAGGTCATCTTCGAAGGCGACCCCAAGGCCCCCTCGAACTCGACC
GCCTGGCAAGCCTACAACGCCGGGGTCAAGATGGGCTGCTGGGGCCTGGTCATTTATGCCGCCACTGGTG
CTATTTGTTCAGCCCTGTTACAGAAGTACTTGGACAACTACGACCTGAGCGTCAGGGTGATCTACGTGCT
GGGGACGCTGGGCTTCTCTGTCGGCACAGCCGTGATGGCCATGTTTCCCAACGTCTACGTCGCCATGGTC
ACCATCAGCACCATGGGCATCGTCTCCATGAGCATCTCCTACTGCCCGTACGCCCTGCTGGGCCAGTACC
ATGACATCAAGCAGTACATCCACCACAGCCCCGGGAACTCCAAGCGAGGGTTTGGCATAGATTGTGCCAT
CCTGTCCTGCCAAGTGTACATCTCGCAGATCCTGGTGGCCTCTGCCCTTGGGGGCGTGGTCGACGCCGTG
GGGACTGTCCGCGTCATCCCCATGGTGGCCTCTGTGGGCTCTTTCCTGGGCTTCCTGACGGCCACATTCC
TGGTGATCTATCCCAACGTGTCAGAGGAGGCCAAGGAGGAGCAGAAAGGCCTGTCTTCCCCGTTGGCCGG
CGAAGGCAGGGCCGGTGGGAACAGCGAAAAGCCCACCGTGCTGAAGCTCACGCGGAAGGAGGGCCTGCAG
GGACCGGTGGAGACAGAGTCCGTGACGCCTGCAGGTATTGACGTCTGTCAGATCTCGTCACATTGGCTGG
TGCCGCAGCTGTTGGAGAGTATTTTTCTTTATGATTATTTTAGAAAAAAAATTTTCTTTTCCACAATGTG
GTTCTCTTAGAAGAATGACGTATCTTCTTTTCCTCAGCGAGTTGGACACATTGTGCCCAGGGCAGCCCTG
TCCTTGGGCAGCGACCGCACACCAAAGCTGGGAGGAGGCTGGTCCGGGGGGCCTGGGCAGAAGACAGTGA
TTTGCAGGGGTGGCTCCCAGACACCCTGCCCAGGGATGGGCTGGGCACCACCTGGGGGCGGAGCGTGAGC
TCCAGACGAGCTCCTGCGTGCGCGTGTGAGTGTGTCTGCGCCCAGCCATGTGACCCCGCTCGTCCCGTCT
GAAGGACTCTCCTAGGAGGCCAGGTTGCCCCTCCAGACCGCTCCCAACGTCAGGGGGAAGGAAACGTTGA
CTTTCACTGCACTTTGATTCGTCTCTAAACCATTTGCTGGGGATTCCTGAGAGCAGAGCTCCCAGCGGGC
CCTGCCTCCCAAGTCCCGCCGCAAGGCTACCTCGGGTGTGTGGATGTGCGAGGGCCTCCCCCGCTTGCGA
AGGGGACATGCGTGCTGGAACCTGTCGGAACTCCATGCCTTCCTCGCCTGCTCACCTGCTCGACGCTGGA
ATCGGGACAGGTGCAAAGGGACGCAGACGTCTGGGACAGCTAAGGCCCGTGTCACCGGAGGGCTCCGCAC
AGTCGTTCTGGTTTCAACGAATAAGCAAAACTCGGGCAAGTACTGCAGCTATTTGGAAATGTTTTCCAAA
CCACAGTCTCTTTAGAACTAAGCCTATTTGAAACGGTCGGTGTAGGCTTACTGAGATCAGGAGACAGGGA
GGCCCCGCACATCACACAGATAAAGTCAGACAATTGTAATTAATACTTTTGCTGCCTCAAGTTGTTTTTT
AAATAAAGTACTTTGAAATGCATGAGAATCATGCTGCAATATGATCATTCTAGAGCAAATATATATATAC
ACGTATATATATTTCAAGATGAAACTAAAGCAGTTTTTAAATAAATTACTTGAATTTTCTGTGTATTTAA
AGGAACGACTGTTTAATGTACTTGATGGGCCTCTGGTCTTGCCGTGTCTCCTGCCGCTGGTGGCACTTTG
TAGATTGTGTGTTTGTGTCCGGGTGGCAGTTGGGTACCTGCTCACGCACGGTGTGTCTGCCAGGCCACGG
TGTCCCAGGATCGCAGAGGGCTGACTTTCAAGACTTCAAGAACATTTTCTGGATGTGTGGAAACTTGAGA
ATGGCCTTGTGAATCTCGTGCTTGGACAGGGCAAGTCCGACTACTGAAAGTGCTGCCAGCTTTGCTGCGA
GCCCTCCGGCCAGCGGGAGCCCCGTGGGCTGGGCACTGTGGCCCTTCTTCTCTGGGGGACGGCACCCCTG
GCTTCCTCACCTCGGCCGGGCGTCCGTGGCAGCTCACTCTATGCAACTTGATCCTCTAGCGGCTTTAAGA
CTGTAGATCCCCTCTCTGAGACCTGGCTGTACTTGTCAGGATCTCGAGGCGCAGCTCCCGTCTTAGCTGG
TTTCTCCGGCTTCTCGTCCTGACGACTATAAAACAGTTGGAGGCAAGAAAGCAGCGGATGTGGGGTGGCA
GTGGCCTGACCCGAATCAAGATCCGACCCAAACCACACCAAATGTGGGTTCATCTGGGGGCCACCCCTTG
CCTGAGGCTTCCCACCCTCATCTGAAGGCCCAGGGGCCGGATCCAGGGCTCACCAAAGCCGATTCCTCGC
CAGCTGGGAGTGCAGAAGTCTCAGGGCCTGGCTGCGACTTGATTTTTAGGAGGAAGAGGGGCTTCGCAAC
CCCCCTCTGAATAGTGTGTTAACCCTTGAGATCCCAGCCTCGACTAATCTGAAGTAAGGACAACAAAGGC
CATTCAGTGCCCTCCACATGGCCTTGCCACAGTCACTGTCAGGGTATGAACGTGCCGGAAGCCAGTGCCA
GCCAGGGACAGGCGTGACTGTTGTGTGCTCCTCGGTGACAGGAGTCGGTGGCTGCACACTTTGTAGACTC
GTCAAGCTGTCAGCACTTCAGGTGTTTGCAAGCAAAGCCCTTCTTAGTGTGCAGGTCAGTGTGCAGAGCC
CAAATGAGGGGACCGCAGGGGCTGGGGTCCAGGGTCAGTAGAGTCGGTTTCTGGAGCTGCCTTCCTGGGA
GGCAGGTGTGGGTGACCGGGGCTCTGGCGGTGCGTGTGGGCCCGGCCTGGCCACAGCGGGGACCAGGTCA
CGACATCTTTGGCCTCAACCCCTCCCCTGCACTGAGATTATTTCCAGATTGCACTCACTTGAAACCGTCC
GTGTCGTCACCTTGTGTCTTAAGGGAAGCCGAGAAGAAGGGCAGACGGGCAGGCTGTCTTGTCTGCAAAG
CGCCATTGCGCCCGCAGCTTGTGTGGGTCAGGCTGCAGCTCGGGTGTCTGTGGCTTCTAACCTTGTACCT
CAGACGGATGCAGTAACAAGGCGGGGCTGGGGACGCCGGTCAGTGTCAAAGGGGAGGTGCTCTGGCTGAT
AGCCTTGCCCGAGAGGGACGAGGAGGCCGTGCGGGTGCCCCCTGGGGAAGCTGGCCAGCCATCCAGTGCT
GAGGACGCAGCTGGAGTTGGGCTCGTGGCACCCTTGGGGTGTGGGCTGTGCAGGCTGGTGGCCTGGGTGC
CTCTGCACACTGAGTGGAGTGTCAGGCAGGGTGTGTCGGTTGGTCGAGTCTTGTGTAATGTGCGCAGACC
AGTTACCAAACTAGGATAATGTTGGTCTCATTTGTGGTGGTTTTGTTCCCTATACAAGTCAGCTAAGTAA
AGACTCTTTTAACGAGCTTCCCCTTAACACATGGCAGAAGTTTCCAGGTGCAGGAATGCGAGCTGGCGGG
AAGGGGCAGAGGCCGTGAGCTCTCAGCTGGGCCGGCCTGCCTGTGTCCCCTTTCCTGGGTCTGTCGGCAG
ACTGGCATCATGACGTTCCCTGGTGGCTGAAGAGCTAGCTTTGGAGTGTTGTTTTTCTCACTCTCAGGCA
GGGGCCTTAGCTGGAATCCTCCAACCTGCCACTGAACACGTCAGTGCTGTGTGCTGCCTCTTGGACACCT
GCCCTTGAAAGCCTCAGGCCCCTGGGAGAAGCACTCTGTCCAGTCCTGTCCCCGGGGGGGAGGCAGGGCC
ACTGAGCCCTCCTCAGATGGTTAGTGGCTTCCAACAGCCATCAGGAGTGTTTCTTGAATGCCCCAGGTGT
GGAGGACTTGGTCTGTGACCACCTAGAACCCCAGAGCTGAACAGGAAGCCGTCCCTGCAGCAACAAGAGG
GCTGGAAGGGGGAGCTGCAGGCCACCCTCGGCTCTCCCACTGCTGGGGCGGTGATGTTCGGGTGACATGT
TTGAAAAATACTCTTAAAGATACCAACTGTTCCCTTATATGGCTAATGGTTTGTGCAGCCACCAGCGATG
GCGGCCCCTATTAGAGACCAGGTTTGTTAAAACACCAAATATTGCTGTCCACACTAGACATTAACCGGCT
TCAGAAAAGATGGACACCTTTTCCCACGCTGTTTCGCTTCTTAACTTTGGTCCAGCTTTAGCCACCACAC
AGCGTGTGAGGGACTGCTGCTGCGGAGTCAGCCTCGTTTGTCCCTCCGCCTCCCACCAGCACGCGCCGCT
TCTGAGAGACACCAGCTCCCTGCCTCCAAGCCTGGTGCCACAGGCCTGTCGTGAGGGACCCCTGCTTCCG
AGAGCTCCTGGGGGGGTTCTGCCCTTCACCACCTGGGAGAGGTGTCAGTTCAGTTCCGAGTTGAACAAGG
CCCGTGCACACAGCATGTTGGGGGCCCAGCCCAAAGTTCTTGTCACCTCCTCATGCAAAGCCAGCCATCA
CCCTCCGGCCAGAGCTCAAGGTGGCCCCTTGGCCAGCCCCTCCTTGGGTCCTCCAGGAGGACTGAGCACC
CCTCCTAGCGGCATCCCTTGCCCTCCACAGTGCTGCCAGGGGCACGTCGCTCTGTGCCGTGGACTGAGAC
CATCCCCTGGTGACAGAATGACCCGTTTGTTGGAAATGCCTCGTTGCCAGAGAAACTCCCCAGGCATCTC
GGAACGAAACTATTTAGTTCCATTGTGAACTGGCCACGGGACAGCTTTTTATCAACTTATTAAGTTGGAG
CACTGTAATCGCGCTTGCTGAGTTAGCAGTGGTGGTAAGCGTGTGTTAAACACATAATGTTACGTTTTAG
GAGAGAGAGGTCGTAAGGAAGTGTCGTGTCGCTCATGACTCTCTTCTATTAGTTGGGTAACAGTGGCCTC
ATGTTTGTGTCTGTGTGTACACAGAGCCCTTAGGTTCTGCTCTGTTTCTTTGCCAGGTGAATGTTTGTGG
CATGCGCTGCTGTCCGCGCCCCTCTGTCCTGCGCAGGGTTCAGCTGTGCGGCGCCCTGATTTCCTCCATG
CACACAGAACCTCCTTGTGTCTGTTTCTCTGTTCCTCTGTGGCTGACTCAATAAACTTTTCCCTCTGA
>NM_001080431.3 SLC45A4, transcript variant 2, mRNA
(SEQ ID NO: 491)
TGATAGGGCAGAGAGGAGCCCATGCTCCAGCTGCGGGGAGTGCCTGCCCGAGGAGCTGGCCAGTGAAGAC
AGGTGCCTGCCAGTGAAGACAGGTGCCCACAAGCTGGACACAGGGTCGGCCCTGAGACAGGACAGCAGCT
GCGGAATGTTCCCGCTGGCTTTCTGGAGCGCCAGCGTCCTGCTTGGGTCTGGCCAGGGGCCTCTAGCCAG
GCTGTTGAGGCGCCCCTCCTTGTTGAAGAGGAGAAACAGTGGCCCCCTGGGAGTTGCTGGGAGGTGCCTG
GCACAGGGCGGCAGGGACAGCATCATCTATGGGGAAAGCAAGCCCAGCCTCAGGGCTGTCCCGTCCAAAG
ACCCTTGTGAGTCCACTGAGGTCAAATCAATGGAGTCTTCAGAAAGGCCTTCCGGAGCAGTACTACAGCC
TCACCTGGTTCCTGAGCCCCATCCTTGGCCTCATCTTCACACCTCTCATTGGGTCTGCGAGTGACCGGTG
CACCCTGAGCTGGGGCCGCCGGCGGCCCTTCATCCTCGCCCTCTGCGTTGGCGTCCTCTTTGGCGTTGCA
CTTTTCCTTAACGGCTCTGCCATCGGTCTGGCCCTCGGCGATGTCCCCAACCGGCAGCCCATTGGCATCG
TGCTCACGGTGCTGGGAGTGGTGGTCCTGGACTTCAGCGCCGATGCCACCGAGGGGCCCATCCGTGCCTA
TCTGCTGGACGTGGTGGACAGCGAGGAGCAGGACATGGCCCTCAACATCCACGCCTTCTCTGCCGGCCTC
GGCGGAGCCATCGGCTACGTGCTGGGTGGGCTGGACTGGACCCAGACCTTCCTGGGCAGCTGGTTCCGGA
CCCAGAACCAGGTGCTCTTCTTCTTTGCCGCCATCATCTTCACGGTGTCCGTGGCCCTGCACCTGTTCAG
CATCGACGAGGAGCAGTACAGCCCGCAGCAGGAGCGCAGCGCTGAGGAGCCCGGCGCCCTGGATGGGGGC
GAGCCGCACGGCGTCCCTGCCTTCCCAGACGAGGTACAGTCGGAGCACGAGCTGGCCCTGGACTACCCGG
ACGTGGACATCATGCGCAGCAAAAGCGACTCGGCATTGCACGTGCCGGACACCGCGCTGGACCTGGAGCC
CGAGCTGCTGTTCCTGCACGACATCGAGCCCTCCATCTTCCACGACGCCTCCTACCCCGCCACCCCCCGC
AGCACCAGCCAGGAGCTCGCCAAGACCAAGCTGCCCCGCCTGGCCACCTTCCTCAAGGAAGCCGCCAAGG
AGGACGAGACCTTGCTGGATAATCACTTGAATGAAGCTAAAGTCCCAAACGGAAGTGGCTCCCCCACAAA
AGACGCCCTCGGCGGCTACACCAGGGTGGACACGAAGCCCTCGGCCACGTCGAGCTCCATGCGGCGGCGG
CGGCACGCGTTCCGCAGGCAGGCCTCCAGCACCTTCTCCTACTACGGCAAGCTTGGGTCCCACTGCTACC
GCTACCGGCGCGCCAACGCCGTGGTGCTGATCAAGCCGTCGCGCAGCATGAGCGACCTGTACGACATGCA
GAAGCGGCAGCGGCAGCACCGGCACCGGAACCAGAGCGGGGCCACCACCTCCAGCGGGGACACCGAGAGT
GAGGAGGGGGAGGGCGAGACCACGGTGCGCCTGCTGTGGCTCTCCATGCTGAAGATGCCCAGGGAGCTGA
TGCGGCTGTGCCTCTGCCACCTCCTCACCTGGTTCTCTGTCATCGCCGAGGCCGTGTTCTACACCGACTT
CATGGGCCAGGTCATCTTCGAAGGCGACCCCAAGGCCCCCTCGAACTCGACCGCCTGGCAAGCCTACAAC
GCCGGGGTCAAGATGGGCTGCTGGGGCCTGGTCATTTATGCCGCCACTGGTGCTATTTGTTCAGCCCTGT
TACAGAAGTACTTGGACAACTACGACCTGAGCGTCAGGGTGATCTACGTGCTGGGGACGCTGGGCTTCTC
TGTCGGCACAGCCGTGATGGCCATGTTTCCCAACGTCTACGTCGCCATGGTCACCATCAGCACCATGGGC
ATCGTCTCCATGAGCATCTCCTACTGCCCGTACGCCCTGCTGGGCCAGTACCATGACATCAAGCAGTACA
TCCACCACAGCCCCGGGAACTCCAAGCGAGGGTTTGGCATAGATTGTGCCATCCTGTCCTGCCAAGTGTA
CATCTCGCAGATCCTGGTGGCCTCTGCCCTTGGGGGCGTGGTCGACGCCGTGGGGACTGTCCGCGTCATC
CCCATGGTGGCCTCTGTGGGCTCTTTCCTGGGCTTCCTGACGGCCACATTCCTGGTGATCTATCCCAACG
TGTCAGAGGAGGCCAAGGAGGAGCAGAAAGGCCTGTCTTCCCCGTTGGCCGGCGAAGGCAGGGCCGGTGG
GAACAGCGAAAAGCCCACCGTGCTGAAGCTCACGCGGAAGGAGGGCCTGCAGGGACCGGTGGAGACAGAA
CGCCTGCAGGTATTGACGTCTGTCAGATCTCGTCACATTGGCTGGTGCCGCAGCTGTTGGAGAGTATTTT
TCTTTATGATTATTTTAGAAAAAAAATTTTCTTTTCCACAATGTGGTTCTCTTAGAAGAATGACGTATCT
TCTTTTCCTCAGCGAGTTGGACACATTGTGCCCAGGGCAGCCCTGTCCTTGGGCAGCGACCGCACACCAA
AGCTGGGAGGAGGCTGGTCCGGGGGGCCTGGGCAGAAGACAGTGATTTGCAGGGGTGGCTCCCAGACACC
CTGCCCAGGGATGGGCTGGGCACCACCTGGGGGCGGAGCGTGAGCTCCAGACGAGCTCCTGCGTGCGCGT
GTGAGTGTGTCTGCGCCCAGCCATGTGACCCCGCTCGTCCCGTCTGAAGGACTCTCCTAGGAGGCCAGGT
TGCCCCTCCAGACCGCTCCCAACGTCAGGGGGAAGGAAACGTTGACTTTCACTGCACTTTGATTCGTCTC
TAAACCATTTGCTGGGGATTCCTGAGAGCAGAGCTCCCAGCGGGCCCTGCCTCCCAAGTCCCGCCGCAAG
GCTACCTCGGGTGTGTGGATGTGCGAGGGCCTCCCCCGCTTGCGAAGGGGACATGCGTGCTGGAACCTGT
CGGAACTCCATGCCTTCCTCGCCTGCTCACCTGCTCGACGCTGGAATCGGGACAGGTGCAAAGGGACGCA
GACGTCTGGGACAGCTAAGGCCCGTGTCACCGGAGGGCTCCGCACAGTCGTTCTGGTTTCAACGAATAAG
CAAAACTCGGGCAAGTACTGCAGCTATTTGGAAATGTTTTCCAAACCACAGTCTCTTTAGAACTAAGCCT
ATTTGAAACGGTCGGTGTAGGCTTACTGAGATCAGGAGACAGGGAGGCCCCGCACATCACACAGATAAAG
TCAGACAATTGTAATTAATACTTTTGCTGCCTCAAGTTGTTTTTTAAATAAAGTACTTTGAAATGCATGA
GAATCATGCTGCAATATGATCATTCTAGAGCAAATATATATATACACGTATATATATTTCAAGATGAAAC
TAAAGCAGTTTTTAAATAAATTACTTGAATTTTCTGTGTATTTAAAGGAACGACTGTTTAATGTACTTGA
TGGGCCTCTGGTCTTGCCGTGTCTCCTGCCGCTGGTGGCACTTTGTAGATTGTGTGTTTGTGTCCGGGTG
GCAGTTGGGTACCTGCTCACGCACGGTGTGTCTGCCAGGCCACGGTGTCCCAGGATCGCAGAGGGCTGAC
TTTCAAGACTTCAAGAACATTTTCTGGATGTGTGGAAACTTGAGAATGGCCTTGTGAATCTCGTGCTTGG
ACAGGGCAAGTCCGACTACTGAAAGTGCTGCCAGCTTTGCTGCGAGCCCTCCGGCCAGCGGGAGCCCCGT
GGGCTGGGCACTGTGGCCCTTCTTCTCTGGGGGACGGCACCCCTGGCTTCCTCACCTCGGCCGGGCGTCC
GTGGCAGCTCACTCTATGCAACTTGATCCTCTAGCGGCTTTAAGACTGTAGATCCCCTCTCTGAGACCTG
GCTGTACTTGTCAGGATCTCGAGGCGCAGCTCCCGTCTTAGCTGGTTTCTCCGGCTTCTCGTCCTGACGA
CTATAAAACAGTTGGAGGCAAGAAAGCAGCGGATGTGGGGTGGCAGTGGCCTGACCCGAATCAAGATCCG
ACCCAAACCACACCAAATGTGGGTTCATCTGGGGGCCACCCCTTGCCTGAGGCTTCCCACCCTCATCTGA
AGGCCCAGGGGCCGGATCCAGGGCTCACCAAAGCCGATTCCTCGCCAGCTGGGAGTGCAGAAGTCTCAGG
GCCTGGCTGCGACTTGATTTTTAGGAGGAAGAGGGGCTTCGCAACCCCCCTCTGAATAGTGTGTTAACCC
TTGAGATCCCAGCCTCGACTAATCTGAAGTAAGGACAACAAAGGCCATTCAGTGCCCTCCACATGGCCTT
GCCACAGTCACTGTCAGGGTATGAACGTGCCGGAAGCCAGTGCCAGCCAGGGACAGGCGTGACTGTTGTG
TGCTCCTCGGTGACAGGAGTCGGTGGCTGCACACTTTGTAGACTCGTCAAGCTGTCAGCACTTCAGGTGT
TTGCAAGCAAAGCCCTTCTTAGTGTGCAGGTCAGTGTGCAGAGCCCAAATGAGGGGACCGCAGGGGCTGG
GGTCCAGGGTCAGTAGAGTCGGTTTCTGGAGCTGCCTTCCTGGGAGGCAGGTGTGGGTGACCGGGGCTCT
GGCGGTGCGTGTGGGCCCGGCCTGGCCACAGCGGGGACCAGGTCACGACATCTTTGGCCTCAACCCCTCC
CCTGCACTGAGATTATTTCCAGATTGCACTCACTTGAAACCGTCCGTGTCGTCACCTTGTGTCTTAAGGG
AAGCCGAGAAGAAGGGCAGACGGGCAGGCTGTCTTGTCTGCAAAGCGCCATTGCGCCCGCAGCTTGTGTG
GGTCAGGCTGCAGCTCGGGTGTCTGTGGCTTCTAACCTTGTACCTCAGACGGATGCAGTAACAAGGCGGG
GCTGGGGACGCCGGTCAGTGTCAAAGGGGAGGTGCTCTGGCTGATAGCCTTGCCCGAGAGGGACGAGGAG
GCCGTGCGGGTGCCCCCTGGGGAAGCTGGCCAGCCATCCAGTGCTGAGGACGCAGCTGGAGTTGGGCTCG
TGGCACCCTTGGGGTGTGGGCTGTGCAGGCTGGTGGCCTGGGTGCCTCTGCACACTGAGTGGAGTGTCAG
GCAGGGTGTGTCGGTTGGTCGAGTCTTGTGTAATGTGCGCAGACCAGTTACCAAACTAGGATAATGTTGG
TCTCATTTGTGGTGGTTTTGTTCCCTATACAAGTCAGCTAAGTAAAGACTCTTTTAACGAGCTTCCCCTT
AACACATGGCAGAAGTTTCCAGGTGCAGGAATGCGAGCTGGCGGGAAGGGGCAGAGGCCGTGAGCTCTCA
GCTGGGCCGGCCTGCCTGTGTCCCCTTTCCTGGGTCTGTCGGCAGACTGGCATCATGACGTTCCCTGGTG
GCTGAAGAGCTAGCTTTGGAGTGTTGTTTTTCTCACTCTCAGGCAGGGGCCTTAGCTGGAATCCTCCAAC
CTGCCACTGAACACGTCAGTGCTGTGTGCTGCCTCTTGGACACCTGCCCTTGAAAGCCTCAGGCCCCTGG
GAGAAGCACTCTGTCCAGTCCTGTCCCCGGGGGGGAGGCAGGGCCACTGAGCCCTCCTCAGATGGTTAGT
GGCTTCCAACAGCCATCAGGAGTGTTTCTTGAATGCCCCAGGTGTGGAGGACTTGGTCTGTGACCACCTA
GAACCCCAGAGCTGAACAGGAAGCCGTCCCTGCAGCAACAAGAGGGCTGGAAGGGGGAGCTGCAGGCCAC
CCTCGGCTCTCCCACTGCTGGGGCGGTGATGTTCGGGTGACATGTTTGAAAAATACTCTTAAAGATACCA
ACTGTTCCCTTATATGGCTAATGGTTTGTGCAGCCACCAGCGATGGCGGCCCCTATTAGAGACCAGGTTT
GTTAAAACACCAAATATTGCTGTCCACACTAGACATTAACCGGCTTCAGAAAAGATGGACACCTTTTCCC
ACGCTGTTTCGCTTCTTAACTTTGGTCCAGCTTTAGCCACCACACAGCGTGTGAGGGACTGCTGCTGCGG
AGTCAGCCTCGTTTGTCCCTCCGCCTCCCACCAGCACGCGCCGCTTCTGAGAGACACCAGCTCCCTGCCT
CCAAGCCTGGTGCCACAGGCCTGTCGTGAGGGACCCCTGCTTCCGAGAGCTCCTGGGGGGGTTCTGCCCT
TCACCACCTGGGAGAGGTGTCAGTTCAGTTCCGAGTTGAACAAGGCCCGTGCACACAGCATGTTGGGGGC
CCAGCCCAAAGTTCTTGTCACCTCCTCATGCAAAGCCAGCCATCACCCTCCGGCCAGAGCTCAAGGTGGC
CCCTTGGCCAGCCCCTCCTTGGGTCCTCCAGGAGGACTGAGCACCCCTCCTAGCGGCATCCCTTGCCCTC
CACAGTGCTGCCAGGGGCACGTCGCTCTGTGCCGTGGACTGAGACCATCCCCTGGTGACAGAATGACCCG
TTTGTTGGAAATGCCTCGTTGCCAGAGAAACTCCCCAGGCATCTCGGAACGAAACTATTTAGTTCCATTG
TGAACTGGCCACGGGACAGCTTTTTATCAACTTATTAAGTTGGAGCACTGTAATCGCGCTTGCTGAGTTA
GCAGTGGTGGTAAGCGTGTGTTAAACACATAATGTTACGTTTTAGGAGAGAGAGGTCGTAAGGAAGTGTC
GTGTCGCTCATGACTCTCTTCTATTAGTTGGGTAACAGTGGCCTCATGTTTGTGTCTGTGTGTACACAGA
GCCCTTAGGTTCTGCTCTGTTTCTTTGCCAGGTGAATGTTTGTGGCATGCGCTGCTGTCCGCGCCCCTCT
GTCCTGCGCAGGGTTCAGCTGTGCGGCGCCCTGATTTCCTCCATGCACACAGAACCTCCTTGTGTCTGTT
TCTCTGTTCCTCTGTGGCTGACTCAATAAACTTTTCCCTCTGA
>NM_001286648.2 SLC45A4, transcript variant 3, mRNA
(SEQ ID NO: 492)
TGATAGGGCAGAGAGGAGCCCATGCTCCAGCTGCGGGGAGTGCCTGCCCGAGGAGCTGGCCAGTGAAGAC
AGGTGCCTGCCAGTGAAGACAGGTGCCCACAAGCTGGACACAGGGTCGGCCCTGAGACAGGACAGCAGCT
GCGGAATGTTCCCGCTGGCTTTCTGGAGCGCCAGCGTCCTGCTTGGGTCTGGCCAGGGGCCTCTAGCCAG
GCTGTTGAGGCGCCCCTCCTTGTTGAAGAGGAGAAACAGTGGCCCCCTGGGAGTTGCTGGGAGGTGCCTG
GCACAGGGCGGCAGGGACAGCATCATCTATGGGGAAAGCAAGCCCAGCCTCAGGGCTGTCCCGTCCAAAG
ACCCTTGTGAGTCCACTGAGGTCAAATCAATGGAGTCTTCAGAAAGGCCTTCCGGAGCAGTACTACAGCC
TCACCTGGTTCCTGAGCCCCATCCTTGGCCTCATCTTCACACCTCTCATTGGGTCTGCGAGTGACCGGTG
CACCCTGAGCTGGGGCCGCCGGCGGCCCTTCATCCTCGCCCTCTGCGTTGGCGTCCTCTTTGGCGTTGCA
CTTTTCCTTAACGGCTCTGCCATCGGTCTGGCCCTCGGCGATGTCCCCAACCGGCAGCCCATTGGCATCG
TGCTCACGGTGCTGGGAGTGGTGGTCCTGGACTTCAGCGCCGATGCCACCGAGGGGCCCATCCGTGCCTA
TCTGCTGGACGTGGTGGACAGCGAGGAGCAGGACATGGCCCTCAACATCCACGCCTTCTCTGCCGGCCTC
GGCGGAGCCATCGGCTACGTGCTGGGTGGGCTGGACTGGACCCAGACCTTCCTGGGCAGCTGGTTCCGGA
CCCAGAACCAGGTGCTCTTCTTCTTTGCCGCCATCATCTTCACGGTGTCCGTGGCCCTGCACCTGTTCAG
CATCGACGAGGAGCAGTACAGCCCGCAGCAGGAGCGCAGCGCTGAGGAGCCCGGCGCCCTGGATGGGGGC
GAGCCGCACGGCGTCCCTGCCTTCCCAGACGAGGTACAGTCGGAGCACGAGCTGGCCCTGGACTACCCGG
ACGTGGACATCATGCGCAGCAAAAGCGACTCGGCATTGCACGTGCCGGACACCGCGCTGGACCTGGAGCC
CGAGCTGCTGTTCCTGCACGACATCGAGCCCTCCATCTTCCACGACGCCTCCTACCCCGCCACCCCCCGC
AGCACCAGCCAGGAGCTCGCCAAGACCAAGCTGCCCCGCCTGGCCACCTTCCTCAAGGAAGCCGCCAAGG
AGGACGAGACCTTGCTGGATAATCACTTGAATGAAGCTAAAGTCCCAAACGGAAGTGGCTCCCCCACAAA
AGACGCCCTCGGCGGCTACACCAGGGTGGACACGAAGCCCTCGGCCACGTCGAGCTCCATGCGGCGGCGG
CGGCACGCGTTCCGCAGGCAGGCCTCCAGCACCTTCTCCTACTACGGCAAGCTTGGGTCCCACTGCTACC
GCTACCGGCGCGCCAACGCCGTGGTGCTGATCAAGCCGTCGCGCAGCATGAGCGACCTGTACGACATGCA
GAAGCGGCAGCGGCAGCACCGGCACCGGAACCAGAGCGGGGCCACCACCTCCAGCGGGGACACCGAGAGT
GAGGAGGGGGAGGGCGAGACCACGGTGCGCCTGCTGTGGCTCTCCATGCTGAAGATGCCCAGGGAGCTGA
TGCGGCTGTGCCTCTGCCACCTCCTCACCTGGTTCTCTGTCATCGCCGAGGCCGTGTTCTACACCGACTT
CATGGGCCAGGTCATCTTCGAAGGCGACCCCAAGGCCCCCTCGAACTCGACCGCCTGGCAAGCCTACAAC
GCCGGGGTCAAGATGGGCTGCTGGGGCCTGGTCATTTATGCCGCCACTGGTGCTATTTGTTCAGCCCTGT
TACAGAAGTACTTGGACAACTACGACCTGAGCGTCAGGGTGATCTACGTGCTGGGGACGCTGGGCTTCTC
TGTCGGCACAGCCGTGATGGCCATGTTTCCCAACGTCTACGTCGCCATGGTCACCATCAGCACCATGGGC
ATCGTCTCCATGAGCATCTCCTACTGCCCGTACGCCCTGCTGGGCCAGTACCATGACATCAAGCAGTACA
TCCACCACAGCCCCGGGAACTCCAAGCGAGGGTTTGGCATAGATTGTGCCATCCTGTCCTGCCAAGTGTA
CATCTCGCAGATCCTGGTGGCCTCTGCCCTTGGGGGCGTGGTCGACGCCGTGGGGACTGTCCGCGTCATC
CCCATGGTGGCCTCTGTGGGCTCTTTCCTGGGCTTCCTGACGGCCACATTCCTGGTGATCTATCCCAACG
TGTCAGAGGAGGCCAAGGAGGAGCAGAAAGGCCTGTCTTCCCCGTTGGCCGGCGAAGGCAGGGCCGGTGG
GAACAGCGAAAAGCCCACCGTGCTGAAGCTCACGCGGAAGGAGGGCCTGCAGGGACCGGTGGAGACAGAG
TCCGTGGTCTGAGCCGCACTCCCGTTTACACACATTCCAGTGGGCGGGTGGGCGGGCGGGCGGGCGGCGG
GGCCAGGCCATGGGCGGGAGCAGAGACACCGCGGAACCCTGCAGATGCTGTGGCCGACCCGGCAGTGCGG
GCCAGAGCCCCTCCGCCCCCATAGCCACAATTCAGTAGTCGTAGGGTAGGTTTGAGCTACTAAGCAAATA
CCACACTAACCACTTTTTCGATAATTAAAAGAATCATTTGAAATATT

Also disclosed is a vector or plasmid comprising one or more of the nucleic acids as described herein.

Also disclosed is a composition or pharmaceutical composition comprising a nucleic acid or a vector or a plasmid as described herein.

Also disclosed is a composition or pharmaceutical composition comprising an agent for modulating the one or more gene or marker as described herein.

Also disclosed is a nucleic acid or a vector or a plasmid as described herein for use in therapy (such as gene therapy).

In yet another aspect, there is provided a composition or a vector or a plasmid comprising one or more of the nucleic acids as disclosed herein.

In yet another aspect, there is provided a composition or a vector or a plasmid comprising one or more of the nucleic acids as described herein for use in therapy.

In yet another aspect, there is provided a kit comprising the nucleic acids as described herein.

Also disclosed is a host cell or host non-human animal expressing the vector or plasmid comprising one or more of the nucleic acids as described herein.

Also disclosed is a kit comprising an agent for modulating the one or more gene or marker as described herein.

Also disclosed is a kit comprising the nucleic acid or protein as described herein.

Also disclosed is a kit for improving liver regeneration and/or preventing liver degeneration comprising an agent for modulating the one or more gene or marker as described herein.

Also disclosed is a kit for improving liver regeneration comprising the nucleic acid as described herein.

Also disclosed is a kit/biomarker panel for predicting hepatocyte degeneration, or determining the improvement of a therapy, wherein the kit/biomarker panel comprises one or more reagents for detecting one or more gene or marker selected from the group consisting of: C1ORF131 (2810004N23Rik), SLC45A4, NFKBIB, ARL6IP5, DBNL, GOLGA7B, FAM117B, MED28, TSC22D4, EIF4EBP1, PFN1, PNRC2, ZNF672 (Zfp672), IER5, ADAMTSL5, COL4A5, MYH15, ILKAP, and the like.

In some examples, the one or more gene or marker may further include, but is not limited to, ABCB10, LSM14A, ACTG1, YTHDF2, RPS6KA1, SEC13, IPO11, CTCF, UBAP2L, P2RY2, TRAF3IP2, FST, TUBG1, ZNF664, PVR, DYX1C1/DNAAF4, TEX264, NIF3L1, RPUSD1, TRIM6, GLRX3, BRD1, CKS1B, C6ORF120, WASF2, ZNF689, B4GALT7, LCORL, NR2F2, BAG6, RNF10, and the like.

In some examples, the one or more gene or marker may further include, but is not limited to, UBA6, BRWD3, FYCO1, ARID5B, C20ORF96 (6820408C15Rik), CD200R1 (Cd200r4), DALRD3, ELF3, ELFN1, FAM160A1, FAM50B, KIAA2026 (9930021J03Rik), KREMEN1, MACC1, NAA25, PROSC/PLPBP, RPS3, SHPK, SLAIN1, TCHP, ZNF503 (Zfp503), BUD13, CD93, PFKFB2, ZDBF2, FOXC2, GBGT1, RGS4, RNASEH1, TSNAXIP1, LYPD2, STIL, TMCO3, TMEM159, RIF1, IFIH1, EPN2, CLP1, RNF220, NOL12, APOL3, GTF3C6, CTPS2, IFITM2, IFITM1, KCNS3, CA2, EIF4H, MRM1, AKAP8, PAPOLA, SECTM1, TYK2, MLLT6, IL1RL2, IPO7, APBB1IP, RABEP2, PLSCR1, PYGO2, COIL, LRRC8E, TNRC6C, CHD3, USP21, SNX27, NOP14, ZC3H15, C15ORF39, UTP20, TRAK1, RAB11FIP2, PHPT1, CUL3, GNL3, and the like.

Also disclosed is a method, or nucleic acid, or vectors, or an apparatus as described herein.

The term “and/or”, e.g., “X and/or Y” is understood to mean either “X and Y” or “X or Y” and should be taken to provide explicit support for both meanings or for either meaning.

Further, in the description herein, the word “substantially” whenever used is understood to include, but not restricted to, “entirely” or “completely” and the like. In addition, terms such as “comprising”, “comprise”, and the like whenever used, are intended to be non-restricting descriptive language in that they broadly include elements/components recited after such terms, in addition to other components not explicitly recited. For example, when “comprising” is used, reference to a “one” feature is also intended to be a reference to “at least one” of that feature. Terms such as “consisting”, “consist”, and the like, may in the appropriate context, be considered as a subset of terms such as “comprising”, “comprise”, and the like. Therefore, in embodiments disclosed herein using the terms such as “comprising”, “comprise”, and the like, it will be appreciated that these embodiments provide teaching for corresponding embodiments using terms such as “consisting”, “consist”, and the like. Further, terms such as “about”, “approximately” and the like whenever used, typically means a reasonable variation, for example a variation of +/−5% of the disclosed value, or a variance of 4% of the disclosed value, or a variance of 3% of the disclosed value, a variance of 2% of the disclosed value or a variance of 1% of the disclosed value.

Furthermore, in the description herein, certain values may be disclosed in a range. The values showing the end points of a range are intended to illustrate a preferred range. Whenever a range has been described, it is intended that the range covers and teaches all possible sub-ranges as well as individual numerical values within that range. That is, the end points of a range should not be interpreted as inflexible limitations. For example, a description of a range of 1% to 5% is intended to have specifically disclosed sub-ranges 1% to 2%, 1% to 3%, 1% to 4%, 2% to 3% etc., as well as individually, values within that range such as 1%, 2%, 3%, 4% and 5%. It is to be appreciated that the individual numerical values within the range also include integers, fractions and decimals. Furthermore, whenever a range has been described, it is also intended that the range covers and teaches values of up to 2 additional decimal places or significant figures (where appropriate) from the shown numerical end points. For example, a description of a range of 1% to 5% is intended to have specifically disclosed the ranges 1.00% to 5.00% and also 1.0% to 5.0% and all their intermediate values (such as 1.01%, 1.02% . . . 4.98%, 4.99%, 5.00% and 1.1%, 1.2% . . . 4.8%, 4.9%, 5.0% etc.) spanning the ranges. The intention of the above specific disclosure is applicable to any depth/breadth of a range.

Additionally, when describing some embodiments, the disclosure may have disclosed a method and/or process as a particular sequence of steps. However, unless otherwise required, it will be appreciated that the method or process should not be limited to the particular sequence of steps disclosed. Other sequences of steps may be possible. The particular order of the steps disclosed herein should not be construed as undue limitations. Unless otherwise required, a method and/or process disclosed herein should not be limited to the steps being carried out in the order written. The sequence of steps may be varied and still remain within the scope of the disclosure.

Furthermore, it will be appreciated that while the present disclosure provides embodiments having one or more of the features/characteristics discussed herein, one or more of these features/characteristics may also be disclaimed in other alternative embodiments and the present disclosure provides support for such disclaimers and these associated alternative embodiments.

### DESCRIPTION OF FIGURES

FIG. 1 shows a diagram with background information NAFLD (global prevalence).

FIG. 2 shows a schematic outline of NAFLD progression and global burden.

FIG. 3 shows a schematic outline of an approach: Bench to bedside RNAi.

FIG. 4 shows a table with diet induced mouse models of NAFLD & NASH.

FIG. 5 shows graphs that feeding C57Bl6 mice “Western Diet” with fructose in the drinking water induces progressive NAFLD, leading to steatosis and after 26 weeks to advanced fibrosis.

FIG. 6 shows a schematic outline of screening setup for fully in vivo functional genomics to identify therapeutic targets.

FIG. 7 shows a table where next generation sequencing of the 32 sub pools of the genome wide shRNA library of the present application before and after injection shows excellent coverage.

FIG. 8 shows a bar graph where next generation sequencing of the 32 sub pools of the genome wide shRNA library of the present application before and after injection shows excellent coverage.

FIG. 9 shows a schematic outline with an overview of Differential Expression Analysis.

FIG. 10 shows box plots with the outline of different analysis pipelines used for Different Expression Analysis.

FIG. 11 shows a graph with Different Expression Analysis results. For translation from therapeutic target to RNAi therapeutic enriched shRNAs are prioritized.

FIG. 12 shows diagram with the primary selection criteria for high confidence targets.

FIG. 13 shows a table and STRING network analysis with top scoring candidates for validation (primary analysis).

FIG. 14 shows schematics of additional selection process I.

FIG. 15 shows a table and STRING network analysis with added scoring targets. Indicated (*) are targets which show dysregulation in a local NAFLD patient cohort and which already entered validation process (#).

FIG. 16 shows diagram of target gene identification (filtration steps) (expanded analysis) Top Hits I.

FIG. 17 shows diagram of target gene identification (filtration steps) (expanded analysis) Top Hits II.

FIG. 18 shows diagram of target gene identification; cross-referencing to EMULSION NAFLD patient cohort transcriptomics.

FIG. 19 shows STRING network analysis and table of extreme stringent selection criteria that prioritizes 49 gene targets.

FIG. 20 shows tables with extended stringent selection criteria that expand prioritized gene target list to 243 genes.

FIG. 21 shows STRING (https://string-db.org/) network analysis of the 242 prioritized gene targets that shows connectivity between some targets.

FIG. 22 shows a table that increasing cut off criteria reduces highest priority list to 28 gene targets.

FIG. 23 shows STRING (https://string-db.org/) network analysis of the 83 prioritized gene targets that shows connectivity between some targets.

FIG. 24 shows STRING network analysis and table, with increasing cut off criteria reducing highest priority list further to 54 gene targets. STRING (https://string-db.org/) network analysis of the 42 prioritized gene targets shows connectivity between some targets.

FIG. 25 shows the results of confluence growth curve and cell doubling time assay for in vitro validation of targets.

FIG. 26 shows the results for a wound healing assay for validation of targets

FIG. 27 and FIG. 28 show the results of EdU incorporation assay for validation of targets.

FIG. 29 shows graphs for the candidate 281004N23Rik. Strong enrichment for shRNA targeting candidate. The local NAFLD patient cohort shows disease stage specific expression changes. TCGA survival analysis shows no adverse relation to cancer survival.

FIG. 30 shows STRING (https://string-db.org/) network analysis of 281004N23Rik, showing connection to several biological processes (chromosome, RNA-binding, CuI4-RING E3 ubiquitin ligase complex).

FIG. 31 shows bar graph with 281004N23Rik amplification associated with liver cancer based on TCGA data analysis, indicating that knockdown of 281004N23Rik in hepatocytes should be safe.

FIG. 32A shows microscopy images and graphs on in vitro validation of target. 281004N23Rik knockdown accelerates cell migration and proliferation. Screen scoring shRNA expression results in efficient knockdown in immortalized mouse liver cell line. Wound healing assay shows knockdown dependent accelerated closing of wound gap.

FIG. 32B shows microscopy images and graph with a repopulation model. The repopulation assay shows accelerated clonal expansion of hepatocytes with target knockdown in vivo, proving faster regeneration. Importantly, this represents hepatocyte expansion in a chronic damaged liver. The FAH−/− hepatocytes are dying and are replaced with the FAH expressing hepatocytes.

FIG. 32C shows microscopy images and graph with a western diet model. Fully repopulated mice, where every hepatocyte expresses the shRNA of interest were exposed to the “Western Diet”, inducing progressive NAFLD. On the left representative images are shown. On the right the quantitative results from blinded fibrosis score as given by a certified pathologist are shown. Knockdown of the target significantly reduced fibrosis level.

FIG. 32D shows microscopy images and graphs with a partial hepatectomy model. Partial hepatectomy (PH), surgical removal of ⅔ of the liver of fully repopulated mice, where every hepatocyte expresses the shRNA of interest was conducted. PH is an acute liver damage inducing synchronic proliferation of hepatocyte to regenerate the liver. On the left side representative images of Ki67 staining are shown. On the right side the quantitative evaluation of staining for Ki67 at time point of PH (0 h) and at 48 h post operation is shown. Knockdown of the target accelerates liver regeneration indicated by higher number of Ki67 positive hepatocytes at 48h. Furthermore, as 0 h represents the time point of full repopulation the comparable basal Ki67 positive level in case of target knockdown vs control shRNA indicates that regeneration termination after repopulation is fully functional. This proves that the safety checkpoint preventing hepatomegaly and “overshooting” regeneration is intact. Knockdown of the target only releases a regeneration break, accelerating regeneration, but does not disable the safety checkpoint. This underlines the safety of knocking down this target.

FIG. 33 shows graphs for candidate Ythdf2. Strong enrichment for shRNA targeting candidate. The local NAFLD patient cohort shows disease stage specific expression changes. TCGA survival analysis shows survival benefits with low target expression for liver cancer.

FIG. 34 shows STRING (https://string-db.org/) network analysis of Ythdf2, shows connection to several biological processes (transcription, deadenylation of mRNA, metabolism of RNA, reversal of alkylation damage by DNA dioxygenase).

FIG. 35 shows bar graph where Ythdf2 does not show significant alterations in liver cancer based on TCGA data analysis, indicating that knockdown of Ythdf2 in hepatocytes should be safe.

FIG. 36 shows microscopy images and graphs of in vitro validation of target. Ythdf2 knockdown accelerates cell migration and proliferation. Screen scoring shRNA expression results in efficient knockdown in immortalized mouse liver cell line. Wound healing assay shows knockdown dependent accelerated closing of wound gap. EdU incorporation assay indicates slight increase in proliferation upon candidate knockdown. Cell confluency assay shows shortened cell doubling time.

FIG. 37 shows a table of GWAS analysis that indicate a link between Ythdf2 and diabetes.

FIG. 38 shows graphs for candidate Golga7B. Strong enrichment for shRNA targeting candidate. The local NAFLD patient cohort shows disease stage specific expression changes. TCGA survival analysis shows survival benefits with low target expression for liver cancer.

FIG. 39 shows STRING (https://string-db.org/) network analysis of Golga7B shows connection to several biological processes (p38-, MAPK-, G protein signaling, EGFR, sphingolipid, glycine, serine and threonine metabolism).

FIG. 40 shows a graph of Golga7B amplification associated with liver cancer based on TCGA data analysis, indicating that knockdown of Golga7B in hepatocytes should be safe.

FIG. 41 shows microscopy images and graphs for In vitro validation of target. Golga7B knockdown accelerates cell migration and proliferation. Screen scoring shRNA expression results in efficient knockdown in immortalized mouse liver cell line. Wound healing assay shows knockdown dependent accelerated closing of wound gap. EdU incorporation assay indicates slight increase in proliferation upon candidate knockdown. Cell confluency assay shows shortened cell doubling time.

FIG. 42 graphs for candidate Sec13. Strong enrichment for shRNA targeting candidate. The local NAFLD patient cohort shows disease stage specific expression changes. TCGA survival analysis shows survival benefits with low target expression for liver cancer.

FIG. 43 shows STRING (https://string-db.org/) network analysis of Sec13 shows connection to several biological processes (COPII-coated vesicle cargo loading, nuclear pore).

FIG. 44 shows a graph of Sec13 amplification associated with liver cancer based on TCGA data analysis, indicating that knockdown of Sec13 in hepatocytes should be safe.

FIG. 45 shows microscopy images and graphs of n vitro validation of target. Sec13 knockdown accelerates cell proliferation. Screen scoring shRNA expression results in knockdown in immortalized mouse liver cell line. EdU incorporation assay indicates slight increase in proliferation upon candidate knockdown.

FIG. 46 shows graphs for candidate MrpI49. Strong enrichment for shRNA targeting candidate. The local NAFLD patient cohort shows disease stage specific expression changes. TCGA survival analysis shows no adverse relation to cancer survival.

FIG. 47 shows STRING (https://string-db.org/) network analysis of MrpI49 shows connection to several biological processes (translation, mitochondrial translation, mitochondrial ribosome).

FIG. 48 shows a graph where MrpI49 does not show specific significant alterations in liver cancer, indicating that knockdown of MrpI49 in hepatocytes should be safe.

FIG. 49 shows graphs of in vitro validation of target. MrpI49 knockdown accelerates cell proliferation. Screen scoring shRNA expression results in efficient knockdown in immortalized mouse liver cell line. EdU incorporation assay indicates slight increase in proliferation upon candidate knockdown.

FIG. 50 shows graphs for Candidate Med28. Strong enrichment for shRNAs targeting candidate. The local NAFLD patient cohort shows no major disease stage specific expression changes. TCGA survival analysis shows survival benefits with low target expression for liver cancer. Med28 amplification is associated with liver cancer based on TCGA data analysis, indicating that knockdown of Med28 in hepatocytes should be safe.

FIG. 51 shows STRING (https://string-db.org/) network analysis of Med28 that shows connection to several biological processes (lipid metabolism, transcription, protein ubiquitination, stem cell maintenance).

FIG. 52 shows microscopy images and bar graphs of in vitro validation of target. Med28 knockdown accelerates cell migration and proliferation. Screen scoring shRNA expression results in efficient knockdown in immortalized mouse liver cell line. Wound healing assay shows knockdown dependent accelerated closing of wound gap. EdU incorporation assay indicates slight increase in proliferation upon candidate knockdown. Cell confluency assay shows shortened cell doubling time.

FIG. 53 shows graphs for candidate Fam117b. Strong enrichment for shRNAs targeting candidate. The local NAFLD patient cohort shows disease stage specific expression changes. TCGA survival analysis shows survival benefits with low target expression for liver cancer. Fam117b amplification is associated with liver cancer based on TCGA data analysis, indicating that knockdown of Fam117b in hepatocytes should be safe.

FIG. 54 shows STRING (https://string-db.org/) network analysis of Fam117b that shows connection to several biological processes (G0 and early G1, cell cycle, DNA replication, beta-catenin).

FIG. 55 shows microscopy images and bar graphs of the in vitro validation of target. Fam117b knockdown accelerates cell migration and proliferation. Screen scoring shRNA expression results in efficient knockdown in immortalized mouse liver cell line. Wound healing assay shows knockdown dependent accelerated closing of wound gap. EdU incorporation assay indicates slight increase in proliferation upon candidate knockdown. Cell confluency assay shows shortened cell doubling time.

FIG. 56 shows graphs for candidate Slc45a4. Strong enrichment for shRNAs targeting candidate. The local NAFLD patient cohort shows disease stage specific expression changes. TCGA survival analysis shows survival benefits with low target expression for liver cancer. Slc45a4 amplification is associated with liver cancer based on TCGA data analysis, indicating that knockdown of Slc45a4 in hepatocytes should be safe.

FIG. 57 shows STRING (https://string-db.org/) network analysis of Slc45a4 that shows connection to several biological processes (TNF-, IL-17-, CD95-signaling, TP53, nucleoside transmembrane transporter activity).

FIG. 58 shows microscopy images and bar graphs of in vitro validation of target. Slc45a4 knockdown accelerates cell migration and proliferation. Screen scoring shRNA expression results in efficient knockdown in immortalized mouse liver cell line. Wound healing assay shows knockdown dependent accelerated closing of wound gap. EdU incorporation assay indicates slight increase in proliferation upon candidate knockdown. Cell confluency assay shows shortened cell doubling time.

FIG. 59 shows graphs for candidate Tsc22d4. Strong enrichment for shRNAs targeting candidate. The local NAFLD patient cohort shows disease stage specific expression changes. TCGA survival analysis shows survival benefits with low target expression for liver cancer. Tsc22d4 amplification is associated with liver cancer based on TCGA data analysis, indicating that knockdown of Tsc22d4 in hepatocytes should be safe.

FIG. 60 shows STRING (https://string-db.org/) network analysis of Tsc22d4 that shows connection to several biological processes (G-proteins, GTPase activity, PI3K-Akt-mTOR signaling, insulin secretion).

FIG. 61 shows graphs of in vitro validation of target. Tsc22d4 knockdown accelerates cell migration and proliferation. Screen scoring shRNA expression results in efficient knockdown in immortalized mouse liver cell line. Wound healing assay shows knockdown dependent accelerated closing of wound gap. EdU incorporation assay indicates slight increase in proliferation upon candidate knockdown. Cell confluency assay shows shortened cell doubling time.

FIG. 62 shows graphs for candidate Arl6ip5. Strong enrichment for shRNAs targeting candidate. The local NAFLD patient cohort shows no major disease stage specific expression changes. TCGA survival analysis shows no liver cancer concern. Arl6ip5 mutations are associated with liver cancer based on TCGA data analysis, indicating that knockdown of Arl6ip5 in hepatocytes should be safe.

FIG. 63 shows STRING (https://string-db.org/) network analysis of Arl6ip5 that shows connection to several biological processes (intracellular protein transport, cell death, cell cycle, hepatitis c, FOXO TFs, inactivation of cyclin B).

FIG. 64 shows graphs for in vitro validation of target. Arl6ip5 knockdown accelerates cell migration and proliferation. Screen scoring shRNA expression results in efficient knockdown in immortalized mouse liver cell line. Wound healing assay shows knockdown dependent accelerated closing of wound gap. EdU incorporation assay indicates slight increase in proliferation upon candidate knockdown. Cell confluency assay shows shortened cell doubling time.

FIG. 65 shows graphs for candidate Nfkbib. Strong enrichment for shRNAs targeting candidate. The local NAFLD patient cohort shows disease stage specific expression changes. TCGA survival analysis shows no liver cancer concern. Nfkbib amplifications are associated with liver cancer based on TCGA data analysis, indicating that knockdown of Nfkbib in hepatocytes should be safe.

FIG. 66 shows STRING (https://string-db.org/) network analysis of Nfkbib that shows connection to several biological processes (I-kappaB/NFkappaB complex, RNA polymerase transcription).

FIG. 67 shows graphs for in vitro validation of target. Arl6ip5 knockdown accelerates cell migration and proliferation. Screen scoring shRNA expression results in efficient knockdown in immortalized mouse liver cell line. Wound healing assay shows knockdown dependent accelerated closing of wound gap. EdU incorporation assay indicates slight increase in proliferation upon candidate knockdown. Cell confluency assay shows shortened cell doubling time.

FIG. 68 shows graphs for candidate DbnI. Strong enrichment for shRNAs targeting candidate. The local NAFLD patient cohort shows disease stage specific expression changes. TCGA survival analysis shows no liver cancer concern. DbnI amplifications and mutations are associated with liver cancer based on TCGA data analysis, indicating that knockdown of DbnI in hepatocytes should be safe.

FIG. 69 shows STRING (https://string-db.org/) network analysis of DbnI that shows connection to several biological processes (Gap junction activity, EPHB-mediated forward signaling, RHO GTPases activate WASPs and WAVEs).

FIG. 70 shows graphs of in vitro validation of target. DbnI knockdown accelerates cell migration and proliferation. Screen scoring shRNA expression results in efficient knockdown in immortalized mouse liver cell line. Wound healing assay shows knockdown dependent accelerated closing of wound gap. EdU incorporation assay indicates slight increase in proliferation upon candidate knockdown. Cell confluency assay shows shortened cell doubling time.

FIG. 71 shows graphs for candidate LoxI2. Strong enrichment for shRNAs targeting candidate. The local NAFLD patient cohort shows disease stage specific expression changes. TCGA survival analysis shows no liver cancer concern. LoxI2 deletions are associated with liver cancer based on TCGA data analysis, indicating that knockdown of LoxI2 has to be carefully monitored.

FIG. 72 shows STRING (https://string-db.org/) network analysis of LoxI2 that shows connection to several biological processes (cell cycle, deltanotch, histone modifications, elastic fiber, peptidyl-lysine oxidation).

FIG. 73 shows graphs of in vitro validation of target. LoxI2 knockdown accelerates cell migration and proliferation. Screen scoring shRNA expression results in efficient knockdown in immortalized mouse liver cell line. EdU incorporation assay indicates slight increase in proliferation upon candidate knockdown. Cell confluency assay shows shortened cell doubling time.

FIG. 74 shows graphs for Candidate Ifih1. Strong enrichment for shRNAs targeting candidate. The local NAFLD patient cohort shows disease stage specific expression changes. TCGA survival analysis shows worse survival with low expression. Ifih1 mutations are associated with liver cancer based on TCGA data analysis, indicating that knockdown of Ifih1 might be save.

FIG. 75 shows STRING (https://string-db.org/) network analysis of Ifih1 that shows connection to several biological processes (Toll-, RIG-1-, NFkappaB-signaling, hepatitis b).

FIG. 76 shows microscopy images and graphs for in vitro validation of target. Ifih1 knockdown accelerates cell migration and proliferation. Screen scoring shRNA expression results in efficient knockdown in immortalized mouse liver cell line. Wound healing assay shows knockdown dependent accelerated closing of wound gap.

FIG. 77 a schematic diagram, microscopy images and dot plot for in vivo validation of prioritized targets. For the first step, the inventors of the present disclosure did investigate for repopulation acceleration of the liver. The inventors of the present disclosure take advantage of FAH knockout mice, which allow liver repopulation. Through hydrodynamic tail vein injection, the inventors of the present disclosure deliver constructs for the expression of the missing enzyme FAH, the marker GFP and the shRNA of interest to the liver. Taking mice of the drug NTBC FAH expressing hepatocytes will expand to repopulate the liver. If the expression of a certain shRNA enhances liver regeneration, the inventors of the present disclosure expect a faster clonal expansion. Shown is the outline of the experiment, an example of tissue samples, and target analysis. The inventors of the present disclosure analyzed the results for shCtrl (shNC), sh2810004N23Rik (shRik), shYthdf2, and shGolga7B. The inventors of the present disclosure identified an increased number of GFP positive cells in case of target knockdown, validating accelerated repopulation and regeneration in vivo.

FIG. 78 shows a schematic diagram with the outline for further ongoing in vivo validation.

FIG. 79 shows a schematic diagram with an in vitro validation pipeline to test the ability of the target knockdown to enhance hepatocyte proliferation for scoring shRNAs. For this purpose a stable hepatocyte cell line with stable expression of the scoring shRNA (1) was first generated. Then the knockdown efficiency of the scoring shRNA (2) was tested. If sufficient knockdown was seen, accelerated wound healing (A), increased cell proliferation by EdU incorporation, cell doubling time and CTG and CCK8 assays (B, C) were then tested. If enhanced regenerative power of hepatocytes in vitro was validated, in vivo validation will then be performed.

FIG. 80 shows a schematic diagram with in vivo validation with the FAH−/− knockout mouse line. A transposon based construct for the expression of the missing enzyme FAH, a marker (e.g. GFP) and the shRNA of interest was generated. 74his construct was delivered together with a plasmid for the expression of the transposase SB13 to the hepatocytes by hydro-dynamic tail vine injection (HDTV). In average 10% of hepatocytes has stable integration. The inventors of the present disclosure then look for accelerated repopulation of the liver, showing faster hepatocyte proliferation in vivo. After full repopulation, basically every hepatocyte expresses the shRNA of interest and the FAH deficiency in the liver is corrected. Therefore, these mice reflect the “normal” mouse condition but with hepatocyte specific knockdown of the target of interest. The inventors of the present disclosure then perform classic partial hepatectomy experiments to evaluate liver regeneration after acute liver damage. Furthermore, the inventors of the present disclosure expose repopulated mice to a NAFLD inducing diet and can check for fibrosis development. Comparison was made with the control group expressing a non-targeting shRNA.

FIG. 81A shows a bar graph with qPCR data that shows target gene knockdown of human homolog of Rik, C1ORF131 in a human liver cell line, HepG2. Two different siRNAs were used, and knockdown confirmed in both using two different primer pairs, primer pair 1 and 2 against ORF131 gene. Values represent mean±SD. ** p<0.01, *** p<0.001 using Student's t test.

FIG. 81B shows a line graph of a luminescent assay called CellTiter-Glo (CTG) that was used to determine the number of viable cells in culture based on quantitation of the ATP present, an indicator of metabolically active cells. The amount of ATP is directly proportional to the number of cells present in culture. Luminescence was measured 3 days post transfection on Day 4, 5 and 6.

FIG. 81C shows a line graph with another proliferation assay for determining cell viability which is the Cell Counting Kit-8 (CCK8) assay which uses a water-soluble tetrazolium salt to quantify the number of live cells by producing an orange formazan dye upon bio-reduction in the presence of an electron carrier. The amount of formazan produced is directly proportional to the number of live cells and is measured by absorbance at 450 nm. Absorbance was measured 3 days post transfection on Day 4, and 5. For both CTG and CCK8 assay, normalized data is presented. More replicates are ongoing to further establish the phenotype.

FIG. 82A shows a bar graph with qPCR data that shows target gene knockdown using two different siRNAs against Rik gene in immortalized mouse liver cell line. Chemically modified siRNAs were generated by an industry collaborator, in that they are not made up of natural RNA nucleotides but a combination of 2′-F and 2′-OMe modified nucleotides. The difference in G101 and G111 is the pattern of 2′-F and 2′-OMe modifications. siRik-010-G111 has the same 2′-F and 2′-OMe modification pattern as 006-G111 but has a different ATGC sequence and hence targets a different region on the mRNA. Both siRNAs show efficient knockdown and will be GalNAc tagged in the future for potential in-vivo experiments.

FIG. 82B shows a line graph of a Cell Counting Kit-8 (CCK8) assay that was used to determine cell viability. Absorbance at 450 nm was measured 3 days post transfection on Day 4, and 5. Both siRNAs show increased activity compared to control. More replicates as well as other cell proliferation assays are ongoing to further establish the phenotype.

FIG. 83A shows a schematic diagram to ascertain whether target knockdown leads to tumor formation, FAH (−/−) animals were injected with control and target of interest and left for a minimum period of 1-year to observe tumor growth or malignant lesions, if any.

FIG. 83B shows images of animal livers that were harvested, n=1 for control and n=2 for target and sent for histology. Representative pictures of the liver are shown. No sign of distress or tumor growth was observed at the end of 1-year timepoint. Animals looked healthy, and liver looked normal. Furthermore, no morphological changes or malignant lesions were observed in hepatocytes stained with Hematoxylin and Eosin (H&E) and imaged at 20×. F1, F2 refers to female animal 1 and 2. Data for additional animals is under progress.

FIG. 84A shows a blot where tumor suppressor gene Pten was used as a positive control and its knockdown confirmed before injection, as seen by western blot for 2 different cell batches.

FIG. 84B shows images where about ~2 million cells were injected subcutaneously into NSG mice with shPten-AML in one flank (left) and shRik-AML on the other flank (right) and response monitored. 36 weeks post transplantation, n=4 shPten-AML animals showed subcutaneous growth compared to shRik-AML where no growth was seen, further confirming that target knockdown has no adverse or toxic effects.

FIG. 85A shows a bar graph of qPCR data that shows target gene knockdown for 4 different Rik hairpins, in immortalized mouse liver cell line. Out of 4 hairpins, Rik-3 shows best knockdown and was used to further validate other phenotypic assays. Values represent mean±SD. ** p<0.01 using Student's t test.

FIG. 85B shows a bar graph with a wound healing assay that shows accelerated wound closure at 12-16 hr compared to control. Values represent mean±SD. * p<0.05, ** p<0.01, *** p<0.001 determined by two-way ANOVA.

FIG. 85C shows a bar graph where Rik-3 was also found to promote DNA synthesis as evident by the increased incorporation of Edu into the DNA of Rik-3 knockdown cells compared to the shC. Proliferating cells were examined using the EdU Alexa Fluor 594® Imaging Kit. EdU-positive cells were counted using Operetta High-Content Analysis System using 25 random fields and plotted as % EdU which is number of EdU stained cells to the total number of cells stained for DAPI. Values represent mean±SD. * p<0.05 using Student's t test.

FIG. 85D shows a line graph where CellTiter-Glo (CTG) was also used to determine the number of viable cells with shRik-3 showing increased activity compared to control. Cells were seeded in a 96-well format and luminescence measured starting at 24 hours up to 96 house post seeding to determine the viability potential. Values represent mean±SD. * p<0.05 determined by two-way ANOVA.

FIG. 86A shows a table with data from RNA-Seq analysis using control and Rik-knockdown liver tissue samples, confirm decreased expression of Rik as seen by negative fold change (fc) value and analysed using DESeq analysis method. FIG. 86B and FIG. 86C shows principal component analysis and clustering plots using each sample's r log transformed value, the similarity between samples is graphically shown in a 2D format as seen by PCA or in a clustering format as seen by Hierarchical clustering. The two plots allow identification of expression patterns between sample groups.

FIG. 86D and FIG. 86E shows a volcano plot and a graph with of the log 2 fold change and p-value obtained from the comparison of the average for each group and shows genes represented by dots across fc and p-value. The number of up and down regulated genes across each comparison pair are also listed, with 767 upregulated and 268 downregulated genes obtained.

FIG. 87A shows a STRING analysis of 140 protein coding genes with FC≤−2 and p-value ≤0.05 shows different interacting genes forming clusters.

FIG. 87B and FIG. 87C shows three different pathway analysis tools-DAVID, CPDB and STRING that were used as a streamlined approach to determine the pathways of interest (POIs). The top overlapping pathways that converge from the three methods are shown in the table, with overlapping genes of interest from all three, highlighted in different colours. Histone genes seem to be taking the top spot with further in-depth analysis ongoing to advance the understanding of the mechanism of action.

FIG. 88 shows the candidate gene: Slc45a4 cluster.

FIG. 89 shows microscopy images and graphs of cell migration and proliferation. Stable expression of target gene is seen in hepatocyte cell line as confirmed by GFP. Knockdown of target gene is seen as confirmed by qPCR. Knockdown of target gene accelerates wound healing leading to wound closure at 16 hr compared to control. Increased hepatocyte proliferation is seen compared to control as confirmed by EdU and cell confluency assay.

FIG. 90 shows microscopy images and graphs of a repopulation assay. The repopulation assay shows accelerated clonal expansion of hepatocytes with target knockdown in vivo, proving faster regeneration. Importantly, this represents hepatocyte expansion in a chronic damaged liver. The FAH−/− hepatocytes are dying and are replaced with the FAH expressing hepatocytes. Red highlighted box shows data for target gene of interest.

FIG. 91 shows microscopy images and graphs of an in vivo model to determine the functional impact of Slc45a4 on a western diet model. Fully repopulated mice, where every hepatocyte expresses the shRNA of interest were exposed to the “Western Diet”, inducing progressive NAFLD. On the left representative images are shown. On the right the quantitative results from blinded fibrosis score as given by a certified pathologist are shown. Knockdown of the target significantly reduced fibrosis level. Evaluation of fibrosis score based on Sirius red staining and graded by pathological expert on a scale of 1-5 with 1 being minimal, 2 mild, 3 moderate, 4 marked, and 5 severe.

FIG. 92A shows a bar graph and dot plot of the enrichment for the shRNA in the genome wide screen of the present disclosure and the efficient knockdown by this shRNA in hepatocyte cell line.

FIG. 92B shows a line graph and microscopy images that show representative pictures of the wound healing assay and the quantification of it. Significant faster wound healing can be seen.

FIG. 92C shows microscopy images and dot plot of the results for EdU assay and cell doubling time. Target knockdown accelerates hepatocyte cell line proliferation.

FIG. 93 shows microscopy images and a bar graph of a repopulation assay. The repopulation assay shows accelerated clonal expansion of hepatocytes with target knockdown in vivo, proving faster regeneration. Importantly, this represents hepatocyte expansion in a chronic damaged liver. The FAH−/− hepatocytes are dying and are replaced with the FAH expressing hepatocytes.

FIG. 94 shows images and bar graph of in vivo validation in NAFLD in a Western diet mouse model. Fully repopulated mice, where every hepatocyte expresses the shRNA of interest were exposed to the “Western Diet”, inducing progressive NAFLD. On the left the experimental layout is shown, in the middle representative images of the macroscopic liver are shown. On the right the quantitative results from blinded fibrosis score scoring by an certified pathologist are shown, as well as the knockdown the efficiency. Knockdown of the target significantly reduced fibrosis level. Experimental set up: Male FAH mice, 8 weeks repopulation and 26 weeks of Western Diet (WD). Reference genes for qPCR were Hprt and Ywhaz. Bars represent median values.

FIG. 95 shows the in vitro functional validation results of Arl6ip5. FIG. 95A shows a bar graph and dot plot that shows the enrichment for the shRNA in the genome wide screen of the present disclosure and the efficient knockdown by this shRNA in hepatocyte cell line. FIG. 95B shows representative pictures of the wound healing assay and the quantification of it in line graph. Significant faster wound healing can be seen. FIG. 95C shows the results of EdU assay and cell doubling time. Target knockdown accelerates hepatocyte cell line proliferation.

FIG. 96 shows microscopy images and bar graph of a repopulation assay conducted on Arl6ip5. The repopulation assay shows accelerated clonal expansion of hepatocytes with target knockdown in vivo, proving faster regeneration. Importantly, this represents hepatocyte expansion in a chronic damaged liver. The FAH−/− hepatocytes are dying and are replaced with the FAH expressing hepatocytes.

FIG. 97 shows images and bar graph of a western diet mouse model. Fully repopulated mice, where every hepatocyte expresses the shRNA of interest were exposed to the “Western Diet”, inducing progressive NAFLD. On the left the experimental layout is shown, in the middle representative images of the macroscopic liver are shown. On the right the quantitative results from blinded fibrosis score scoring by a certified pathologist are shown, as well as the knockdown the efficiency. Knockdown of the target significantly reduced fibrosis level.

FIG. 98 shows a dot plot result of on an in vivo model to determine the functional impact of Nfkbib and Arl6ip5 on a partial hepatectomy model. Partial hepatectomy (PH), surgical removal of ⅔ of the liver of fully repopulated mice, where every hepatocyte expresses the shRNA of interest was conducted. PH is an acute liver damage inducing synchronic proliferation of hepatocyte to regenerate the liver. The quantitative evaluation of staining for Ki67 at time point of PH (0 h) and at 48 h post operation is shown. Knockdown of the target accelerates liver regeneration indicated by higher number of Ki67 positive hepatocytes at 48h. Furthermore, as 0 h represents the time point of full repopulation the comparable basal Ki67 positive level in case of target knockdown vs control shRNA indicates that regeneration termination after repopulation is fully functional. This proves that the safety checkpoint preventing hepatomegaly and “overshooting” regeneration is intact. Knockdown of the target only releases a regeneration break, accelerating regeneration, but does not disable the safety checkpoint. This underlines the safety of knocking down this target.

FIG. 99 shows the in vitro functional validation on the investigation of DbnI. FIG. 99A shows a bar graph and dot plot that shows the enrichment for the shRNA in the genome wide screen of the present disclosure and the efficient knockdown by this shRNA in hepatocyte cell line. FIG. 99B shows representative pictures of the wound healing assay and the quantification of it in line graph. Significant faster wound healing can be seen. FIG. 99C shows the results of EdU assay and cell doubling time. Target knockdown accelerates hepatocyte cell line proliferation.

FIG. 100 shows the schematic and results of investigation of DbnI in vivo hepatocyte clonal expansion study. FIG. 100 shows microscopy images and a bar graph of a repopulation assay. The repopulation assay shows accelerated clonal expansion of hepatocytes with target knockdown in vivo, proving faster regeneration. Importantly, this represents hepatocyte expansion in a chronic damaged liver. The FAH−/− hepatocytes are dying and are replaced with the FAH expressing hepatocytes.

FIG. 101 shows data from emulsion patient cohort showing different disease stages from ~150 patients. The target gene shows significant increased expression during disease progression. Survival analysis from TCGA RNA patient samples showed low expression of the target is not associated with worse survival, suggesting that knockdown of the target does not drive liver cancer.

FIG. 102 shows the schematic and results of investigation of in vivo model to determine the functional impact of Golga7b (repopulation model). FIG. 102 shows microscopy images and a bar graph of a repopulation assay. The repopulation assay shows accelerated clonal expansion of hepatocytes with target knockdown in vivo, proving faster regeneration. Importantly, this represents hepatocyte expansion in a chronic damaged liver. The FAH−/− hepatocytes are dying and are replaced with the FAH expressing hepatocytes.

FIG. 103 shows genetic clustering of gene Fam117b. Family with sequence similarity 117 member B. Enriched shRNA score across different animals were presented as Log 2(CPM) where Log counts per million computed from total count analysis method. Diamond refers to median. Circles represent each animals. Survival analysis data from TCGA RNA patient sample shows low expression of gene leads to high survival probability in liver cancer patients.

FIG. 104 shows Fam117b knockdown accelerates cell migration and proliferation. FIG. 104 shows microscopy images and graphs of cell migration and proliferation. Stable expression of target gene is seen in hepatocyte cell line as confirmed by GFP. Knockdown of target gene is seen as confirmed by qPCR. Knockdown of target gene accelerates wound healing leading to wound closure at 16 hr compared to control. Increased hepatocyte proliferation is seen compared to control as confirmed by EdU and cell confluency assay.

FIG. 105 shows the schematic and results of investigation of in vivo model used to determine the functional impact of Fam117b (repopulation model). FIG. 105 shows microscopy images and a bar graph of a repopulation assay. The repopulation assay shows accelerated clonal expansion of hepatocytes with target knockdown in vivo, proving faster regeneration. Importantly, this represents hepatocyte expansion in a chronic damaged liver. The FAH−/− hepatocytes are dying and are replaced with the FAH expressing hepatocytes. Box shows data for target gene of interest.

FIG. 106 shows genetic clustering of gene Med28 (mediator complex subunit 28). Enriched shRNA score across different animals were presented as Log 2(CPM) where Log counts per million computed from total count analysis method. Diamond refers to median. Circles represent each animal. Survival analysis data from TCGA RNA patient sample shows low expression of gene leads to high survival probability in liver cancer patients.

FIG. 107 shows Med28 knockdown accelerates cell migration and proliferation. FIG. 107 shows the microscopy images and graphs of cell migration and proliferation. Stable expression of target gene is seen in hepatocyte cell line as confirmed by GFP. Knockdown of target gene is seen as confirmed by qPCR. Knockdown of target gene accelerates wound healing leading to wound closure at 16 hr compared to control. Increased hepatocyte proliferation is seen compared to control as confirmed by EdU and cell confluency assay.

FIG. 108 shows the schematic and results of investigation of in vivo model to determine the functional impact of Med28. FIG. 108 microscopy images and a bar graph of a repopulation assay. The repopulation assay shows accelerated clonal expansion of hepatocytes with target knockdown in vivo, proving faster regeneration. Importantly, this represents hepatocyte expansion in a chronic damaged liver. The FAH−/− hepatocytes are dying and are replaced with the FAH expressing hepatocytes. Box shows data for target gene of interest.

FIG. 109 shows in vitro functional validation of Eif4ebp1.

FIG. 110 shows the schematic and results of investigation of in vivo validation of Eif4ebp1 in NAFLD (Western diet mouse model). FIG. 110 shows a diagram and bar graph of a western diet mouse model. Fully repopulated mice, where every hepatocyte expresses the shRNA of interest were exposed to the “Western Diet”, inducing progressive NAFLD. On the left the experimental layout is shown, in the middle representative images of the macroscopic liver are shown. On the right the quantitative results from blinded fibrosis score scoring by a certified pathologist are shown, as well as the knockdown the efficiency. Knockdown of the target significantly reduced fibrosis level.

FIG. 111 shows in vitro functional validation of Pfn1.

FIG. 112 shows a table and dot plots results of in vivo model to determine the functional impact of other target genes. In-vitro cell migration and cell proliferation phenotype for different targets are shown and depicted using + for strong phenotype and * for p-value, where 0.05 is significant. The repopulation assay shows accelerated clonal expansion of hepatocytes with target knockdown in vivo, proving faster regeneration. Importantly, this represents hepatocyte expansion in a chronic damaged liver. The FAH−/− hepatocytes are dying and are replaced with FAH expressing hepatocytes. Data for different targets are shown.

FIG. 113 shows a dot plot result of in vivo model to determine the functional impact of Fam117b and AdamtI5 (in a Western diet mouse model). Fully repopulated mice, where every hepatocyte expresses the shRNA of interest were exposed to the “Western Diet”, inducing progressive NAFLD. On the left the experimental layout is shown, in the middle representative images of the macroscopic liver are shown. On the right the quantitative results from blinded fibrosis score scoring by a certified pathologist are shown, as well as the knockdown the efficiency. Knockdown of the target significantly reduced fibrosis level. Evaluation of fibrosis score based on Sirius red staining and graded by pathological expert on a scale of 1-5 with 1 being minimal, 2 mild, 3 moderate, 4 marked, and 5 severe.

FIG. 114 shows target marker list 1, which includes targets that have passed In-vitro QC and have undergone or undergoing in-vivo validation inclusive of clonal expansion, diet treatment and partial hepatectomy. Some of the targets have been included in in-vivo secondary screen as controls.

FIG. 115 shows target marker list 2, which includes targets that have passed literature review and have undergone/currently undergoing in-vitro QC or are in the queue. Some of these targets have passed in-vitro QC and are included in the in-vivo secondary analysis. Some of these will also be shortlisted based on phenotype for in-vivo primary validation.

FIG. 116 shows target list 3, which includes targets that have passed literature review inclusive of human homolog, target being novel, no association with NAFLD pathologies, high survival probability in liver cancer patients and in-house patient transcriptomics data.

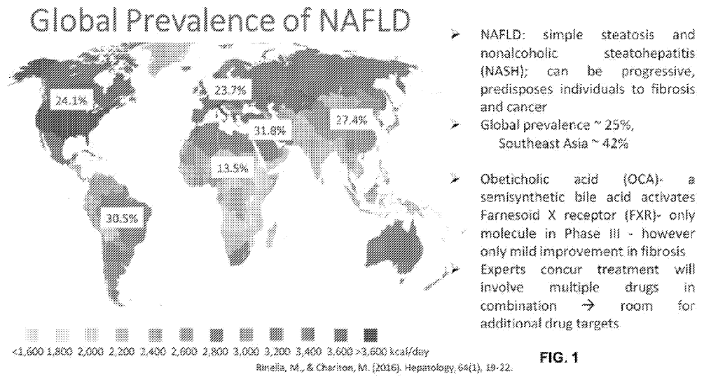

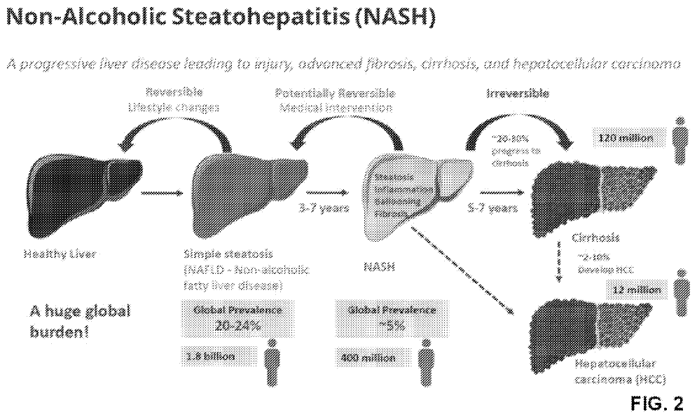

Example embodiments of the disclosure will be better understood and readily apparent to one of ordinary skill in the art from the following discussions and if applicable, in conjunction with the figures. It should be appreciated that other modifications may be made without deviating from the scope of the invention. Example embodiments are not necessarily mutually exclusive as some may be combined with one or more embodiments to form new exemplary embodiments. The example embodiments should not be construed as limiting the scope of the disclosure.

### EXPERIMENTAL SECTION

Disclosed herein is a technology that addresses the needs for providing an alternative diagnostic methods and targets for therapy. By conducting a genome wide in vivo functional genetic screen, the inventors identified modulators and therapeutic targets in NAFLD mouse model. The inventors of the present disclosure then did a fully in vivo functional genomics screen of roughly 80,0000 shRNAs in an intervention set up for Western Diet induced NASH. A multiplex, layered in silico target identification pipeline was applied for identification and prioritization of high-confidence targets. About ~200 high-confidence targets were shortlisted, which were further prioritized in ~50 targets passing highest level of stringency and 80 targets passing high level of stringency. The inventors then went on to run the top targets through our multiplex in vitro validation pipeline.

The ultimate project objective addressed is the identification of therapeutic targets to intervene in Non-Alcoholic fatty Liver Disease (NAFLD) progression, especially from simple steatosis to Non-Alcoholic SteatoHepatitis (NASH). The fundamental premise is that increasing the endogenous regenerative capacity of the liver can compensate any liver damage and thereby attenuating fibrosis and progressive disease. For reaching this goal, a genome wide in vivo functional genetic intervention screen was conducted in the “Western Diet” (WD) mouse model. The library of ~80 k shRNAs was subdivided into 32 pools and the shRNAs were cloned into a transposon-based construct. Sub-pools were delivered by hydrodynamic tail-vein injection to the liver of mice as “naked” plasmids. Through the combination of transposon-based constructs with a transposase coding plasmid we reach stable integration in about 5-10% of hepatocytes. Expression of shRNAs was induced after simple steatosis is reached. Mouse livers were harvested after 26 weeks of feeding either the WD or control chow. From the livers genomic DNA was isolated and a PCR to amplify the expression cassette was conducted. PCR products were sequenced using Illumina based Next Generation Sequencing (NGS) on a HiSeq4000 machine.

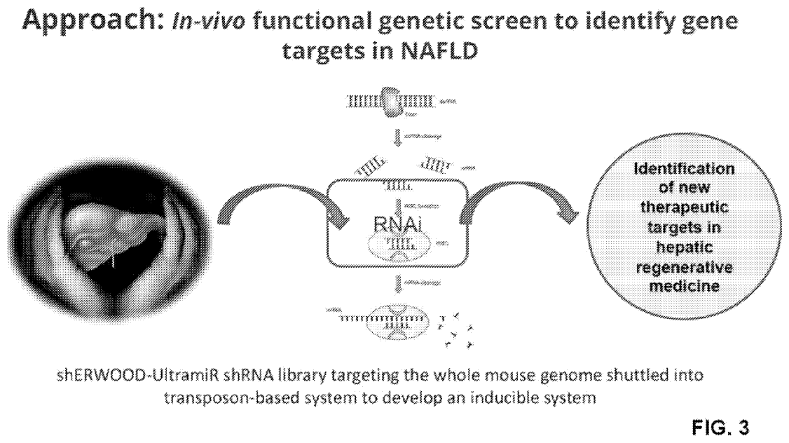

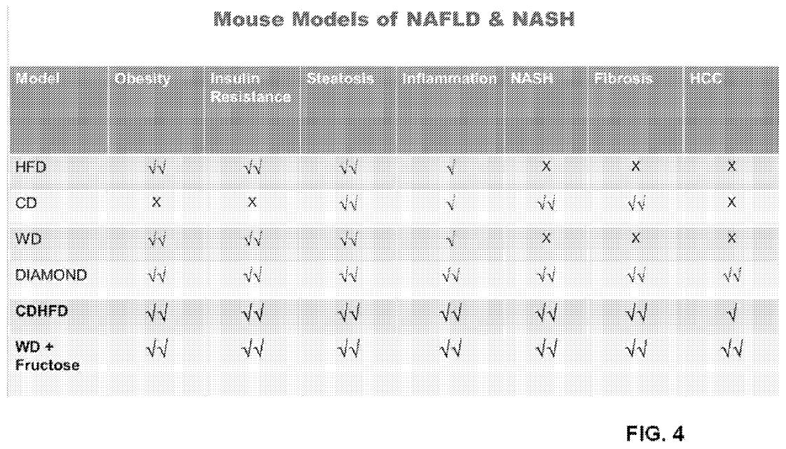

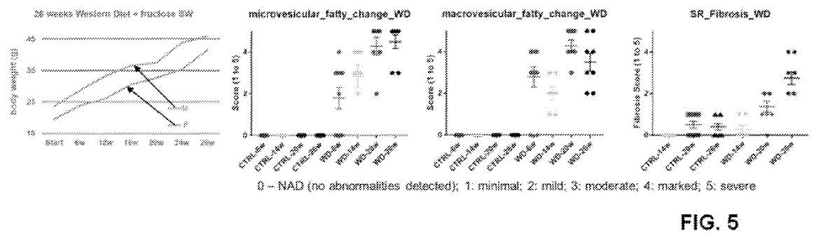

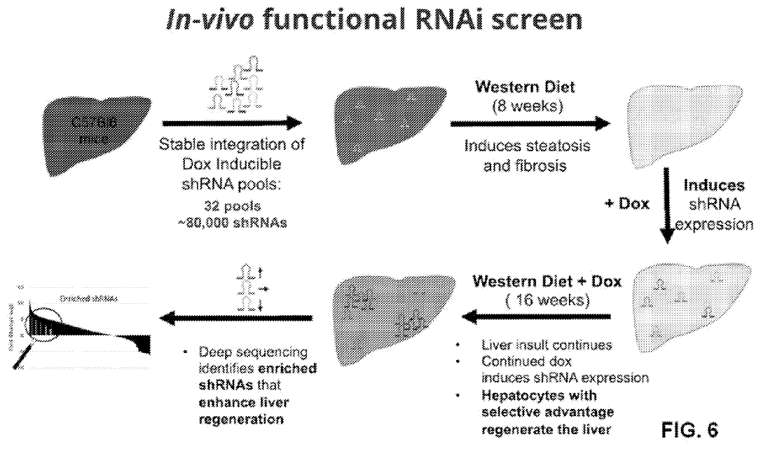

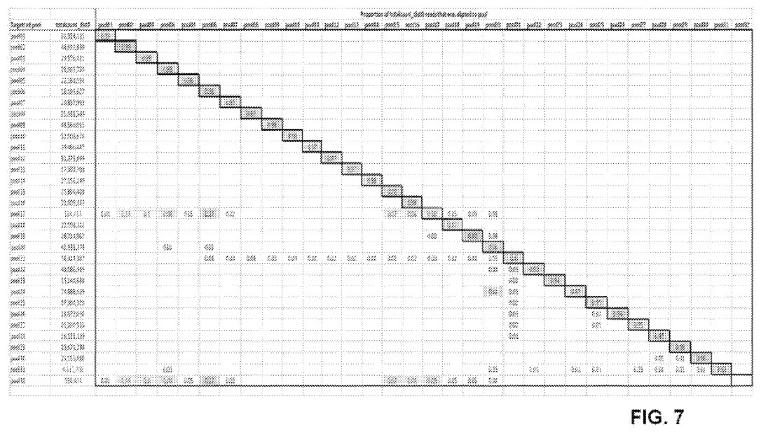

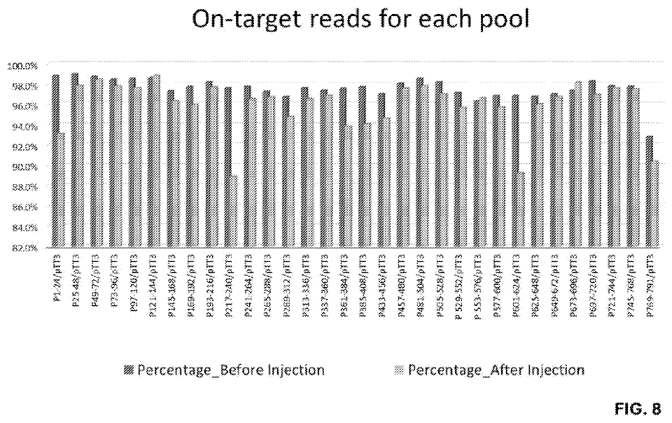

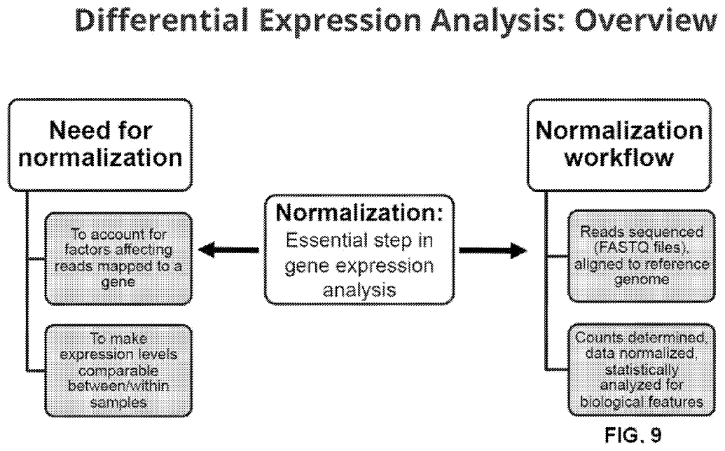

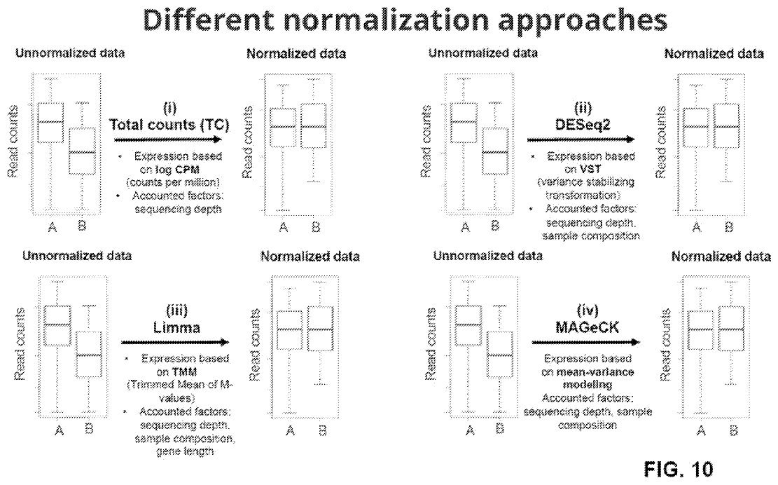

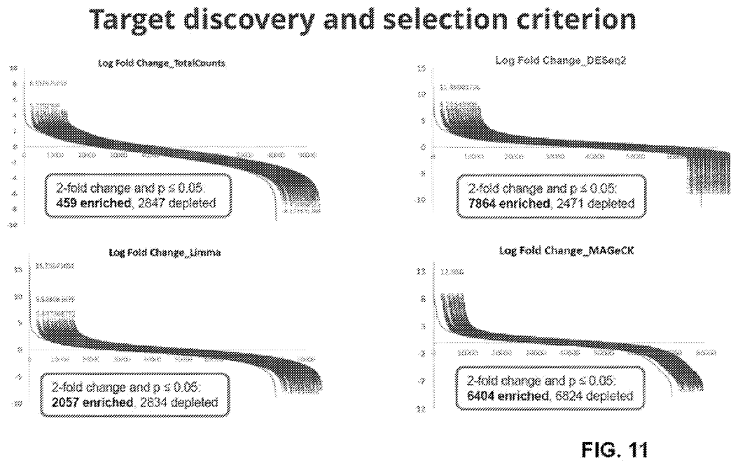

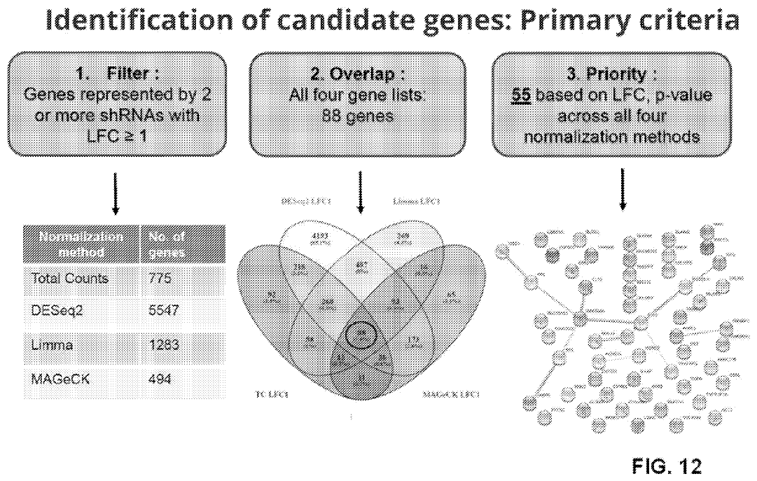

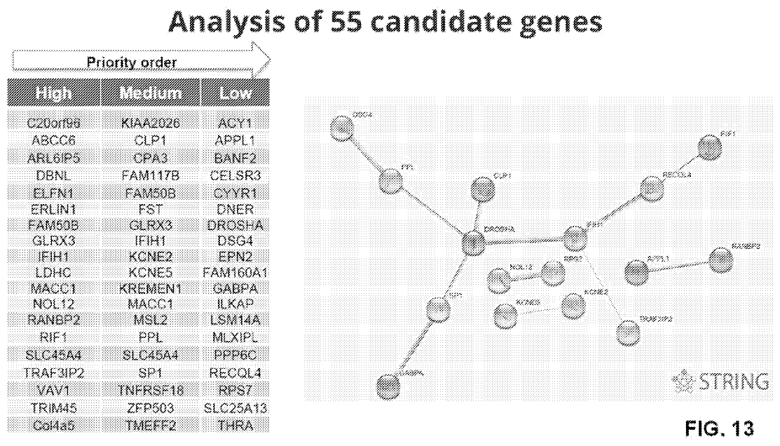

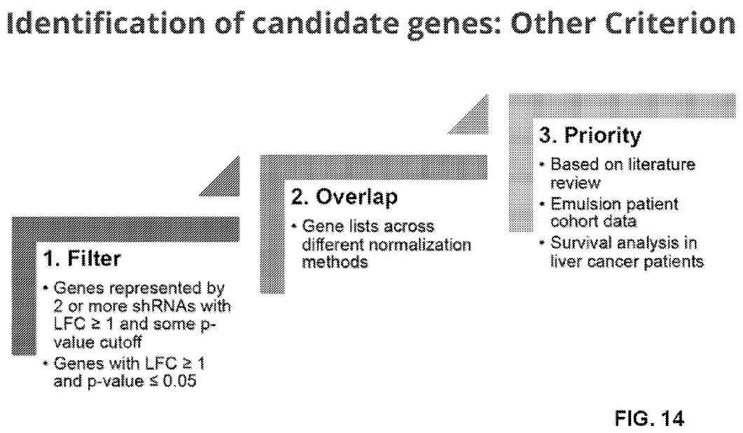

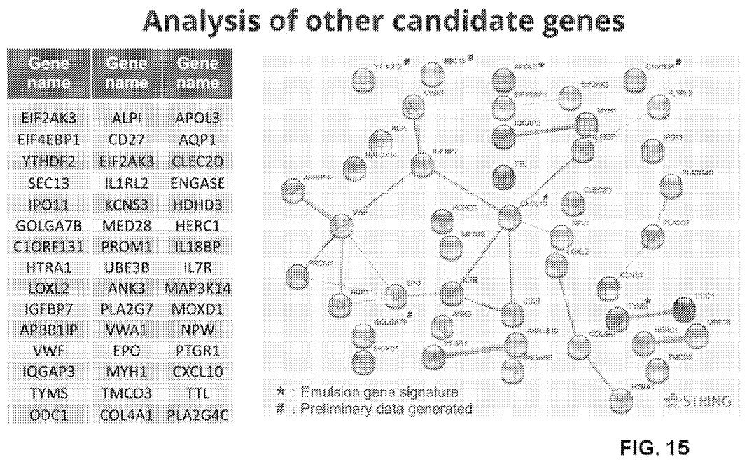

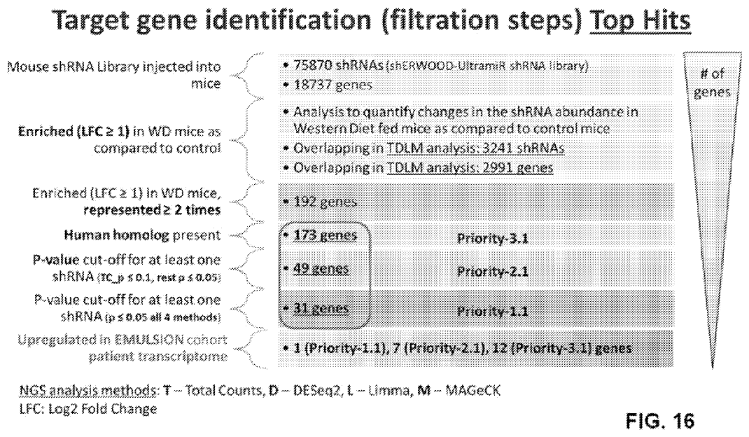

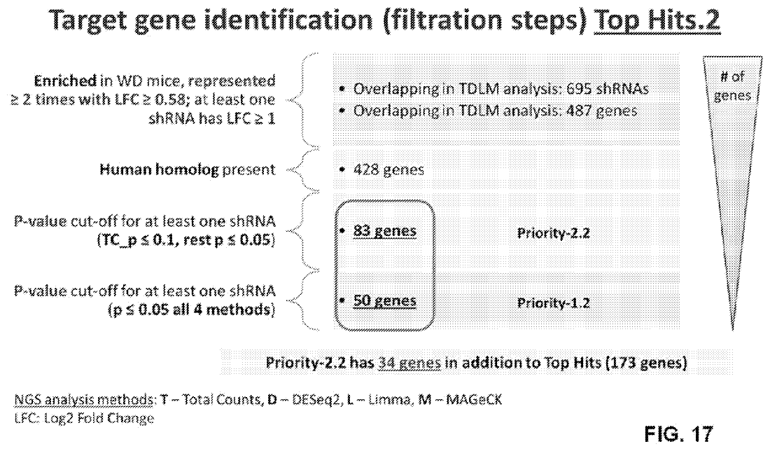

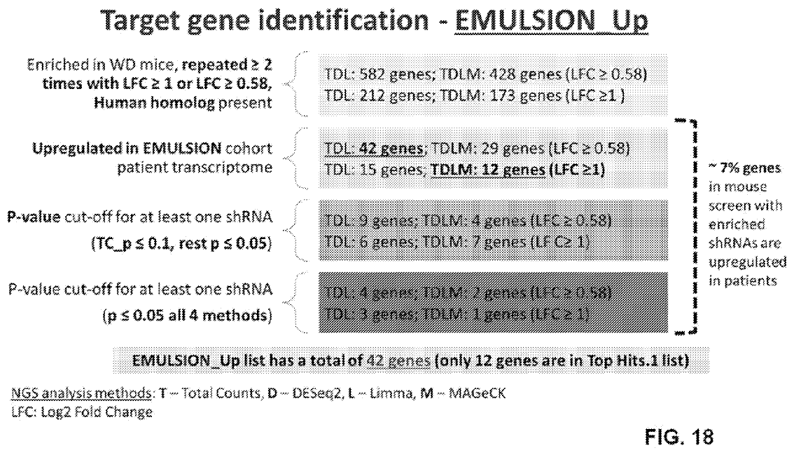

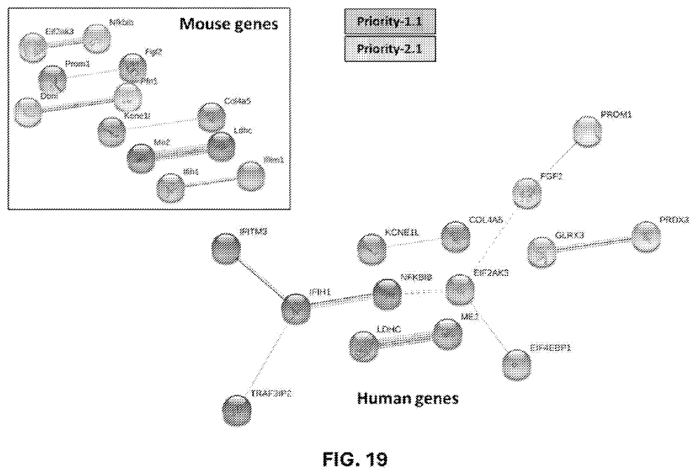

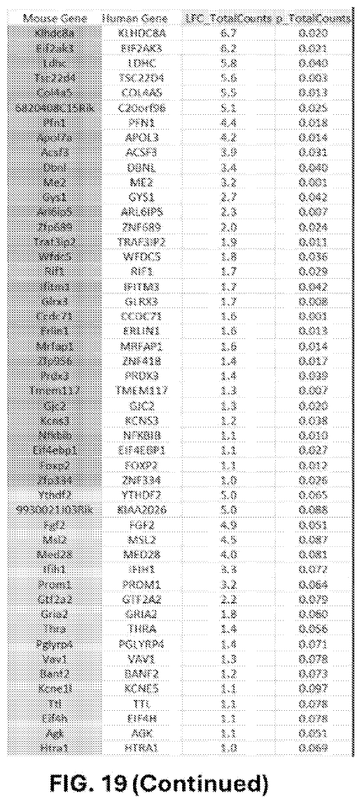

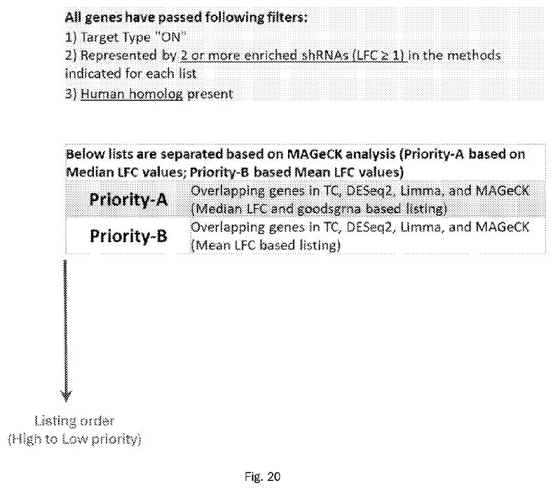

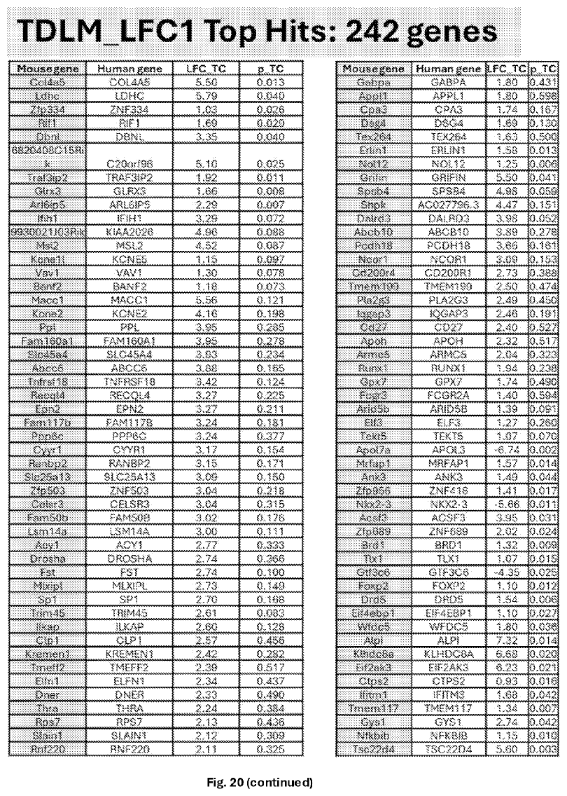

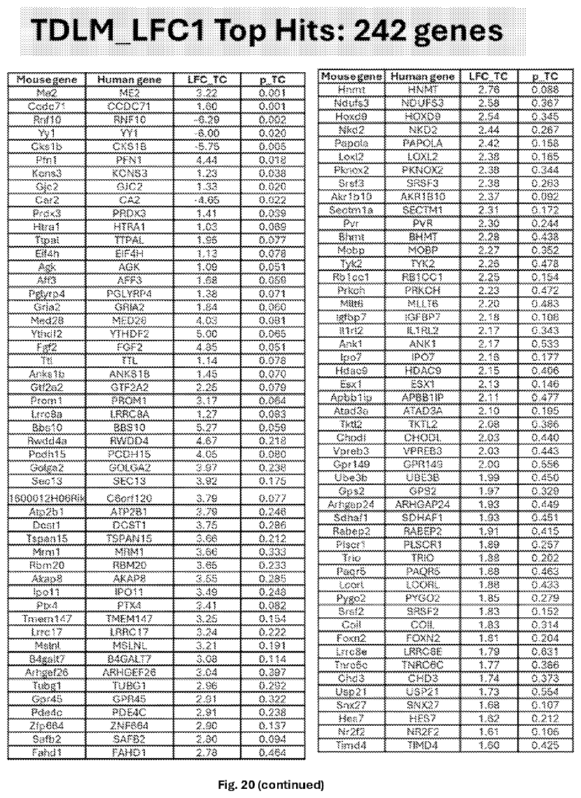

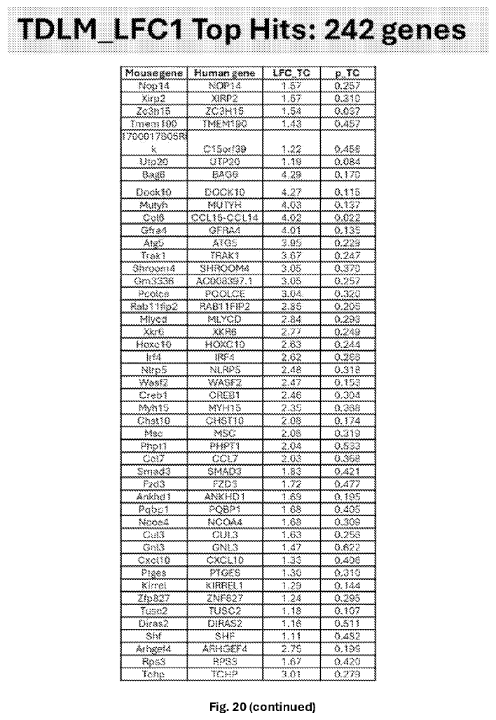

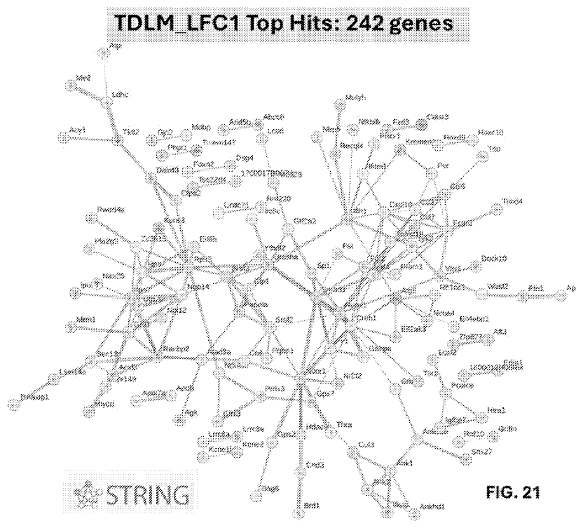

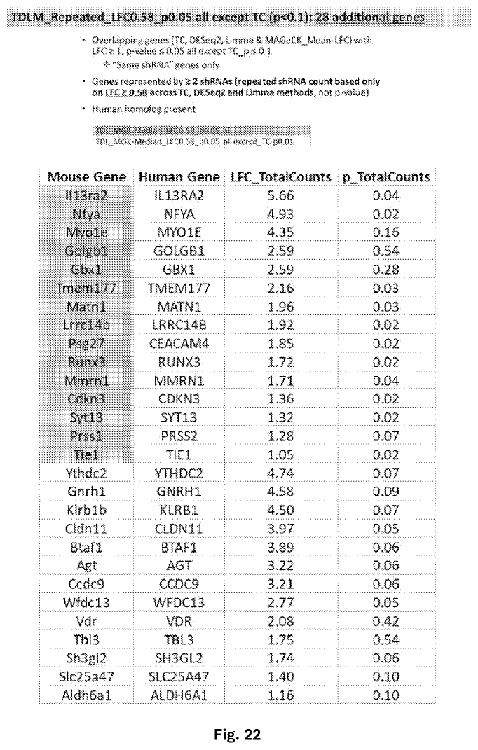

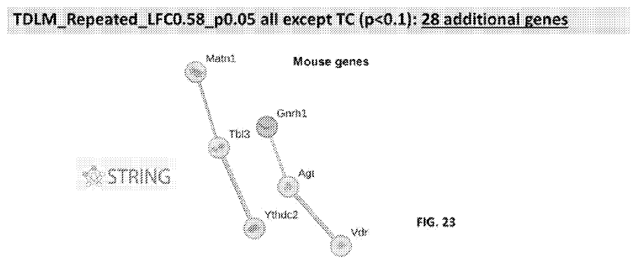

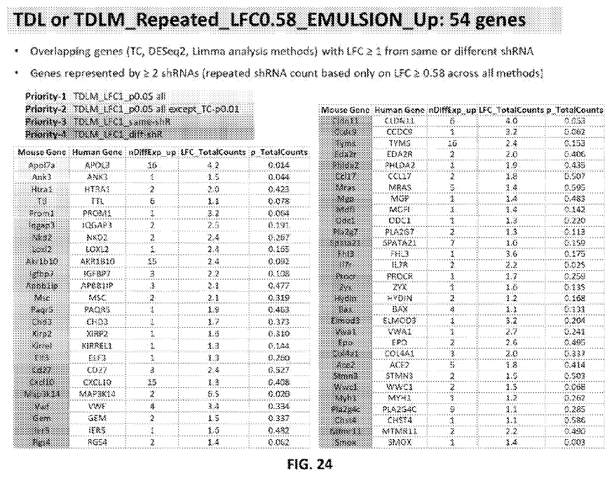

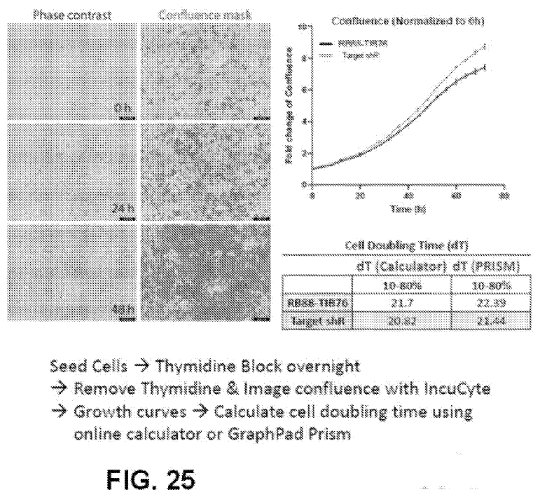

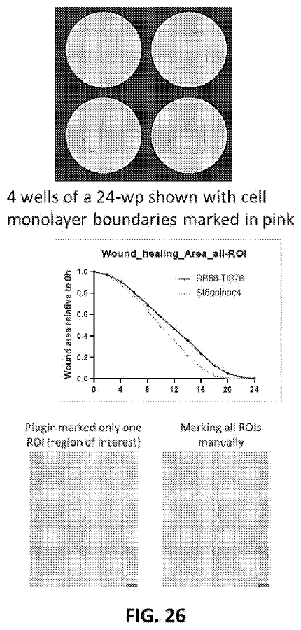

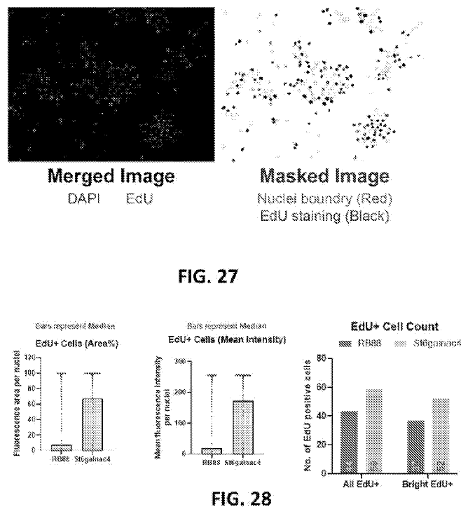

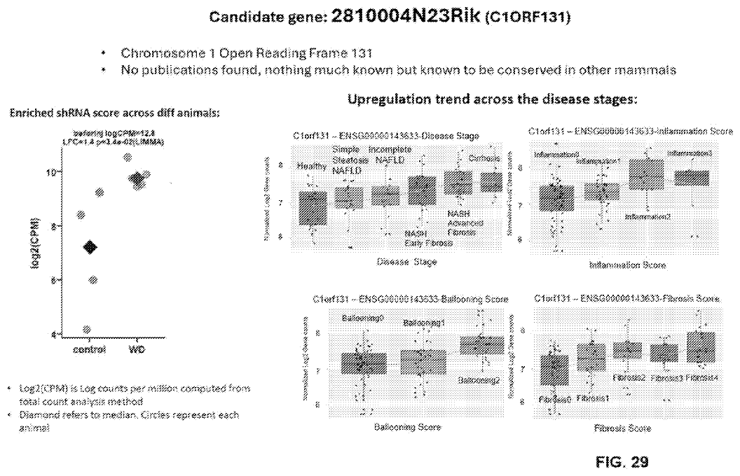

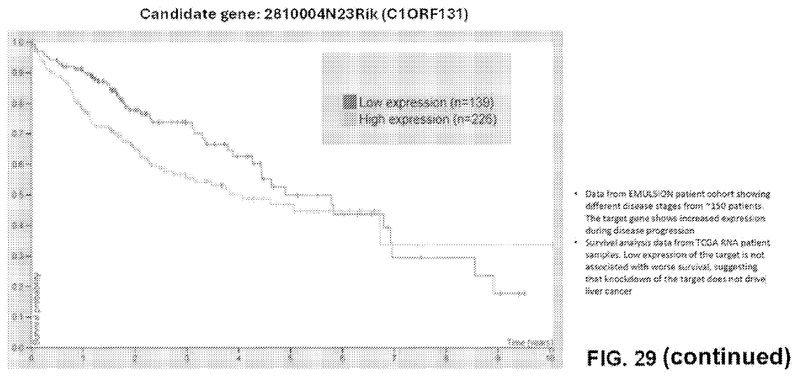

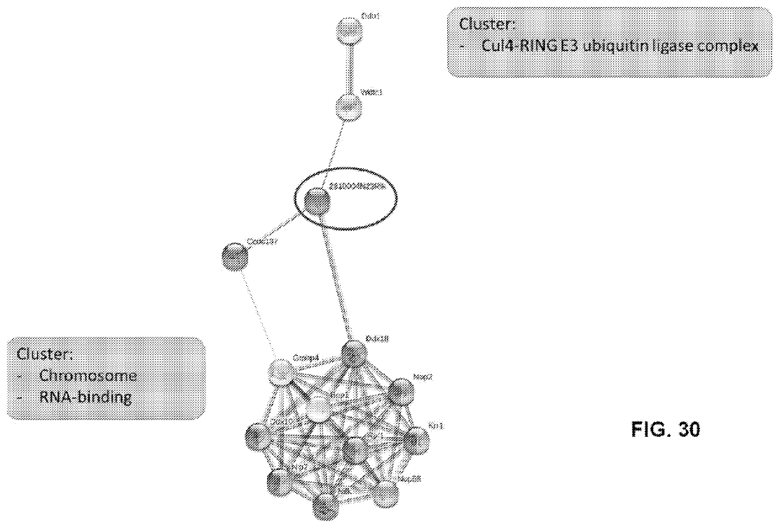

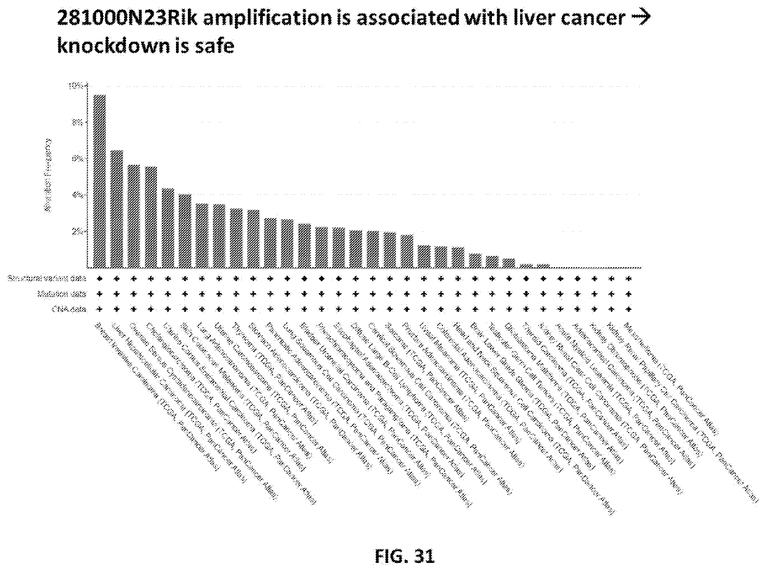

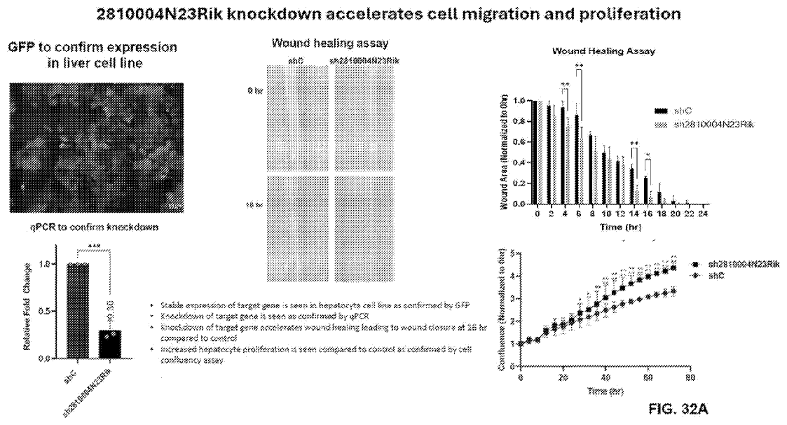

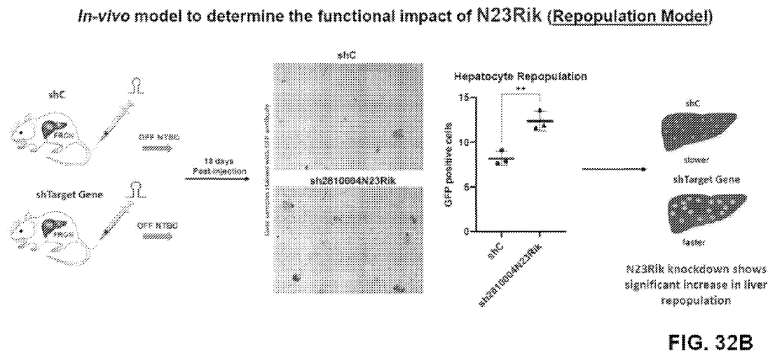

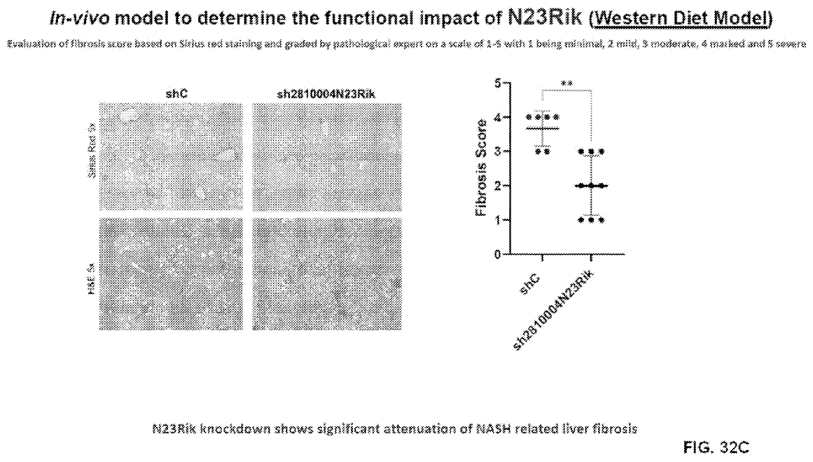

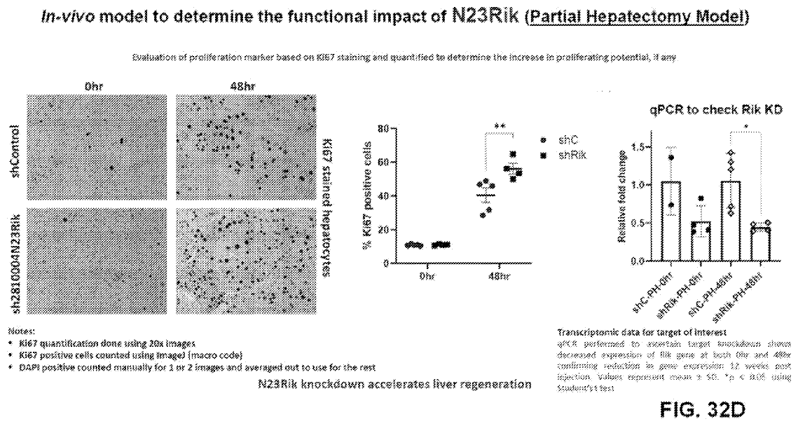

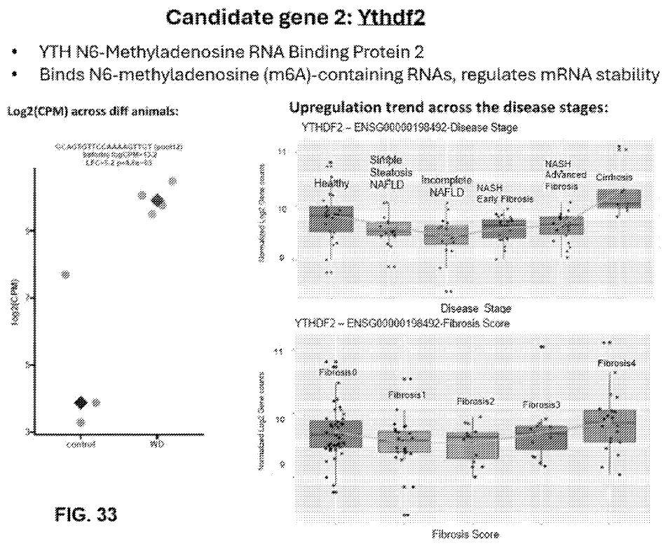

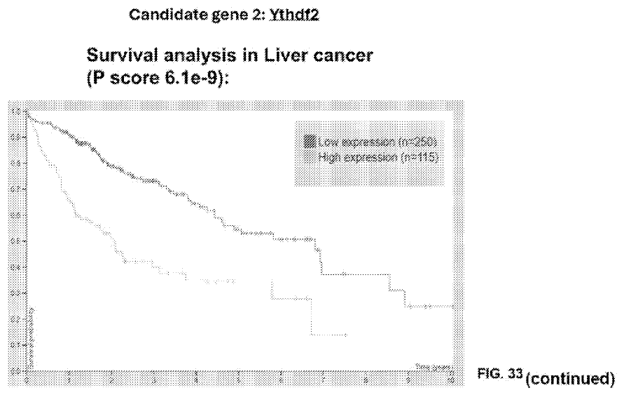

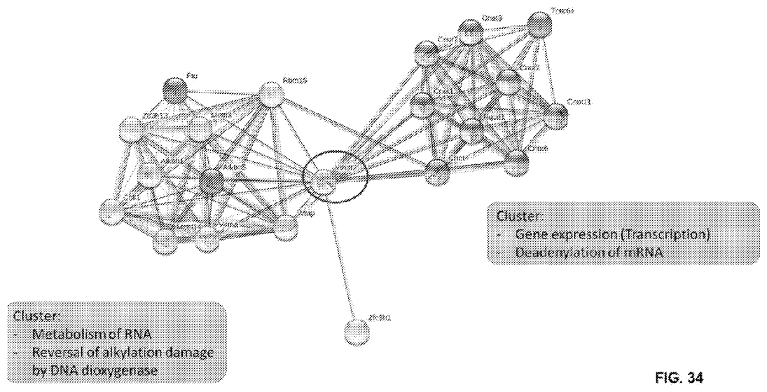

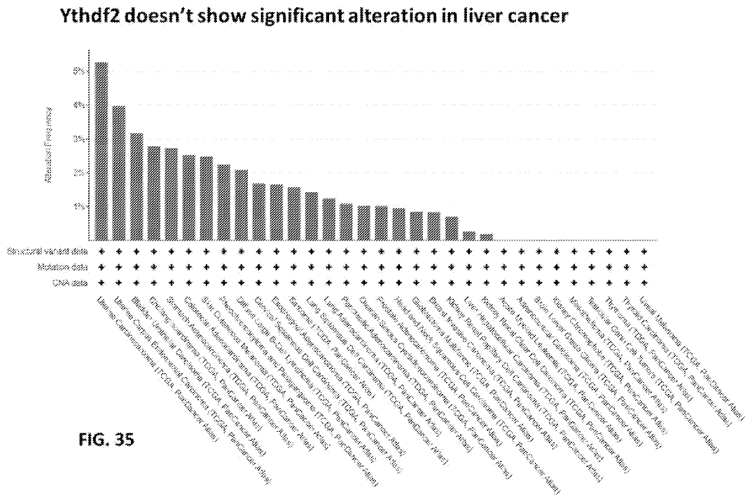

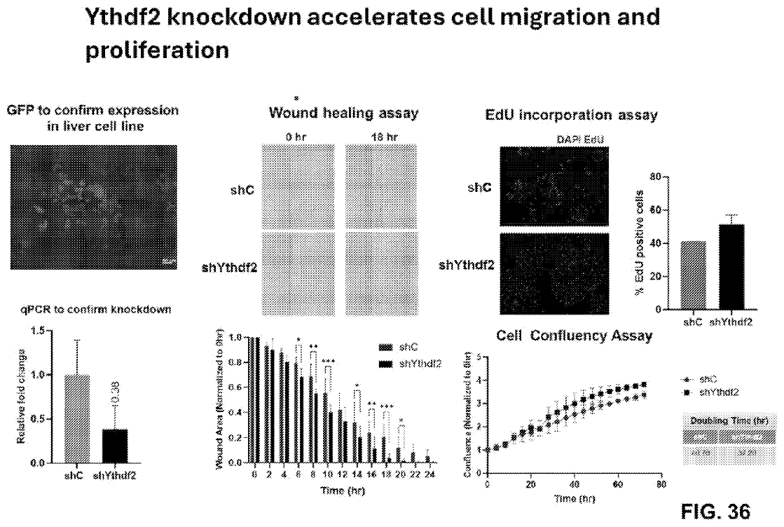

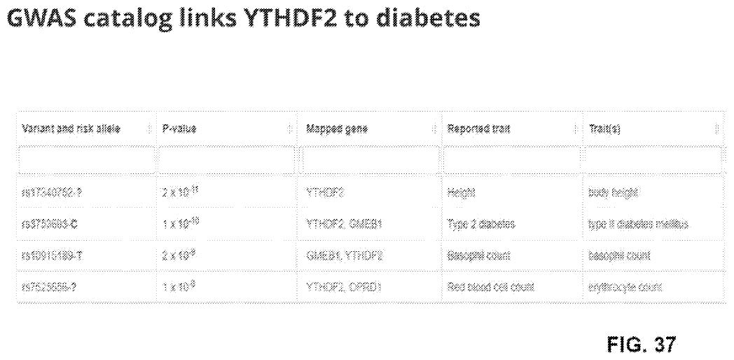

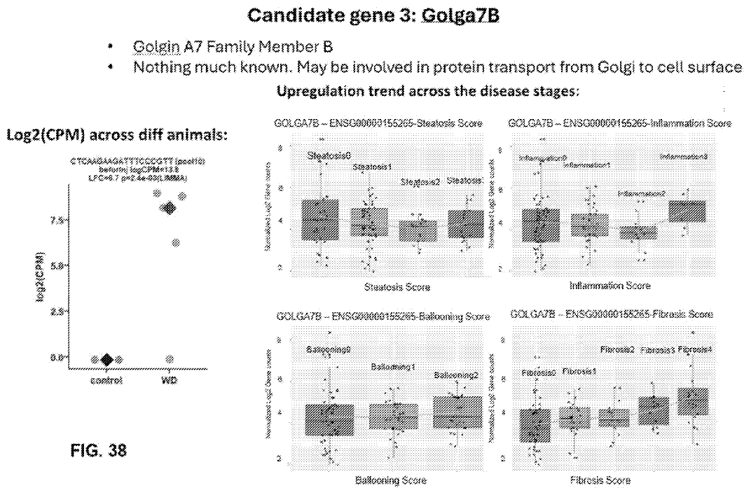

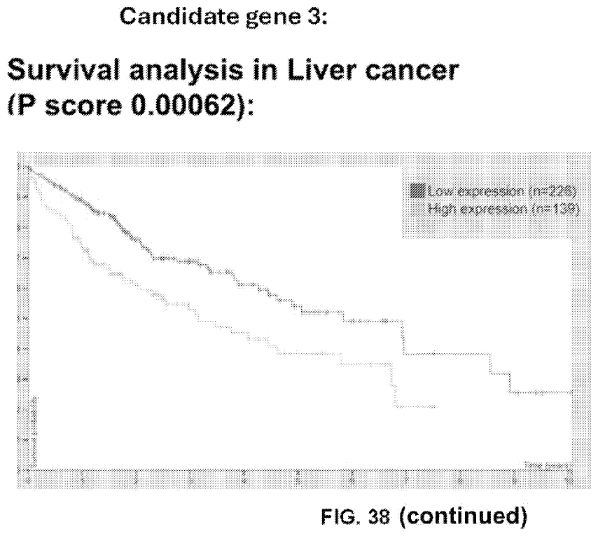

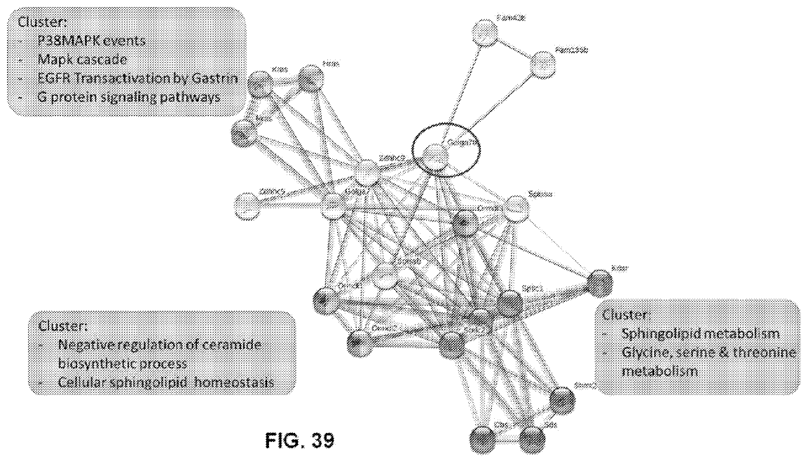

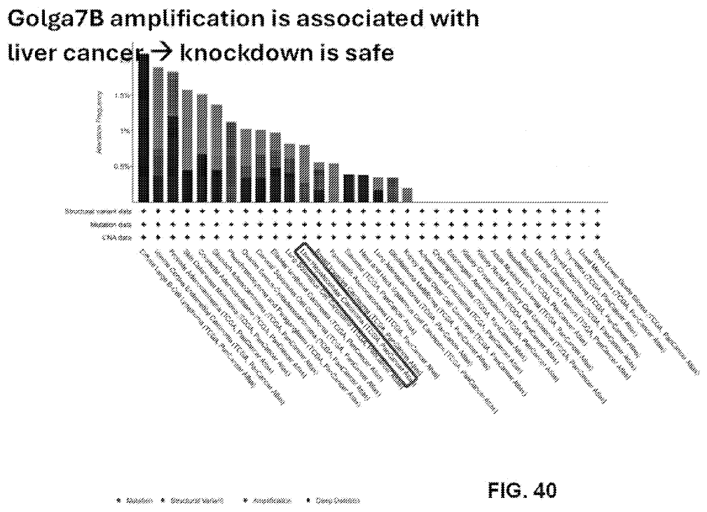

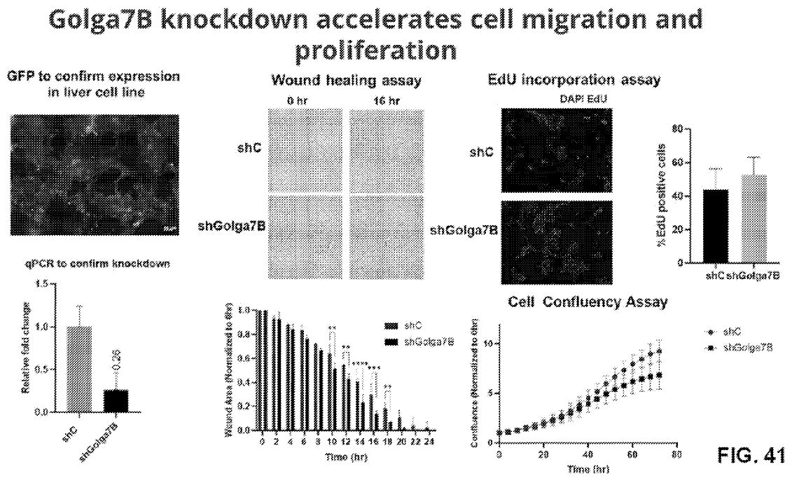

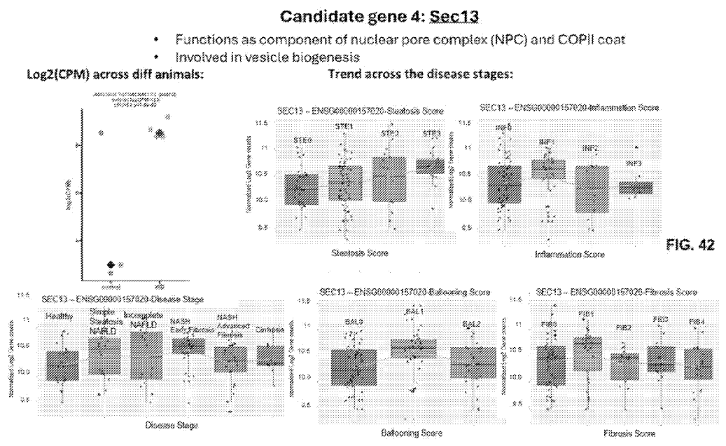

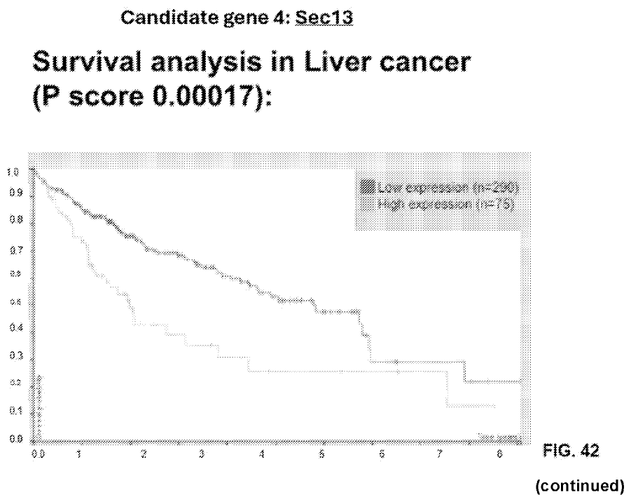

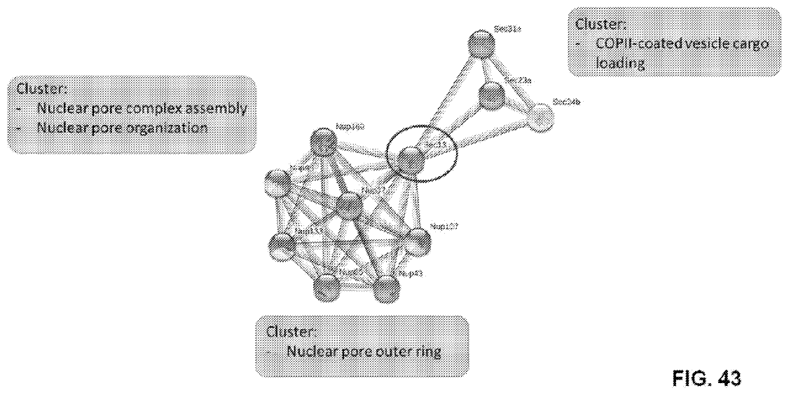

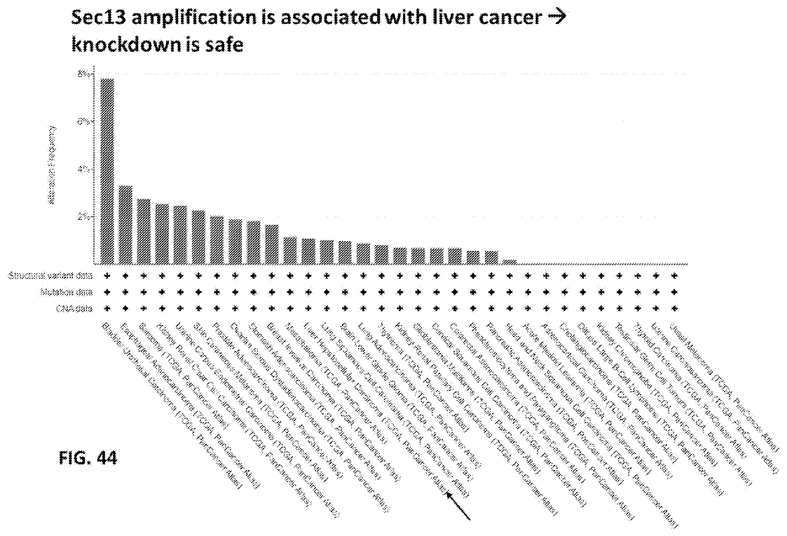

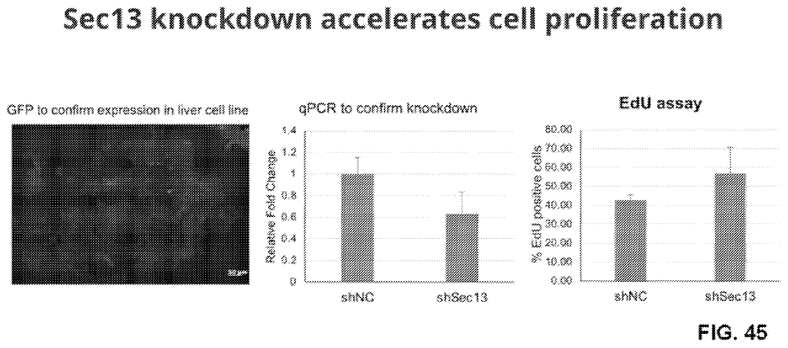

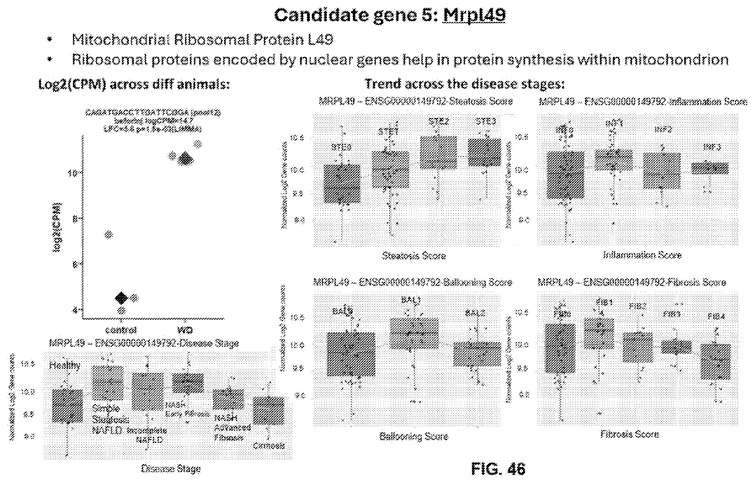

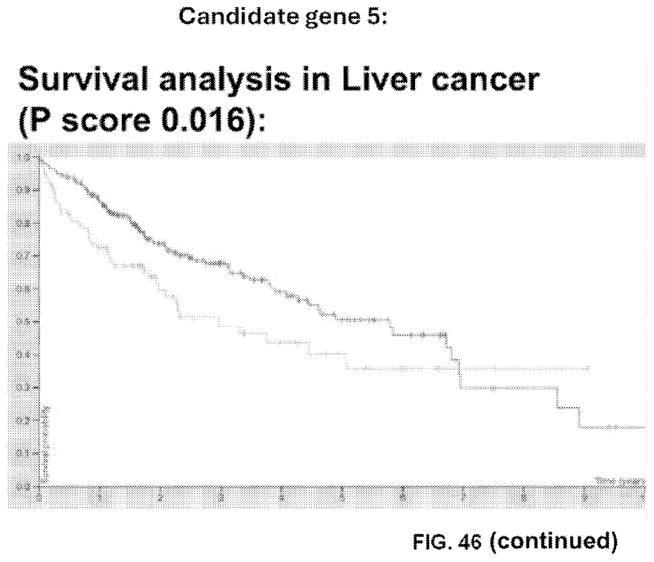

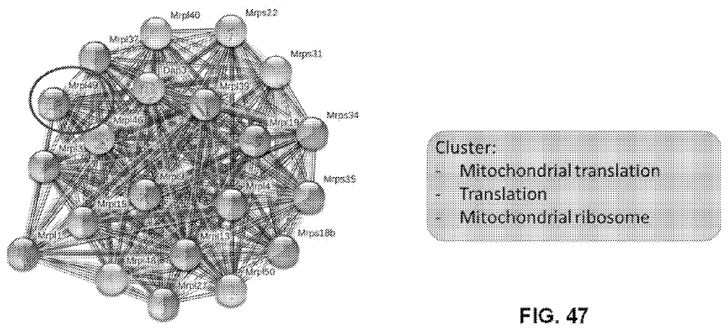

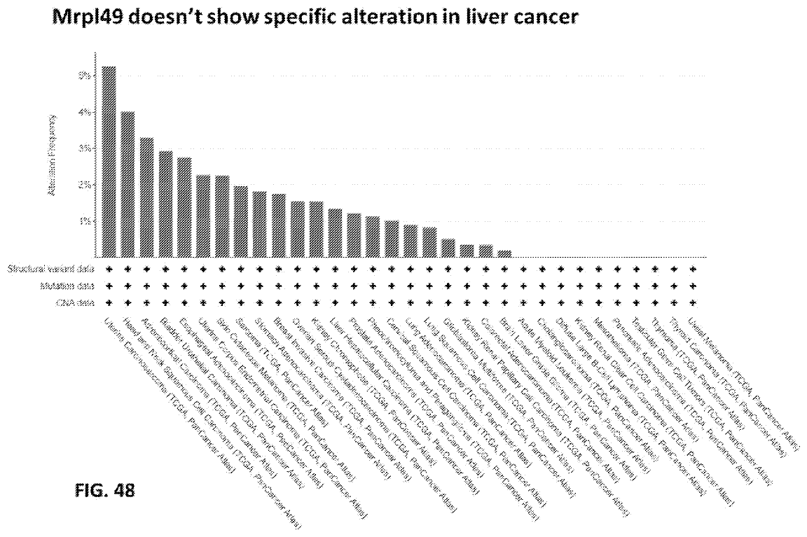

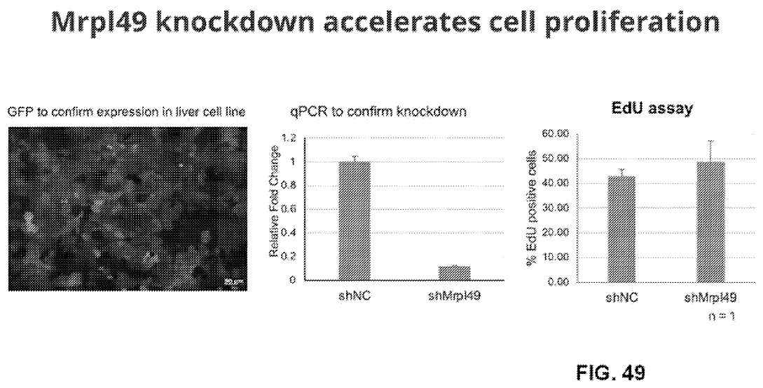

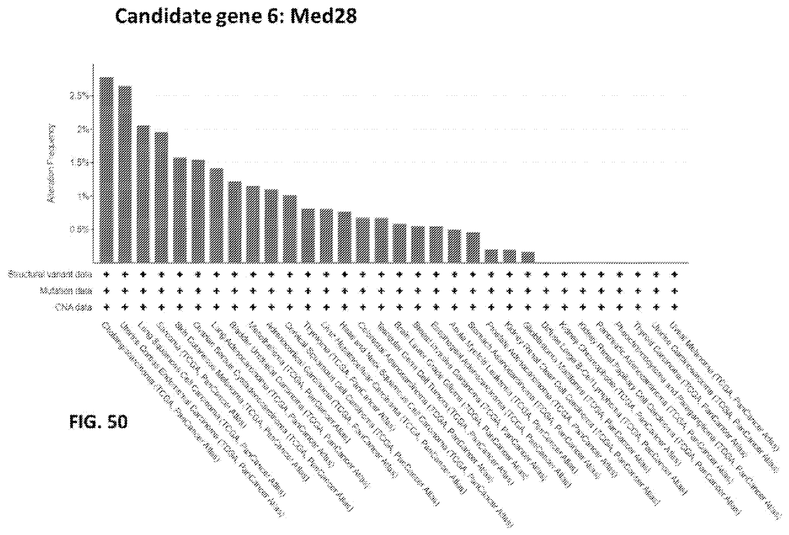

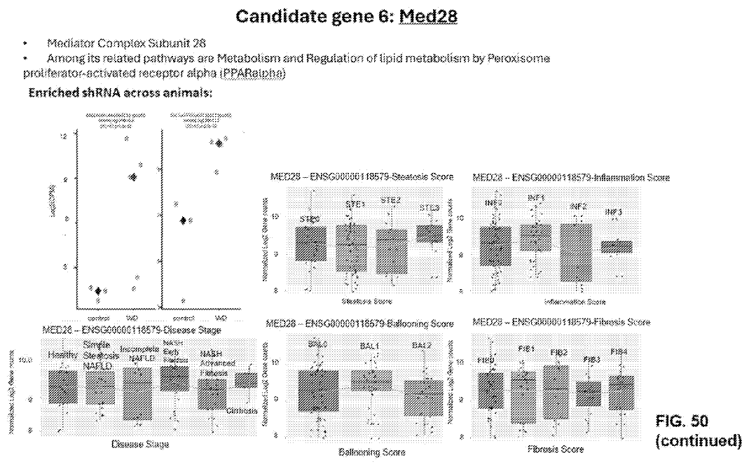

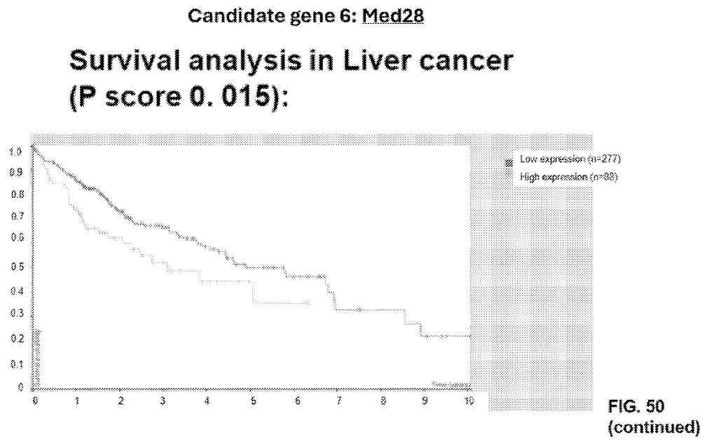

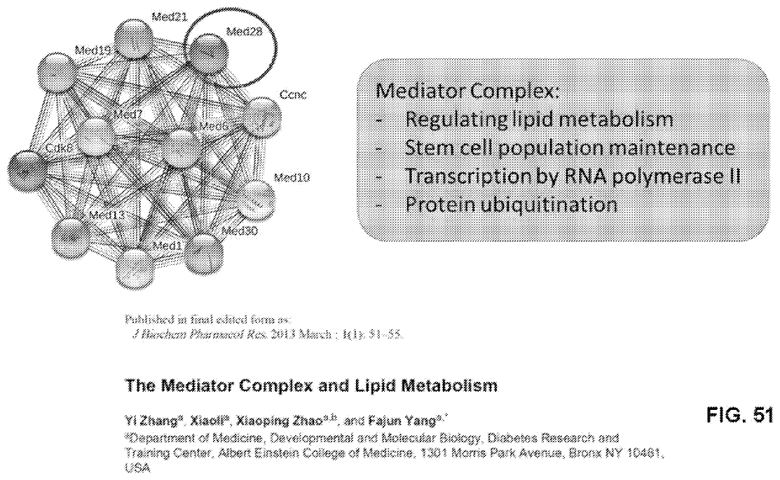

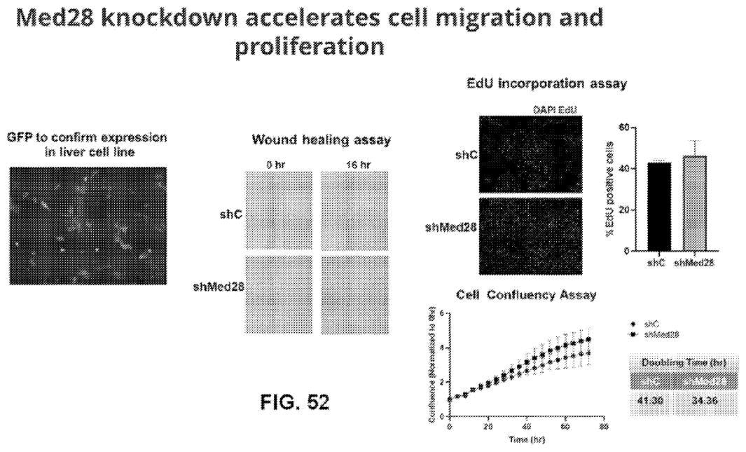

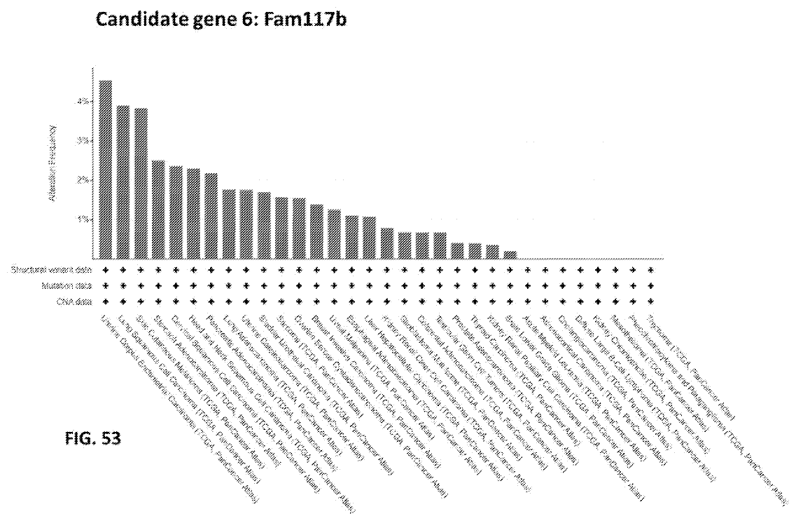

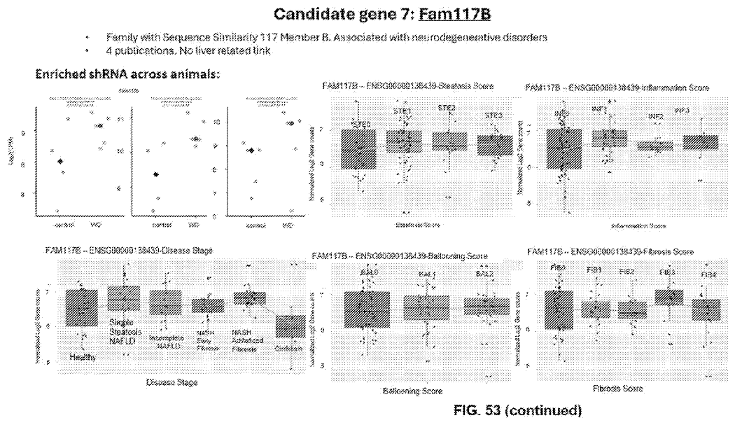

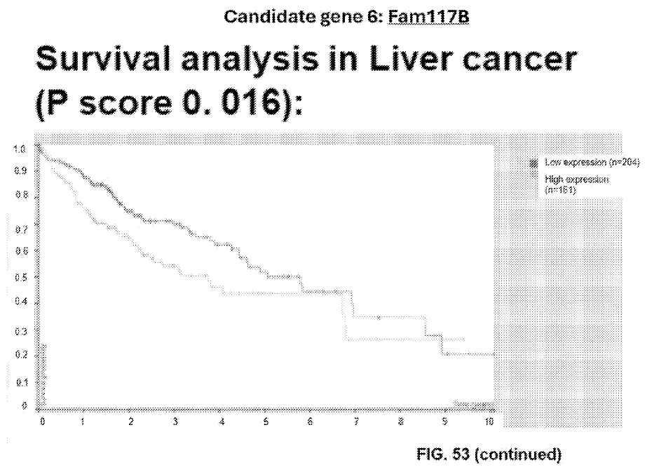

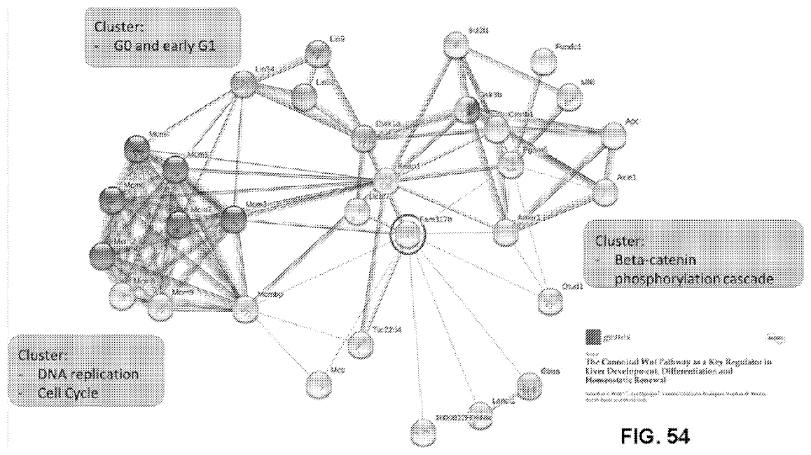

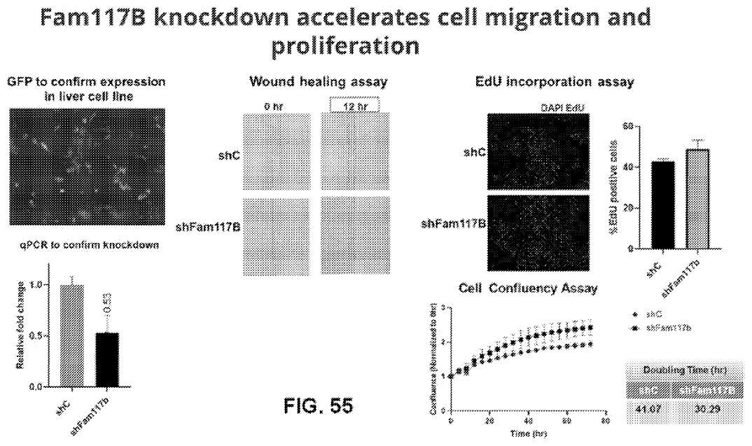

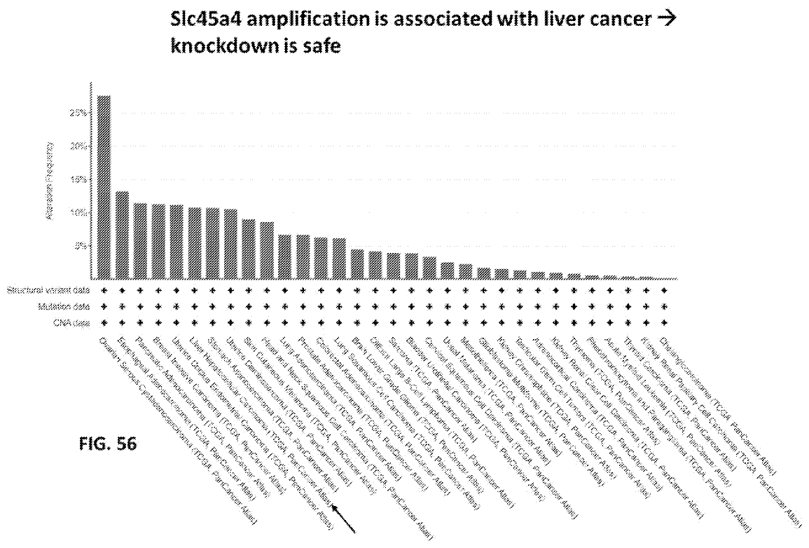

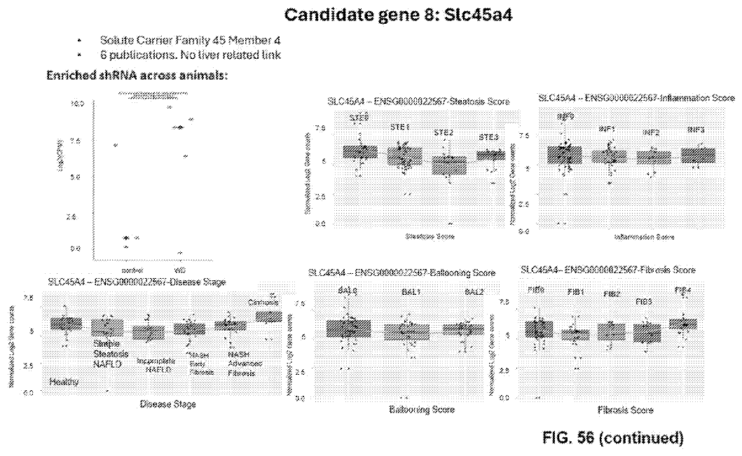

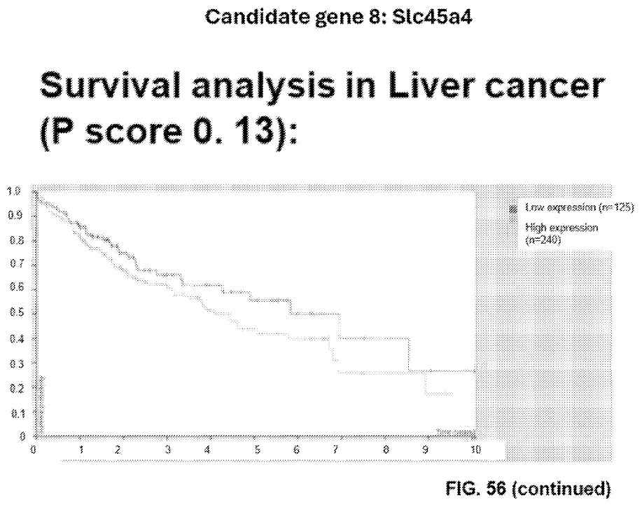

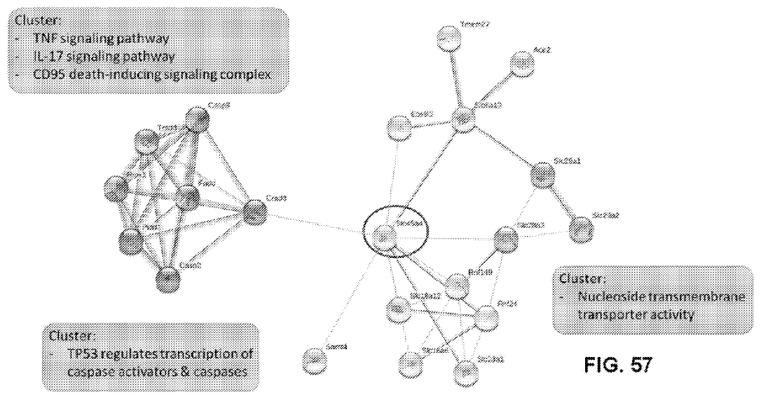

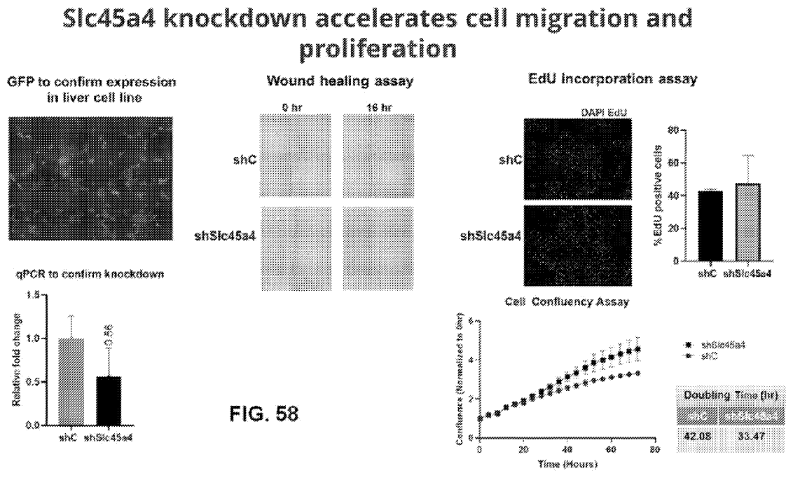

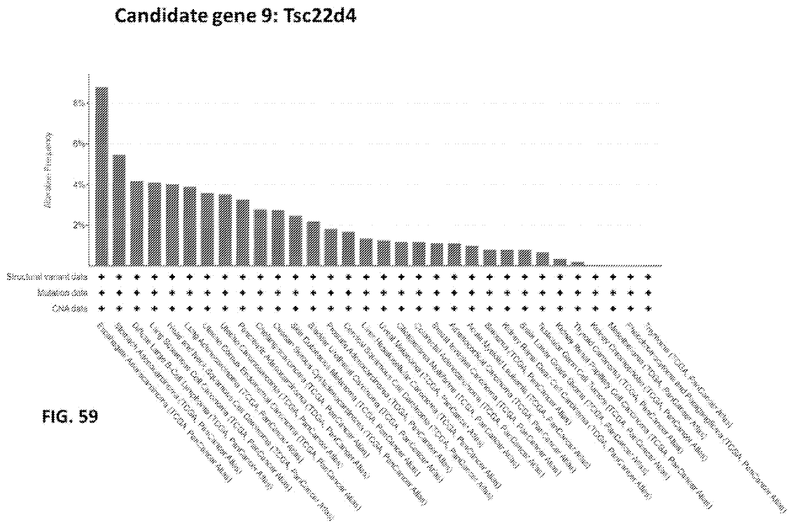

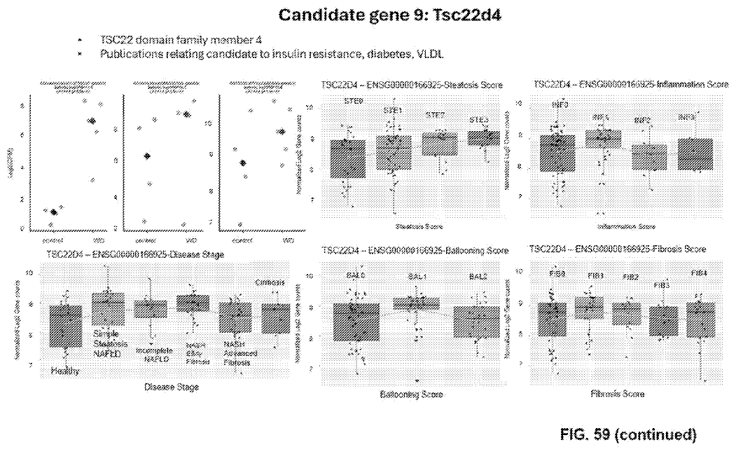

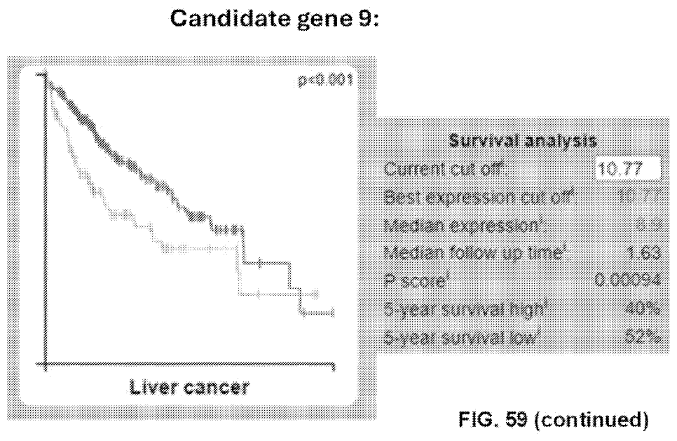

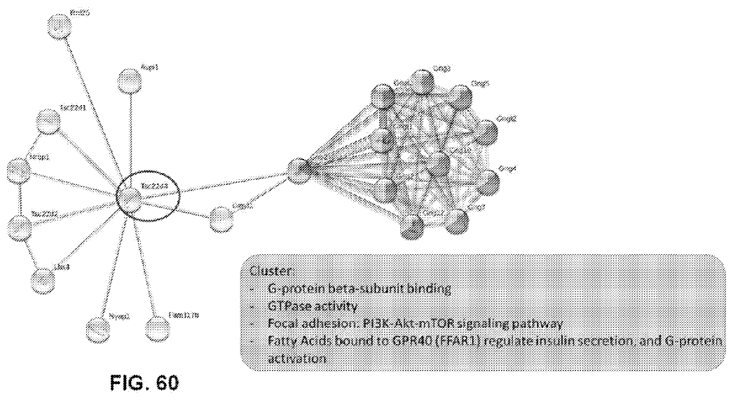

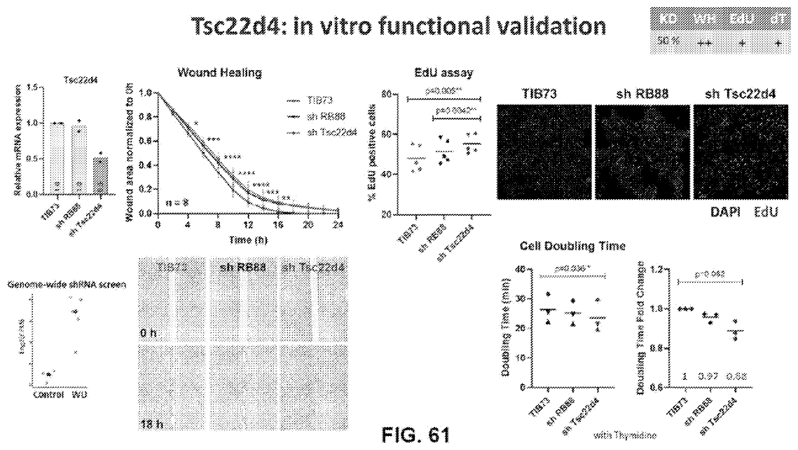

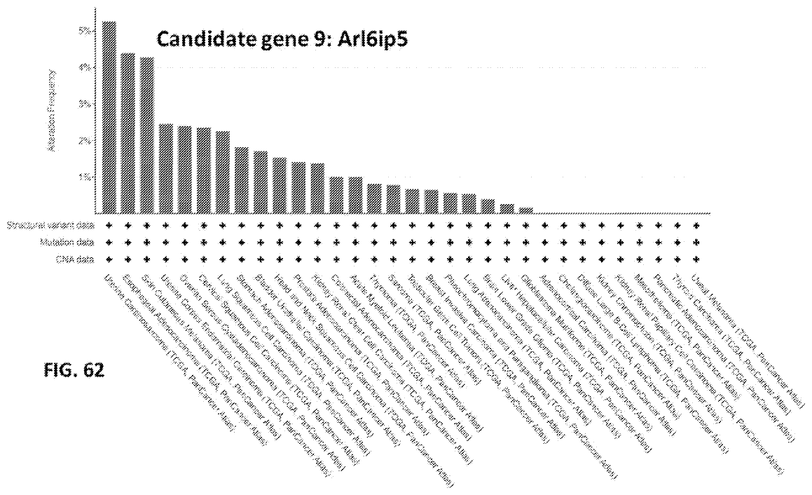

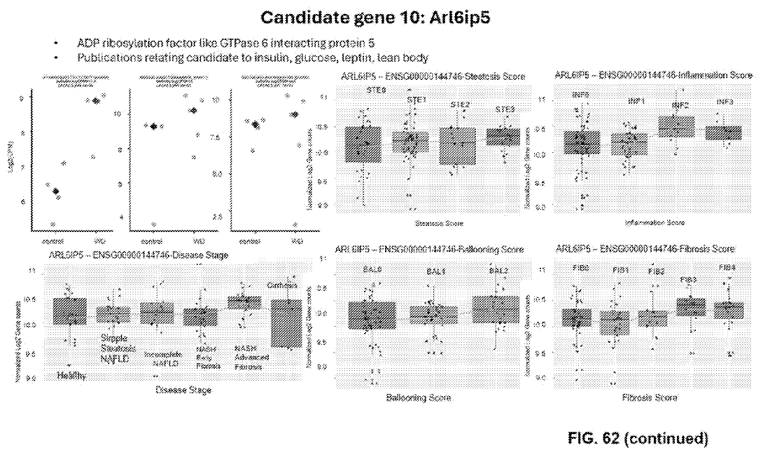

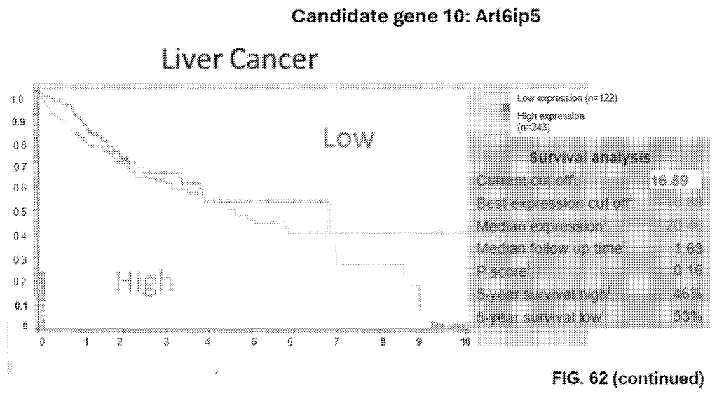

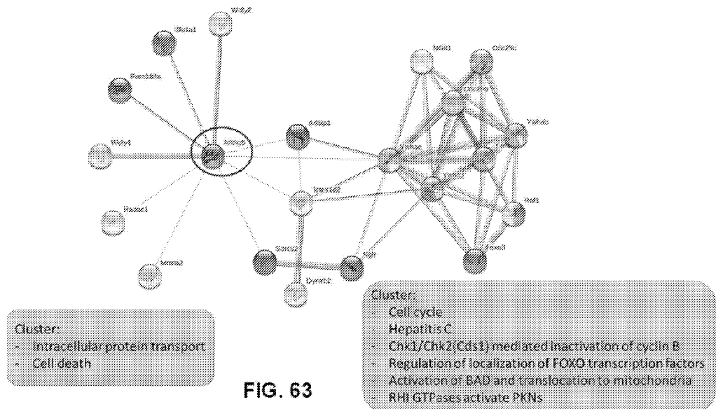

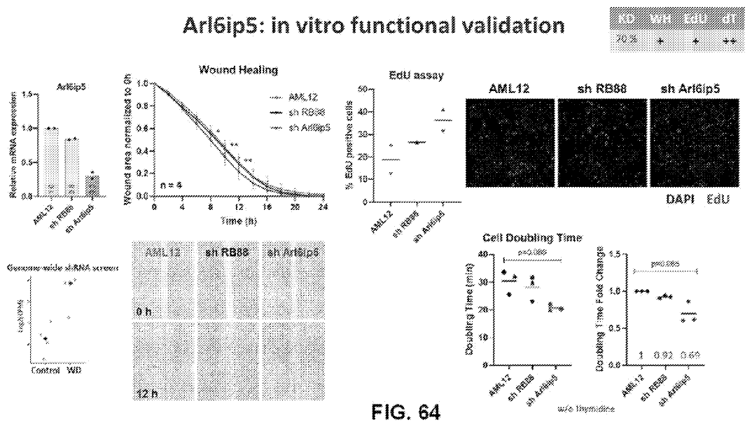

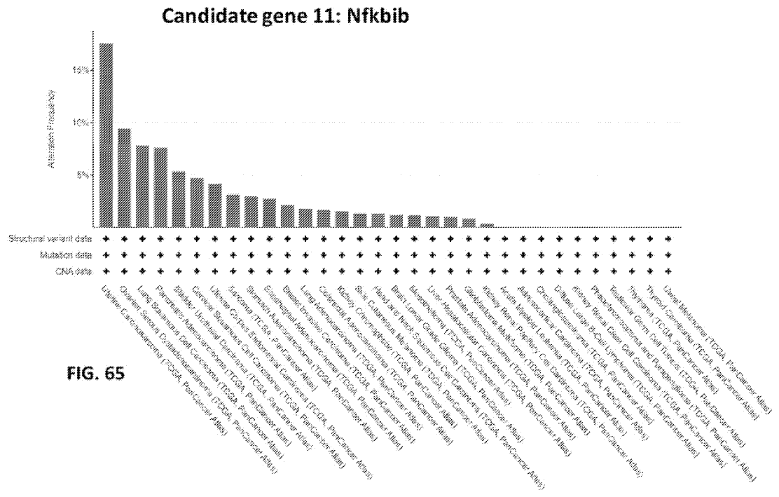

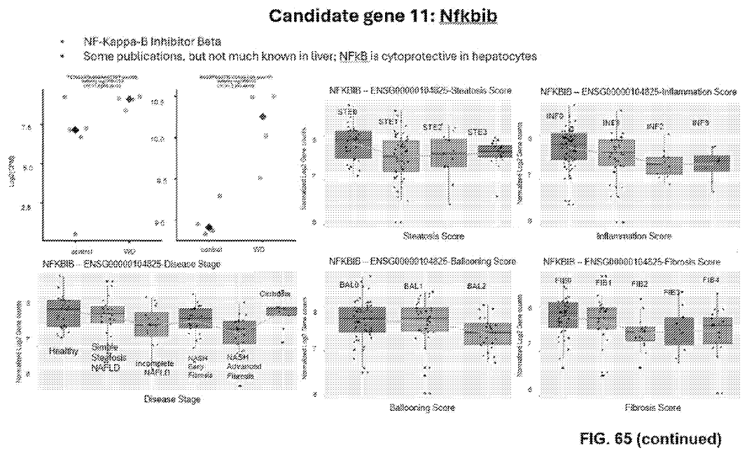

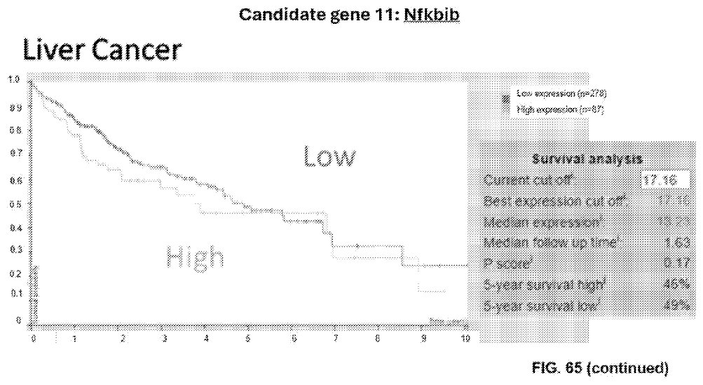

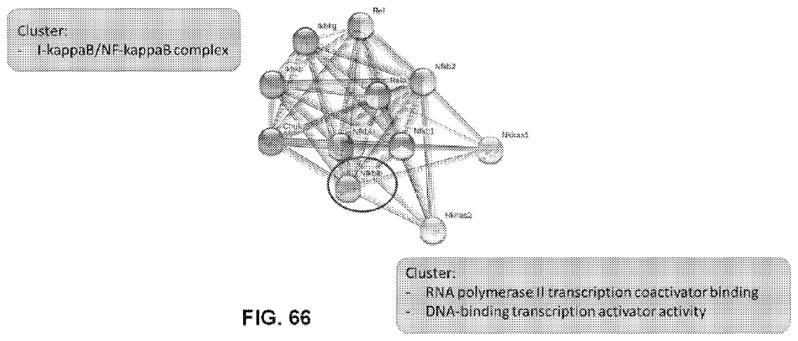

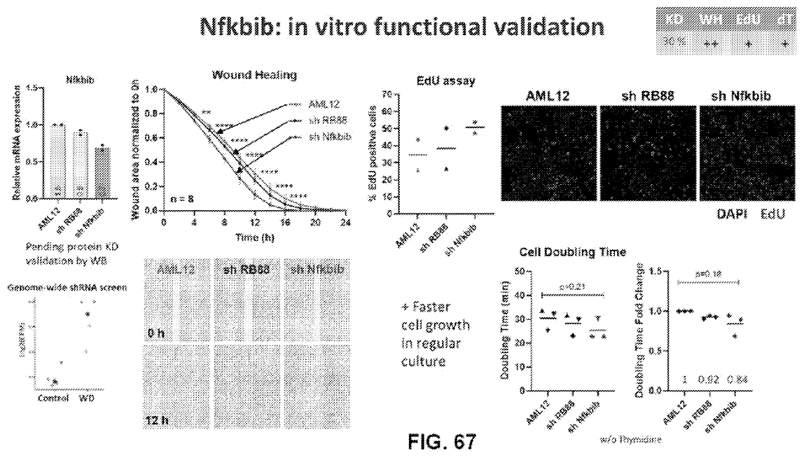

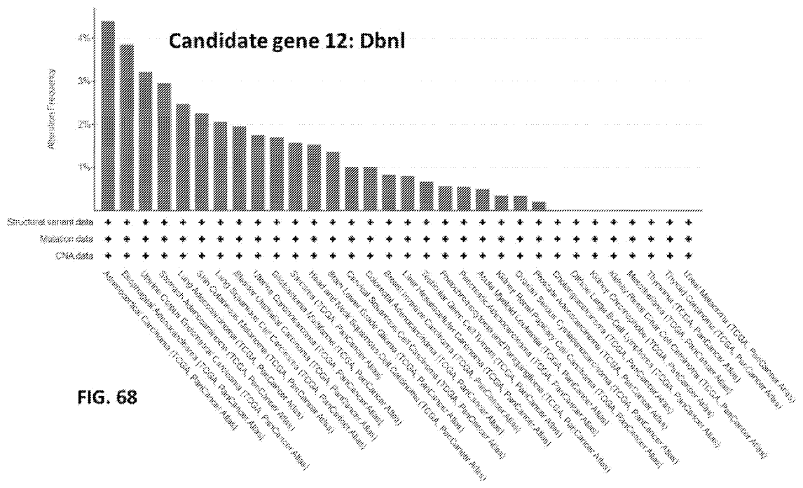

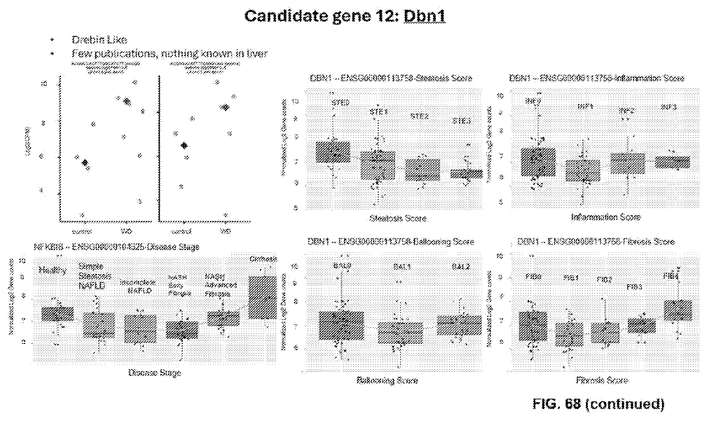

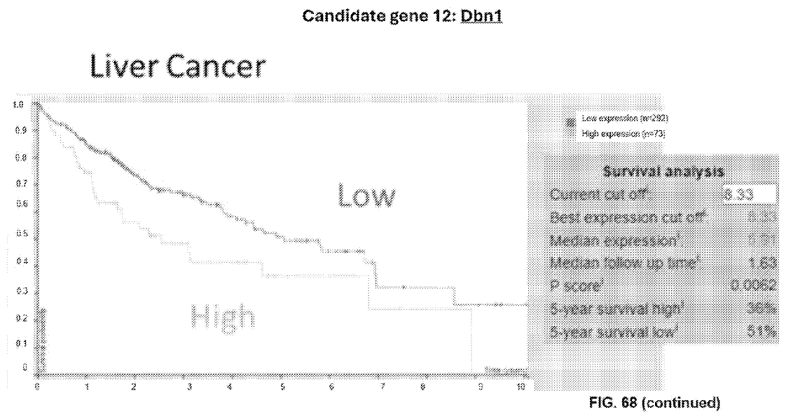

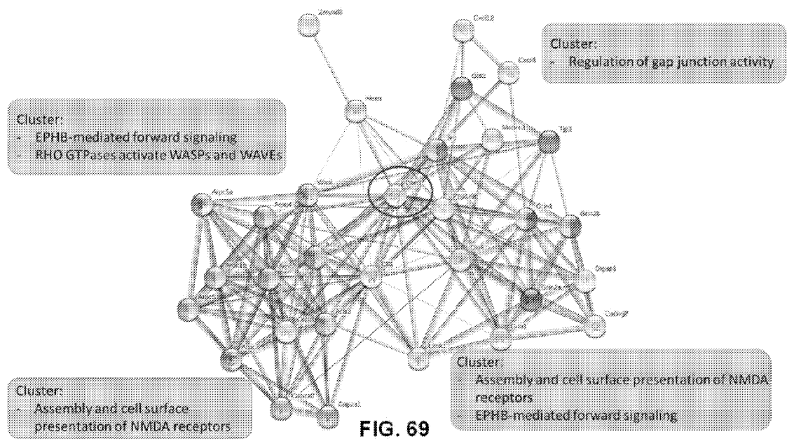

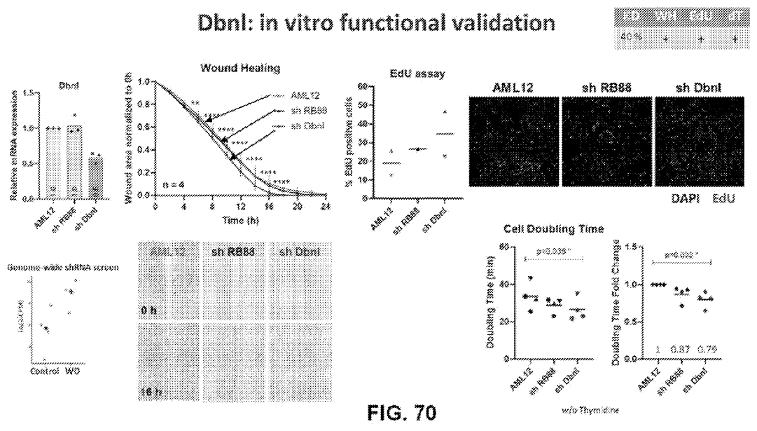

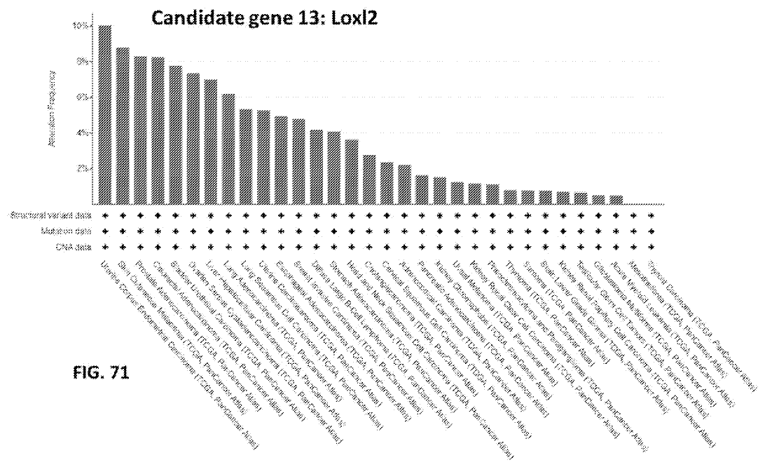

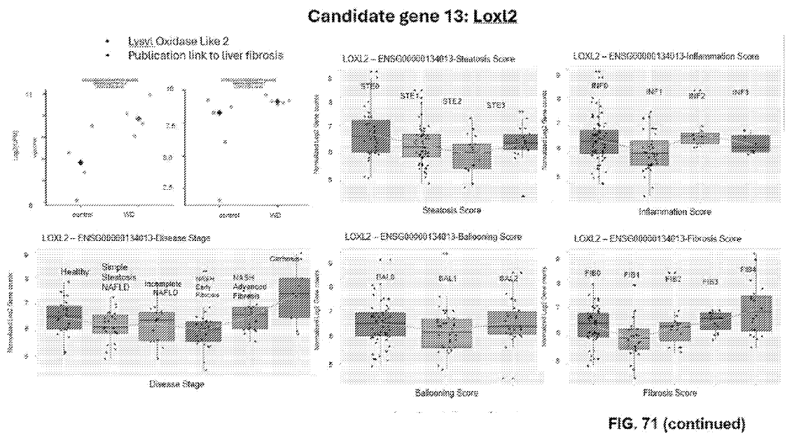

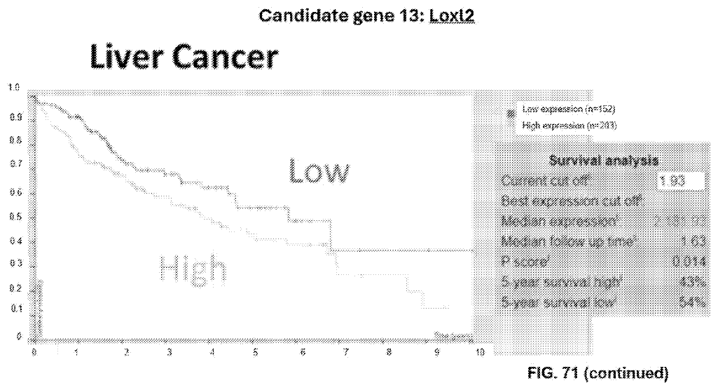

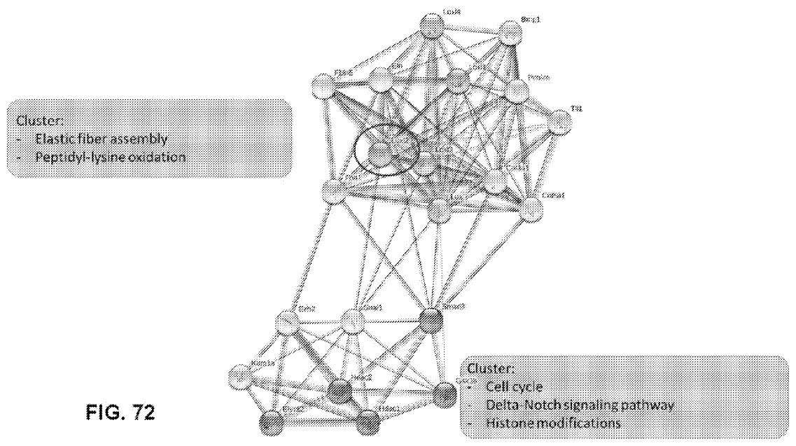

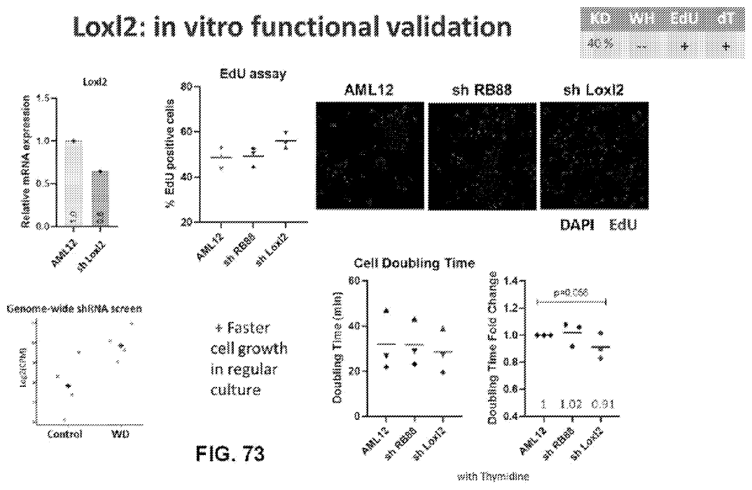

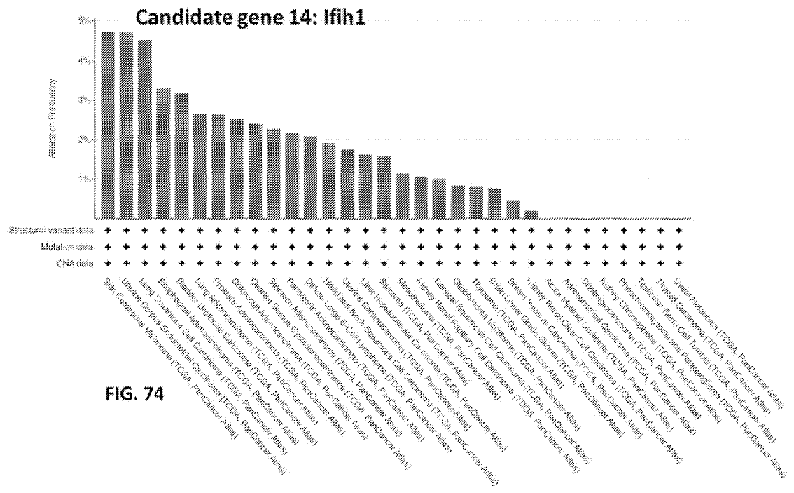

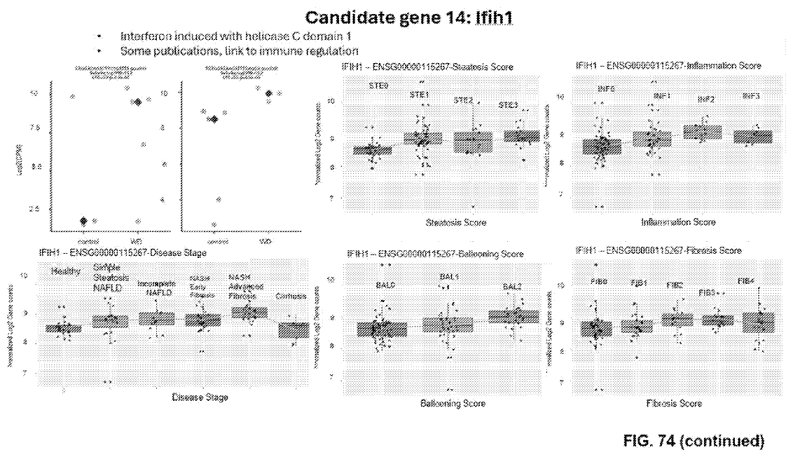

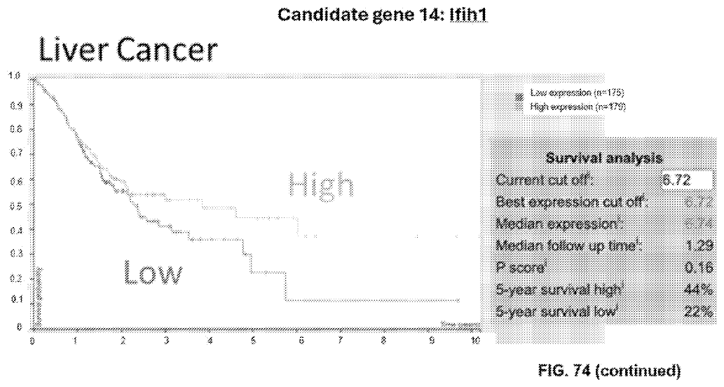

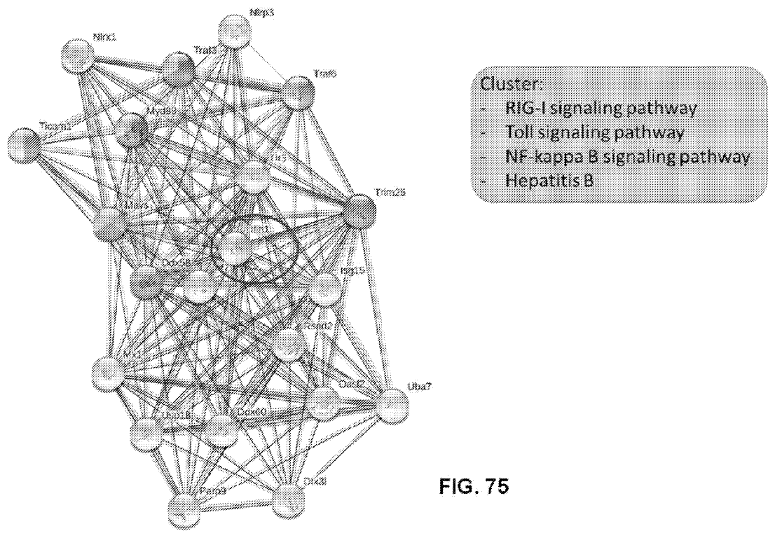

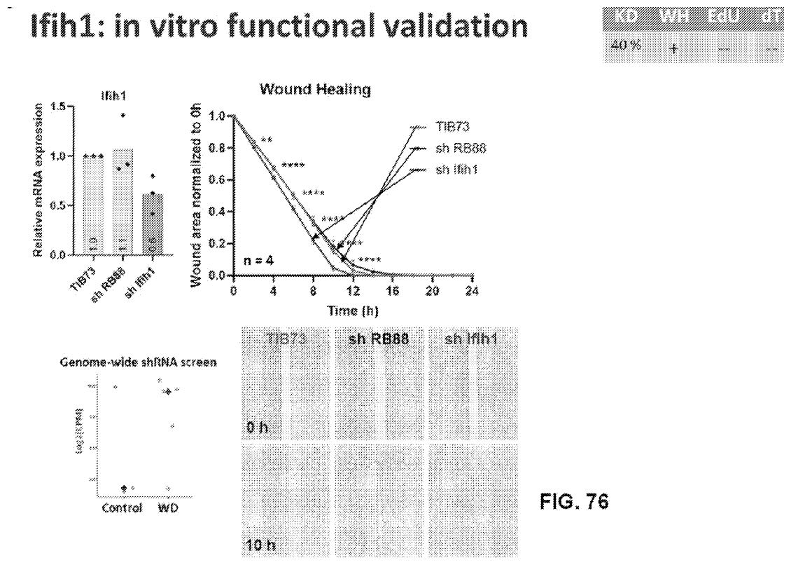

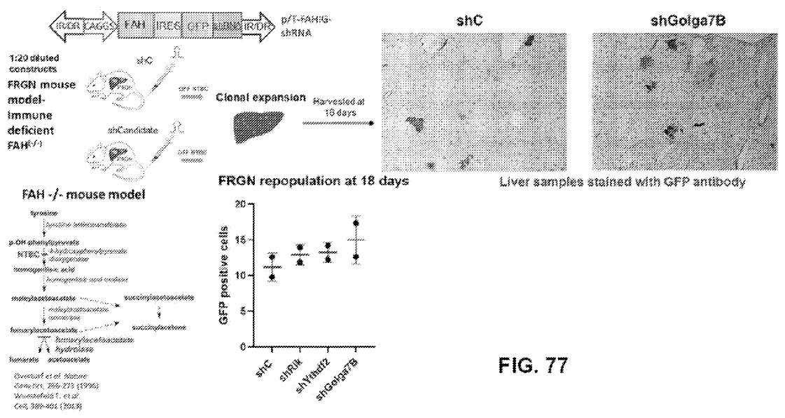

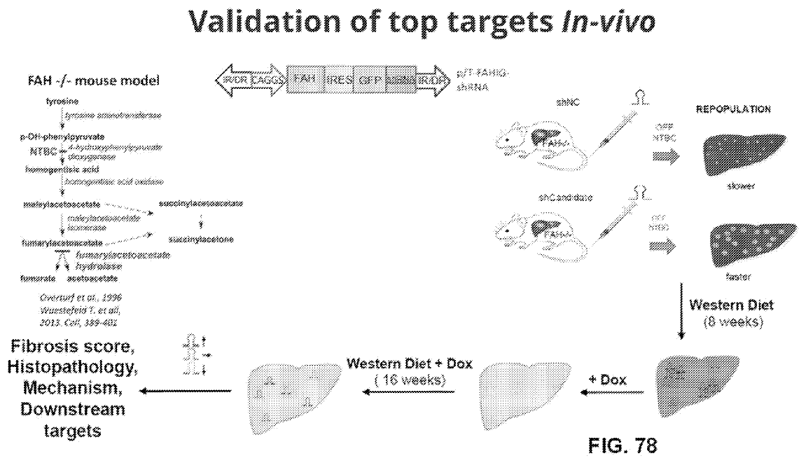

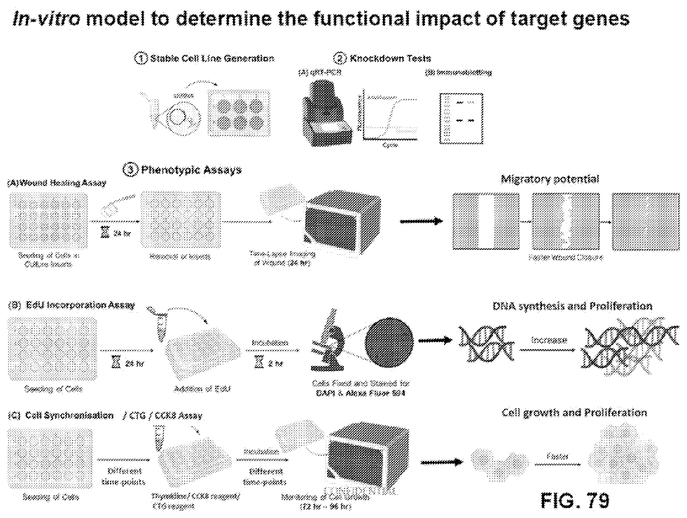

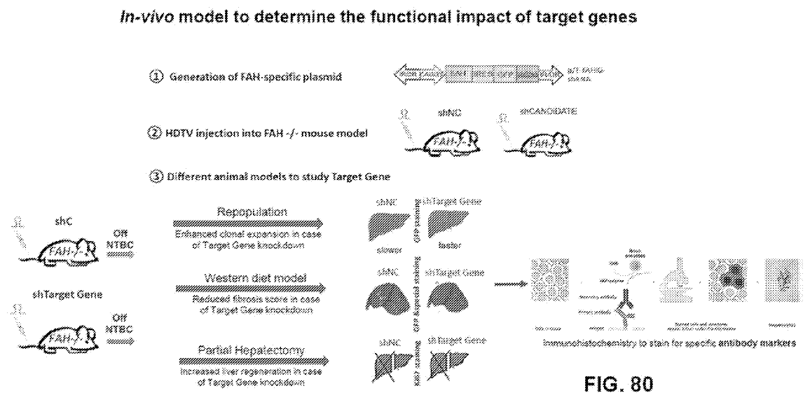

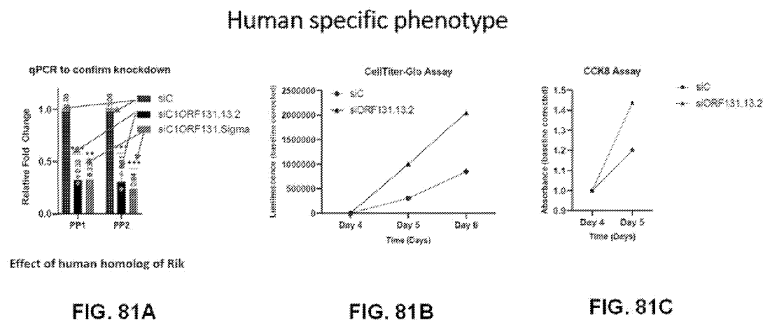

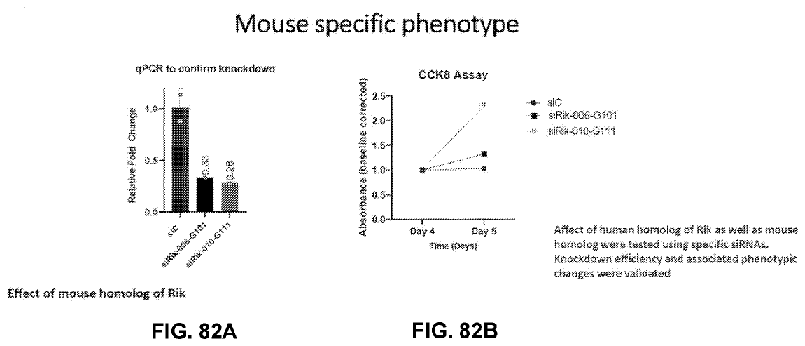

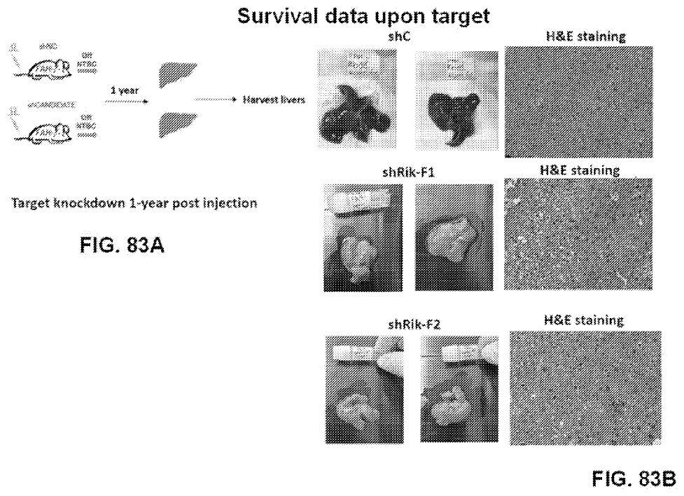

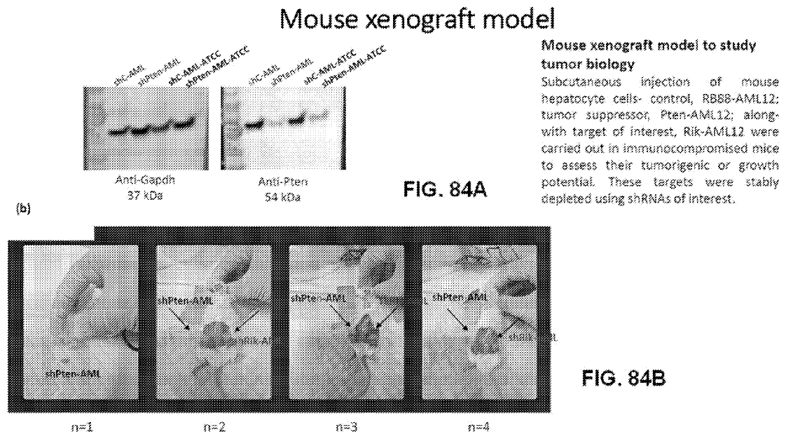

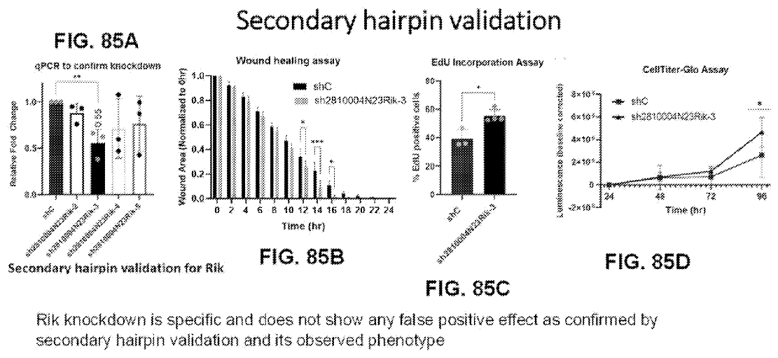

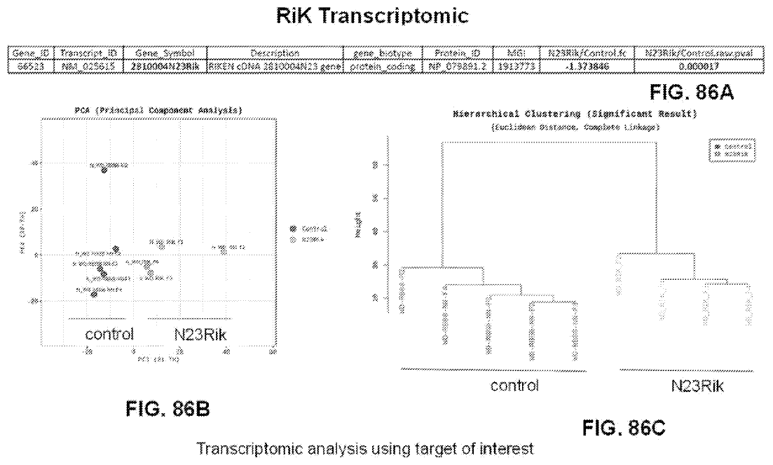

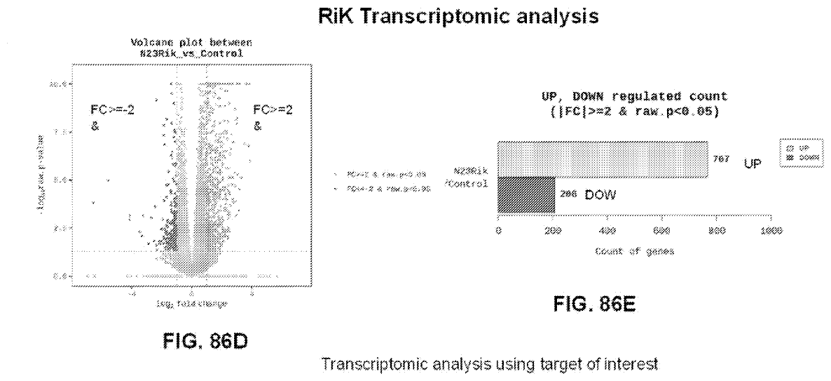

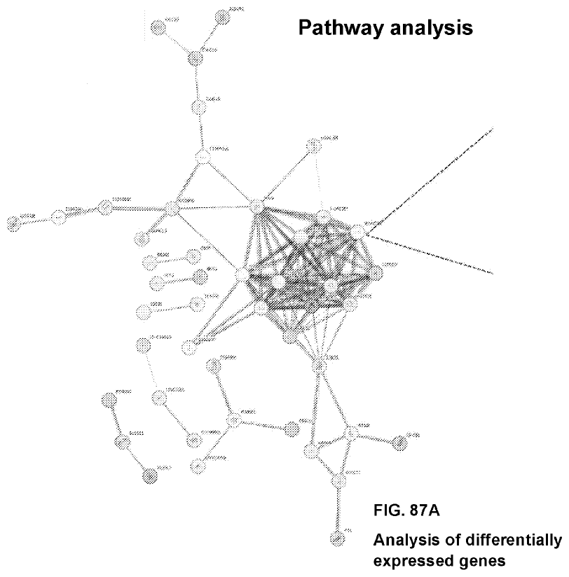

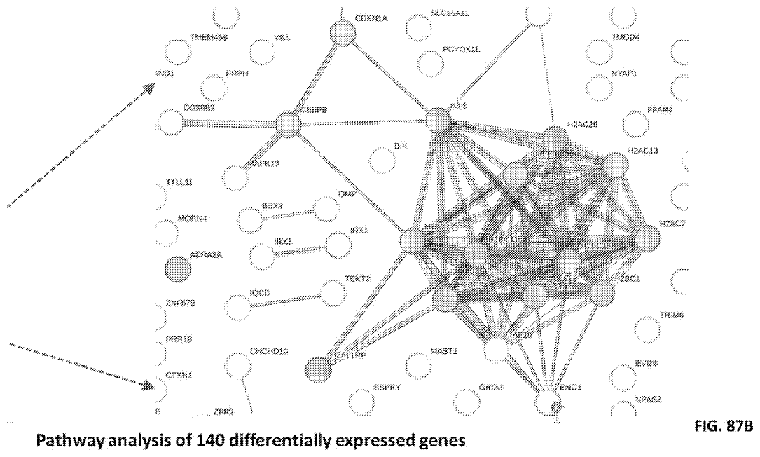

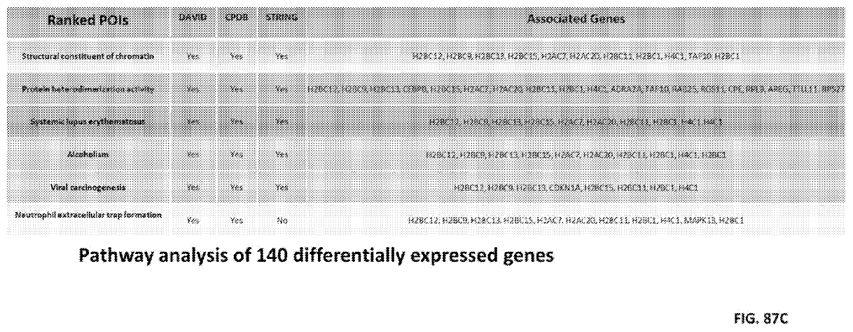

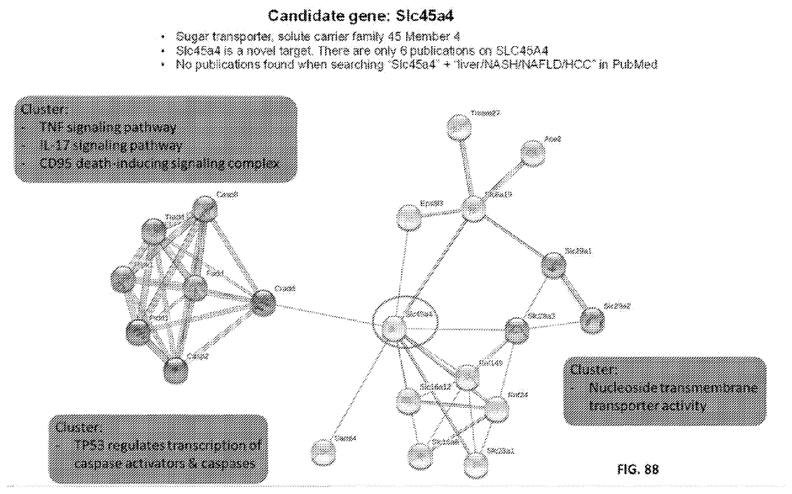

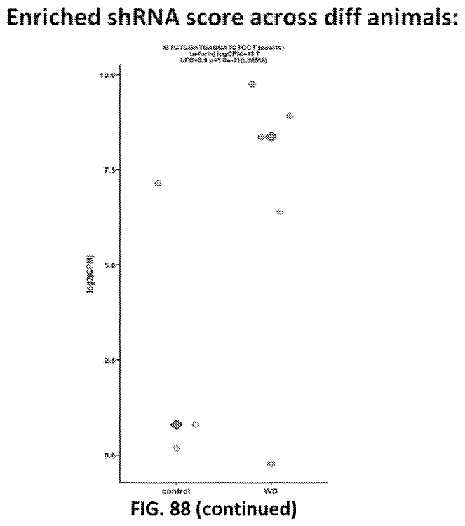

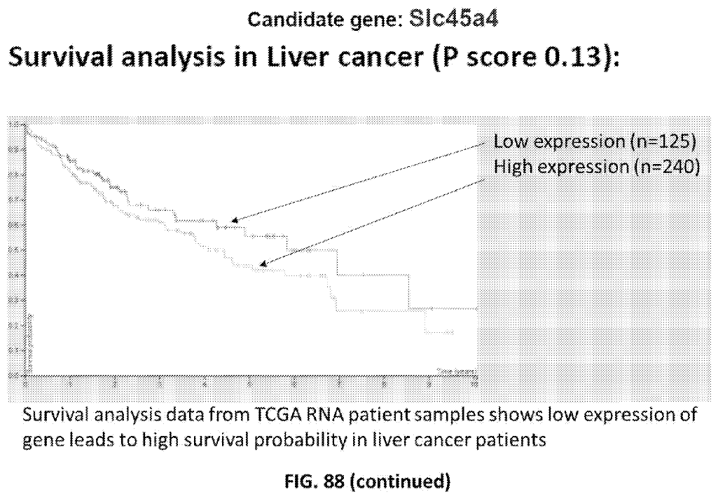

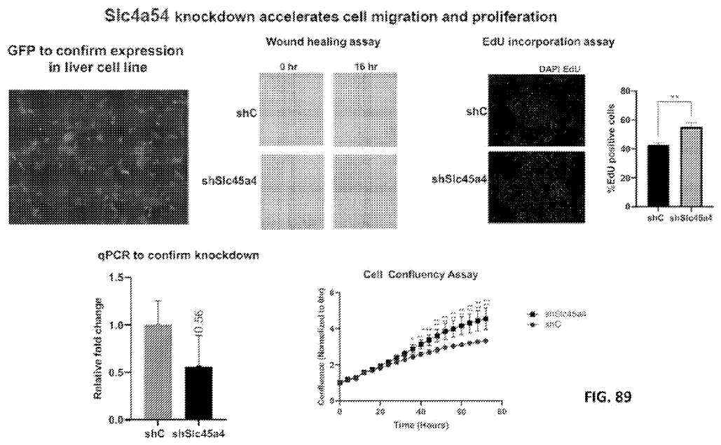

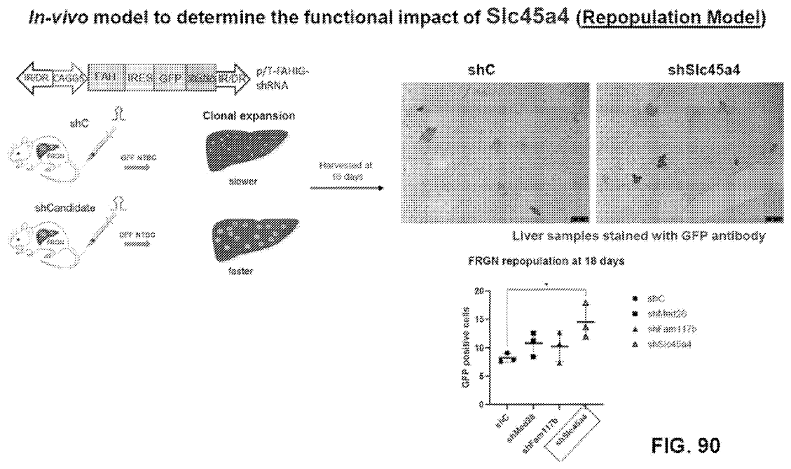

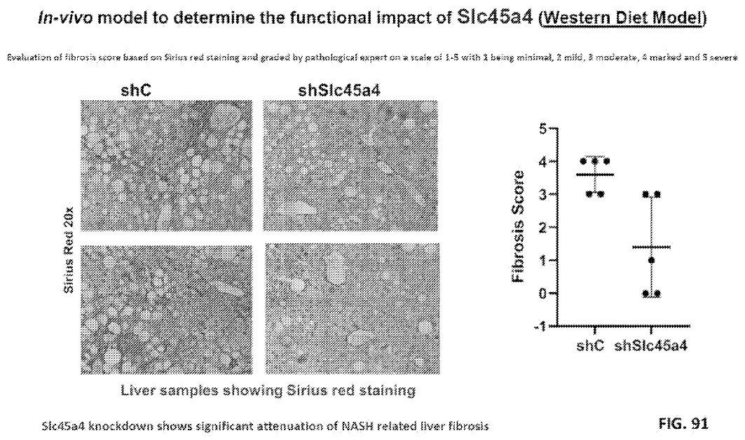

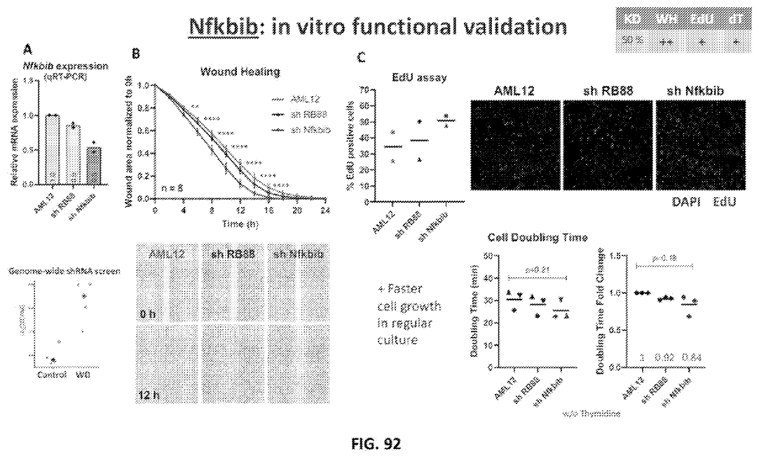

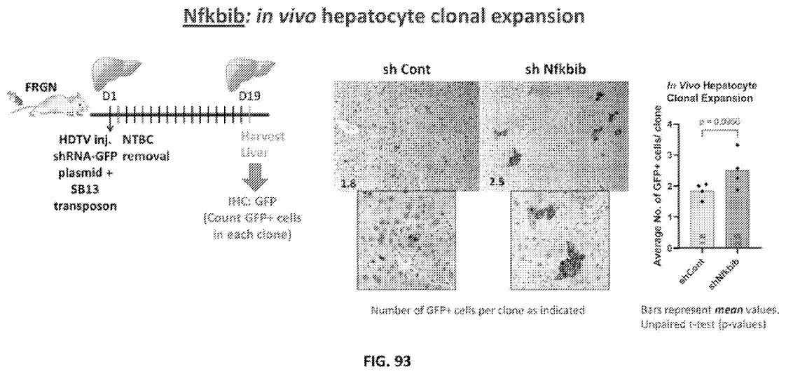

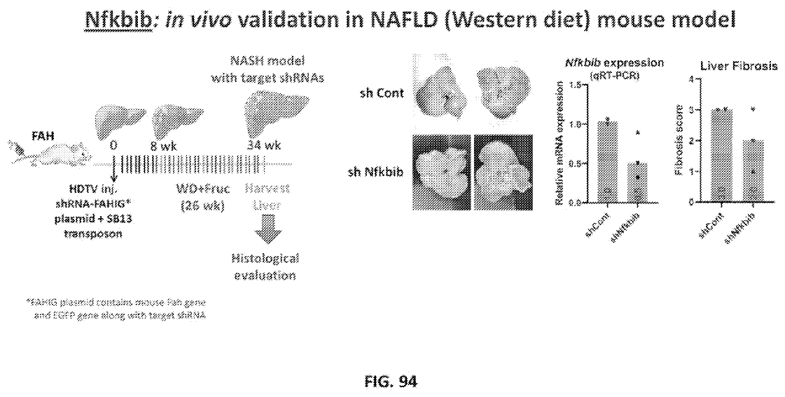

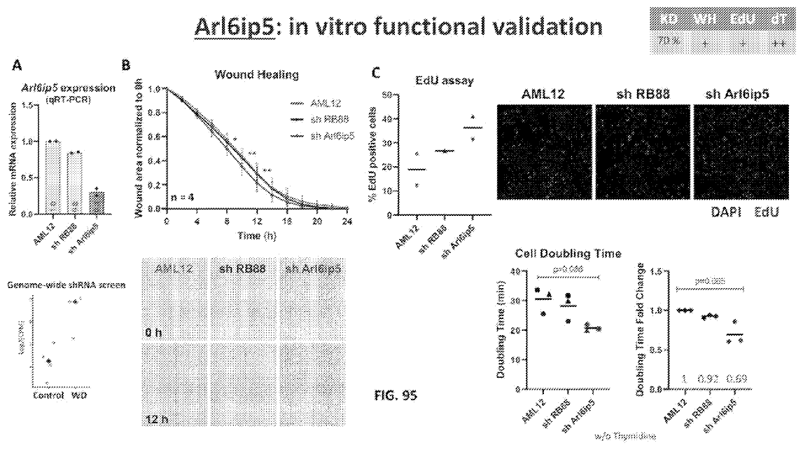

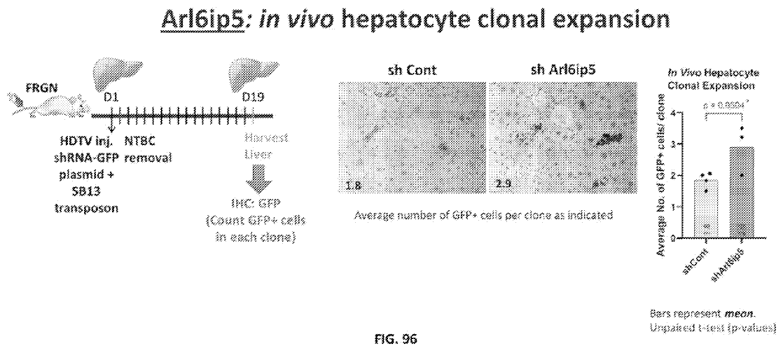

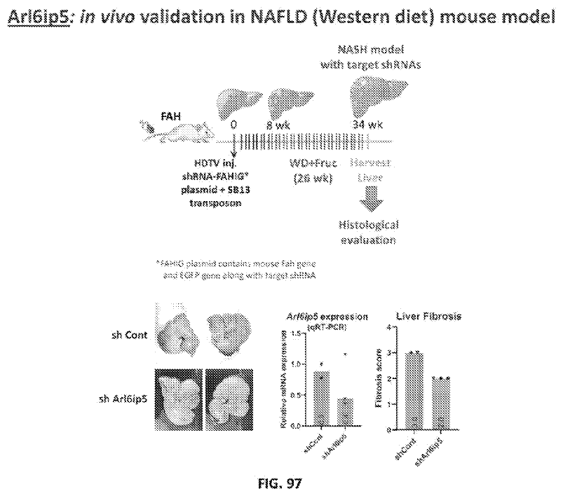

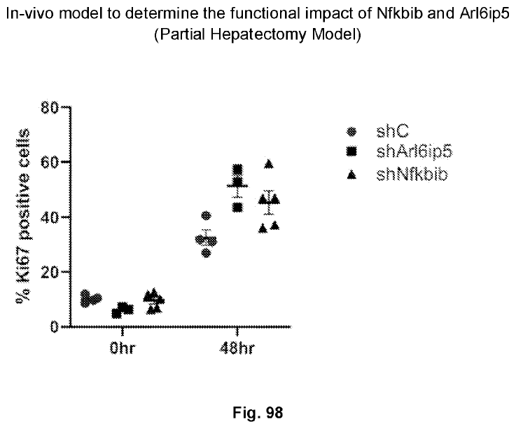

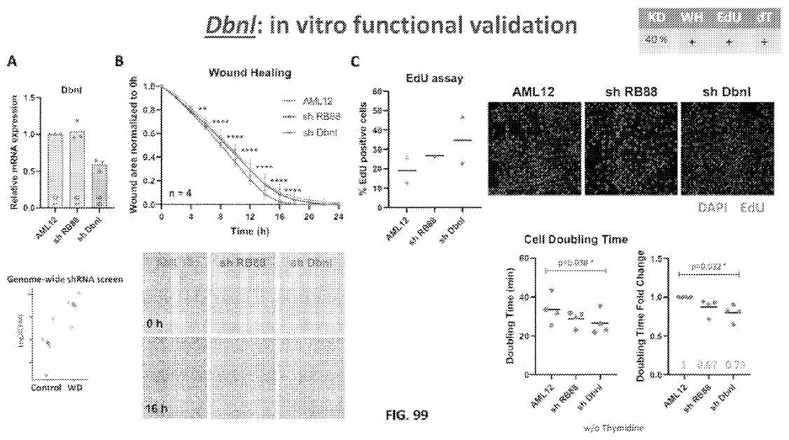

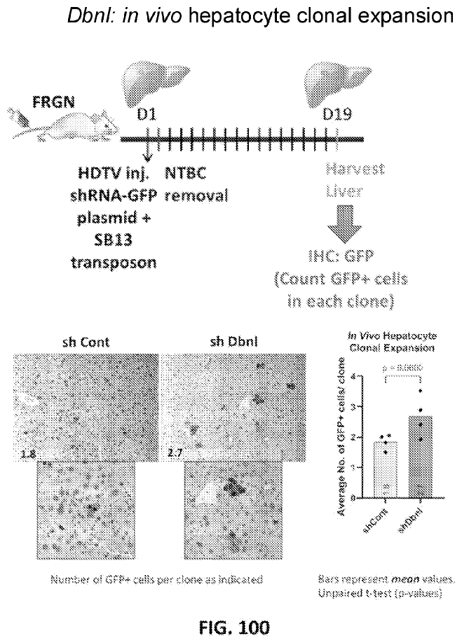

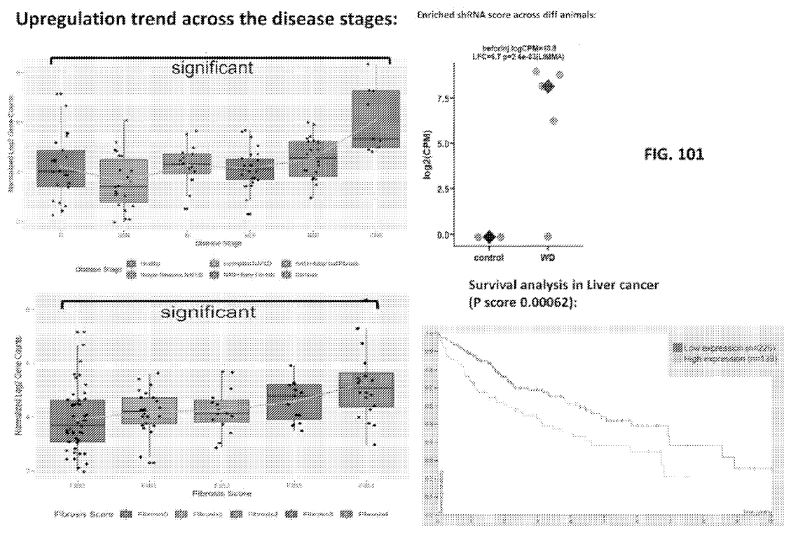

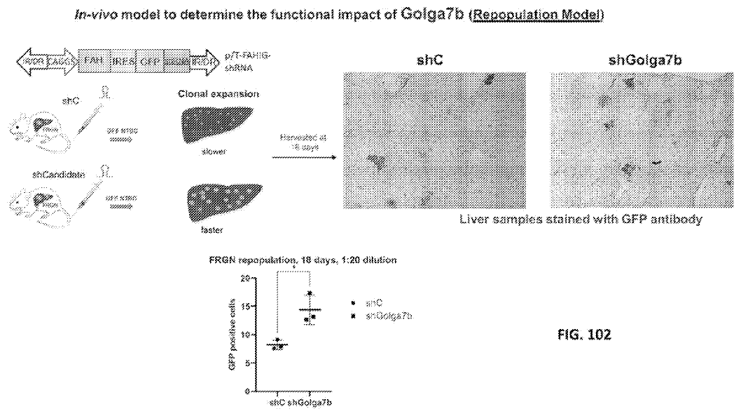

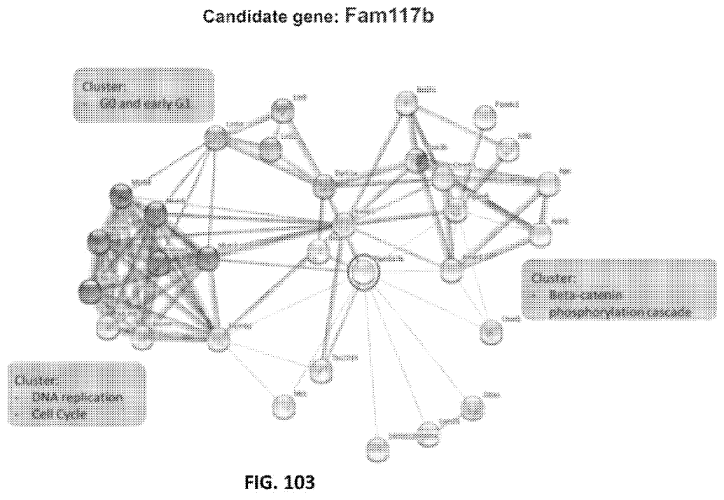

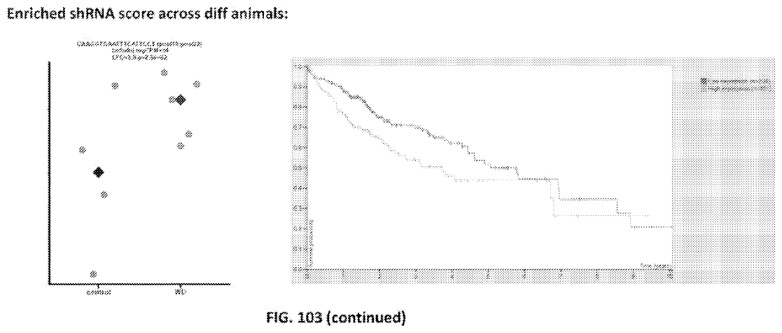

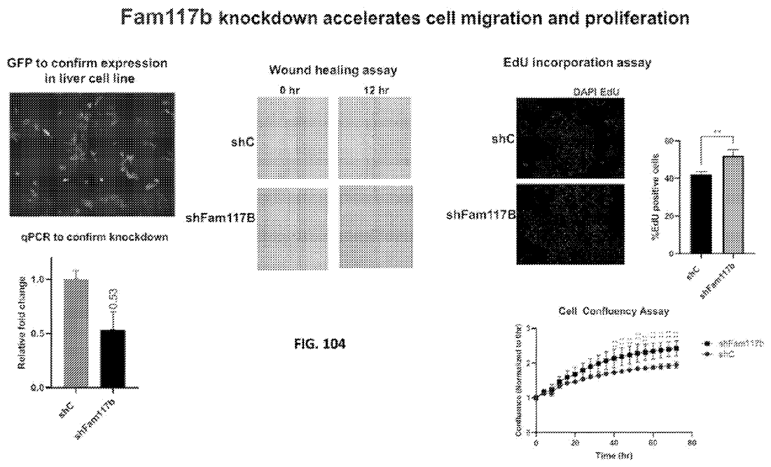

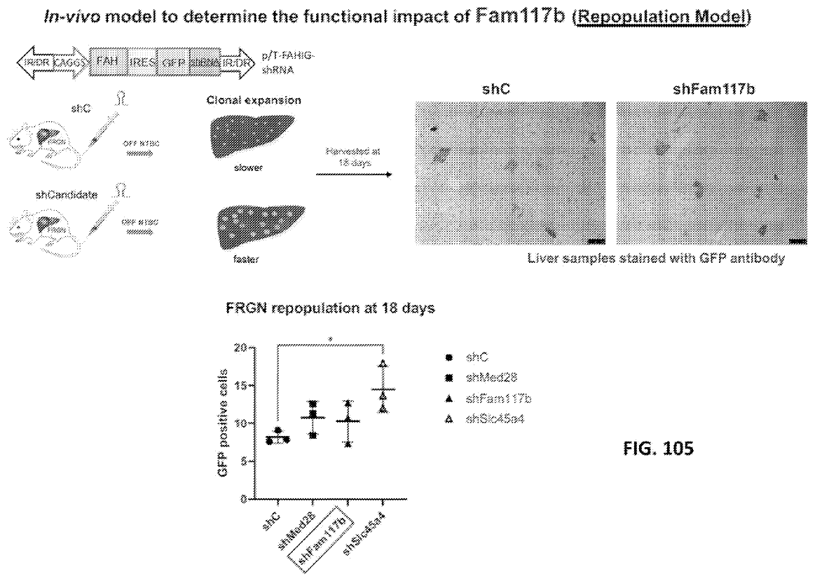

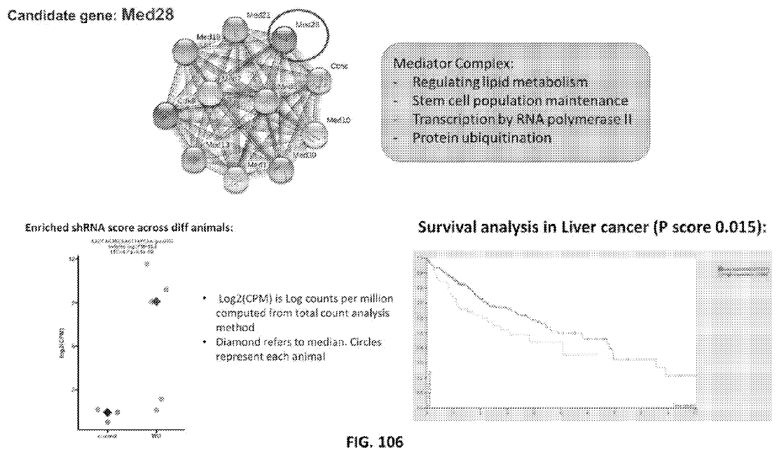

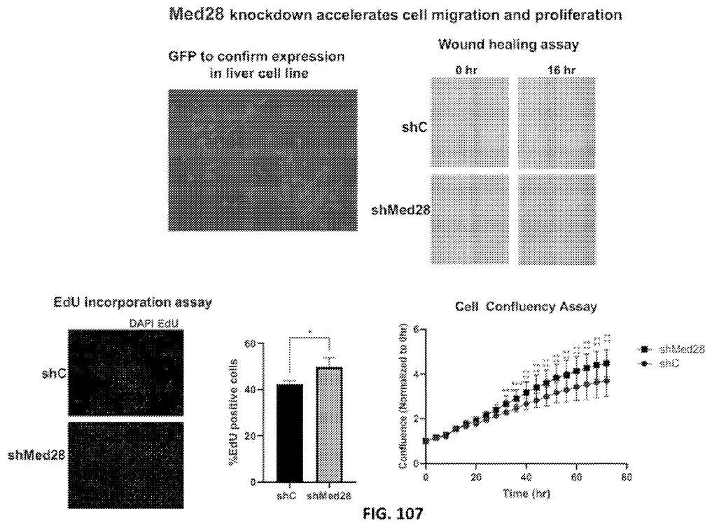

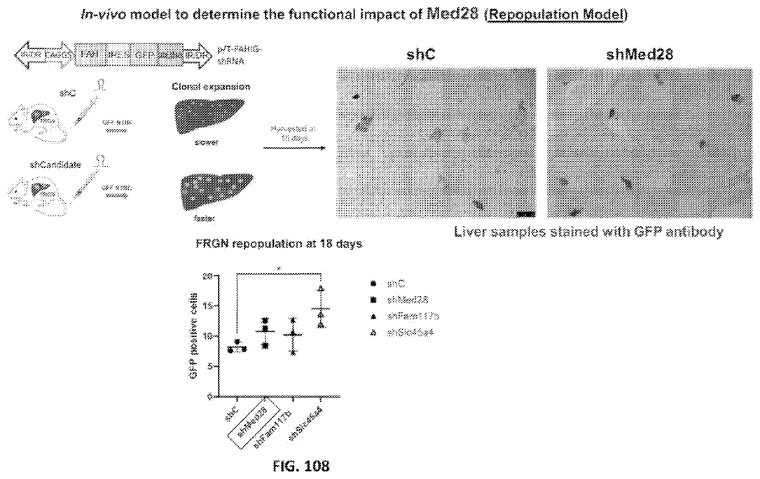

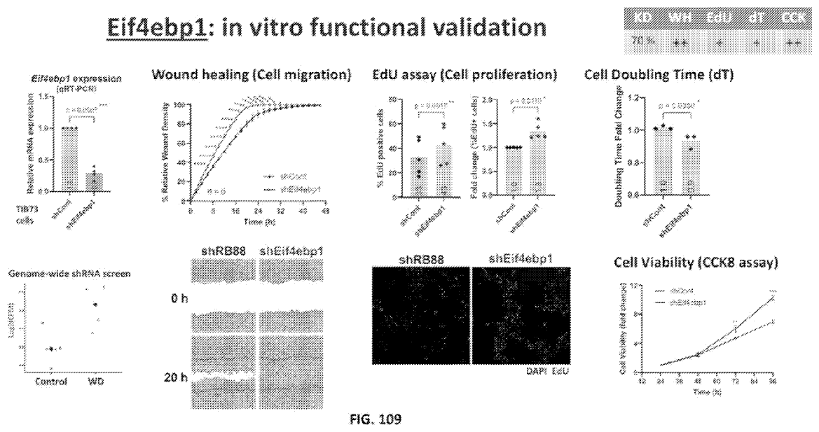

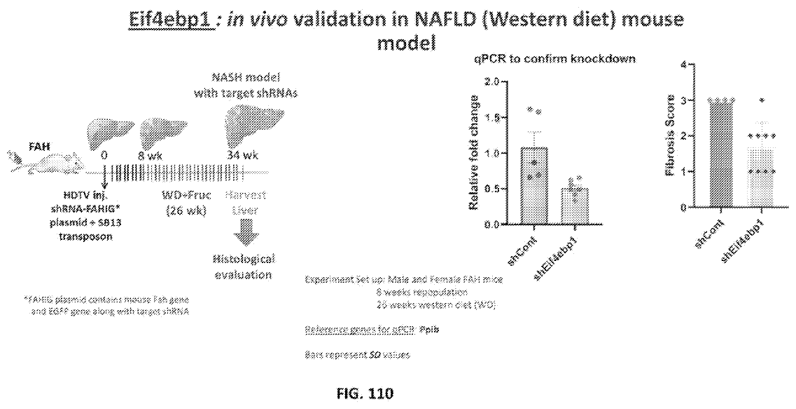

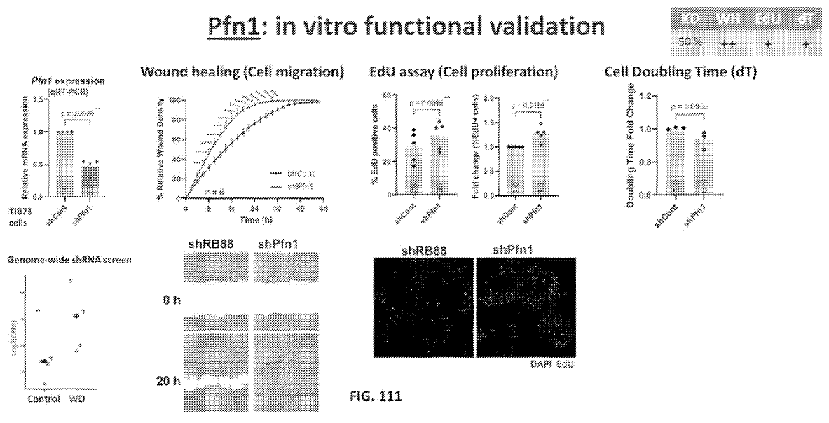

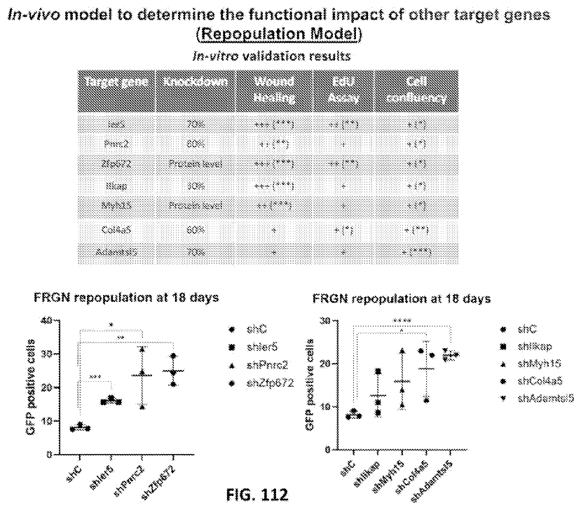

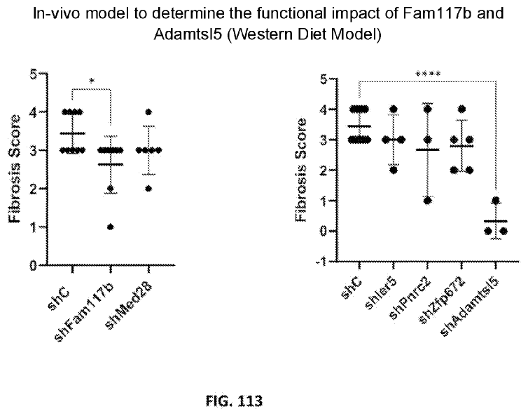

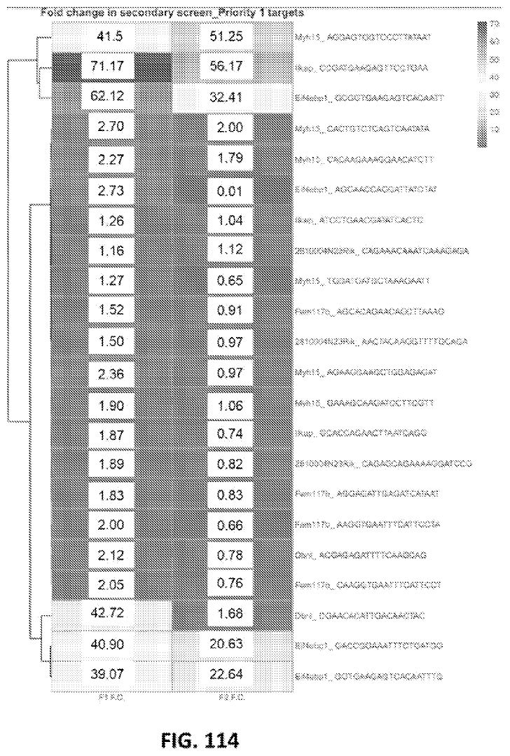

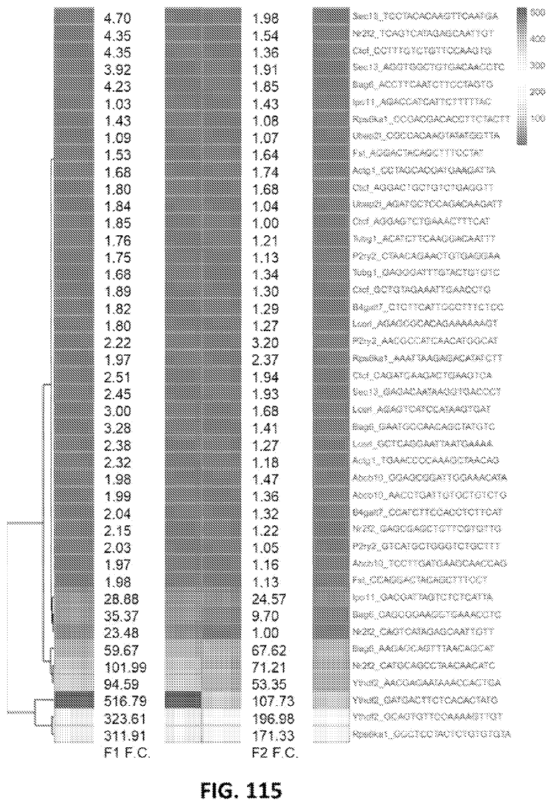

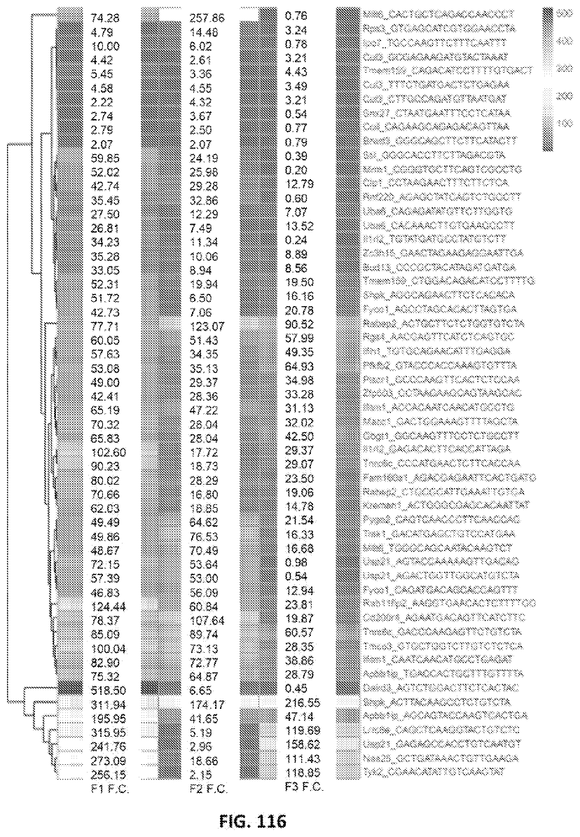

An outline of the screening approach and information to the animal model can be seen in FIGS. 3 to 5. All sub-pools and mice which were exposed to WD or normal chow for 26 weeks were sequenced. The percentage of pool coverage before injection and after injection can be seen in FIGS. 7 & 8. A good coverage over all pools for the genome wide screen was reached. The inventors then run multiplex in silico target identification pipeline, combining 4 different analysis tools: Total Counts, DESeq2, Limma, and MAGeCK (FIGS. 9 to 24). The difference lies mainly in the data normalization methods. In addition, variable stringency cut-offs was used. Dependent on the data analysis tool and stringency parameters variations in the number of significantly enriched shRNAs was seen. To identify the targets with highest confidence and prioritize these targets for validation experiments, the data of the different tools and settings were combined and overlaps were investigated. Applying this multiplex, layered in silico target identification pipeline ~200 high confidence targets were shortlisted, which were further prioritized in ~50 targets passing highest level of stringency and 80 targets passing high level of stringency. The inventors went on to run the top targets through the multiplex in vitro validation pipeline. In this pipeline wound healing, Edu incorporation and cell confluency/cell doubling assays were combined to proof accelerated proliferation/enhanced regeneration in vitro. So far, 14 targets were tested, which passed in vitro validation (FIGS. 29 to 76). From these 14, already three passed the first steps of multiplex in vivo validation pipeline (FIG. 77). This also clearly underlines the quality of the in silico target selection pipeline.

The inventors of the present disclosure have found the following markers that have been grouped as list 1, list 2, and list 3.

### Target Marker List 1

Target marker list 1

C1ORF131 (2810004N23Rik)
SLC45A4
NFKBIB
ARL6IP5
DBNL
GOLGA7B
FAM117B
MED28
TSC22D4
EIF4EBP1
PFN1
PNRC2
ZNF672 (Zfp672)
IER5
ADAMTSL5
COL4A5
MYH15

Target marker list 1 includes targets that have passed In-vitro QC and have undergone or undergoing in-vivo validation inclusive of clonal expansion, diet treatment and partial hepatectomy. Some of the targets have been included in in-vivo secondary screen as controls.

| GENES | F1 F.C. | F2 F.C. |
|---|---|---|
| Ilkap_CCGATGAAGAGTTCCTGAA | 71.16937 | 56.17377 |
| Eif4ebp1_ | 62.12092 | 32.41183 |
| GCGGTGAAGAGTCACAATT | | |
| DbnI_CGAACACATTGACAACTAC | 42.72429 | 1.684746 |
| Myh15_AGGAGTGGTCCCTTATAAT | 41.49919 | 51.25407 |
| Eif4ebp1_ | 40.90381 | 20.62804 |
| GACCGGAAATTTCTGATGG | | |
| Eif4ebp1_GGTGAAGAGTCACAATTTG | 39.07343 | 22.63986 |
| Eif4ebp1_AGGAACCAGGATTATCTAT | 2.734232 | 0.01114 |
| Myh15_CACTGTCTCAGTCAATATA | 2.698067 | 2.004588 |
| Myh15_AGAAGGAAGCTGGAGAGAT | 2.355612 | 0.972286 |
| Myh15_CACAAGAAAGGAACATCTT | 2.269259 | 1.793805 |
| DbnI_ACGAGAGATTTTCAAGCAG | 2.117905 | 0.777234 |
| Fam117b_CAAGGTGAATTTCATTCCT | 2.051211 | 0.77532 |
| Fam117b_AAGGTGAATTTCATTCCTA | 2.003654 | 0.656478 |
| Myh15_GAAAGCAAGATCCTTCGTT | 1.901933 | 1.056701 |
| 2810004N23Rik_ | 1.888808 | 0.816936 |
| CAGAGCAGAAAAGGATCCG | | |
| Ilkap_GCACCAGAACTTAATCAGG | 1.868151 | 0.741484 |
| Fam117b_ | 1.831435 | 0.826052 |
| AGGAGATTGAGATCATAAT | | |
| Fam117b_ | 1.517245 | 0.908751 |
| AGCACAGAACAGCTTAAAG | | |
| 2810004N23Rik_ | 1.500818 | 0.968078 |
| AACTACAAGGTTTTGCAGA | | |
| Myh15_TGGATGATGCTAAAGAATT | 1.265075 | 0.645202 |
| Ilkap_ATCCTGAACGATATCACTC | 1.258622 | 1.038222 |
| 2810004N23Rik_ | 1.159836 | 1.122654 |
| CAGAAACAAATCAAAGAGA | | |

### Target Marker List 2

Target marker list 2

ABCB10
LSM14A
ACTG1
YTHDF2
RPS6KA1
SEC13
IPO11
CTCF
UBAP2L
P2RY2
TRAF3IP2
FST
TUBG1
ZNF664
PVR
DYX1C1/DNAAF4
TEX264
NIF3L1
RPUSD1
TRIM6
GLRX3
BRD1
CKS1B
C6ORF120
WASF2
ZNF689
B4GALT7
LCORL
NR2F2
BAG6
RNF10

Target marker list 2 includes targets that have passed literature review and have undergone/currently undergoing in-vitro QC or are in the queue. Some of these targets have passed in-vitro QC and are included in the in-vivo secondary analysis. Some of these will also be shortlisted based on phenotype for in-vivo primary validation.

| GENES | F1 F.C. | F2 F.C. |
|---|---|---|
| Ythdf2_GCAGTGTTCCAAAAGTTGT | 323.6118252 | 196.9755784 |
| Rps6ka1_GGCTCCTACTCTGTGTGTA | 311.9075297 | 171.327609 |
| Ythdf2_GATGACTTCTCACACTATG | 516.7938462 | 107.7261538 |
| Nr2f2_CATGCAGCCTAACAACATC | 101.9881446 | 71.20509781 |
| Bag6_AAGAGCAGTTTAACAGCAT | 59.66911765 | 67.62263655 |
| Ythdf2_AACGAGAATAAACCAGTGA | 94.59166248 | 53.3480663 |
| Ipo11_GACGATTAGTCTCTCATTA | 28.88043217 | 24.57070828 |
| Bag6_CAGCGGAAGGTGAAACCTC | 35.37283162 | 9.696297575 |
| P2ry2_AACGCCATCAACATGGCAT | 2.215235792 | 3.20274083 |
| Rps6ka1_AAATTAAGAGACATATCTT | 1.970227363 | 2.370988497 |
| Sec13_TCCTACACAAGTTCAATGA | 4.700680778 | 1.981263098 |
| Ctcf_CAGATGAAGACTGAAGTCA | 2.511429031 | 1.936728454 |
| Sec13_GAGACAATAAGGTGACCCT | 2.45141039 | 1.934210276 |
| Sec13_AGGTGGCTGTGACAACCTC | 3.918172592 | 1.905152271 |
| Bag6_ACCTTCAATCTTCCTAGTG | 4.22899897 | 1.84611441 |
| Actg1_CCTAGCACGATGAAGATTA | 1.678343821 | 1.74083943 |
| Ctcf_AGGACTGCTGTCTGAGGTT | 1.79615866 | 1.682429961 |
| LcorI_AGAGTCATCCATAAGTGAT | 3.003873758 | 1.680759767 |
| Fst_AGGACTACAGCTTTCCTAT | 1.52611465 | 1.637579618 |
| Nr2f2_TCAGTCATAGAGCAATTGT | 4.347978104 | 1.537170779 |
| Abcb10_GGAGCGGATTGGAAACATA | 1.983056952 | 1.446815255 |
| Ipo11_AGACCATCATTCTTTTTAC | 1.028403693 | 1.434681005 |
| Bag6_GAATGCCAACAGCTATGTC | 3.284298069 | 1.413394803 |
| Abcb10_AACCTGATTGTGCTGTCTG | 1.992931189 | 1.361539509 |
| Ctcf_CCTTTGTCTGTTCCAAGTG | 4.352729399 | 1.356402712 |
| Tubg1_GAGGGATTTGTACTGTGTC | 1.694124419 | 1.335486274 |
| B4galt7_CCATCTTCCACCTCTTCAT | 2.043403479 | 1.323158538 |
| Ctcf_GCTGTAGAAATTGAACCTG | 1.887826574 | 1.299526828 |
| B4galt7_CTCTTCATTGCCTTTCTCC | 1.821647902 | 1.29458222 |
| LcorI_AGAGCGCACAGAAAAAAGT | 1.804829362 | 1.274731357 |
| LcorI_GCTCAGGAATTAATGAAAA | 2.383177186 | 1.267778991 |
| Nr2f2_GAGCGAGCTGTTCGTGTTG | 2.14797401 | 1.220384636 |
| Tubg1_ACATCTTCAAGGACAATTT | 1.763147183 | 1.211843262 |
| Actg1_TGAACCCCAAAGCTAACAG | 2.32209083 | 1.179223854 |
| Abcb10_TCCTTGATGAAGCAACCAG | 1.969727727 | 1.156525282 |
| Fst_CCAGGACTACAGCTTTCCT | 1.981153224 | 1.133513816 |
| P2ry2_CTAACAGAACTGTGAGGAA | 1.747743957 | 1.126606796 |
| Rps6ka1_CCGACGACACCTTCTACTT | 1.427863934 | 1.082680837 |
| Ubap21_CGCCACAAGTATATGGTTA | 1.091934969 | 1.070714455 |
| P2ry2_GTCATGCTGGGTCTGCTTT | 2.029678811 | 1.047501396 |
| Ubap2I_AGATGCTCCAGACAAGATT | 1.836338577 | 1.035913722 |
| Ctcf_AGGAGTCTGAAACTTTCAT | 1.653500391 | 1.002655383 |
| Nr2f2_CAGTCATAGAGCAATTGTT | 23.4787234 | 1 |

### Target Marker List 3

Target marker list 3

Uba6
Brwd3
Fyco1
ARID5B
C20ORF96 (6820408C15Rik)
CD200R1 (Cd200r4)
DALRD3
ELF3
ELFN1
FAM160A1
FAM50B
KIAA2026 (9930021J03Rik)
KREMEN1
MACC1
NAA25
PROSC/PLPBP
RPS3
SHPK
SLAIN1
TCHP
ZNF503 (Zfp503)
BUD13
CD93
PFKFB2
ZDBF2
FOXC2
GBGT1
RGS4
RNASEH1
TSNAXIP1
LYPD2
STIL
TMCO3
TMEM159
RIF1
IFIH1
EPN2
CLP1
RNF220
NOL12
APOL3
GTF3C6
CTPS2
IFITM2, IFITM1
KCNS3
CA2
EIF4H
MRM1
AKAP8
PAPOLA
SECTM1
TYK2
MLLT6
IL1RL2
IPO7
APBB1IP
RABEP2
PLSCR1
PYGO2
COIL
LRRC8E
TNRC6C
CHD3
USP21
SNX27
NOP14
ZC3H15
C15orf39
UTP20
TRAK1
RAB11FIP2
PHPT1
CUL3
GNL3
Uba6
Brwd3
Fyco1
ARID5B
C20ORF96 (6820408C15Rik)
CD200R1 (Cd200r4)
DALRD3
ELF3
ELFN1
FAM160A1
FAM50B
KIAA2026 (9930021J03Rik)
KREMEN1
MACC1
NAA25
PROSC/PLPBP
RPS3
SHPK
SLAIN1
TCHP
ZNF503 (Zfp503)
BUD13
CD93
PFKFB2
ZDBF2
FOXC2
GBGT1
RGS4
RNASEH1
TSNAXIP1
LYPD2
STIL
TMCO3
TMEM159
RIF1
IFIH1
EPN2
CLP1
RNF220
NOL12
APOL3
GTF3C6
CTPS2
IFITM2, IFITM1
KCNS3
CA2
EIF4H
MRM1
AKAP8
PAPOLA
SECTM1
TYK2
MLLT6
IL1RL2
IPO7
APBB1IP
RABEP2
PLSCR1
PYGO2
COIL
LRRC8E
TNRC6C
CHD3
USP21
SNX27
NOP14
ZC3H15
C15orf39
UTP20
TRAK1
RAB11FIP2
PHPT1
CUL3
GNL3

Target list 3 includes targets that have passed literature review inclusive of human homolog, target being novel, no association with NAFLD pathologies, high survival probability in liver cancer patients and in-house patient transcriptomics data.

| GENES | F1 F.C. | F2 F.C. | F3 F.C. |
|---|---|---|---|
| Pfkfb2_GCCTCTGTCCAGTTCGAAC | 2811.368 | 5529.838 | 1206.5 |
| Macc1_GAAGACAAGAGAGTGCCTT | 2606.159 | 2238.333 | 2262.406 |
| Dalrd3_AGTCTGGACTTCTCACTAC | 518.5 | 6.65 | 0.45 |
| Lrrc8e_CAGCTCAAGGTACTGTCTC | 315.9495 | 5.191336 | 119.6886 |
| Shpk_ACTTACAAGCCTCTGTCTA | 311.9401 | 174.1723 | 216.5468 |
| Naa25_GCTGATAAACTGTTGAAGA | 273.0907 | 18.66499 | 111.432 |
| Tyk2_CGAACATATTGTCAAGTAT | 256.15 | 2.15 | 118.85 |
| Usp21_GAGAGCCACCTGTCAATGT | 241.7563 | 2.963554 | 158.6196 |
| Apbb1ip_AGCAGTACCAAGTCACTGA | 195.9463 | 41.64952 | 47.136 |
| Rab11fip2_AAGGTGAACACTCTTTTGG | 124.4399 | 60.83575 | 23.80868 |
| Il1rl2_GAGACACTTCACCATTAGA | 102.6027 | 17.72156 | 29.36842 |
| Tmco3_GTGCTGGTCTTGTCTCTCA | 100.0366 | 73.12618 | 28.35026 |
| Tnrc6c_CCCATGAACTCTTCACCAA | 90.2308 | 18.731 | 29.07242 |
| Tnrc6c_GACCCAAGAGTTCTGTCTA | 85.09497 | 89.73911 | 60.56983 |
| Ifitm1_CAATCAACATGCCTGAGAT | 82.89735 | 72.76875 | 38.86125 |
| Fam160a1_AGACGAGAATTCACTGATG | 80.01539 | 28.28841 | 23.50218 |
| Cd200r4_AGAATGACAGTTCATCTTC | 78.37394 | 107.6368 | 19.87197 |
| Rabep2_ACTGCTTCTCTGGTGTCTA | 77.70673 | 123.0673 | 90.52003 |
| Apbb1ip_TGACCACTGCTTTGTTTTA | 75.31885 | 64.87187 | 28.79308 |
| Mllt6_CACTGCTCAGACCAACCCT | 74.27586 | 257.8621 | 0.758621 |
| Usp21_AGTACCAAAAAGTTGACAG | 72.15341 | 53.64254 | 0.976236 |
| Rabep2_CTGCGCATTGAAATTGTGA | 70.66484 | 16.79639 | 19.05605 |
| Macc1_GACTGGAAAGTTTTAGCTA | 70.31569 | 28.0411 | 32.01619 |
| Gbgt1_GGCAAGTTTCCTCTGCCTT | 65.83058 | 28.0394 | 42.49645 |
| Ifitm1_ACCACAATCAACATGCCTG | 65.19199 | 47.2168 | 31.1263 |
| Kremen1_ACTGGGCGAGCACAATTAT | 62.03295 | 18.85407 | 14.78437 |
| Rgs4_AACGAGTTCATCTCAGTGC | 60.05083 | 51.42682 | 57.9902 |
| Stil_GGGCACCTTCTTAGACGTA | 59.85125 | 24.18967 | 0.391135 |
| Ifih1_TGTGCAGAACATTTGAGGA | 57.6279 | 34.35246 | 49.35268 |
| Usp21_AGACTGGTTGGCATGTCTA | 57.38624 | 53.00246 | 0.537415 |
| Fyco1_CAGATGACAGCACCAGTTT | 56.82848 | 56.0942 | 12.93779 |
| Pfkfb2_GTACCCACCAAAGTGTTTA | 53.07582 | 35.12876 | 64.9299 |
| Tmem159_CTGGACAGACATCCTTTTG | 52.31029 | 19.93931 | 19.49533 |
| Mrm1_CGGGTGCTTCAGTCGCCTG | 52.02304 | 25.97763 | 0.201888 |
| Shpk_AGGCAGAACTTCTCACACA | 51.72047 | 6.495629 | 16.16162 |
| Trak1_GACATGAGCTGTCCATGAA | 49.85706 | 76.52638 | 16.3334 |
| Pygo2_CAGTCAACCCTTCAACCAG | 49.4942 | 64.62184 | 21.53686 |
| Plscr1_GCCCAAGTTCACTCTCCAA | 49.00109 | 29.37336 | 34.98035 |
| Mllt6_TGGGCAGCAATACAAGTCT | 48.66963 | 70.49421 | 16.67943 |
| Clp1_CCTAAGAACTTTCTTCTCA | 42.74434 | 29.28216 | 12.79378 |
| Fyco1_AGCCTAGCACACTTAGTGA | 42.72668 | 7.063616 | 20.77998 |
| Zfp503_CCTAAGAAGCAGTAAGCAC | 42.40757 | 28.35751 | 33.27592 |
| Rnf220_AGAGCTATCAGTCTGCCTT | 35.44796 | 32.85622 | 0.601395 |
| Zc3h15_GAACTAGAAGAGGAATTGA | 35.2766 | 10.05708 | 8.887538 |
| Il1rl2_TGTATGATGCCTATGTCTT | 34.22727 | 11.34416 | 0.24026 |
| Bud13_CCCGCTACATAGATGATGA | 33.04822 | 8.944585 | 8.564378 |
| Uba6_CAGAGATATGTTCTTGGTG | 27.50114 | 12.28764 | 7.069942 |
| Uba6_CACAAACTTGTGAAGCCTT | 26.81099 | 7.487589 | 13.52145 |
| Ipo7_TGCCAAGTTCTTTCAATTT | 10.00045 | 6.023087 | 0.779991 |
| Tmem159_CAGACATCCTTTTGTGACT | 5.448087 | 3.355191 | 4.431694 |
| Rps3_GTGAGCATCGTGGAACCTA | 4.791334 | 14.47712 | 3.241588 |
| Cul3_TTTCTGATGACTCTGAGAA | 4.579033 | 4.547917 | 3.48904 |
| Cul3_GCGAGAAGATGTACTAAAT | 4.424184 | 2.607246 | 3.210746 |
| Coil_CAGAAGCAGAGACAGTTAA | 2.79485 | 2.499169 | 0.771 |
| Snx27_CTAATGAATTTCCTCATAA | 2.738317 | 3.670535 | 0.536576 |
| Cul3_CTTGCCAGATGTTAATGAT | 2.223106 | 4.32408 | 3.213408 |
| Brwd3_GGGCAGCTTCTTCATACTT | 2.067931 | 2.071999 | 0.787542 |

### Materials & Methods

### Confluence Growth Curve and Cell Doubling Time (dT)

### Day 1

Seeded cells at ~10% confluence (20,000-30,000 cells per 24-wp well in 0.5 ml media). Note similar assay could be performed in a 96-well plate format. Ensure the plate used for seeding cells was compatible with the IncuCyte. Check the “IncuCyte S3 Supported Vessel List” available online.
Allowed cells to adhere to the plate surface (1-2 hr), then add Thymidine (1-2 mM depending on the cell type). Incubated for 18 hours.

### Day 2

Washed cells twice with PBS and replace fresh grown media.
Put plates in the IncuCyte typically imaging every 4 hours for 3-5 days. Either of the two settings were used:
   Whole well, 4×
   Standard at 4× with up to 5 images per well.
Allowed wells to achieve >80% confluence before terminating the scans.
Analyse images with IncuCyte software to create a mask that most accurately detects the cells. Measure the “Cell Confluence”, i.e. the area covered by the cells in each well. Export data and analyse in Excel/GraphPad Prism.
Calculated the Cell Doubling Time (dT), use the following two methods:
   Doubling time cell calculator++ at www.doubling-time.com/compute_more.php.
   GraphPad Prism using Exponential Growth Least squares fit model from raw data.
   Calculate dT from above two methods using values between 10-80% confluence only.

### Wound Healing Assay

On day 1, the Culture-Insert 2-well were transferred (Ibidi #80209) into a 24-well plate well (Falcon #353047). This protocol is for imaging using IncuCyte. For manual imaging at various time points, the Culture-inserts preinstalled in a μ-dish (ibidi #81176) could be used instead of a manually installing the inserts in a 24-wp. Cells were counted and made a suspension of 7×10⁵ cells/ml. Cell seeding density were optimised to get a monolayer on the next day. Typical cells count of 5-10×10⁵ cells/ml resulted in a monolayer for most cell lines. 70 μl of cell suspension were pipetted into each well of the insert. Shaking was avoided and let the suspension stand undisturbed for ~10 min on a flat surface. Culture was incubated for 18-24 hrs till a well-formed cell monolayer was visible.

On day 2, the insert was gently removed by pulling with an ethanol/UV sterilized tweezer. The inserts could be washed/sterilized with ethanol and reused. Wells were rinsed with PBS/media once (Optional step but helped to get rid of the dead floating cells) Complete growth media (0.5 ml) were added per well. Imaging protocol for IncuCyte was set up for whole well at 4×; and snapped every 2 hours for 1-1.5 days. Typically, 24 hours was enough for the wound closure if we start with a well-formed cell monolayer. The plate used for seeding cells was compatible with the IncuCyte.

On day 3, as illustrated in PLoS One. 2020; 15(7): e0232565, export cropped images from the IC software and analyse data using ImageJ plugin “Wound healing size tool”. Plugin gives following two measurements.
1) Area (i.e. wound area not covered by cells)
2) Area % (fraction of wound area not covered by cells relative to the full image area)

There is a limitation in the plugin. For example, when quantifying images that have multiple points where wound has closed, the Area % measurement is only given for the largest open area instead of measuring area from all open spaces (eg below images). This can be corrected by manually selecting to show “all ROI” and setting measurements to calculate the uncovered area from all ROIs, which can be summed up to get a more accurate value.

### EdU Incorporation Assay

### Day-1

Seed cells in 0.5 ml media per 24 well plate well with a round glass coverslip in each well
   50,000-100,000 cells per well (optimize the cell seeding density depending on the cell line so as to achieve 40-50% confluence on the next day)·
Incubate overnight

### Day-2

Prepare 2×EdU solution (final 10 μM) diluted in the media. Add equal volume of the EdU+media to the cells (do not remove the culture media from the plate completely)—eg. add 250 μl of 20 μM EdU+media to wells with 250 μl previous media
Incubate for 2 hours
Fix cells with PFA and keep at +4 C in PBS until required or proceed with the staining on the same day.

### For Coverslips in a 24 Well Plate

1. Removed media from each well and wash cells once with 0.5 ml PBS
2. Added 250 μl of 4% Paraformaldehyde in PBS (premade solution kept at +4 C; ChemCruz SC-281692). Incubated at room temperature (RT) for 15 min.
3. Removed the fixative and washed cells twice with 250 μL of 3% BSA in PBS (wash-buffer).
4. Removed wash-buffer. Added 300 μL of 0.5% Triton X-100 in PBS. Incubated at RT for 20 min.
5. Prepared Click-iT® reaction cocktail according to Table. It is important to add the ingredients in the order listed in the table; otherwise, the reaction will not proceed optimally. Use the Click-iT® reaction cocktail within 15 minutes of preparation.
6. Removed the permeabilization buffer (step 3.3), then washed the cells in each well twice with 300 μL of 3% BSA in PBS. Removed the wash solution.
7. Added 140 μL of Click-iT® reaction cocktail to each well containing a coverslip. Rocked the plate briefly to ensure that the reaction cocktail is distributed evenly over the coverslip. Incubated the plate for 30 minutes at room temperature, protected from light.
8. Removed the reaction cocktail, then wash each well once with 300 μL of 3% BSA in PBS. Removed the wash solution.
9. Washed each well with 0.5 mL of PBS. Removed the wash solution.
10. Diluted the Hoechst 33342 (Component G) solution 1:2000 in PBS to obtain a 1× Hoechst 33342 solution (the final concentration is 5 μg/mL).
11. Added 250 μL of 1× Hoechst 33342 solution per well. Incubated for 30 minutes at room temperature, protected from light. Remove the Hoechst 33342 solution.
12. Washed each well twice with 0.5 mL of PBS. Removed the wash solution.
13. Washed each well with MQ water immediately before mounting.
14. Mounted each coverslip on a microscope glass slide using a drop of mounting media. FluorSave (#345789 Millipore) solidify at RT in 1 hour.
15. Keep slides at +4 C in dark for long term storage. After an initial storage in the horizontal position overnight, the slides can be kept in a slide box in vertical position if required.

### EdU Staining Protocol

### Buffers & Solutions:

Wash-buffer. 3% BSA in PBS→0.9 μm BSA powder in 30 mL
Permeabilization buffer. 0.5% Triton X-100 in PBS→dilute 50 μL in 10 mL.
DNA stain: Hoechst 33342 (1:2000 dilution)→1 μL in 2 mL PBS
Click-IT® reaction cocktail (as in the table below):
   NOTE: Prepare fresh cocktail and use within 15 minutes.

| | 1 well (24-wp) | | 7 wells (24-wp) | | 24-wells (24-wp) | |
|---|---|---|---|---|---|---|
| MQ Water | 216 | μL | 864 | μL | 5184 | μL |
| 10X Reaction buffer | 21.5 | μL | 86 | μL | 516 | μL |
| CuSO₄ | 10 | μL | 40 | μL | 240 | μL |
| Alexa Fluor- 594 | 0.6 | μL | 2.4 | μL | 14.4 | μL |
| azide | | | | | | |
| 10X Rxn buffer | 2.5 | μL | 10 | μL | 60 | μL |
| Additive | | | | | | |
| Total Vol. | 250 | μL | 1 | mL | 6 | mL |

### Quantification Using ImageJ.

Data analyzed by ImageJ to measure the EdU+ fluorescence (area % and mean intensity values) per nuclei. Cell count was based on either all positive (values >0) or only bright positive (area %>50) cells. Similar count was obtained when using Mean intensity instead of Area % (cutoff for bright cells is 127.5).

### Quantification Steps:

1. DAPI channel image→Created mask of nuclei boundaries.
2. Overlayed the Nuclei_Mask image on EdU channel image (thresholded).
3. Measured the EdU signal within each nuclei (Area % & Mean intensity).
4. Exported this data to excel and apply cutoffs to count all EdU positive or “Bright EdU positive” cells.

### APPLICATIONS

Embodiments of methods disclosed herein provide therapeutic targets for the intervention of NAFLD/NASH. Advantageously, the targets disclosed herein came from an unbiased genome wide in vivo functional genetic screen. Even more advantageously, the targets pave the way for nucleic acid based therapies.

Embodiments of the disclosed methods and compositions also seek to overcome the problems of providing an alternative diagnosis method that does not rely on liver biopsy.

Advantageously, the methods as disclosed herein led to an alternative liver disease treatment. The methods also provide for regenerative medicine.

It will be appreciated by a person skilled in the art that other variations and/or modifications may be made to the embodiments disclosed herein without departing from the spirit or scope of the disclosure as broadly described. For example, in the description herein, features of different exemplary embodiments may be mixed, combined, interchanged, incorporated, adopted, modified, included etc. or the like across different exemplary embodiments. The present embodiments are, therefore, to be considered in all respects to be illustrative and not restrictive.

1. A method of regenerating a liver cell in a subject having a liver cell degeneration,
comprising administering an agent that modulates one or more gene or marker comprising C1ORF131 (2810004N23Rik), SLC45A4, NFKBIB, ARL6IP5, DBNL, GOLGA7B, FAM117B, MED28, TSC22D4, EIF4EBP1, PFN1, PNRC2, ZNF672 (Zfp672), IER5, ADAMTSL5, COL4A5, MYH15, and ILKAP,
wherein the agent counteracts the liver cell degeneration and/or enhances a liver cell regeneration.

2-19. (canceled)

20. The method of claim 1, wherein the one or more gene or marker further comprises ABCB10, LSM14A, ACTG1, YTHDF2, RPS6KA1, SEC13, IPO11, CTCF, UBAP2L, P2RY2, TRAF3IP2, FST, TUBG1, ZNF664, PVR, DYX1C1/DNAAF4, TEX264, NIF3L1, RPUSD1, TRIM6, GLRX3, BRD1, CKS1B, C6ORF120, WASF2, ZNF689, B4GALT7, LCORL, NR2F2, BAG6, and RNF10.

21. The method of claim 1, wherein the one or more gene or marker further comprises Uba6, Brwd3, Fyco1, ARID5B, C20ORF96 (6820408C15Rik), CD200R1 (Cd200r4), DALRD3, ELF3, ELFN1, FAM160A1, FAM50B, KIAA2026 (9930021J03Rik), KREMEN1, MACC1, NAA25, PROSC/PLPBP, RPS3, SHPK, SLAIN1, TCHP, ZNF503 (Zfp503), BUD13, CD93, PFKFB2, ZDBF2, FOXC2, GBGT1, RGS4, RNASEH1, TSNAXIP1, LYPD2, STIL, TMCO3, TMEM159, RIF1, IFIH1, EPN2, CLP1, RNF220, NOL12, APOL3, GTF3C6, CTPS2, IFITM2, IFITM1, KCNS3, CA2, EIF4H, MRM1, AKAP8, PAPOLA, SECTM1, TYK2, MLLT6, IL1RL2, IPO7, APBB1IP, RABEP2, PLSCR1, PYGO2, COIL, LRRC8E, TNRC6C, CHD3, USP21, SNX27, NOP14, ZC3H15, C15orf39, UTP20, TRAK1, RAB11FIP2, PHPT1, CUL3, and GNL3.

22. The method of claim 1, wherein the liver cell is a hepatocyte.

23. The method of claim 1, wherein the liver degeneration is non-alcoholic fatty liver disease (NAFLD) and/or non-alcoholic steatohepatitis (NASH).

24. The method of claim 1, wherein the agent reduces or inhibits the expression of the gene or marker.

25. The method of claim 1, wherein the agent is a nucleic acid capable of interfering with the expression of specific gene.

26. A nucleic acid encoding an agent for inhibiting the one or more gene or marker comprises C1ORF131 (2810004N23Rik), SLC45A4, NFKBIB, ARL6IP5, DBNL, GOLGA7B, FAM117B, MED28, TSC22D4, EIF4EBP1, PFN1, PNRC2, ZNF672 (Zfp672), IER5, ADAMTSL5, COL4A5, MYH15, and ILKAP; or a composition or a vector or a plasmid comprising the one or more of the nucleic acids; or a kit comprising the nucleic acids.

27. The nucleic acid of claim 26, wherein the one or more gene or marker further comprises ABCB10, LSM14A, ACTG1, YTHDF2, RPS6KA1, SEC13, IPO11, CTCF, UBAP2L, P2RY2, TRAF3IP2, FST, TUBG1, ZNF664, PVR, DYX1C1/DNAAF4, TEX264, NIF3L1, RPUSD1, TRIM6, GLRX3, BRD1, CKS1B, C6ORF120, WASF2, ZNF689, B4GALT7, LCORL, NR2F2, BAG6, and RNF10.

28. The nucleic acid of claim 26, wherein the one or more gene or marker may further comprises UBA6, BRWD3, FYCO1, ARID5B, C20ORF96 (6820408C15Rik), CD200R1 (Cd200r4), DALRD3, ELF3, ELFN1, FAM160A1, FAM50B, KIAA2026 (9930021J03Rik), KREMEN1, MACC1, NAA25, PROSC/PLPBP, RPS3, SHPK, SLAIN1, TCHP, ZNF503 (Zfp503), BUD13, CD93, PFKFB2, ZDBF2, FOXC2, GBGT1, RGS4, RNASEH1, TSNAXIP1, LYPD2, STIL, TMCO3, TMEM159, RIF1, IFIH1, EPN2, CLP1, RNF220, NOL12, APOL3, GTF3C6, CTPS2, IFITM2, IFITM1, KCNS3, CA2, EIF4H, MRM1, AKAP8, PAPOLA, SECTM1, TYK2, MLLT6, IL1RL2, IPO7, APBB1IP, RABEP2, PLSCR1, PYGO2, COIL, LRRC8E, TNRC6C, CHD3, USP21, SNX27, NOP14, ZC3H15, C15ORF39, UTP20, TRAK1, RAB11FIP2, PHPT1, CUL3, and GNL3.

29. The nucleic acid of claim 26, wherein the nucleic acid comprises one or more of an RNAi (RNA interference), a short hairpin RNA (shRNA), an antisense oligonucleotide (ASO), a gapmer, a short hairpin antisense oligonucleotide (shASO), a lipid nanoparticle, an adeno-associated virus vector, a gene editing agent, a ribozyme, or a nucleic acid-based nanoparticle.

30. The nucleic acid of claim 26, wherein the nucleic acid is a shRNA.

31. The nucleic acid of 26, wherein the nucleic acid is a shRNA and comprises the sequence selected from the group consisting of

| | | | SEQ |
|---|---|---|---|
| | Entrez | | ID |
| shRNA | ID | shRNA 97 mer sequence | NO: |
| Clorf131. | 128061 | TGCTGTTGACAGTGAGCGAAACCAAGAATCTAGTAATTTATAGT | 1 |
| 1320 | | GAAGCCACAGATGTATAAATTACTAGATTCTTGGTTCTGCCTAC | |
| | | TGCCTCGGA | |
| Clorf131. | 128061 | TGCTGTTGACAGTGAGCGCACAGAAGAAGATCATAAAGAATAG | 2 |
| 179 | | TGAAGCCACAGATGTATTCTTTATGATCTTCTTCTGTTTGCCTAC | |
| | | TGCCTCGGA | |
| Clorf131. | 128061 | TGCTGTTGACAGTGAGCGCCAGAAGAAGATCATAAAGAAATAG | 3 |
| 180 | | TGAAGCCACAGATGTATTTCTTTATGATCTTCTTCTGTTGCCTAC | |
| | | TGCCTCGGA | |
| Clorf131. | 128061 | TGCTGTTGACAGTGAGCGACAGATCATAACAAGAACACAATAG | 4 |
| 466 | | TGAAGCCACAGATGTATTGTGTTCTTGTTATGATCTGGTGCCTAC | |
| | | TGCCTCGGA | |
| Clorf131. | 128061 | TGCTGTTGACAGTGAGCGAAGAGATGTGGATACACAAGAATAG | 5 |
| 513 | | TGAAGCCACAGATGTATTCTTGTGTATCCACATCTCTCTGCCTAC | |
| | | TGCCTCGGA | |
| Clorf131. | 128061 | TGCTGTTGACAGTGAGCGCAAAGAGTTATGTGAATTACAATAGT | 6 |
| 653 | | GAAGCCACAGATGTATTGTAATTCACATAACTCTTTTTGCCTACT | |
| | | GCCTCGGA | |
| SLC45A4. | 57210 | TGCTGTTGACAGTGAGCGCACAGAAGTACTTGGACAACTATAGT | 7 |
| 1893 | | GAAGCCACAGATGTATAGTTGTCCAAGTACTTCTGTATGCCTAC | |
| | | TGCCTCGGA | |
| SLC45A4. | 57210 | TGCTGTTGACAGTGAGCGACACAATGTGGTTCTCTTAGAATAGT | 8 |
| 2558 | | GAAGCCACAGATGTATTCTAAGAGAACCACATTGTGGTGCCTAC | |
| | | TGCCTCGGA | |
| SLC45A4. | 57210 | TGCTGTTGACAGTGAGCGCCGGGCAAGTACTGCAGCTATTTAGT | 9 |
| 3229 | | GAAGCCACAGATGTAAATAGCTGCAGTACTTGCCCGATGCCTAC | |
| | | TGCCTCGGA | |
| SLC45A4. | 57210 | TGCTGTTGACAGTGAGCGCACCACAGTCTCTTTAGAACTATAGT | 10 |
| 3266 | | GAAGCCACAGATGTATAGTTCTAAAGAGACTGTGGTTTGCCTAC | |
| | | TGCCTCGGA | |
| SLC45A4. | 57210 | TGCTGTTGACAGTGAGCGACCCGCACATCACACAGATAAATAGT | 11 |
| 3340 | | GAAGCCACAGATGTATTTATCTGTGTGATGTGCGGGGTGCCTAC | |
| | | TGCCTCGGA | |
| SLC45A4. | 57210 | TGCTGTTGACAGTGAGCGCCACACAGATAAAGTCAGACAATAG | 12 |
| 3349 | | TGAAGCCACAGATGTATTGTCTGACTTTATCTGTGTGATGCCTA | |
| | | CTGCCTCGGA | |

32. The nucleic acid of claim 26, wherein the nucleic acid is a siRNA.

33. The nucleic acid of claim 26, wherein the nucleic acid is a siRNA and comprises the sequence selected from the group consisting of
C1ORF131 (Variant 1) Isoform a

| | | | SEQ | | SEQ | | |
|---|---|---|---|---|---|---|---|
| siRNA_ | | | ID | | ID | | Corrected_ |
| id | Position | SS Sequence | NO: | AS Sequence | NO: | Score | Score |
| 1 | 1251 | GCUAGUAGUUUAC | 13 | UUUAAUGUAAAC | 63 | 104.1 | 92.1 |
| | | AUUAAAUU | | UACUAGCAU | | | |
| 2 | 1316 | CCAAGAAUCUAGU | 14 | UAAAUUACUAGA | 64 | 102.8 | 89.8 |
| | | AAUUUAUC | | UUCUUGGUU | | | |
| 3 | 649 | AGAGUUAUGUGA | 15 | UUGUAAUUCACA | 65 | 99.8 | 93.8 |
| | | AUUACAAGG | | UAACUCUUU | | | |
| 4 | 171 | GAACAGAAGAAG | 16 | UUAUGAUCUUCU | 66 | 99.1 | 98.1 |
| | | AUCAUAAAG | | UCUGUUCUG | | | |
| 5 | 562 | GGUUUGGUAUCAC | 17 | UAACCCGUGAUAC | 67 | 99 | 94 |
| | | GGGUUAUG | | CAAACCGG | | | |
| 6 | 986 | CUAGAUAGAUCGU | 18 | UUAAGAACGAUC | 68 | 98.4 | 89.4 |
| | | UCUUAACA | | UAUCUAGAG | | | |
| 7 | 143 | CGACUUUGGAGGU | 19 | UUCUGUACCUCCA | 69 | 98.2 | 97.2 |
| | | ACAGAAGG | | AAGUCGUA | | | |
| 8 | 1201 | GGUGCAUUUGCUU | 20 | UAUUACAAGCAA | 70 | 98.2 | 86.2 |
| | | GUAAUACA | | AUGCACCAA | | | |
| 9 | 1250 | UGCUAGUAGUUU | 21 | UUAAUGUAAACU | 71 | 96.4 | 84.4 |
| | | ACAUUAAAU | | ACUAGCAUU | | | |
| 10 | 1244 | UGUAAAUGCUAG | 22 | UAAACUACUAGC | 72 | 96.3 | 84.3 |
| | | UAGUUUACA | | AUUUACAAA | | | |
| 11 | 503 | GGAGAGAGAUGU | 23 | UGUAUCCACAUCU | 73 | 96.2 | 91.2 |
| | | GGAUACACA | | CUCUCCAA | | | |
| 12 | 407 | GGAACAAGUAGA | 24 | UACCACUUCUACU | 74 | 96.1 | 92.1 |
| | | AGUGGUAGA | | UGUUCCCU | | | |
| 13 | 976 | CUGUGCGACUCUA | 25 | UCUAUCUAGAGU | 75 | 95.7 | 86.7 |
| | | GAUAGAUC | | CGCACAGGG | | | |
| 14 | 1110 | AGUGCAGCAUGAA | 26 | UUCACAUUCAUGC | 76 | 95.5 | 84.5 |
| | | UGUGAAGU | | UGCACUGG | | | |
| 15 | 162 | GGUGAAACAGAAC | 27 | UCUUCUGUUCUG | 77 | 95.5 | 94.5 |
| | | AGAAGAAG | | UUUCACCUU | | | |
| 16 | 385 | CCUCUUCCCUAAA | 28 | UUGUUCUUUAGG | 78 | 94.3 | 91.3 |
| | | GAACAAUA | | GAAGAGGGA | | | |
| 17 | 150 | GGAGGUACAGAA | 29 | UUUCACCUUCUGU | 79 | 93.7 | 92.7 |
| | | GGUGAAACA | | ACCUCCAA | | | |
| 18 | 1390 | CUUCAUAAGAAGU | 30 | UAUAUAACUUCU | 80 | 93.6 | 80.6 |
| | | UAUAUAAA | | UAUGAAGUA | | | |
| 19 | 170 | AGAACAGAAGAA | 31 | UAUGAUCUUCUU | 81 | 93.6 | 92.6 |
| | | GAUCAUAAA | | CUGUUCUGU | | | |
| 20 | 255 | CUACCUGGUUCUC | 32 | UUAUGAGAGAAC | 82 | 93.5 | 91.5 |
| | | UCAUAAGA | | CAGGUAGGG | | | |
| 21 | 254 | CCUACCUGGUUCU | 33 | UAUGAGAGAACC | 83 | 93.1 | 91.1 |
| | | CUCAUAAG | | AGGUAGGGG | | | |
| 22 | 398 | GAACAAUAGGGA | 34 | UACUUGUUCCCUA | 84 | 93 | 90 |
| | | ACAAGUAGA | | UUGUUCUU | | | |
| 23 | 1317 | CAAGAAUCUAGUA | 35 | AUAAAUUACUAG | 85 | 92.8 | 79.8 |
| | | AUUUAUCA | | AUUCUUGGU | | | |
| 24 | 423 | GUAGAAUUUCACA | 36 | UAUUGCUGUGAA | 86 | 92.5 | 88.5 |
| | | GCAAUAAA | | AUUCUACCA | | | |
| 25 | 910 | AAUGAAGUACUU | 37 | UUUGACAAAGUA | 87 | 92.1 | 83.1 |
| | | UGUCAAAUA | | CUUCAUUUG | | | |
| 26 | 40 | CAAUAGGGAACAA | 38 | UUCUACUUGUUCC | 88 | 92.1 | 88.1 |
| | | GUAGAAGU | | CUAUUGUU | | | |
| 27 | 912 | UGAAGUACUUUG | 39 | UAUUUGACAAAG | 89 | 92 | 83 |
| | | UCAAAUAAA | | UACUUCAUU | | | |
| 28 | 909 | AAAUGAAGUACU | 40 | UUGACAAAGUAC | 90 | 91.8 | 82.8 |
| | | UUGUCAAAU | | UUCAUUUGG | | | |
| 29 | 1294 | AUGUUAUGAUAU | 41 | UUUCAUGAUAUC | 91 | 91.8 | 79.8 |
| | | CAUGAAAGA | | AUAACAUUA | | | |
| 30 | 1313 | GAACCAAGAAUCU | 42 | AUUACUAGAUUC | 92 | 91.6 | 78.6 |
| | | AGUAAUUU | | UUGGUUCUU | | | |
| 31 | 392 | CCUAAAGAACAAU | 43 | UUCCCUAUUGUUC | 93 | 91.3 | 88.3 |
| | | AGGGAACA | | UUUAGGGA | | | |
| 32 | 1180 | GUUUCAUGUUCAC | 44 | AUACCAGUGAAC | 94 | 91.3 | 80.3 |
| | | UGGUAUUU | | AUGAAACAG | | | |
| 33 | 289 | GCGCUUCGAGCUU | 45 | UUGAAGAAGCUC | 95 | 91.3 | 89.3 |
| | | CUUCAAGG | | GAAGCGCUC | | | |
| 34 | 934 | CGAGGAUGGGAAC | 46 | UCAGUUGUUCCCA | 96 | 91.2 | 82.2 |
| | | AACUGAGU | | UCCUCGUU | | | |
| 35 | 1286 | AUGAUUUAAUGU | 47 | UAUCAUAACAUU | 97 | 91 | 79 |
| | | UAUGAUAUC | | AAAUCAUUA | | | |
| 36 | 1393 | CAUAAGAAGUUA | 48 | AUUUAUAUAACU | 98 | 90.9 | 77.9 |
| | | UAUAAAUAA | | UCUUAUGAA | | | |
| 37 | 413 | AGUAGAAGUGGU | 49 | AAAUUCUACCACU | 99 | 90.9 | 86.9 |
| | | AGAAUUUCA | | UCUACUUG | | | |
| 38 | 378 | GUUCCUCCCUCUU | 50 | UUAGGGAAGAGG | 100 | 90.9 | 87.9 |
| | | CCCUAAAG | | GAGGAACUG | | | |
| 39 | 764 | AGGACAGGAGGAC | 51 | UUUCCUGUCCUCC | 101 | 90.6 | 83.6 |
| | | AGGAAAUC | | UGUCCUUU | | | |
| 40 | 972 | GACCCUGUGCGAC | 52 | UCUAGAGUCGCAC | 102 | 90.5 | 81.5 |
| | | UCUAGAUA | | AGGGUCCA | | | |
| 41 | 422 | GGUAGAAUUUCAC | 53 | AUUGCUGUGAAA | 103 | 90.4 | 86.4 |
| | | AGCAAUAA | | UUCUACCAC | | | |
| 42 | 172 | AACAGAAGAAGA | 54 | UUUAUGAUCUUC | 104 | 90.2 | 89.2 |
| | | UCAUAAAGA | | UUCUGUUCU | | | |
| 43 | 1386 | UCUACUUCAUAAG | 55 | UAACUUCUUAUG | 105 | 90.1 | 77.1 |
| | | AAGUUAUA | | AAGUAGAUU | | | |
| 44 | 974 | CCCUGUGCGACUC | 56 | UAUCUAGAGUCG | 106 | 89.9 | 80.9 |
| | | UAGAUAGA | | CACAGGGUC | | | |
| 45 | 552 | GAAGUGCACCGGU | 57 | UACCAAACCGGUG | 107 | 89.7 | 84.7 |
| | | UUGGUAUC | | CACUUCUA | | | |
| 46 | 1394 | AUAAGAAGUUAU | 58 | UAUUUAUAUAAC | 108 | 89.7 | 76.7 |
| | | AUAAAUAAA | | UUCUUAUGA | | | |
| 47 | 648 | AAGAGUUAUGUG | 59 | UGUAAUUCACAU | 109 | 89.6 | 83.6 |
| | | AAUUACAAG | | AACUCUUUU | | | |
| 48 | 765 | GGACAGGAGGACA | 60 | AUUUCCUGUCCUC | 110 | 89.5 | 82.5 |
| | | GGAAAUCC | | CUGUCCUU | | | |
| 49 | 359 | AGAGAUCCUUGCU | 61 | UGCAGCAGCAAG | 111 | 89.4 | 86.4 |
| | | GCUGCAGU | | GAUCUCUGG | | | |
| 50 | 1249 | AUGCUAGUAGUU | 62 | UAAUGUAAACUA | 112 | 89.4 | 77.4 |
| | | UACAUUAAA | | CUAGCAUUU | | | |

C1ORF131 (Variant 2) Isoform b

| | | | SEQ | | SEQ | | |
|---|---|---|---|---|---|---|---|
| siRNA_ | | | ID | | ID | | Corrected_ |
| id | Position | SS Sequence | NO: | AS Sequence | NO: | Score | Score |
| 1 | 1248 | GCUAGUAGUUUAC | 113 | UUUAAUGUAAACU | 163 | 104.1 | 92.1 |
| | | AUUAAAUU | | ACUAGCAU | | | |
| 2 | 1313 | CCAAGAAUCUAGU | 114 | UAAAUUACUAGAU | 164 | 102.8 | 89.8 |
| | | AAUUUAUC | | UCUUGGUU | | | |
| 3 | 646 | AGAGUUAUGUGAA | 115 | UUGUAAUUCACAU | 165 | 99.8 | 93.8 |
| | | UUACAAGG | | AACUCUUU | | | |
| 4 | 171 | GAACAGAAGAAGA | 116 | UUAUGAUCUUCUU | 166 | 99.1 | 98.1 |
| | | UCAUAAAG | | CUGUUCUG | | | |
| 5 | 559 | GGUUUGGUAUCAC | 117 | UAACCCGUGAUAC | 167 | 99 | 94 |
| | | GGGUUAUG | | CAAACCGG | | | |
| 6 | 983 | CUAGAUAGAUCGU | 118 | UUAAGAACGAUCU | 168 | 98.4 | 89.4 |
| | | UCUUAACA | | AUCUAGAG | | | |
| 7 | 143 | CGACUUUGGAGGU | 119 | UUCUGUACCUCCA | 169 | 98.2 | 97.2 |
| | | ACAGAAGG | | AAGUCGUA | | | |
| 8 | 1198 | GGUGCAUUUGCUU | 120 | UAUUACAAGCAAA | 170 | 98.2 | 87.2 |
| | | GUAAUACA | | UGCACCAA | | | |
| 9 | 1247 | UGCUAGUAGUUUA | 121 | UUAAUGUAAACUA | 171 | 96.4 | 84.4 |
| | | CAUUAAAU | | CUAGCAUU | | | |
| 10 | 1241 | UGUAAAUGCUAGU | 122 | UAAACUACUAGCA | 172 | 96.3 | 84.3 |
| | | AGUUUACA | | UUUACAAA | | | |
| 11 | 500 | GGAGAGAGAUGUG | 123 | UGUAUCCACAUCU | 173 | 96.2 | 91.2 |
| | | GAUACACA | | CUCUCCAA | | | |
| 12 | 407 | GGAACAAGUAGAA | 124 | UACCACUUCUACU | 174 | 96.1 | 92.1 |
| | | GUGGUAGA | | UGUUCCCU | | | |
| 13 | 973 | CUGUGCGACUCUA | 125 | UCUAUCUAGAGUC | 175 | 95.7 | 86.7 |
| | | GAUAGAUC | | GCACAGGG | | | |
| 14 | 1107 | AGUGCAGCAUGAA | 126 | UUCACAUUCAUGC | 176 | 95.5 | 84.5 |
| | | UGUGAAGU | | UGCACUGG | | | |
| 15 | 162 | GGUGAAACAGAAC | 127 | UCUUCUGUUCUGU | 177 | 95.5 | 94.5 |
| | | AGAAGAAG | | UUCACCUU | | | |
| 16 | 385 | CCUCUUCCCUAAA | 128 | UUGUUCUUUAGGG | 178 | 94.3 | 91.3 |
| | | GAACAAUA | | AAGAGGGA | | | |
| 17 | 150 | GGAGGUACAGAAG | 129 | UUUCACCUUCUGU | 179 | 93.7 | 92.7 |
| | | GUGAAACA | | ACCUCCAA | | | |
| 18 | 1387 | CUUCAUAAGAAGU | 130 | UAUAUAACUUCUU | 180 | 93.6 | 80.6 |
| | | UAUAUAAA | | AUGAAGUA | | | |
| 19 | 170 | AGAACAGAAGAAG | 131 | UAUGAUCUUCUUC | 181 | 93.6 | 92.6 |
| | | AUCAUAAA | | UGUUCUGU | | | |
| 20 | 255 | CUACCUGGUUCUC | 132 | UUAUGAGAGAACC | 182 | 93.5 | 91.5 |
| | | UCAUAAGA | | AGGUAGGG | | | |
| 21 | 254 | CCUACCUGGUUCU | 133 | UAUGAGAGAACCA | 183 | 93.1 | 91.1 |
| | | CUCAUAAG | | GGUAGGGG | | | |
| 22 | 398 | GAACAAUAGGGAA | 134 | UACUUGUUCCCUA | 184 | 93 | 90 |
| | | CAAGUAGA | | UUGUUCUU | | | |
| 23 | 1314 | CAAGAAUCUAGUA | 135 | AUAAAUUACUAGA | 185 | 92.8 | 79.8 |
| | | AUUUAUCA | | UUCUUGGU | | | |
| 24 | 423 | GUAGAAUUUCACA | 136 | UAUUGCUGUGAAA | 186 | 92.5 | 88.5 |
| | | GCAAUAAA | | UUCUACCA | | | |
| 25 | 907 | AAUGAAGUACUUU | 137 | UUUGACAAAGUAC | 187 | 92.1 | 83.1 |
| | | GUCAAAUA | | UUCAUUUG | | | |
| 26 | 40 | CAAUAGGGAACAA | 138 | UUCUACUUGUUCC | 188 | 92.1 | 88.1 |
| | | GUAGAAGU | | CUAUUGUU | | | |
| 27 | 909 | UGAAGUACUUUGU | 139 | UAUUUGACAAAGU | 189 | 92 | 83 |
| | | CAAAUAAA | | ACUUCAUU | | | |
| 28 | 906 | AAAUGAAGUACUU | 140 | UUGACAAAGUACU | 190 | 91.8 | 82.8 |
| | | UGUCAAAU | | UCAUUUGG | | | |
| 29 | 1291 | AUGUUAUGAUAUC | 141 | UUUCAUGAUAUCA | 191 | 91.8 | 79.8 |
| | | AUGAAAGA | | UAACAUUA | | | |
| 30 | 1310 | GAACCAAGAAUCU | 142 | AUUACUAGAUUCU | 192 | 91.6 | 78.6 |
| | | AGUAAUUU | | UGGUUCUU | | | |
| 31 | 392 | CCUAAAGAACAAU | 143 | UUCCCUAUUGUUC | 193 | 91.3 | 88.3 |
| | | AGGGAACA | | UUUAGGGA | | | |
| 32 | 1177 | GUUUCAUGUUCAC | 144 | AUACCAGUGAACA | 194 | 91.3 | 80.3 |
| | | UGGUAUUU | | UGAAACAG | | | |
| 33 | 289 | GCGCUUCGAGCUU | 145 | UUGAAGAAGCUCG | 195 | 91.3 | 89.3 |
| | | CUUCAAGG | | AAGCGCUC | | | |
| 34 | 931 | CGAGGAUGGGAAC | 146 | UCAGUUGUUCCCA | 196 | 91.2 | 82.2 |
| | | AACUGAGU | | UCCUCGUU | | | |
| 35 | 1283 | AUGAUUUAAUGUU | 147 | UAUCAUAACAUUA | 197 | 91 | 79 |
| | | AUGAUAUC | | AAUCAUUA | | | |
| 36 | 1390 | CAUAAGAAGUUAU | 148 | AUUUAUAUAACUU | 198 | 90.9 | 77.9 |
| | | AUAAAUAA | | CUUAUGAA | | | |
| 37 | 413 | AGUAGAAGUGGUA | 149 | AAAUUCUACCACU | 199 | 90.9 | 86.9 |
| | | GAAUUUCA | | UCUACUUG | | | |
| 38 | 378 | GUUCCUCCCUCUU | 150 | UUAGGGAAGAGGG | 200 | 90.9 | 87.9 |
| | | CCCUAAAG | | AGGAACUG | | | |
| 39 | 761 | AGGACAGGAGGAC | 151 | UUUCCUGUCCUCC | 201 | 90.6 | 83.6 |
| | | AGGAAAUC | | UGUCCUUU | | | |
| 40 | 969 | GACCCUGUGCGAC | 152 | UCUAGAGUCGCAC | 202 | 90.5 | 81.5 |
| | | UCUAGAUA | | AGGGUCCA | | | |
| 41 | 422 | GGUAGAAUUUCAC | 153 | AUUGCUGUGAAAU | 203 | 90.4 | 86.4 |
| | | AGCAAUAA | | UCUACCAC | | | |
| 42 | 172 | AACAGAAGAAGAU | 154 | UUUAUGAUCUUCU | 204 | 90.2 | 89.2 |
| | | CAUAAAGA | | UCUGUUCU | | | |
| 43 | 1383 | UCUACUUCAUAAG | 155 | UAACUUCUUAUGA | 205 | 90.1 | 77.1 |
| | | AAGUUAUA | | AGUAGAUU | | | |
| 44 | 971 | CCCUGUGCGACUC | 156 | UAUCUAGAGUCGC | 206 | 89.9 | 80.9 |
| | | UAGAUAGA | | ACAGGGUC | | | |
| 45 | 549 | GAAGUGCACCGGU | 157 | UACCAAACCGGUG | 207 | 897 | 84.7 |
| | | UUGGUAUC | | CACUUCUA | | | |
| 46 | 1391 | AUAAGAAGUUAUA | 158 | UAUUUAUAUAACU | 208 | 89.7 | 76.7 |
| | | UAAAUAAA | | UCUUAUGA | | | |
| 47 | 645 | AAGAGUUAUGUGA | 159 | UGUAAUUCACAUA | 209 | 89.6 | 83.6 |
| | | AUUACAAG | | ACUCUUUU | | | |
| 48 | 762 | GGACAGGAGGACA | 160 | AUUUCCUGUCCUC | 210 | 89.5 | 82.5 |
| | | GGAAAUCC | | CUGUCCUU | | | |
| 49 | 359 | AGAGAUCCUUGCU | 161 | UGCAGCAGCAAGG | 211 | 89.4 | 86.4 |
| | | GCUGCAGU | | AUCUCUGG | | | |
| 50 | 1246 | AUGCUAGUAGUUU | 162 | UAAUGUAAACUAC | 212 | 89.4 | 77.4 |
| | | ACAUUAAA | | UAGCAUUU | | | |

SLC45A4 (Variant 1) Isoform 1

| | | | SEQ | | SEQ | | |
|---|---|---|---|---|---|---|---|
| siRNA_ | | | ID | | ID | | Corrected_ |
| id | Position | SS Sequence | NO: | AS Sequence | NO: | Score | Score |
| 1 | 553 | GAUAGAAUAUAUA | 213 | UUUAUCUAUAUAU | 263 | 105.5 | 100.5 |
| | | GAUAAAUA | | UCUAUCUA | | | |
| 2 | 621 | GGAAUCUAUGCAA | 214 | UUGAACUUGCAUA | 264 | 102.8 | 96.8 |
| | | GUUCAAGA | | GAUUCCGG | | | |
| 3 | 3990 | GGAACGACUGUUU | 215 | UACAUUAAACAGU | 265 | 102.2 | 63.2 |
| | | AAUGUACU | | CGUUCCUU | | | |
| 4 | 3803 | GUCAGACAAUUGU | 216 | UUAAUUACAAUUG | 266 | 102 | 64 |
| | | AAUUAAUA | | UCUGACUU | | | |
| 5 | 4761 | CCCUCUGAAUAGU | 217 | UAACACACUAUUC | 267 | 100.9 | 53.9 |
| | | GUGUUAAC | | AGAGGGGG | | | |
| 6 | 6695 | AGUUCAGUUCCGA | 218 | UUCAACUCGGAAC | 268 | 100.7 | 34.7 |
| | | GUUGAACA | | UGAACUGA | | | |
| 7 | 620 | CGGAAUCUAUGCA | 219 | UGAACUUGCAUAG | 269 | 100.7 | 94.7 |
| | | AGUUCAAG | | AUUCCGGG | | | |
| 8 | 501 | GAGAAGACGUCUU | 220 | UAAAUUAAGACGU | 270 | 99.5 | 94.5 |
| | | AAUUUAUC | | CUUCUCUU | | | |
| 9 | 3002 | CAAUGUGGUUCUC | 221 | UUCUAAGAGAACC | 271 | 99.4 | 69.4 |
| | | UUAGAAGA | | ACAUUGUG | | | |
| 10 | 2330 | AGAAGUACUUGGA | 222 | UAGUUGUCCAAGU | 272 | 99.2 | 76.2 |
| | | CAACUACG | | ACUUCUGU | | | |
| 11 | 552 | AGAUAGAAUAUAU | 223 | UUAUCUAUAUAUU | 273 | 98.7 | 93.7 |
| | | AGAUAAAU | | CUAUCUAU | | | |
| 12 | 5682 | GGAUAAUGUUGGU | 224 | AAUGAGACCAACA | 274 | 98.7 | 42.7 |
| | | CUCAUUUG | | UUAUCCUA | | | |
| 13 | 6168 | GGUCUGUGACCAC | 225 | UUCUAGGUGGUCA | 275 | 98.4 | 37.4 |
| | | CUAGAACC | | CAGACCAA | | | |
| 14 | 4561 | GAAUCAAGAUCCG | 226 | UUGGGUCGGAUCU | 276 | 98.3 | 53.3 |
| | | ACCCAAAC | | UGAUUCGG | | | |
| 15 | 4228 | GGCAAGUCCGACU | 227 | UUCAGUAGUCGGA | 277 | 98.1 | 56.1 |
| | | ACUGAAAG | | CUUGCCCU | | | |
| 16 | 4955 | CACUUUGUAGACU | 228 | UUGACGAGUCUAC | 278 | 97.5 | 48.5 |
| | | CGUCAAGC | | AAAGUGUG | | | |
| 17 | 5627 | GGGUGUGUCGGUU | 229 | UCGACCAACCGAC | 279 | 97.4 | 41.4 |
| | | GGUCGAGU | | ACACCCUG | | | |
| 18 | 1346 | GCAUCGACGAGGA | 230 | UACUGCUCCUCGU | 280 | 97 | 84 |
| | | GCAGUACA | | CGAUGCUG | | | |
| 19 | 5716 | CCCUAUACAAGUC | 231 | UUAGCUGACUUGU | 281 | 97 | 40 |
| | | AGCUAAGU | | AUAGGGAA | | | |
| 20 | 4981 | GCACUUCAGGUGU | 232 | UUGCAAACACCUG | 282 | 96.9 | 47.9 |
| | | UUGCAAGC | | AAGUGCUG | | | |
| 21 | 3884 | GCAAUAUGAUCAU | 233 | UCUAGAAUGAUCA | 283 | 96.6 | 58.6 |
| | | UCUAGAGC | | UAUUGCAG | | | |
| 22 | 3643 | GUUCUGGUUUCAA | 234 | UAUUCGUUGAAAC | 284 | 96.5 | 60.5 |
| | | CGAAUAAG | | CAGAACGA | | | |
| 23 | 1713 | GGAUAAUCACUUG | 235 | UUCAUUCAAGUGA | 285 | 96.1 | 79.1 |
| | | AAUGAAGC | | UUAUCCAG | | | |
| 24 | 3995 | GACUGUUUAAUGU | 236 | UCAAGUACAUUAA | 286 | 96.1 | 57.1 |
| | | ACUUGAUG | | ACAGUCGU | | | |
| 25 | 2999 | CCACAAUGUGGUU | 237 | UAAGAGAACCACA | 287 | 95.9 | 66.9 |
| | | CUCUUAGA | | UUGUGGAA | | | |
| 26 | 801 | CGCUCUGGUCACA | 238 | UAUUGGUGUGACC | 288 | 95.7 | 87.7 |
| | | CCAAUACU | | AGAGCGGU | | | |
| 27 | 181 | GGAGGAGACAGAU | 239 | UGGAUGAUCUGUC | 289 | 95.5 | 94.5 |
| | | CAUCCAGU | | UCCUCCGG | | | |
| 28 | 4054 | CUUUGUAGAUUGU | 240 | AAACACACAAUCU | 290 | 95.5 | 55.5 |
| | | GUGUUUGU | | ACAAAGUG | | | |
| 29 | 1265 | GCUGGUUCCGGAC | 241 | UUCUGGGUCCGGA | 291 | 95.4 | 83.4 |
| | | CCAGAACC | | ACCAGCUG | | | |
| 30 | 557 | GAAUAUAUAGAUA | 242 | UGUAUUUAUCUAU | 292 | 95.4 | 90.4 |
| | | AAUACAUA | | AUAUUCUA | | | |
| 31 | 1952 | GCAUGAGCGACCU | 243 | UCGUACAGGUCGC | 293 | 95.1 | 76.1 |
| | | GUACGACA | | UCAUGCUG | | | |
| 32 | 284 | CUUCAGUAAUAAU | 244 | UUCAUCAUUAUUA | 294 | 95.1 | 93.1 |
| | | GAUGAAAC | | CUGAAGAG | | | |
| 33 | 526 | CAGAUAUAUGAAA | 245 | UAAUGCUUUCAUA | 295 | 95 | 90 |
| | | GCAUUAUU | | UAUCUGUA | | | |
| 34 | 1862 | CCAGCACCUUCUC | 246 | UAGUAGGAGAAGG | 296 | 95 | 77 |
| | | CUACUACG | | UGCUGGAG | | | |
| 35 | 5668 | CAGUUACCAAACU | 247 | UAUCCUAGUUUGG | 297 | 95 | 39 |
| | | AGGAUAAU | | UAACUGGU | | | |
| 36 | 497 | GAAAGAGAAGACG | 248 | UUAAGACGUCUUC | 298 | 95 | 91 |
| | | UCUUAAUU | | UCUUUCUG | | | |
| 37 | 627 | UAUGCAAGUUCAA | 249 | UAACUCUUGAACU | 299 | 94.9 | 88.9 |
| | | GAGUUAUC | | UGCAUAGA | | | |
| 38 | 486 | CAGCAGAAGCAGA | 250 | UCUCUUUCUGCUU | 300 | 94.9 | 90.9 |
| | | AAGAGAAG | | CUGCUGUG | | | |
| 39 | 487 | AGCAGAAGCAGAA | 251 | UUCUCUUUCUGCU | 301 | 94.8 | 90.8 |
| | | AGAGAAGA | | UCUGCUGU | | | |
| 40 | 3710 | CACAGUCUCUUUA | 252 | UAGUUCUAAAGAG | 302 | 94.8 | 57.8 |
| | | GAACUAAG | | ACUGUGGU | | | |
| 41 | 3673 | GGCAAGUACUGCA | 253 | AAUAGCUGCAGUA | 303 | 94.7 | 58.7 |
| | | GCUAUUUG | | CUUGCCCG | | | |
| 42 | 278 | GCACCUCUUCAGU | 254 | AUUAUUACUGAAG | 304 | 94.7 | 92.7 |
| | | AAUAAUGA | | AGGUGCUG | | | |
| 43 | 812 | CACCAAUACUGUU | 255 | AUCUGCAACAGUA | 305 | 94.6 | 86.6 |
| | | GCAGAUUG | | UUGGUGUG | | | |
| 44 | 5224 | AGAUUGCACUCAC | 256 | UUUCAAGUGAGUG | 306 | 94.5 | 42.5 |
| | | UUGAAACC | | CAAUCUGG | | | |
| 45 | 452 | AAGAGGAAUGUAA | 257 | UAACACUUACAUU | 307 | 94.5 | 90.5 |
| | | GUGUUACC | | CCUCUUUG | | | |
| 46 | 3284 | CGUCUGAAGGACU | 258 | UAGGAGAGUCCUU | 308 | 94.4 | 62.4 |
| | | CUCCUAGG | | CAGACGGG | | | |
| 47 | 5663 | CAGACCAGUUACC | 259 | UAGUUUGGUAACU | 309 | 94.3 | 38.3 |
| | | AAACUAGG | | GGUCUGCG | | | |
| 48 | 4141 | GCAGAGGGCUGAC | 260 | UUGAAAGUCAGCC | 310 | 94.2 | 53.2 |
| | | UUUCAAGA | | CUCUGCGA | | | |
| 49 | 3461 | CGGGUGUGUGGAU | 261 | UCGCACAUCCACA | 311 | 94 | 60 |
| | | GUGCGAGG | | CACCCGAG | | | |
| 50 | 4227 | GGGCAAGUCCGAC | 262 | UCAGUAGUCGGAC | 312 | 93.9 | 51.9 |
| | | UACUGAAA | | UUGCCCUG | | | |

SLC45A4 (Variant 2) Isoform 2

| | | | SEQ | | SEQ | | |
|---|---|---|---|---|---|---|---|
| siRNA_ | | | ID | | ID | | Corrected_ |
| id | Position | SS Sequence | NO: | AS Sequence | NO: | Score | Score |
| 1 | 3545 | GGAACGACUGUUU | 313 | UACAUUAAACAGU | 363 | 102.2 | 67.2 |
| | | AAUGUACU | | CGUUCCUU | | | |
| 2 | 3358 | GUCAGACAAUUGU | 314 | UUAAUUACAAUUG | 364 | 102 | 69 |
| | | AAUUAAUA | | UCUGACUU | | | |
| 3 | 4316 | CCCUCUGAAUAGU | 315 | UAACACACUAUUC | 365 | 100.9 | 57.9 |
| | | GUGUUAAC | | AGAGGGGG | | | |
| 4 | 6250 | AGUUCAGUUCCGA | 316 | UUCAACUCGGAAC | 366 | 100.7 | 38.7 |
| | | GUUGAACA | | UGAACUGA | | | |
| 5 | 2557 | CAAUGUGGUUCUC | 317 | UUCUAAGAGAACC | 367 | 99.4 | 74.4 |
| | | UUAGAAGA | | ACAUUGUG | | | |
| 6 | 1892 | AGAAGUACUUGGA | 318 | UAGUUGUCCAAGU | 368 | 99.2 | 81.2 |
| | | CAACUACG | | ACUUCUGU | | | |
| 7 | 5237 | GGAUAAUGUUGGU | 319 | AAUGAGACCAACA | 339 | 98.7 | 46.7 |
| | | CUCAUUUG | | UUAUCCUA | | | |
| 8 | 5723 | GGUCUGUGACCAC | 320 | UUCUAGGUGGUCA | 340 | 98.4 | 41.4 |
| | | CUAGAACC | | CAGACCAA | | | |
| 9 | 4116 | GAAUCAAGAUCCG | 321 | UUGGGUCGGAUCU | 341 | 98.3 | 57.3 |
| | | ACCCAAAC | | UGAUUCGG | | | |
| 10 | 3783 | GGCAAGUCCGACU | 322 | UUCAGUAGUCGGA | 342 | 98.1 | 61.1 |
| | | ACUGAAAG | | CUUGCCCU | | | |
| 11 | 4510 | CACUUUGUAGACU | 323 | UUGACGAGUCUAC | 343 | 97.5 | 52.5 |
| | | CGUCAAGC | | AAAGUGUG | | | |
| 12 | 5182 | GGGUGUGUCGGUU | 324 | UCGACCAACCGAC | 344 | 97.4 | 46.4 |
| | | GGUCGAGU | | ACACCCUG | | | |
| 13 | 908 | GCAUCGACGAGGA | 325 | UACUGCUCCUCGU | 345 | 97 | 88 |
| | | GCAGUACA | | CGAUGCUG | | | |
| 14 | 5271 | CCCUAUACAAGUC | 326 | UUAGCUGACUUGU | 346 | 97 | 45 |
| | | AGCUAAGU | | AUAGGGAA | | | |
| 15 | 4536 | GCACUUCAGGUGU | 327 | UUGCAAACACCUG | 347 | 96.9 | 51.9 |
| | | UUGCAAGC | | AAGUGCUG | | | |
| 16 | 3439 | GCAAUAUGAUCAU | 328 | UCUAGAAUGAUCA | 348 | 96.6 | 62.6 |
| | | UCUAGAGC | | UAUUGCAG | | | |
| 17 | 3198 | GUUCUGGUUUCAA | 329 | UAUUCGUUGAAAC | 349 | 96.5 | 65.5 |
| | | CGAAUAAG | | CAGAACGA | | | |
| 18 | 1275 | GGAUAAUCACUUG | 330 | UUCAUUCAAGUGA | 350 | 96.1 | 84.1 |
| | | AAUGAAGC | | UUAUCCAG | | | |
| 19 | 3550 | GACUGUUUAAUGU | 331 | UCAAGUACAUUAA | 351 | 96.1 | 61.1 |
| | | ACUUGAUG | | ACAGUCGU | | | |
| 20 | 2554 | CCACAAUGUGGUU | 332 | UAAGAGAACCACA | 352 | 95.9 | 70.9 |
| | | CUCUUAGA | | UUGUGGAA | | | |
| 21 | 3609 | CUUUGUAGAUUGU | 333 | AAACACACAAUCU | 353 | 95.5 | 59.5 |
| | | GUGUUUGU | | ACAAAGUG | | | |
| 22 | 827 | GCUGGUUCCGGAC | 334 | UUCUGGGUCCGGA | 354 | 954 | 87.4 |
| | | CCAGAACC | | ACCAGCUG | | | |
| 23 | 1514 | GCAUGAGCGACCU | 335 | UCGUACAGGUCGC | 355 | 95.1 | 80.1 |
| | | GUACGACA | | UCAUGCUG | | | |
| 24 | 1424 | CCAGCACCUUCUC | 336 | UAGUAGGAGAAGG | 356 | 95 | 81 |
| | | CUACUACG | | UGCUGGAG | | | |
| 25 | 5223 | CAGUUACCAAACU | 337 | UAUCCUAGUUUGG | 357 | 95 | 43 |
| | | AGGAUAAU | | UAACUGGU | | | |
| 26 | 3265 | CACAGUCUCUUUA | 338 | UAGUUCUAAAGAG | 358 | 94.8 | 62.8 |
| | | GAACUAAG | | ACUGUGGU | | | |
| 27 | 3228 | GGCAAGUACUGCA | 339 | AAUAGCUGCAGUA | 359 | 94.7 | 62.7 |
| | | GCUAUUUG | | CUUGCCCG | | | |
| 28 | 4779 | AGAUUGCACUCAC | 340 | UUUCAAGUGAGUG | 360 | 94.5 | 47.5 |
| | | UUGAAACC | | CAAUCUGG | | | |
| 29 | 2839 | CGUCUGAAGGACU | 341 | UAGGAGAGUCCUU | 361 | 94.4 | 66.4 |
| | | CUCCUAGG | | CAGACGGG | | | |
| 30 | 5218 | CAGACCAGUUACC | 342 | UAGUUUGGUAACU | 362 | 94.3 | 42.3 |
| | | AAACUAGG | | GGUCUGCG | | | |
| 31 | 3696 | GCAGAGGGCUGAC | 343 | UUGAAAGUCAGCC | 363 | 94.2 | 58.2 |
| | | UUUCAAGA | | CUCUGCGA | | | |
| 32 | 226 | CCUUGUUGAAGAG | 344 | UUUCUCCUCUUCA | 364 | 94.2 | 92.2 |
| | | GAGAAACA | | ACAAGGAG | | | |
| 33 | 3016 | CGGGUGUGUGGAU | 345 | UCGCACAUCCACA | 365 | 94 | 64 |
| | | GUGCGAGG | | CACCCGAG | | | |
| 34 | 3782 | GGGCAAGUCCGAC | 346 | UCAGUAGUCGGAC | 366 | 93.9 | 56.9 |
| | | UACUGAAA | | UUGCCCUG | | | |
| 35 | 3456 | GAGCAAAUAUAUA | 347 | UGUAUAUAUAUAU | 367 | 93.9 | 59.9 |
| | | UAUACACG | | UUGCUCUA | | | |
| 36 | 4625 | GGGUCAGUAGAGU | 348 | AAACCGACUCUAC | 368 | 93.7 | 47.7 |
| | | CGGUUUCU | | UGACCCUG | | | |
| 37 | 4317 | CCUCUGAAUAGUG | 349 | UUAACACACUAUU | 369 | 93.7 | 50.7 |
| | | UGUUAACC | | CAGAGGGG | | | |
| 38 | 5872 | GAUACCAACUGUU | 350 | UAAGGGAACAGUU | 370 | 93.5 | 35.5 |
| | | CCCUUAUA | | GGUAUCUU | | | |
| 39 | 1430 | CCUUCUCCUACUA | 351 | UUGCCGUAGUAGG | 371 | 93.3 | 79.3 |
| | | CGGCAAGC | | AGAAGGUG | | | |
| 40 | 3706 | GACUUUCAAGACU | 352 | UCUUGAAGUCUUG | 372 | 93.3 | 56.3 |
| | | UCAAGAAC | | AAAGUCAG | | | |
| 41 | 1877 | GUUCAGCCCUGUU | 353 | UUCUGUAACAGGG | 373 | 93.3 | 75.3 |
| | | ACAGAAGU | | CUGAACAA | | | |
| 42 | 289 | GCAGGGACAGCAU | 354 | UAGAUGAUGCUGU | 374 | 93.1 | 91.1 |
| | | CAUCUAUG | | CCCUGCCG | | | |
| 43 | 3426 | UGAGAAUCAUGCU | 355 | UAUUGCAGCAUGA | 375 | 93 | 59 |
| | | GCAAUAUG | | UUCUCAUG | | | |
| 44 | 1709 | GGUUCUCUGUCAU | 356 | UCGGCGAUGACAG | 376 | 93 | 76 |
| | | CGCCGAGG | | AGAACCAG | | | |
| 45 | 3484 | AUUUCAAGAUGAA | 357 | UUUAGUUUCAUCU | 378 | 93 | 59 |
| | | ACUAAAGC | | UGAAAUAU | | | |
| 46 | 2457 | GGUAUUGACGUCU | 358 | UCUGACAGACGUC | 379 | 93 | 69 |
| | | GUCAGAUC | | AAUACCUG | | | |
| 47 | 4127 | CGACCCAAACCAC | 359 | UUUGGUGUGGUUU | 380 | 92.9 | 51.9 |
| | | ACCAAAUG | | GGGUCGGA | | | |
| 48 | 3446 | GAUCAUUCUAGAG | 360 | UAUUUGCUCUAGA | 381 | 92.8 | 58.8 |
| | | CAAAUAUA | | AUGAUCAU | | | |
| 49 | 1294 | GCUAAAGUCCCAA | 361 | UUCCGUUUGGGAC | 382 | 92.8 | 80.8 |
| | | ACGGAAGU | | UUUAGCUU | | | |
| 50 | 4541 | UCAGGUGUUUGCA | 362 | UUUGCUUGCAAAC | 383 | 92.7 | 47.7 |
| | | AGCAAAGC | | ACCUGAAG | | | |

and SLC45A4 (Variant 3) Isoform 3

| | | | SEQ | | SEQ | | |
|---|---|---|---|---|---|---|---|
| siRNA_ | | | ID | | ID | | Corrected_ |
| id | Position | SS Sequence | NO: | AS Sequence | NO: | Score | Score |
| 1 | 2637 | GGUUUGAGCUACU | 384 | UUGCUUAGUAGCU | 434 | 106.5 | 80.5 |
| | | AAGCAAAU | | CAAACCUA | | | |
| 2 | 1892 | AGAAGUACUUGGA | 385 | UAGUUGUCCAAGU | 435 | 99.2 | 81.2 |
| | | CAACUACG | | ACUUCUGU | | | |
| 3 | 2633 | GGUAGGUUUGAGC | 386 | UUAGUAGCUCAAA | 436 | 98.3 | 72.3 |
| | | UACUAAGC | | CCUACCCU | | | |
| 4 | 2632 | GGGUAGGUUUGAG | 387 | UAGUAGCUCAAAC | 437 | 97.9 | 71.9 |
| | | CUACUAAG | | CUACCCUA | | | |
| 5 | 908 | GCAUCGACGAGGA | 388 | UACUGCUCCUCGU | 438 | 97 | 88 |
| | | GCAGUACA | | CGAUGCUG | | | |
| 6 | 1275 | GGAUAAUCACUUG | 389 | UUCAUUCAAGUGA | 439 | 96.1 | 84.1 |
| | | AAUGAAGC | | UUAUCCAG | | | |
| 7 | 827 | GCUGGUUCCGGAC | 390 | UUCUGGGUCCGGA | 440 | 95.4 | 87.4 |
| | | CCAGAACC | | ACCAGCUG | | | |
| 8 | 1514 | GCAUGAGCGACCU | 391 | UCGUACAGGUCGC | 441 | 95.1 | 80.1 |
| | | GUACGACA | | UCAUGCUG | | | |
| 9 | 1424 | CCAGCACCUUCUC | 392 | UAGUAGGAGAAGG | 442 | 95 | 81 |
| | | CUACUACG | | UGCUGGAG | | | |
| 10 | 226 | CCUUGUUGAAGAG | 393 | UUUCUCCUCUUCA | 443 | 94.2 | 92.2 |
| | | GAGAAACA | | ACAAGGAG | | | |
| 11 | 2638 | GUUUGAGCUACUA | 394 | UUUGCUUAGUAGC | 444 | 93.5 | 67.5 |
| | | AGCAAAUA | | UCAAACCU | | | |
| 12 | 1430 | CCUUCUCCUACUA | 395 | UUGCCGUAGUAGG | 445 | 93.3 | 79.3 |
| | | CGGCAAGC | | AGAAGGUG | | | |
| 13 | 1877 | GUUCAGCCCUGUU | 396 | UUCUGUAACAGGG | 446 | 93.3 | 75.3 |
| | | ACAGAAGU | | CUGAACAA | | | |
| 14 | 289 | GCAGGGACAGCAU | 397 | UAGAUGAUGCUGU | 447 | 93.1 | 91.1 |
| | | CAUCUAUG | | CCCUGCCG | | | |
| 15 | 1709 | GGUUCUCUGUCAU | 398 | UCGGCGAUGACAG | 448 | 93 | 76 |
| | | CGCCGAGG | | AGAACCAG | | | |
| 16 | 1294 | GCUAAAGUCCCAA | 399 | UUCCGUUUGGGAC | 449 | 92.8 | 80.8 |
| | | ACGGAAGU | | UUUAGCUU | | | |
| 17 | 1261 | GACGAGACCUUGC | 400 | UAUCCAGCAAGGU | 450 | 92.4 | 80.4 |
| | | UGGAUAAU | | CUCGUCCU | | | |
| 18 | 2648 | CUAAGCAAAUACC | 401 | UAGUGUGGUAUUU | 451 | 92.3 | 66.3 |
| | | ACACUAAC | | GCUUAGUA | | | |
| 19 | 1484 | ACGCCGUGGUGCU | 402 | UUGAUCAGCACCA | 452 | 91.6 | 77.6 |
| | | GAUCAAGC | | CGGCGUUG | | | |
| 20 | 1511 | GCAGCAUGAGCGA | 403 | UACAGGUCGCUCA | 453 | 91.5 | 76.5 |
| | | CCUGUACG | | UGCUGCGC | | | |
| 21 | 1757 | AGGUCAUCUUCGA | 404 | UCGCCUUCGAAGA | 454 | 91.1 | 74.1 |
| | | AGGCGACC | | UGACCUGG | | | |
| 22 | 1004 | CAGACGAGGUACA | 405 | UCCGACUGUACCU | 455 | 91 | 81 |
| | | GUCGGAGC | | CGUCUGGG | | | |
| 23 | 1880 | CAGCCCUGUUACA | 406 | UACUUCUGUAACA | 456 | 90.9 | 72.9 |
| | | GAAGUACU | | GGGCUGAA | | | |
| 24 | 403 | GAGCAGUACUACA | 407 | UGAGGCUGUAGUA | 457 | 90.9 | 86.9 |
| | | GCCUCACC | | CUGCUCCG | | | |
| 25 | 1272 | GCUGGAUAAUCAC | 408 | AUUCAAGUGAUUA | 458 | 90.4 | 78.4 |
| | | UUGAAUGA | | UCCAGCAA | | | |
| 26 | 1523 | ACCUGUACGACAU | 409 | UUCUGCAUGUCGU | 459 | 90.3 | 75.3 |
| | | GCAGAAGC | | ACAGGUCG | | | |
| 27 | 1287 | GAAUGAAGCUAAA | 410 | UGGGACUUUAGCU | 460 | 90.2 | 78.2 |
| | | GUCCCAAA | | UCAUUCAA | | | |
| 28 | 355 | GUGAGUCCACUGA | 411 | UUGACCUCAGUGG | 461 | 89.7 | 86.7 |
| | | GGUCAAAU | | ACUCACAA | | | |
| 29 | 1281 | UCACUUGAAUGAA | 412 | UUUAGCUUCAUUC | 462 | 89.6 | 77.6 |
| | | GCUAAAGU | | AAGUGAUU | | | |
| 30 | 1265 | AGACCUUGCUGGA | 413 | UGAUUAUCCAGCA | 463 | 88.8 | 76.8 |
| | | UAAUCACU | | AGGUCUCG | | | |
| 31 | 2629 | GUAGGGUAGGUUU | 414 | UAGCUCAAACCUA | 464 | 88.7 | 62.7 |
| | | GAGCUACU | | CCCUACGA | | | |
| 32 | 1882 | GCCCUGUUACAGA | 415 | AGUACUUCUGUAA | 465 | 88.5 | 70.5 |
| | | AGUACUUG | | CAGGGCUG | | | |
| 33 | 1883 | CCCUGUUACAGAA | 416 | AAGUACUUCUGUA | 466 | 88.4 | 70.4 |
| | | GUACUUGG | | ACAGGGCU | | | |
| 34 | 1517 | UGAGCGACCUGUA | 417 | AUGUCGUACAGGU | 467 | 88.4 | 73.4 |
| | | CGACAUGC | | CGCUCAUG | | | |
| 35 | 1121 | AGCUGCUGUUCCU | 418 | UCGUGCAGGAACA | 468 | 88.2 | 77.2 |
| | | GCACGACA | | GCAGCUCG | | | |
| 36 | 1346 | ACACCAGGGUGGA | 419 | UUCGUGUCCACCC | 469 | 88.2 | 75.2 |
| | | CACGAAGC | | UGGUGUAG | | | |
| 37 | 340 | CGUCCAAAGACCC | 420 | UCACAAGGGUCUU | 470 | 87.9 | 84.9 |
| | | UUGUGAGU | | UGGACGGG | | | |
| 38 | 331 | GGGCUGUCCCGUC | 421 | UCUUUGGACGGGA | 471 | 87.8 | 84.8 |
| | | CAAAGACC | | CAGCCCUG | | | |
| 39 | 1454 | GGUCCCACUGCUA | 422 | UAGCGGUAGCAGU | 472 | 87.7 | 73.7 |
| | | CCGCUACC | | GGGACCCA | | | |
| 40 | 1732 | GUGUUCUACACCG | 423 | UGAAGUCGGUGUA | 473 | 87.6 | 70.6 |
| | | ACUUCAUG | | GAACACGG | | | |
| 41 | 902 | UGUUCAGCAUCGA | 424 | UCCUCGUCGAUGC | 474 | 87.6 | 78.6 |
| | | CGAGGAGC | | UGAACAGG | | | |
| 42 | 1733 | UGUUCUACACCGA | 425 | AUGAAGUCGGUGU | 475 | 87.4 | 70.4 |
| | | CUUCAUGG | | AGAACACG | | | |
| 43 | 375 | UCAAUGGAGUCUU | 426 | UUUCUGAAGACUC | 476 | 87 | 84 |
| | | CAGAAAGG | | CAUUGAUU | | | |
| 44 | 1448 | AGCUUGGGUCCCA | 427 | UAGCAGUGGGACC | 477 | 86.7 | 72.7 |
| | | CUGCUACC | | CAAGCUUG | | | |
| 45 | 1727 | AGGCCGUGUUCUA | 428 | UCGGUGUAGAACA | 478 | 86.5 | 69.5 |
| | | CACCGACU | | CGGCCUCG | | | |
| 46 | 1262 | ACGAGACCUUGCU | 429 | UUAUCCAGCAAGG | 479 | 86 | 74 |
| | | GGAUAAUC | | UCUCGUCC | | | |
| 47 | 1916 | UGAGCGUCAGGGU | 430 | UAGAUCACCCUGA | 480 | 86 | 67 |
| | | GAUCUACG | | CGCUCAGG | | | |
| 48 | 364 | CUGAGGUCAAAUC | 431 | UCCAUUGAUUUGA | 481 | 85.9 | 82.9 |
| | | AAUGGAGU | | CCUCAGUG | | | |
| 49 | 2535 | GGAGCAGAGACAC | 432 | UCCGCGGUGUCUC | 482 | 85.7 | 60.7 |
| | | CGCGGAAC | | UGCUCCCG | | | |
| 50 | 830 | GGUUCCGGACCCA | 433 | UGGUUCUGGGUCC | 483 | 85.5 | 77.5 |
| | | GAACCAGG | | GGAACCAG | | | |

34. A method of treating or preventing a liver cell degeneration in a subject in need thereof, comprising administering a composition or a vector or a plasmid comprising one or more of the nucleic acids of claim 26.


SEQUENCE LISTING (492 sequences)

SEQ ID NO: 1 - RNA, 97 nt, synthetic construct
  source 1..97: mol_type = other RNA
tgctgttgacagtgagcgaaaccaagaatctagtaatttatagtgaagccacagatgtataaattactagattcttggttctgcctactgcctcgga

SEQ ID NO: 2 - RNA, 97 nt, synthetic construct
  source 1..97: mol_type = other RNA
tgctgttgacagtgagcgcacagaagaagatcataaagaatagtgaagccacagatgtattctttatgatcttcttctgtttgcctactgcctcgga

SEQ ID NO: 3 - RNA, 97 nt, synthetic construct
  source 1..97: mol_type = other RNA
tgctgttgacagtgagcgccagaagaagatcataaagaaatagtgaagccacagatgtatttctttatgatcttcttctgttgcctactgcctcgga

SEQ ID NO: 4 - RNA, 97 nt, synthetic construct
  source 1..97: mol_type = other RNA
tgctgttgacagtgagcgacagatcataacaagaacacaatagtgaagccacagatgtattgtgttcttgttatgatctggtgcctactgcctcgga

SEQ ID NO: 5 - RNA, 97 nt, synthetic construct
  source 1..97: mol_type = other RNA
tgctgttgacagtgagcgaagagatgtggatacacaagaatagtgaagccacagatgtattcttgtgtatccacatctctctgcctactgcctcgga

SEQ ID NO: 6 - RNA, 97 nt, synthetic construct
  source 1..97: mol_type = other RNA
tgctgttgacagtgagcgcaaagagttatgtgaattacaatagtgaagccacagatgtattgtaattcacataactctttttgcctactgcctcgga

SEQ ID NO: 7 - RNA, 97 nt, synthetic construct
  source 1..97: mol_type = other RNA
tgctgttgacagtgagcgcacagaagtacttggacaactatagtgaagccacagatgtatagttgtccaagtacttctgtatgcctactgcctcgga

SEQ ID NO: 8 - RNA, 97 nt, synthetic construct
  source 1..97: mol_type = other RNA
tgctgttgacagtgagcgacacaatgtggttctcttagaatagtgaagccacagatgtattctaagagaaccacattgtggtgcctactgcctcgga

SEQ ID NO: 9 - RNA, 97 nt, synthetic construct
  source 1..97: mol_type = other RNA
tgctgttgacagtgagcgccgggcaagtactgcagctatttagtgaagccacagatgtaaatagctgcagtacttgcccgatgcctactgcctcgga

SEQ ID NO: 10 - RNA, 97 nt, synthetic construct
  source 1..97: mol_type = other RNA
tgctgttgacagtgagcgcaccacagtctctttagaactatagtgaagccacagatgtatagttctaaagagactgtggtttgcctactgcctcgga

SEQ ID NO: 11 - RNA, 97 nt, synthetic construct
  source 1..97: mol_type = other RNA
tgctgttgacagtgagcgacccgcacatcacacagataaatagtgaagccacagatgtatttatctgtgtgatgtgcggggtgcctactgcctcgga

SEQ ID NO: 12 - RNA, 97 nt, synthetic construct
  source 1..97: mol_type = other RNA
tgctgttgacagtgagcgccacacagataaagtcagacaatagtgaagccacagatgtattgtctgactttatctgtgtgatgcctactgcctcgga

SEQ ID NO: 13 - RNA, 21 nt, synthetic construct
  misc_feature 1..21: note = Synthetic sequence
  source 1..21: mol_type = other RNA
gctagtagtttacattaaatt

SEQ ID NO: 14 - RNA, 21 nt, synthetic construct
  misc_feature 1..21: note = Synthetic sequence
  source 1..21: mol_type = other RNA
ccaagaatctagtaatttatc

SEQ ID NO: 15 - RNA, 21 nt, synthetic construct
  misc_feature 1..21: note = Synthetic sequence
  source 1..21: mol_type = other RNA
agagttatgtgaattacaagg

SEQ ID NO: 16 - RNA, 21 nt, synthetic construct
  misc_feature 1..21: note = Synthetic sequence
  source 1..21: mol_type = other RNA
gaacagaagaagatcataaag

SEQ ID NO: 17 - RNA, 21 nt, synthetic construct
  misc_feature 1..21: note = Synthetic sequence
  source 1..21: mol_type = other RNA
ggtttggtatcacgggttatg

SEQ ID NO: 18 - RNA, 21 nt, synthetic construct
  misc_feature 1..21: note = Synthetic sequence
  source 1..21: mol_type = other RNA
ctagatagatcgttcttaaca

SEQ ID NO: 19 - RNA, 21 nt, synthetic construct
  misc_feature 1..21: note = Synthetic sequence
  source 1..21: mol_type = other RNA
cgactttggaggtacagaagg

SEQ ID NO: 20 - RNA, 21 nt, synthetic construct
  misc_feature 1..21: note = Synthetic sequence
  source 1..21: mol_type = other RNA
ggtgcatttgcttgtaataca

SEQ ID NO: 21 - RNA, 21 nt, synthetic construct
  misc_feature 1..21: note = Synthetic sequence
  source 1..21: mol_type = other RNA
tgctagtagtttacattaaat

SEQ ID NO: 22 - RNA, 21 nt, synthetic construct
  misc_feature 1..21: note = Synthetic sequence
  source 1..21: mol_type = other RNA
tgtaaatgctagtagtttaca

SEQ ID NO: 23 - RNA, 21 nt, synthetic construct
  misc_feature 1..21: note = Synthetic sequence
  source 1..21: mol_type = other RNA
ggagagagatgtggatacaca

SEQ ID NO: 24 - RNA, 21 nt, synthetic construct
  misc_feature 1..21: note = Synthetic sequence
  source 1..21: mol_type = other RNA
ggaacaagtagaagtggtaga

SEQ ID NO: 25 - RNA, 21 nt, synthetic construct
  misc_feature 1..21: note = Synthetic sequence
  source 1..21: mol_type = other RNA
ctgtgcgactctagatagatc

SEQ ID NO: 26 - RNA, 21 nt, synthetic construct
  misc_feature 1..21: note = Synthetic sequence
  source 1..21: mol_type = other RNA
agtgcagcatgaatgtgaagt

SEQ ID NO: 27 - RNA, 21 nt, synthetic construct
  misc_feature 1..21: note = Synthetic sequence
  source 1..21: mol_type = other RNA
ggtgaaacagaacagaagaag

SEQ ID NO: 28 - RNA, 21 nt, synthetic construct
  misc_feature 1..21: note = Synthetic sequence
  source 1..21: mol_type = other RNA
cctcttccctaaagaacaata

SEQ ID NO: 29 - RNA, 21 nt, synthetic construct
  misc_feature 1..21: note = Synthetic sequence
  source 1..21: mol_type = other RNA
ggaggtacagaaggtgaaaca

SEQ ID NO: 30 - RNA, 21 nt, synthetic construct
  misc_feature 1..21: note = Synthetic sequence
  source 1..21: mol_type = other RNA
cttcataagaagttatataaa

SEQ ID NO: 31 - RNA, 21 nt, synthetic construct
  misc_feature 1..21: note = Synthetic sequence
  source 1..21: mol_type = other RNA
agaacagaagaagatcataaa

SEQ ID NO: 32 - RNA, 21 nt, synthetic construct
  misc_feature 1..21: note = Synthetic sequence
  source 1..21: mol_type = other RNA
ctacctggttctctcataaga

SEQ ID NO: 33 - RNA, 21 nt, synthetic construct
  misc_feature 1..21: note = Synthetic sequence
  source 1..21: mol_type = other RNA
cctacctggttctctcataag

SEQ ID NO: 34 - RNA, 21 nt, synthetic construct
  misc_feature 1..21: note = Synthetic sequence
  source 1..21: mol_type = other RNA
gaacaatagggaacaagtaga

SEQ ID NO: 35 - RNA, 21 nt, synthetic construct
  misc_feature 1..21: note = Synthetic sequence
  source 1..21: mol_type = other RNA
caagaatctagtaatttatca

SEQ ID NO: 36 - RNA, 21 nt, synthetic construct
  misc_feature 1..21: note = Synthetic sequence
  source 1..21: mol_type = other RNA
gtagaatttcacagcaataaa

SEQ ID NO: 37 - RNA, 21 nt, synthetic construct
  misc_feature 1..21: note = Synthetic sequence
  source 1..21: mol_type = other RNA
aatgaagtactttgtcaaata

SEQ ID NO: 38 - RNA, 21 nt, synthetic construct
  misc_feature 1..21: note = Synthetic sequence
  source 1..21: mol_type = other RNA
caatagggaacaagtagaagt

SEQ ID NO: 39 - RNA, 21 nt, synthetic construct
  misc_feature 1..21: note = Synthetic sequence
  source 1..21: mol_type = other RNA
tgaagtactttgtcaaataaa

SEQ ID NO: 40 - RNA, 21 nt, synthetic construct
  misc_feature 1..21: note = Synthetic sequence
  source 1..21: mol_type = other RNA
aaatgaagtactttgtcaaat

SEQ ID NO: 41 - RNA, 21 nt, synthetic construct
  misc_feature 1..21: note = Synthetic sequence
  source 1..21: mol_type = other RNA
atgttatgatatcatgaaaga

SEQ ID NO: 42 - RNA, 21 nt, synthetic construct
  misc_feature 1..21: note = Synthetic sequence
  source 1..21: mol_type = other RNA
gaaccaagaatctagtaattt

SEQ ID NO: 43 - RNA, 21 nt, synthetic construct
  misc_feature 1..21: note = Synthetic sequence
  source 1..21: mol_type = other RNA
cctaaagaacaatagggaaca

SEQ ID NO: 44 - RNA, 21 nt, synthetic construct
  misc_feature 1..21: note = Synthetic sequence
  source 1..21: mol_type = other RNA
gtttcatgttcactggtattt

SEQ ID NO: 45 - RNA, 21 nt, synthetic construct
  misc_feature 1..21: note = Synthetic sequence
  source 1..21: mol_type = other RNA
gcgcttcgagcttcttcaagg

SEQ ID NO: 46 - RNA, 21 nt, synthetic construct
  misc_feature 1..21: note = Synthetic sequence
  source 1..21: mol_type = other RNA
cgaggatgggaacaactgagt

SEQ ID NO: 47 - RNA, 21 nt, synthetic construct
  misc_feature 1..21: note = Synthetic sequence
  source 1..21: mol_type = other RNA
atgatttaatgttatgatatc

SEQ ID NO: 48 - RNA, 21 nt, synthetic construct
  misc_feature 1..21: note = Synthetic sequence
  source 1..21: mol_type = other RNA
cataagaagttatataaataa

SEQ ID NO: 49 - RNA, 21 nt, synthetic construct
  misc_feature 1..21: note = Synthetic sequence
  source 1..21: mol_type = other RNA
agtagaagtggtagaatttca

SEQ ID NO: 50 - RNA, 21 nt, synthetic construct
  misc_feature 1..21: note = Synthetic sequence
  source 1..21: mol_type = other RNA
gttcctccctcttccctaaag

SEQ ID NO: 51 - RNA, 21 nt, synthetic construct
  misc_feature 1..21: note = Synthetic sequence
  source 1..21: mol_type = other RNA
aggacaggaggacaggaaatc

SEQ ID NO: 52 - RNA, 21 nt, synthetic construct
  misc_feature 1..21: note = Synthetic sequence
  source 1..21: mol_type = other RNA
gaccctgtgcgactctagata

SEQ ID NO: 53 - RNA, 21 nt, synthetic construct
  misc_feature 1..21: note = Synthetic sequence
  source 1..21: mol_type = other RNA
ggtagaatttcacagcaataa

SEQ ID NO: 54 - RNA, 21 nt, synthetic construct
  misc_feature 1..21: note = Synthetic sequence
  source 1..21: mol_type = other RNA
aacagaagaagatcataaaga

SEQ ID NO: 55 - RNA, 21 nt, synthetic construct
  misc_feature 1..21: note = Synthetic sequence
  source 1..21: mol_type = other RNA
tctacttcataagaagttata

SEQ ID NO: 56 - RNA, 21 nt, synthetic construct
  misc_feature 1..21: note = Synthetic sequence
  source 1..21: mol_type = other RNA
ccctgtgcgactctagataga

SEQ ID NO: 57 - RNA, 21 nt, synthetic construct
  misc_feature 1..21: note = Synthetic sequence
  source 1..21: mol_type = other RNA
gaagtgcaccggtttggtatc

SEQ ID NO: 58 - RNA, 21 nt, synthetic construct
  misc_feature 1..21: note = Synthetic sequence
  source 1..21: mol_type = other RNA
ataagaagttatataaataaa

SEQ ID NO: 59 - RNA, 21 nt, synthetic construct
  misc_feature 1..21: note = Synthetic sequence
  source 1..21: mol_type = other RNA
aagagttatgtgaattacaag

SEQ ID NO: 60 - RNA, 21 nt, synthetic construct
  misc_feature 1..21: note = Synthetic sequence
  source 1..21: mol_type = other RNA
ggacaggaggacaggaaatcc

SEQ ID NO: 61 - RNA, 21 nt, synthetic construct
  misc_feature 1..21: note = Synthetic sequence
  source 1..21: mol_type = other RNA
agagatccttgctgctgcagt

SEQ ID NO: 62 - RNA, 21 nt, synthetic construct
  misc_feature 1..21: note = Synthetic sequence
  source 1..21: mol_type = other RNA
atgctagtagtttacattaaa

SEQ ID NO: 63 - RNA, 21 nt, synthetic construct
  misc_feature 1..21: note = Synthetic sequence
  source 1..21: mol_type = other RNA
tttaatgtaaactactagcat

SEQ ID NO: 64 - RNA, 21 nt, synthetic construct
  misc_feature 1..21: note = Synthetic sequence
  source 1..21: mol_type = other RNA
taaattactagattcttggtt

SEQ ID NO: 65 - RNA, 21 nt, synthetic construct
  misc_feature 1..21: note = Synthetic sequence
  source 1..21: mol_type = other RNA
ttgtaattcacataactcttt

SEQ ID NO: 66 - RNA, 21 nt, synthetic construct
  misc_feature 1..21: note = Synthetic sequence
  source 1..21: mol_type = other RNA
ttatgatcttcttctgttctg

SEQ ID NO: 67 - RNA, 21 nt, synthetic construct
  misc_feature 1..21: note = Synthetic sequence
  source 1..21: mol_type = other RNA
taacccgtgataccaaaccgg

SEQ ID NO: 68 - RNA, 21 nt, synthetic construct
  misc_feature 1..21: note = Synthetic sequence
  source 1..21: mol_type = other RNA
ttaagaacgatctatctagag

SEQ ID NO: 69 - RNA, 21 nt, synthetic construct
  misc_feature 1..21: note = Synthetic sequence
  source 1..21: mol_type = other RNA
ttctgtacctccaaagtcgta

SEQ ID NO: 70 - RNA, 21 nt, synthetic construct
  misc_feature 1..21: note = Synthetic sequence
  source 1..21: mol_type = other RNA
tattacaagcaaatgcaccaa

SEQ ID NO: 71 - RNA, 21 nt, synthetic construct
  misc_feature 1..21: note = Synthetic sequence
  source 1..21: mol_type = other RNA
ttaatgtaaactactagcatt

SEQ ID NO: 72 - RNA, 21 nt, synthetic construct
  misc_feature 1..21: note = Synthetic sequence
  source 1..21: mol_type = other RNA
taaactactagcatttacaaa

SEQ ID NO: 73 - RNA, 21 nt, synthetic construct
  misc_feature 1..21: note = Synthetic sequence
  source 1..21: mol_type = other RNA
tgtatccacatctctctccaa

SEQ ID NO: 74 - RNA, 21 nt, synthetic construct
  misc_feature 1..21: note = Synthetic sequence
  source 1..21: mol_type = other RNA
taccacttctacttgttccct

SEQ ID NO: 75 - RNA, 21 nt, synthetic construct
  misc_feature 1..21: note = Synthetic sequence
  source 1..21: mol_type = other RNA
tctatctagagtcgcacaggg

SEQ ID NO: 76 - RNA, 21 nt, synthetic construct
  misc_feature 1..21: note = Synthetic sequence
  source 1..21: mol_type = other RNA
ttcacattcatgctgcactgg

SEQ ID NO: 77 - RNA, 21 nt, synthetic construct
  misc_feature 1..21: note = Synthetic sequence
  source 1..21: mol_type = other RNA
tcttctgttctgtttcacctt

SEQ ID NO: 78 - RNA, 21 nt, synthetic construct
  misc_feature 1..21: note = Synthetic sequence
  source 1..21: mol_type = other RNA
ttgttctttagggaagaggga

SEQ ID NO: 79 - RNA, 21 nt, synthetic construct
  misc_feature 1..21: note = Synthetic sequence
  source 1..21: mol_type = other RNA
tttcaccttctgtacctccaa

SEQ ID NO: 80 - RNA, 21 nt, synthetic construct
  misc_feature 1..21: note = Synthetic sequence
  source 1..21: mol_type = other RNA
tatataacttcttatgaagta

SEQ ID NO: 81 - RNA, 21 nt, synthetic construct
  misc_feature 1..21: note = Synthetic sequence
  source 1..21: mol_type = other RNA
tatgatcttcttctgttctgt

SEQ ID NO: 82 - RNA, 21 nt, synthetic construct
  misc_feature 1..21: note = Synthetic sequence
  source 1..21: mol_type = other RNA
ttatgagagaaccaggtaggg

SEQ ID NO: 83 - RNA, 21 nt, synthetic construct
  misc_feature 1..21: note = Synthetic sequence
  source 1..21: mol_type = other RNA
tatgagagaaccaggtagggg

SEQ ID NO: 84 - RNA, 21 nt, synthetic construct
  misc_feature 1..21: note = Synthetic sequence
  source 1..21: mol_type = other RNA
tacttgttccctattgttctt

SEQ ID NO: 85 - RNA, 21 nt, synthetic construct
  misc_feature 1..21: note = Synthetic sequence
  source 1..21: mol_type = other RNA
ataaattactagattcttggt

SEQ ID NO: 86 - RNA, 21 nt, synthetic construct
  misc_feature 1..21: note = Synthetic sequence
  source 1..21: mol_type = other RNA
tattgctgtgaaattctacca

SEQ ID NO: 87 - RNA, 21 nt, synthetic construct
  misc_feature 1..21: note = Synthetic sequence
  source 1..21: mol_type = other RNA
tttgacaaagtacttcatttg

SEQ ID NO: 88 - RNA, 21 nt, synthetic construct
  misc_feature 1..21: note = Synthetic sequence
  source 1..21: mol_type = other RNA
ttctacttgttccctattgtt

SEQ ID NO: 89 - RNA, 21 nt, synthetic construct
  misc_feature 1..21: note = Synthetic sequence
  source 1..21: mol_type = other RNA
tatttgacaaagtacttcatt

SEQ ID NO: 90 - RNA, 21 nt, synthetic construct
  misc_feature 1..21: note = Synthetic sequence
  source 1..21: mol_type = other RNA
ttgacaaagtacttcatttgg

SEQ ID NO: 91 - RNA, 21 nt, synthetic construct
  misc_feature 1..21: note = Synthetic sequence
  source 1..21: mol_type = other RNA
tttcatgatatcataacatta

SEQ ID NO: 92 - RNA, 21 nt, synthetic construct
  misc_feature 1..21: note = Synthetic sequence
  source 1..21: mol_type = other RNA
attactagattcttggttctt

SEQ ID NO: 93 - RNA, 21 nt, synthetic construct
  misc_feature 1..21: note = Synthetic sequence
  source 1..21: mol_type = other RNA
ttccctattgttctttaggga

SEQ ID NO: 94 - RNA, 21 nt, synthetic construct
  misc_feature 1..21: note = Synthetic sequence
  source 1..21: mol_type = other RNA
ataccagtgaacatgaaacag

SEQ ID NO: 95 - RNA, 21 nt, synthetic construct
  misc_feature 1..21: note = Synthetic sequence
  source 1..21: mol_type = other RNA
ttgaagaagctcgaagcgctc

SEQ ID NO: 96 - RNA, 21 nt, synthetic construct
  misc_feature 1..21: note = Synthetic sequence
  source 1..21: mol_type = other RNA
tcagttgttcccatcctcgtt

SEQ ID NO: 97 - RNA, 21 nt, synthetic construct
  misc_feature 1..21: note = Synthetic sequence
  source 1..21: mol_type = other RNA
tatcataacattaaatcatta

SEQ ID NO: 98 - RNA, 21 nt, synthetic construct
  misc_feature 1..21: note = Synthetic sequence
  source 1..21: mol_type = other RNA
atttatataacttcttatgaa

SEQ ID NO: 99 - RNA, 21 nt, synthetic construct
  misc_feature 1..21: note = Synthetic sequence
  source 1..21: mol_type = other RNA
aaattctaccacttctacttg

SEQ ID NO: 100 - RNA, 21 nt, synthetic construct
  misc_feature 1..21: note = Synthetic sequence
  source 1..21: mol_type = other RNA
ttagggaagagggaggaactg

SEQ ID NO: 101 - RNA, 21 nt, synthetic construct
  misc_feature 1..21: note = Synthetic sequence
  source 1..21: mol_type = other RNA
tttcctgtcctcctgtccttt

SEQ ID NO: 102 - RNA, 21 nt, synthetic construct
  misc_feature 1..21: note = Synthetic sequence
  source 1..21: mol_type = other RNA
tctagagtcgcacagggtcca

SEQ ID NO: 103 - RNA, 21 nt, synthetic construct
  misc_feature 1..21: note = Synthetic sequence
  source 1..21: mol_type = other RNA
attgctgtgaaattctaccac

SEQ ID NO: 104 - RNA, 21 nt, synthetic construct
  misc_feature 1..21: note = Synthetic sequence
  source 1..21: mol_type = other RNA
tttatgatcttcttctgttct

SEQ ID NO: 105 - RNA, 21 nt, synthetic construct
  misc_feature 1..21: note = Synthetic sequence
  source 1..21: mol_type = other RNA
taacttcttatgaagtagatt

SEQ ID NO: 106 - RNA, 21 nt, synthetic construct
  misc_feature 1..21: note = Synthetic sequence
  source 1..21: mol_type = other RNA
tatctagagtcgcacagggtc

SEQ ID NO: 107 - RNA, 21 nt, synthetic construct
  misc_feature 1..21: note = Synthetic sequence
  source 1..21: mol_type = other RNA
taccaaaccggtgcacttcta

SEQ ID NO: 108 - RNA, 21 nt, synthetic construct
  misc_feature 1..21: note = Synthetic sequence
  source 1..21: mol_type = other RNA
tatttatataacttcttatga

SEQ ID NO: 109 - RNA, 21 nt, synthetic construct
  misc_feature 1..21: note = Synthetic sequence
  source 1..21: mol_type = other RNA
tgtaattcacataactctttt

SEQ ID NO: 110 - RNA, 21 nt, synthetic construct
  misc_feature 1..21: note = Synthetic sequence
  source 1..21: mol_type = other RNA
atttcctgtcctcctgtcctt

SEQ ID NO: 111 - RNA, 21 nt, synthetic construct
  misc_feature 1..21: note = Synthetic sequence
  source 1..21: mol_type = other RNA
tgcagcagcaaggatctctgg

SEQ ID NO: 112 - RNA, 21 nt, synthetic construct
  misc_feature 1..21: note = Synthetic sequence
  source 1..21: mol_type = other RNA
taatgtaaactactagcattt

SEQ ID NO: 113 - RNA, 21 nt, synthetic construct
  misc_feature 1..21: note = Synthetic sequence
  source 1..21: mol_type = other RNA
gctagtagtttacattaaatt

SEQ ID NO: 114 - RNA, 21 nt, synthetic construct
  misc_feature 1..21: note = Synthetic sequence
  source 1..21: mol_type = other RNA
ccaagaatctagtaatttatc

SEQ ID NO: 115 - RNA, 21 nt, synthetic construct
  misc_feature 1..21: note = Synthetic sequence
  source 1..21: mol_type = other RNA
agagttatgtgaattacaagg

SEQ ID NO: 116 - RNA, 21 nt, synthetic construct
  misc_feature 1..21: note = Synthetic sequence
  source 1..21: mol_type = other RNA
gaacagaagaagatcataaag

SEQ ID NO: 117 - RNA, 21 nt, synthetic construct
  misc_feature 1..21: note = Synthetic sequence
  source 1..21: mol_type = other RNA
ggtttggtatcacgggttatg

SEQ ID NO: 118 - RNA, 21 nt, synthetic construct
  misc_feature 1..21: note = Synthetic sequence
  source 1..21: mol_type = other RNA
ctagatagatcgttcttaaca

SEQ ID NO: 119 - RNA, 21 nt, synthetic construct
  misc_feature 1..21: note = Synthetic sequence
  source 1..21: mol_type = other RNA
cgactttggaggtacagaagg

SEQ ID NO: 120 - RNA, 21 nt, synthetic construct
  misc_feature 1..21: note = Synthetic sequence
  source 1..21: mol_type = other RNA
ggtgcatttgcttgtaataca

SEQ ID NO: 121 - RNA, 21 nt, synthetic construct
  misc_feature 1..21: note = Synthetic sequence
  source 1..21: mol_type = other RNA
tgctagtagtttacattaaat

SEQ ID NO: 122 - RNA, 21 nt, synthetic construct
  misc_feature 1..21: note = Synthetic sequence
  source 1..21: mol_type = other RNA
tgtaaatgctagtagtttaca

SEQ ID NO: 123 - RNA, 21 nt, synthetic construct
  misc_feature 1..21: note = Synthetic sequence
  source 1..21: mol_type = other RNA
ggagagagatgtggatacaca

SEQ ID NO: 124 - RNA, 21 nt, synthetic construct
  misc_feature 1..21: note = Synthetic sequence
  source 1..21: mol_type = other RNA
ggaacaagtagaagtggtaga

SEQ ID NO: 125 - RNA, 21 nt, synthetic construct
  misc_feature 1..21: note = Synthetic sequence
  source 1..21: mol_type = other RNA
ctgtgcgactctagatagatc

SEQ ID NO: 126 - RNA, 21 nt, synthetic construct
  misc_feature 1..21: note = Synthetic sequence
  source 1..21: mol_type = other RNA
agtgcagcatgaatgtgaagt

SEQ ID NO: 127 - RNA, 21 nt, synthetic construct
  misc_feature 1..21: note = Synthetic sequence
  source 1..21: mol_type = other RNA
ggtgaaacagaacagaagaag

SEQ ID NO: 128 - RNA, 21 nt, synthetic construct
  misc_feature 1..21: note = Synthetic sequence
  source 1..21: mol_type = other RNA
cctcttccctaaagaacaata

SEQ ID NO: 129 - RNA, 21 nt, synthetic construct
  misc_feature 1..21: note = Synthetic sequence
  source 1..21: mol_type = other RNA
ggaggtacagaaggtgaaaca

SEQ ID NO: 130 - RNA, 21 nt, synthetic construct
  misc_feature 1..21: note = Synthetic sequence
  source 1..21: mol_type = other RNA
cttcataagaagttatataaa

SEQ ID NO: 131 - RNA, 21 nt, synthetic construct
  misc_feature 1..21: note = Synthetic sequence
  source 1..21: mol_type = other RNA
agaacagaagaagatcataaa

SEQ ID NO: 132 - RNA, 21 nt, synthetic construct
  misc_feature 1..21: note = Synthetic sequence
  source 1..21: mol_type = other RNA
ctacctggttctctcataaga

SEQ ID NO: 133 - RNA, 21 nt, synthetic construct
  misc_feature 1..21: note = Synthetic sequence
  source 1..21: mol_type = other RNA
cctacctggttctctcataag

SEQ ID NO: 134 - RNA, 21 nt, synthetic construct
  misc_feature 1..21: note = Synthetic sequence
  source 1..21: mol_type = other RNA
gaacaatagggaacaagtaga

SEQ ID NO: 135 - RNA, 21 nt, synthetic construct
  misc_feature 1..21: note = Synthetic sequence
  source 1..21: mol_type = other RNA
caagaatctagtaatttatca

SEQ ID NO: 136 - RNA, 21 nt, synthetic construct
  misc_feature 1..21: note = Synthetic sequence
  source 1..21: mol_type = other RNA
gtagaatttcacagcaataaa

SEQ ID NO: 137 - RNA, 21 nt, synthetic construct
  misc_feature 1..21: note = Synthetic sequence
  source 1..21: mol_type = other RNA
aatgaagtactttgtcaaata

SEQ ID NO: 138 - RNA, 21 nt, synthetic construct
  misc_feature 1..21: note = Synthetic sequence
  source 1..21: mol_type = other RNA
caatagggaacaagtagaagt

SEQ ID NO: 139 - RNA, 21 nt, synthetic construct
  misc_feature 1..21: note = Synthetic sequence
  source 1..21: mol_type = other RNA
tgaagtactttgtcaaataaa

SEQ ID NO: 140 - RNA, 21 nt, synthetic construct
  misc_feature 1..21: note = Synthetic sequence
  source 1..21: mol_type = other RNA
aaatgaagtactttgtcaaat

SEQ ID NO: 141 - RNA, 21 nt, synthetic construct
  misc_feature 1..21: note = Synthetic sequence
  source 1..21: mol_type = other RNA
atgttatgatatcatgaaaga

SEQ ID NO: 142 - RNA, 21 nt, synthetic construct
  misc_feature 1..21: note = Synthetic sequence
  source 1..21: mol_type = other RNA
gaaccaagaatctagtaattt

SEQ ID NO: 143 - RNA, 21 nt, synthetic construct
  misc_feature 1..21: note = Synthetic sequence
  source 1..21: mol_type = other RNA
cctaaagaacaatagggaaca

SEQ ID NO: 144 - RNA, 21 nt, synthetic construct
  misc_feature 1..21: note = Synthetic sequence
  source 1..21: mol_type = other RNA
gtttcatgttcactggtattt

SEQ ID NO: 145 - RNA, 21 nt, synthetic construct
  misc_feature 1..21: note = Synthetic sequence
  source 1..21: mol_type = other RNA
gcgcttcgagcttcttcaagg

SEQ ID NO: 146 - RNA, 21 nt, synthetic construct
  misc_feature 1..21: note = Synthetic sequence
  source 1..21: mol_type = other RNA
cgaggatgggaacaactgagt

SEQ ID NO: 147 - RNA, 21 nt, synthetic construct
  misc_feature 1..21: note = Synthetic sequence
  source 1..21: mol_type = other RNA
atgatttaatgttatgatatc

SEQ ID NO: 148 - RNA, 21 nt, synthetic construct
  misc_feature 1..21: note = Synthetic sequence
  source 1..21: mol_type = other RNA
cataagaagttatataaataa

SEQ ID NO: 149 - RNA, 21 nt, synthetic construct
  misc_feature 1..21: note = Synthetic sequence
  source 1..21: mol_type = other RNA
agtagaagtggtagaatttca

SEQ ID NO: 150 - RNA, 21 nt, synthetic construct
  misc_feature 1..21: note = Synthetic sequence
  source 1..21: mol_type = other RNA
gttcctccctcttccctaaag

SEQ ID NO: 151 - RNA, 21 nt, synthetic construct
  misc_feature 1..21: note = Synthetic sequence
  source 1..21: mol_type = other RNA
aggacaggaggacaggaaatc

SEQ ID NO: 152 - RNA, 21 nt, synthetic construct
  misc_feature 1..21: note = Synthetic sequence
  source 1..21: mol_type = other RNA
gaccctgtgcgactctagata

SEQ ID NO: 153 - RNA, 21 nt, synthetic construct
  misc_feature 1..21: note = Synthetic sequence
  source 1..21: mol_type = other RNA
ggtagaatttcacagcaataa

SEQ ID NO: 154 - RNA, 21 nt, synthetic construct
  misc_feature 1..21: note = Synthetic sequence
  source 1..21: mol_type = other RNA
aacagaagaagatcataaaga

SEQ ID NO: 155 - RNA, 21 nt, synthetic construct
  misc_feature 1..21: note = Synthetic sequence
  source 1..21: mol_type = other RNA
tctacttcataagaagttata

SEQ ID NO: 156 - RNA, 21 nt, synthetic construct
  misc_feature 1..21: note = Synthetic sequence
  source 1..21: mol_type = other RNA
ccctgtgcgactctagataga

SEQ ID NO: 157 - RNA, 21 nt, synthetic construct
  misc_feature 1..21: note = Synthetic sequence
  source 1..21: mol_type = other RNA
gaagtgcaccggtttggtatc

SEQ ID NO: 158 - RNA, 21 nt, synthetic construct
  misc_feature 1..21: note = Synthetic sequence
  source 1..21: mol_type = other RNA
ataagaagttatataaataaa

SEQ ID NO: 159 - RNA, 21 nt, synthetic construct
  misc_feature 1..21: note = Synthetic sequence
  source 1..21: mol_type = other RNA
aagagttatgtgaattacaag

SEQ ID NO: 160 - RNA, 21 nt, synthetic construct
  misc_feature 1..21: note = Synthetic sequence
  source 1..21: mol_type = other RNA
ggacaggaggacaggaaatcc

SEQ ID NO: 161 - RNA, 21 nt, synthetic construct
  misc_feature 1..21: note = Synthetic sequence
  source 1..21: mol_type = other RNA
agagatccttgctgctgcagt

SEQ ID NO: 162 - RNA, 21 nt, synthetic construct
  misc_feature 1..21: note = Synthetic sequence
  source 1..21: mol_type = other RNA
atgctagtagtttacattaaa

SEQ ID NO: 163 - RNA, 21 nt, synthetic construct
  misc_feature 1..21: note = Synthetic sequence
  source 1..21: mol_type = other RNA
tttaatgtaaactactagcat

SEQ ID NO: 164 - RNA, 21 nt, synthetic construct
  misc_feature 1..21: note = Synthetic sequence
  source 1..21: mol_type = other RNA
taaattactagattcttggtt

SEQ ID NO: 165 - RNA, 21 nt, synthetic construct
  misc_feature 1..21: note = Synthetic sequence
  source 1..21: mol_type = other RNA
ttgtaattcacataactcttt

SEQ ID NO: 166 - RNA, 21 nt, synthetic construct
  misc_feature 1..21: note = Synthetic sequence
  source 1..21: mol_type = other RNA
ttatgatcttcttctgttctg

SEQ ID NO: 167 - RNA, 21 nt, synthetic construct
  misc_feature 1..21: note = Synthetic sequence
  source 1..21: mol_type = other RNA
taacccgtgataccaaaccgg

SEQ ID NO: 168 - RNA, 21 nt, synthetic construct
  misc_feature 1..21: note = Synthetic sequence
  source 1..21: mol_type = other RNA
ttaagaacgatctatctagag

SEQ ID NO: 169 - RNA, 21 nt, synthetic construct
  misc_feature 1..21: note = Synthetic sequence
  source 1..21: mol_type = other RNA
ttctgtacctccaaagtcgta

SEQ ID NO: 170 - RNA, 21 nt, synthetic construct
  misc_feature 1..21: note = Synthetic sequence
  source 1..21: mol_type = other RNA
tattacaagcaaatgcaccaa

SEQ ID NO: 171 - RNA, 21 nt, synthetic construct
  misc_feature 1..21: note = Synthetic sequence
  source 1..21: mol_type = other RNA
ttaatgtaaactactagcatt

SEQ ID NO: 172 - RNA, 21 nt, synthetic construct
  misc_feature 1..21: note = Synthetic sequence
  source 1..21: mol_type = other RNA
taaactactagcatttacaaa

SEQ ID NO: 173 - RNA, 21 nt, synthetic construct
  misc_feature 1..21: note = Synthetic sequence
  source 1..21: mol_type = other RNA
tgtatccacatctctctccaa

SEQ ID NO: 174 - RNA, 21 nt, synthetic construct
  misc_feature 1..21: note = Synthetic sequence
  source 1..21: mol_type = other RNA
taccacttctacttgttccct

SEQ ID NO: 175 - RNA, 21 nt, synthetic construct
  misc_feature 1..21: note = Synthetic sequence
  source 1..21: mol_type = other RNA
tctatctagagtcgcacaggg

SEQ ID NO: 176 - RNA, 21 nt, synthetic construct
  misc_feature 1..21: note = Synthetic sequence
  source 1..21: mol_type = other RNA
ttcacattcatgctgcactgg

SEQ ID NO: 177 - RNA, 21 nt, synthetic construct
  misc_feature 1..21: note = Synthetic sequence
  source 1..21: mol_type = other RNA
tcttctgttctgtttcacctt

SEQ ID NO: 178 - RNA, 21 nt, synthetic construct
  misc_feature 1..21: note = Synthetic sequence
  source 1..21: mol_type = other RNA
ttgttctttagggaagaggga

SEQ ID NO: 179 - RNA, 21 nt, synthetic construct
  misc_feature 1..21: note = Synthetic sequence
  source 1..21: mol_type = other RNA
tttcaccttctgtacctccaa

SEQ ID NO: 180 - RNA, 21 nt, synthetic construct
  misc_feature 1..21: note = Synthetic sequence
  source 1..21: mol_type = other RNA
tatataacttcttatgaagta

SEQ ID NO: 181 - RNA, 21 nt, synthetic construct
  misc_feature 1..21: note = Synthetic sequence
  source 1..21: mol_type = other RNA
tatgatcttcttctgttctgt

SEQ ID NO: 182 - RNA, 21 nt, synthetic construct
  misc_feature 1..21: note = Synthetic sequence
  source 1..21: mol_type = other RNA
ttatgagagaaccaggtaggg

SEQ ID NO: 183 - RNA, 21 nt, synthetic construct
  misc_feature 1..21: note = Synthetic sequence
  source 1..21: mol_type = other RNA
tatgagagaaccaggtagggg

SEQ ID NO: 184 - RNA, 21 nt, synthetic construct
  misc_feature 1..21: note = Synthetic sequence
  source 1..21: mol_type = other RNA
tacttgttccctattgttctt

SEQ ID NO: 185 - RNA, 21 nt, synthetic construct
  misc_feature 1..21: note = Synthetic sequence
  source 1..21: mol_type = other RNA
ataaattactagattcttggt

SEQ ID NO: 186 - RNA, 21 nt, synthetic construct
  misc_feature 1..21: note = Synthetic sequence
  source 1..21: mol_type = other RNA
tattgctgtgaaattctacca

SEQ ID NO: 187 - RNA, 21 nt, synthetic construct
  misc_feature 1..21: note = Synthetic sequence
  source 1..21: mol_type = other RNA
tttgacaaagtacttcatttg

SEQ ID NO: 188 - RNA, 21 nt, synthetic construct
  misc_feature 1..21: note = Synthetic sequence
  source 1..21: mol_type = other RNA
ttctacttgttccctattgtt

SEQ ID NO: 189 - RNA, 21 nt, synthetic construct
  misc_feature 1..21: note = Synthetic sequence
  source 1..21: mol_type = other RNA
tatttgacaaagtacttcatt

SEQ ID NO: 190 - RNA, 21 nt, synthetic construct
  misc_feature 1..21: note = Synthetic sequence
  source 1..21: mol_type = other RNA
ttgacaaagtacttcatttgg

SEQ ID NO: 191 - RNA, 21 nt, synthetic construct
  misc_feature 1..21: note = Synthetic sequence
  source 1..21: mol_type = other RNA
tttcatgatatcataacatta

SEQ ID NO: 192 - RNA, 21 nt, synthetic construct
  misc_feature 1..21: note = Synthetic sequence
  source 1..21: mol_type = other RNA
attactagattcttggttctt

SEQ ID NO: 193 - RNA, 21 nt, synthetic construct
  misc_feature 1..21: note = Synthetic sequence
  source 1..21: mol_type = other RNA
ttccctattgttctttaggga

SEQ ID NO: 194 - RNA, 21 nt, synthetic construct
  misc_feature 1..21: note = Synthetic sequence
  source 1..21: mol_type = other RNA
ataccagtgaacatgaaacag

SEQ ID NO: 195 - RNA, 21 nt, synthetic construct
  misc_feature 1..21: note = Synthetic sequence
  source 1..21: mol_type = other RNA
ttgaagaagctcgaagcgctc

SEQ ID NO: 196 - RNA, 21 nt, synthetic construct
  misc_feature 1..21: note = Synthetic sequence
  source 1..21: mol_type = other RNA
tcagttgttcccatcctcgtt

SEQ ID NO: 197 - RNA, 21 nt, synthetic construct
  misc_feature 1..21: note = Synthetic sequence
  source 1..21: mol_type = other RNA
tatcataacattaaatcatta

SEQ ID NO: 198 - RNA, 21 nt, synthetic construct
  misc_feature 1..21: note = Synthetic sequence
  source 1..21: mol_type = other RNA
atttatataacttcttatgaa

SEQ ID NO: 199 - RNA, 21 nt, synthetic construct
  misc_feature 1..21: note = Synthetic sequence
  source 1..21: mol_type = other RNA
aaattctaccacttctacttg

SEQ ID NO: 200 - RNA, 21 nt, synthetic construct
  misc_feature 1..21: note = Synthetic sequence
  source 1..21: mol_type = other RNA
ttagggaagagggaggaactg

SEQ ID NO: 201 - RNA, 21 nt, synthetic construct
  misc_feature 1..21: note = Synthetic sequence
  source 1..21: mol_type = other RNA
tttcctgtcctcctgtccttt

SEQ ID NO: 202 - RNA, 21 nt, synthetic construct
  misc_feature 1..21: note = Synthetic sequence
  source 1..21: mol_type = other RNA
tctagagtcgcacagggtcca

SEQ ID NO: 203 - RNA, 21 nt, synthetic construct
  misc_feature 1..21: note = Synthetic sequence
  source 1..21: mol_type = other RNA
attgctgtgaaattctaccac

SEQ ID NO: 204 - RNA, 21 nt, synthetic construct
  misc_feature 1..21: note = Synthetic sequence
  source 1..21: mol_type = other RNA
tttatgatcttcttctgttct

SEQ ID NO: 205 - RNA, 21 nt, synthetic construct
  misc_feature 1..21: note = Synthetic sequence
  source 1..21: mol_type = other RNA
taacttcttatgaagtagatt

SEQ ID NO: 206 - RNA, 21 nt, synthetic construct
  misc_feature 1..21: note = Synthetic sequence
  source 1..21: mol_type = other RNA
tatctagagtcgcacagggtc

SEQ ID NO: 207 - RNA, 21 nt, synthetic construct
  misc_feature 1..21: note = Synthetic sequence
  source 1..21: mol_type = other RNA
taccaaaccggtgcacttcta

SEQ ID NO: 208 - RNA, 21 nt, synthetic construct
  misc_feature 1..21: note = Synthetic sequence
  source 1..21: mol_type = other RNA
tatttatataacttcttatga

SEQ ID NO: 209 - RNA, 21 nt, synthetic construct
  misc_feature 1..21: note = Synthetic sequence
  source 1..21: mol_type = other RNA
tgtaattcacataactctttt

SEQ ID NO: 210 - RNA, 21 nt, synthetic construct
  misc_feature 1..21: note = Synthetic sequence
  source 1..21: mol_type = other RNA
atttcctgtcctcctgtcctt

SEQ ID NO: 211 - RNA, 21 nt, synthetic construct
  misc_feature 1..21: note = Synthetic sequence
  source 1..21: mol_type = other RNA
tgcagcagcaaggatctctgg

SEQ ID NO: 212 - RNA, 21 nt, synthetic construct
  misc_feature 1..21: note = Synthetic sequence
  source 1..21: mol_type = other RNA
taatgtaaactactagcattt

SEQ ID NO: 213 - RNA, 21 nt, synthetic construct
  misc_feature 1..21: note = Synthetic sequence
  source 1..21: mol_type = other RNA
gatagaatatatagataaata

SEQ ID NO: 214 - RNA, 21 nt, synthetic construct
  misc_feature 1..21: note = Synthetic sequence
  source 1..21: mol_type = other RNA
ggaatctatgcaagttcaaga

SEQ ID NO: 215 - RNA, 21 nt, synthetic construct
  misc_feature 1..21: note = Synthetic sequence
  source 1..21: mol_type = other RNA
ggaacgactgtttaatgtact

SEQ ID NO: 216 - RNA, 21 nt, synthetic construct
  misc_feature 1..21: note = Synthetic sequence
  source 1..21: mol_type = other RNA
gtcagacaattgtaattaata

SEQ ID NO: 217 - RNA, 21 nt, synthetic construct
  misc_feature 1..21: note = Synthetic sequence
  source 1..21: mol_type = other RNA
ccctctgaatagtgtgttaac

SEQ ID NO: 218 - RNA, 21 nt, synthetic construct
  misc_feature 1..21: note = Synthetic sequence
  source 1..21: mol_type = other RNA
agttcagttccgagttgaaca

SEQ ID NO: 219 - RNA, 21 nt, synthetic construct
  misc_feature 1..21: note = Synthetic sequence
  source 1..21: mol_type = other RNA
cggaatctatgcaagttcaag

SEQ ID NO: 220 - RNA, 21 nt, synthetic construct
  misc_feature 1..21: note = Synthetic sequence
  source 1..21: mol_type = other RNA
gagaagacgtcttaatttatc

SEQ ID NO: 221 - RNA, 21 nt, synthetic construct
  misc_feature 1..21: note = Synthetic sequence
  source 1..21: mol_type = other RNA
caatgtggttctcttagaaga

SEQ ID NO: 222 - RNA, 21 nt, synthetic construct
  misc_feature 1..21: note = Synthetic sequence
  source 1..21: mol_type = other RNA
agaagtacttggacaactacg

SEQ ID NO: 223 - RNA, 21 nt, synthetic construct
  misc_feature 1..21: note = Synthetic sequence
  source 1..21: mol_type = other RNA
agatagaatatatagataaat

SEQ ID NO: 224 - RNA, 21 nt, synthetic construct
  misc_feature 1..21: note = Synthetic sequence
  source 1..21: mol_type = other RNA
ggataatgttggtctcatttg

SEQ ID NO: 225 - RNA, 21 nt, synthetic construct
  misc_feature 1..21: note = Synthetic sequence
  source 1..21: mol_type = other RNA
ggtctgtgaccacctagaacc

SEQ ID NO: 226 - RNA, 21 nt, synthetic construct
  misc_feature 1..21: note = Synthetic sequence
  source 1..21: mol_type = other RNA
gaatcaagatccgacccaaac

SEQ ID NO: 227 - RNA, 21 nt, synthetic construct
  misc_feature 1..21: note = Synthetic sequence
  source 1..21: mol_type = other RNA
ggcaagtccgactactgaaag

SEQ ID NO: 228 - RNA, 21 nt, synthetic construct
  misc_feature 1..21: note = Synthetic sequence
  source 1..21: mol_type = other RNA
cactttgtagactcgtcaagc

SEQ ID NO: 229 - RNA, 21 nt, synthetic construct
  misc_feature 1..21: note = Synthetic sequence
  source 1..21: mol_type = other RNA
gggtgtgtcggttggtcgagt

SEQ ID NO: 230 - RNA, 21 nt, synthetic construct
  misc_feature 1..21: note = Synthetic sequence
  source 1..21: mol_type = other RNA
gcatcgacgaggagcagtaca

SEQ ID NO: 231 - RNA, 21 nt, synthetic construct
  misc_feature 1..21: note = Synthetic sequence
  source 1..21: mol_type = other RNA
ccctatacaagtcagctaagt

SEQ ID NO: 232 - RNA, 21 nt, synthetic construct
  misc_feature 1..21: note = Synthetic sequence
  source 1..21: mol_type = other RNA
gcacttcaggtgtttgcaagc

SEQ ID NO: 233 - RNA, 21 nt, synthetic construct
  misc_feature 1..21: note = Synthetic sequence
  source 1..21: mol_type = other RNA
gcaatatgatcattctagagc

SEQ ID NO: 234 - RNA, 21 nt, synthetic construct
  misc_feature 1..21: note = Synthetic sequence
  source 1..21: mol_type = other RNA
gttctggtttcaacgaataag

SEQ ID NO: 235 - RNA, 21 nt, synthetic construct
  misc_feature 1..21: note = Synthetic sequence
  source 1..21: mol_type = other RNA
ggataatcacttgaatgaagc

SEQ ID NO: 236 - RNA, 21 nt, synthetic construct
  misc_feature 1..21: note = Synthetic sequence
  source 1..21: mol_type = other RNA
gactgtttaatgtacttgatg

SEQ ID NO: 237 - RNA, 21 nt, synthetic construct
  misc_feature 1..21: note = Synthetic sequence
  source 1..21: mol_type = other RNA
ccacaatgtggttctcttaga

SEQ ID NO: 238 - RNA, 21 nt, synthetic construct
  misc_feature 1..21: note = Synthetic sequence
  source 1..21: mol_type = other RNA
cgctctggtcacaccaatact

SEQ ID NO: 239 - RNA, 21 nt, synthetic construct
  misc_feature 1..21: note = Synthetic sequence
  source 1..21: mol_type = other RNA
ggaggagacagatcatccagt

SEQ ID NO: 240 - RNA, 21 nt, synthetic construct
  misc_feature 1..21: note = Synthetic sequence
  source 1..21: mol_type = other RNA
ctttgtagattgtgtgtttgt

SEQ ID NO: 241 - RNA, 21 nt, synthetic construct
  misc_feature 1..21: note = Synthetic sequence
  source 1..21: mol_type = other RNA
gctggttccggacccagaacc

SEQ ID NO: 242 - RNA, 21 nt, synthetic construct
  misc_feature 1..21: note = Synthetic sequence
  source 1..21: mol_type = other RNA
gaatatatagataaatacata

SEQ ID NO: 243 - RNA, 21 nt, synthetic construct
  misc_feature 1..21: note = Synthetic sequence
  source 1..21: mol_type = other RNA
gcatgagcgacctgtacgaca

SEQ ID NO: 244 - RNA, 21 nt, synthetic construct
  misc_feature 1..21: note = Synthetic sequence
  source 1..21: mol_type = other RNA
cttcagtaataatgatgaaac

SEQ ID NO: 245 - RNA, 21 nt, synthetic construct
  misc_feature 1..21: note = Synthetic sequence
  source 1..21: mol_type = other RNA
cagatatatgaaagcattatt

SEQ ID NO: 246 - RNA, 21 nt, synthetic construct
  misc_feature 1..21: note = Synthetic sequence
  source 1..21: mol_type = other RNA
ccagcaccttctcctactacg

SEQ ID NO: 247 - RNA, 21 nt, synthetic construct
  misc_feature 1..21: note = Synthetic sequence
  source 1..21: mol_type = other RNA
cagttaccaaactaggataat

SEQ ID NO: 248 - RNA, 21 nt, synthetic construct
  misc_feature 1..21: note = Synthetic sequence
  source 1..21: mol_type = other RNA
gaaagagaagacgtcttaatt

SEQ ID NO: 249 - RNA, 21 nt, synthetic construct
  misc_feature 1..21: note = Synthetic sequence
  source 1..21: mol_type = other RNA
tatgcaagttcaagagttatc

SEQ ID NO: 250 - RNA, 21 nt, synthetic construct
  misc_feature 1..21: note = Synthetic sequence
  source 1..21: mol_type = other RNA
cagcagaagcagaaagagaag

SEQ ID NO: 251 - RNA, 21 nt, synthetic construct
  misc_feature 1..21: note = Synthetic sequence
  source 1..21: mol_type = other RNA
agcagaagcagaaagagaaga

SEQ ID NO: 252 - RNA, 21 nt, synthetic construct
  misc_feature 1..21: note = Synthetic sequence
  source 1..21: mol_type = other RNA
cacagtctctttagaactaag

SEQ ID NO: 253 - RNA, 21 nt, synthetic construct
  misc_feature 1..21: note = Synthetic sequence
  source 1..21: mol_type = other RNA
ggcaagtactgcagctatttg

SEQ ID NO: 254 - RNA, 21 nt, synthetic construct
  misc_feature 1..21: note = Synthetic sequence
  source 1..21: mol_type = other RNA
gcacctcttcagtaataatga

SEQ ID NO: 255 - RNA, 21 nt, synthetic construct
  misc_feature 1..21: note = Synthetic sequence
  source 1..21: mol_type = other RNA
gcacctcttcagtaataatga

SEQ ID NO: 256 - RNA, 21 nt, synthetic construct
  misc_feature 1..21: note = Synthetic sequence
  source 1..21: mol_type = other RNA
agattgcactcacttgaaacc

SEQ ID NO: 257 - RNA, 21 nt, synthetic construct
  misc_feature 1..21: note = Synthetic sequence
  source 1..21: mol_type = other RNA
aagaggaatgtaagtgttacc

SEQ ID NO: 258 - RNA, 21 nt, synthetic construct
  misc_feature 1..21: note = Synthetic sequence
  source 1..21: mol_type = other RNA
cgtctgaaggactctcctagg

SEQ ID NO: 259 - RNA, 21 nt, synthetic construct
  misc_feature 1..21: note = Synthetic sequence
  source 1..21: mol_type = other RNA
cagaccagttaccaaactagg

SEQ ID NO: 260 - RNA, 21 nt, synthetic construct
  misc_feature 1..21: note = Synthetic sequence
  source 1..21: mol_type = other RNA
gcagagggctgactttcaaga

SEQ ID NO: 261 - RNA, 21 nt, synthetic construct
  misc_feature 1..21: note = Synthetic sequence
  source 1..21: mol_type = other RNA
cgggtgtgtggatgtgcgagg

SEQ ID NO: 262 - RNA, 21 nt, synthetic construct
  misc_feature 1..21: note = Synthetic sequence
  source 1..21: mol_type = other RNA
gggcaagtccgactactgaaa

SEQ ID NO: 263 - RNA, 21 nt, synthetic construct
  misc_feature 1..21: note = Synthetic sequence
  source 1..21: mol_type = other RNA
tttatctatatattctatcta

SEQ ID NO: 264 - RNA, 21 nt, synthetic construct
  misc_feature 1..21: note = Synthetic sequence
  source 1..21: mol_type = other RNA
ttgaacttgcatagattccgg

SEQ ID NO: 265 - RNA, 21 nt, synthetic construct
  misc_feature 1..21: note = Synthetic sequence
  source 1..21: mol_type = other RNA
tacattaaacagtcgttcctt

SEQ ID NO: 266 - RNA, 21 nt, synthetic construct
  misc_feature 1..21: note = Synthetic sequence
  source 1..21: mol_type = other RNA
ttaattacaattgtctgactt

SEQ ID NO: 267 - RNA, 21 nt, synthetic construct
  misc_feature 1..21: note = Synthetic sequence
  source 1..21: mol_type = other RNA
taacacactattcagaggggg

SEQ ID NO: 268 - RNA, 21 nt, synthetic construct
  misc_feature 1..21: note = Synthetic sequence
  source 1..21: mol_type = other RNA
ttcaactcggaactgaactga

SEQ ID NO: 269 - RNA, 21 nt, synthetic construct
  misc_feature 1..21: note = Synthetic sequence
  source 1..21: mol_type = other RNA
tgaacttgcatagattccggg

SEQ ID NO: 270 - RNA, 21 nt, synthetic construct
  misc_feature 1..21: note = Synthetic sequence
  source 1..21: mol_type = other RNA
taaattaagacgtcttctctt

SEQ ID NO: 271 - RNA, 21 nt, synthetic construct
  misc_feature 1..21: note = Synthetic sequence
  source 1..21: mol_type = other RNA
ttctaagagaaccacattgtg

SEQ ID NO: 272 - RNA, 21 nt, synthetic construct
  misc_feature 1..21: note = Synthetic sequence
  source 1..21: mol_type = other RNA
tagttgtccaagtacttctgt

SEQ ID NO: 273 - RNA, 21 nt, synthetic construct
  misc_feature 1..21: note = Synthetic sequence
  source 1..21: mol_type = other RNA
ttatctatatattctatctat

SEQ ID NO: 274 - RNA, 21 nt, synthetic construct
  misc_feature 1..21: note = Synthetic sequence
  source 1..21: mol_type = other RNA
aatgagaccaacattatccta

SEQ ID NO: 275 - RNA, 21 nt, synthetic construct
  misc_feature 1..21: note = Synthetic sequence
  source 1..21: mol_type = other RNA
ttctaggtggtcacagaccaa

SEQ ID NO: 276 - RNA, 21 nt, synthetic construct
  misc_feature 1..21: note = Synthetic sequence
  source 1..21: mol_type = other RNA
ttgggtcggatcttgattcgg

SEQ ID NO: 277 - RNA, 21 nt, synthetic construct
  misc_feature 1..21: note = Synthetic sequence
  source 1..21: mol_type = other RNA
ttcagtagtcggacttgccct

SEQ ID NO: 278 - RNA, 21 nt, synthetic construct
  misc_feature 1..21: note = Synthetic sequence
  source 1..21: mol_type = other RNA
ttgacgagtctacaaagtgtg

SEQ ID NO: 279 - RNA, 21 nt, synthetic construct
  misc_feature 1..21: note = Synthetic sequence
  source 1..21: mol_type = other RNA
tcgaccaaccgacacaccctg

SEQ ID NO: 280 - RNA, 21 nt, synthetic construct
  misc_feature 1..21: note = Synthetic sequence
  source 1..21: mol_type = other RNA
tactgctcctcgtcgatgctg

SEQ ID NO: 281 - RNA, 21 nt, synthetic construct
  misc_feature 1..21: note = Synthetic sequence
  source 1..21: mol_type = other RNA
ttagctgacttgtatagggaa

SEQ ID NO: 282 - RNA, 21 nt, synthetic construct
  misc_feature 1..21: note = Synthetic sequence
  source 1..21: mol_type = other RNA
ttgcaaacacctgaagtgctg

SEQ ID NO: 283 - RNA, 21 nt, synthetic construct
  misc_feature 1..21: note = Synthetic sequence
  source 1..21: mol_type = other RNA
tctagaatgatcatattgcag

SEQ ID NO: 284 - RNA, 21 nt, synthetic construct
  misc_feature 1..21: note = Synthetic sequence
  source 1..21: mol_type = other RNA
tattcgttgaaaccagaacga

SEQ ID NO: 285 - RNA, 21 nt, synthetic construct
  misc_feature 1..21: note = Synthetic sequence
  source 1..21: mol_type = other RNA
ttcattcaagtgattatccag

SEQ ID NO: 286 - RNA, 21 nt, synthetic construct
  misc_feature 1..21: note = Synthetic sequence
  source 1..21: mol_type = other RNA
tcaagtacattaaacagtcgt

SEQ ID NO: 287 - RNA, 21 nt, synthetic construct
  misc_feature 1..21: note = Synthetic sequence
  source 1..21: mol_type = other RNA
taagagaaccacattgtggaa

SEQ ID NO: 288 - RNA, 21 nt, synthetic construct
  misc_feature 1..21: note = Synthetic sequence
  source 1..21: mol_type = other RNA
tattggtgtgaccagagcggt

SEQ ID NO: 289 - RNA, 21 nt, synthetic construct
  misc_feature 1..21: note = Synthetic sequence
  source 1..21: mol_type = other RNA
tggatgatctgtctcctccgg

SEQ ID NO: 290 - RNA, 21 nt, synthetic construct
  misc_feature 1..21: note = Synthetic sequence
  source 1..21: mol_type = other RNA
aaacacacaatctacaaagtg

SEQ ID NO: 291 - RNA, 21 nt, synthetic construct
  misc_feature 1..21: note = Synthetic sequence
  source 1..21: mol_type = other RNA
ttctgggtccggaaccagctg

SEQ ID NO: 292 - RNA, 21 nt, synthetic construct
  misc_feature 1..21: note = Synthetic sequence
  source 1..21: mol_type = other RNA
tgtatttatctatatattcta

SEQ ID NO: 293 - RNA, 21 nt, synthetic construct
  misc_feature 1..21: note = Synthetic sequence
  source 1..21: mol_type = other RNA
tcgtacaggtcgctcatgctg

SEQ ID NO: 294 - RNA, 21 nt, synthetic construct
  misc_feature 1..21: note = Synthetic sequence
  source 1..21: mol_type = other RNA
ttcatcattattactgaagag

SEQ ID NO: 295 - RNA, 21 nt, synthetic construct
  misc_feature 1..21: note = Synthetic sequence
  source 1..21: mol_type = other RNA
taatgctttcatatatctgta

SEQ ID NO: 296 - RNA, 21 nt, synthetic construct
  misc_feature 1..21: note = Synthetic sequence
  source 1..21: mol_type = other RNA
tagtaggagaaggtgctggag

SEQ ID NO: 297 - RNA, 21 nt, synthetic construct
  misc_feature 1..21: note = Synthetic sequence
  source 1..21: mol_type = other RNA
tatcctagtttggtaactggt

SEQ ID NO: 298 - RNA, 21 nt, synthetic construct
  misc_feature 1..21: note = Synthetic sequence
  source 1..21: mol_type = other RNA
ttaagacgtcttctctttctg

SEQ ID NO: 299 - RNA, 21 nt, synthetic construct
  misc_feature 1..21: note = Synthetic sequence
  source 1..21: mol_type = other RNA
taactcttgaacttgcataga

SEQ ID NO: 300 - RNA, 21 nt, synthetic construct
  misc_feature 1..21: note = Synthetic sequence
  source 1..21: mol_type = other RNA
tctctttctgcttctgctgtg

SEQ ID NO: 301 - RNA, 21 nt, synthetic construct
  misc_feature 1..21: note = Synthetic sequence
  source 1..21: mol_type = other RNA
ttctctttctgcttctgctgt

SEQ ID NO: 302 - RNA, 21 nt, synthetic construct
  misc_feature 1..21: note = Synthetic sequence
  source 1..21: mol_type = other RNA
tagttctaaagagactgtggt

SEQ ID NO: 303 - RNA, 21 nt, synthetic construct
  misc_feature 1..21: note = Synthetic sequence
  source 1..21: mol_type = other RNA
aatagctgcagtacttgcccg

SEQ ID NO: 304 - RNA, 21 nt, synthetic construct
  misc_feature 1..21: note = Synthetic sequence
  source 1..21: mol_type = other RNA
attattactgaagaggtgctg

SEQ ID NO: 305 - RNA, 21 nt, synthetic construct
  misc_feature 1..21: note = Synthetic sequence
  source 1..21: mol_type = other RNA
atctgcaacagtattggtgtg

SEQ ID NO: 306 - RNA, 21 nt, synthetic construct
  misc_feature 1..21: note = Synthetic sequence
  source 1..21: mol_type = other RNA
tttcaagtgagtgcaatctgg

SEQ ID NO: 307 - RNA, 21 nt, synthetic construct
  misc_feature 1..21: note = Synthetic sequence
  source 1..21: mol_type = other RNA
taacacttacattcctctttg

SEQ ID NO: 308 - RNA, 21 nt, synthetic construct
  misc_feature 1..21: note = Synthetic sequence
  source 1..21: mol_type = other RNA
taggagagtccttcagacggg

SEQ ID NO: 309 - RNA, 21 nt, synthetic construct
  misc_feature 1..21: note = Synthetic sequence
  source 1..21: mol_type = other RNA
tagtttggtaactggtctgcg

SEQ ID NO: 310 - RNA, 21 nt, synthetic construct
  misc_feature 1..21: note = Synthetic sequence
  source 1..21: mol_type = other RNA
ttgaaagtcagccctctgcga

SEQ ID NO: 311 - RNA, 21 nt, synthetic construct
  misc_feature 1..21: note = Synthetic sequence
  source 1..21: mol_type = other RNA
tcgcacatccacacacccgag

SEQ ID NO: 312 - RNA, 21 nt, synthetic construct
  misc_feature 1..21: note = Synthetic sequence
  source 1..21: mol_type = other RNA
tcagtagtcggacttgccctg

SEQ ID NO: 313 - RNA, 21 nt, synthetic construct
  misc_feature 1..21: note = Synthetic sequence
  source 1..21: mol_type = other RNA
ggaacgactgtttaatgtact

SEQ ID NO: 314 - RNA, 21 nt, synthetic construct
  misc_feature 1..21: note = Synthetic sequence
  source 1..21: mol_type = other RNA
gtcagacaattgtaattaata

SEQ ID NO: 315 - RNA, 21 nt, synthetic construct
  misc_feature 1..21: note = Synthetic sequence
  source 1..21: mol_type = other RNA
ccctctgaatagtgtgttaac

SEQ ID NO: 316 - RNA, 21 nt, synthetic construct
  misc_feature 1..21: note = Synthetic sequence
  source 1..21: mol_type = other RNA
agttcagttccgagttgaaca

SEQ ID NO: 317 - RNA, 21 nt, synthetic construct
  misc_feature 1..21: note = Synthetic sequence
  source 1..21: mol_type = other RNA
caatgtggttctcttagaaga

SEQ ID NO: 318 - RNA, 21 nt, synthetic construct
  misc_feature 1..21: note = Synthetic sequence
  source 1..21: mol_type = other RNA
agaagtacttggacaactacg

SEQ ID NO: 319 - RNA, 21 nt, synthetic construct
  misc_feature 1..21: note = Synthetic sequence
  source 1..21: mol_type = other RNA
ggataatgttggtctcatttg

SEQ ID NO: 320 - RNA, 21 nt, synthetic construct
  misc_feature 1..21: note = Synthetic sequence
  source 1..21: mol_type = other RNA
ggtctgtgaccacctagaacc

SEQ ID NO: 321 - RNA, 21 nt, synthetic construct
  misc_feature 1..21: note = Synthetic sequence
  source 1..21: mol_type = other RNA
gaatcaagatccgacccaaac

SEQ ID NO: 322 - RNA, 21 nt, synthetic construct
  misc_feature 1..21: note = Synthetic sequence
  source 1..21: mol_type = other RNA
ggcaagtccgactactgaaag

SEQ ID NO: 323 - RNA, 21 nt, synthetic construct
  misc_feature 1..21: note = Synthetic sequence
  source 1..21: mol_type = other RNA
cactttgtagactcgtcaagc

SEQ ID NO: 324 - RNA, 21 nt, synthetic construct
  misc_feature 1..21: note = Synthetic sequence
  source 1..21: mol_type = other RNA
gggtgtgtcggttggtcgagt

SEQ ID NO: 325 - RNA, 21 nt, synthetic construct
  misc_feature 1..21: note = Synthetic sequence
  source 1..21: mol_type = other RNA
gcatcgacgaggagcagtaca

SEQ ID NO: 326 - RNA, 21 nt, synthetic construct
  misc_feature 1..21: note = Synthetic sequence
  source 1..21: mol_type = other RNA
ccctatacaagtcagctaagt

SEQ ID NO: 327 - RNA, 21 nt, synthetic construct
  misc_feature 1..21: note = Synthetic sequence
  source 1..21: mol_type = other RNA
gcacttcaggtgtttgcaagc

SEQ ID NO: 328 - RNA, 21 nt, synthetic construct
  misc_feature 1..21: note = Synthetic sequence
  source 1..21: mol_type = other RNA
gcaatatgatcattctagagc

SEQ ID NO: 329 - RNA, 21 nt, synthetic construct
  misc_feature 1..21: note = Synthetic sequence
  source 1..21: mol_type = other RNA
gttctggtttcaacgaataag

SEQ ID NO: 330 - RNA, 21 nt, synthetic construct
  misc_feature 1..21: note = Synthetic sequence
  source 1..21: mol_type = other RNA
ggataatcacttgaatgaagc

SEQ ID NO: 331 - RNA, 21 nt, synthetic construct
  misc_feature 1..21: note = Synthetic sequence
  source 1..21: mol_type = other RNA
gactgtttaatgtacttgatg

SEQ ID NO: 332 - RNA, 21 nt, synthetic construct
  misc_feature 1..21: note = Synthetic sequence
  source 1..21: mol_type = other RNA
ccacaatgtggttctcttaga

SEQ ID NO: 333 - RNA, 21 nt, synthetic construct
  misc_feature 1..21: note = Synthetic sequence
  source 1..21: mol_type = other RNA
ctttgtagattgtgtgtttgt

SEQ ID NO: 334 - RNA, 21 nt, synthetic construct
  misc_feature 1..21: note = Synthetic sequence
  source 1..21: mol_type = other RNA
gctggttccggacccagaacc

SEQ ID NO: 335 - RNA, 21 nt, synthetic construct
  misc_feature 1..21: note = Synthetic sequence
  source 1..21: mol_type = other RNA
gcatgagcgacctgtacgaca

SEQ ID NO: 336 - RNA, 21 nt, synthetic construct
  misc_feature 1..21: note = Synthetic sequence
  source 1..21: mol_type = other RNA
ccagcaccttctcctactacg

SEQ ID NO: 337 - RNA, 21 nt, synthetic construct
  misc_feature 1..21: note = Synthetic sequence
  source 1..21: mol_type = other RNA
cagttaccaaactaggataat

SEQ ID NO: 338 - RNA, 21 nt, synthetic construct
  misc_feature 1..21: note = Synthetic sequence
  source 1..21: mol_type = other RNA
cacagtctctttagaactaag

SEQ ID NO: 339 - RNA, 21 nt, synthetic construct
  misc_feature 1..21: note = Synthetic sequence
  source 1..21: mol_type = other RNA
ggcaagtactgcagctatttg

SEQ ID NO: 340 - RNA, 21 nt, synthetic construct
  misc_feature 1..21: note = Synthetic sequence
  source 1..21: mol_type = other RNA
agattgcactcacttgaaacc

SEQ ID NO: 341 - RNA, 21 nt, synthetic construct
  misc_feature 1..21: note = Synthetic sequence
  source 1..21: mol_type = other RNA
cgtctgaaggactctcctagg

SEQ ID NO: 342 - RNA, 21 nt, synthetic construct
  misc_feature 1..21: note = Synthetic sequence
  source 1..21: mol_type = other RNA
cagaccagttaccaaactagg

SEQ ID NO: 343 - RNA, 21 nt, synthetic construct
  misc_feature 1..21: note = Synthetic sequence
  source 1..21: mol_type = other RNA
gcagagggctgactttcaaga

SEQ ID NO: 344 - RNA, 21 nt, synthetic construct
  misc_feature 1..21: note = Synthetic sequence
  source 1..21: mol_type = other RNA
ccttgttgaagaggagaaaca

SEQ ID NO: 345 - RNA, 21 nt, synthetic construct
  misc_feature 1..21: note = Synthetic sequence
  source 1..21: mol_type = other RNA
cgggtgtgtggatgtgcgagg

SEQ ID NO: 346 - RNA, 21 nt, synthetic construct
  misc_feature 1..21: note = Synthetic sequence
  source 1..21: mol_type = other RNA
gggcaagtccgactactgaaa

SEQ ID NO: 347 - RNA, 21 nt, synthetic construct
  misc_feature 1..21: note = Synthetic sequence
  source 1..21: mol_type = other RNA
gagcaaatatatatatacacg

SEQ ID NO: 348 - RNA, 21 nt, synthetic construct
  misc_feature 1..21: note = Synthetic sequence
  source 1..21: mol_type = other RNA
gggtcagtagagtcggtttct

SEQ ID NO: 349 - RNA, 21 nt, synthetic construct
  misc_feature 1..21: note = Synthetic sequence
  source 1..21: mol_type = other RNA
cctctgaatagtgtgttaacc

SEQ ID NO: 350 - RNA, 21 nt, synthetic construct
  misc_feature 1..21: note = Synthetic sequence
  source 1..21: mol_type = other RNA
gataccaactgttcccttata

SEQ ID NO: 351 - RNA, 21 nt, synthetic construct
  misc_feature 1..21: note = Synthetic sequence
  source 1..21: mol_type = other RNA
ccttctcctactacggcaagc

SEQ ID NO: 352 - RNA, 21 nt, synthetic construct
  misc_feature 1..21: note = Synthetic sequence
  source 1..21: mol_type = other RNA
gactttcaagacttcaagaac

SEQ ID NO: 353 - RNA, 21 nt, synthetic construct
  misc_feature 1..21: note = Synthetic sequence
  source 1..21: mol_type = other RNA
gttcagccctgttacagaagt

SEQ ID NO: 354 - RNA, 21 nt, synthetic construct
  misc_feature 1..21: note = Synthetic sequence
  source 1..21: mol_type = other RNA
gcagggacagcatcatctatg

SEQ ID NO: 355 - RNA, 21 nt, synthetic construct
  misc_feature 1..21: note = Synthetic sequence
  source 1..21: mol_type = other RNA
tgagaatcatgctgcaatatg

SEQ ID NO: 356 - RNA, 21 nt, synthetic construct
  misc_feature 1..21: note = Synthetic sequence
  source 1..21: mol_type = other RNA
ggttctctgtcatcgccgagg

SEQ ID NO: 357 - RNA, 21 nt, synthetic construct
  misc_feature 1..21: note = Synthetic sequence
  source 1..21: mol_type = other RNA
atttcaagatgaaactaaagc

SEQ ID NO: 358 - RNA, 21 nt, synthetic construct
  misc_feature 1..21: note = Synthetic sequence
  source 1..21: mol_type = other RNA
ggtattgacgtctgtcagatc

SEQ ID NO: 359 - RNA, 21 nt, synthetic construct
  misc_feature 1..21: note = Synthetic sequence
  source 1..21: mol_type = other RNA
cgacccaaaccacaccaaatg

SEQ ID NO: 360 - RNA, 21 nt, synthetic construct
  misc_feature 1..21: note = Synthetic sequence
  source 1..21: mol_type = other RNA
gatcattctagagcaaatata

SEQ ID NO: 361 - RNA, 21 nt, synthetic construct
  misc_feature 1..21: note = Synthetic sequence
  source 1..21: mol_type = other RNA
gctaaagtcccaaacggaagt

SEQ ID NO: 362 - RNA, 21 nt, synthetic construct
  misc_feature 1..21: note = Synthetic sequence
  source 1..21: mol_type = other RNA
tcaggtgtttgcaagcaaagc

SEQ ID NO: 363 - RNA, 21 nt, synthetic construct
  misc_feature 1..21: note = Synthetic sequence
  source 1..21: mol_type = other RNA
tacattaaacagtcgttcctt

SEQ ID NO: 364 - RNA, 21 nt, synthetic construct
  misc_feature 1..21: note = Synthetic sequence
  source 1..21: mol_type = other RNA
ttaattacaattgtctgactt

SEQ ID NO: 365 - RNA, 21 nt, synthetic construct
  misc_feature 1..21: note = Synthetic sequence
  source 1..21: mol_type = other RNA
taacacactattcagaggggg

SEQ ID NO: 366 - RNA, 21 nt, synthetic construct
  misc_feature 1..21: note = Synthetic sequence
  source 1..21: mol_type = other RNA
ttcaactcggaactgaactga

SEQ ID NO: 367 - RNA, 21 nt, synthetic construct
  misc_feature 1..21: note = Synthetic sequence
  source 1..21: mol_type = other RNA
ttctaagagaaccacattgtg

SEQ ID NO: 368 - RNA, 21 nt, synthetic construct
  misc_feature 1..21: note = Synthetic sequence
  source 1..21: mol_type = other RNA
tagttgtccaagtacttctgt

SEQ ID NO: 369 - RNA, 21 nt, synthetic construct
  misc_feature 1..21: note = Synthetic sequence
  source 1..21: mol_type = other RNA
ttaacacactattcagagggg

SEQ ID NO: 370 - RNA, 21 nt, synthetic construct
  misc_feature 1..21: note = Synthetic sequence
  source 1..21: mol_type = other RNA
taagggaacagttggtatctt

SEQ ID NO: 371 - RNA, 21 nt, synthetic construct
  misc_feature 1..21: note = Synthetic sequence
  source 1..21: mol_type = other RNA
ttgccgtagtaggagaaggtg

SEQ ID NO: 372 - RNA, 21 nt, synthetic construct
  misc_feature 1..21: note = Synthetic sequence
  source 1..21: mol_type = other RNA
tcttgaagtcttgaaagtcag

SEQ ID NO: 373 - RNA, 21 nt, synthetic construct
  misc_feature 1..21: note = Synthetic sequence
  source 1..21: mol_type = other RNA
ttctgtaacagggctgaacaa

SEQ ID NO: 374 - RNA, 21 nt, synthetic construct
  misc_feature 1..21: note = Synthetic sequence
  source 1..21: mol_type = other RNA
tagatgatgctgtccctgccg

SEQ ID NO: 375 - RNA, 21 nt, synthetic construct
  misc_feature 1..21: note = Synthetic sequence
  source 1..21: mol_type = other RNA
tattgcagcatgattctcatg

SEQ ID NO: 376 - RNA, 21 nt, synthetic construct
  misc_feature 1..21: note = Synthetic sequence
  source 1..21: mol_type = other RNA
tcggcgatgacagagaaccag

SEQ ID NO: 377 - RNA, 21 nt, synthetic construct
  misc_feature 1..21: note = Synthetic sequence
  source 1..21: mol_type = other RNA
tttagtttcatcttgaaatat

SEQ ID NO: 378 - RNA, 21 nt, synthetic construct
  misc_feature 1..21: note = Synthetic sequence
  source 1..21: mol_type = other RNA
tctgacagacgtcaatacctg

SEQ ID NO: 379 - RNA, 21 nt, synthetic construct
  misc_feature 1..21: note = Synthetic sequence
  source 1..21: mol_type = other RNA
tttggtgtggtttgggtcgga

SEQ ID NO: 380 - RNA, 21 nt, synthetic construct
  misc_feature 1..21: note = Synthetic sequence
  source 1..21: mol_type = other RNA
tatttgctctagaatgatcat

SEQ ID NO: 381 - RNA, 21 nt, synthetic construct
  misc_feature 1..21: note = Synthetic sequence
  source 1..21: mol_type = other RNA
ttccgtttgggactttagctt

SEQ ID NO: 382 - RNA, 21 nt, synthetic construct
  misc_feature 1..21: note = Synthetic sequence
  source 1..21: mol_type = other RNA
tttgcttgcaaacacctgaag

SEQ ID NO: 383 - RNA, 21 nt, synthetic construct
  misc_feature 1..21: note = Synthetic sequence
  source 1..21: mol_type = other RNA
ggtttgagctactaagcaaat

SEQ ID NO: 384 - RNA, 21 nt, synthetic construct
  misc_feature 1..21: note = Synthetic sequence
  source 1..21: mol_type = other RNA
agaagtacttggacaactacg

SEQ ID NO: 385 - RNA, 21 nt, synthetic construct
  misc_feature 1..21: note = Synthetic sequence
  source 1..21: mol_type = other RNA
ggtaggtttgagctactaagc

SEQ ID NO: 386 - RNA, 21 nt, synthetic construct
  misc_feature 1..21: note = Synthetic sequence
  source 1..21: mol_type = other RNA
gggtaggtttgagctactaag

SEQ ID NO: 387 - RNA, 21 nt, synthetic construct
  misc_feature 1..21: note = Synthetic sequence
  source 1..21: mol_type = other RNA
gcatcgacgaggagcagtaca

SEQ ID NO: 388 - RNA, 21 nt, synthetic construct
  misc_feature 1..21: note = Synthetic sequence
  source 1..21: mol_type = other RNA
ggataatcacttgaatgaagc

SEQ ID NO: 389 - RNA, 21 nt, synthetic construct
  misc_feature 1..21: note = Synthetic sequence
  source 1..21: mol_type = other RNA
gctggttccggacccagaacc

SEQ ID NO: 390 - RNA, 21 nt, synthetic construct
  misc_feature 1..21: note = Synthetic sequence
  source 1..21: mol_type = other RNA
gcatgagcgacctgtacgaca

SEQ ID NO: 391 - RNA, 21 nt, synthetic construct
  misc_feature 1..21: note = Synthetic sequence
  source 1..21: mol_type = other RNA
ccagcaccttctcctactacg

SEQ ID NO: 392 - RNA, 21 nt, synthetic construct
  misc_feature 1..21: note = Synthetic sequence
  source 1..21: mol_type = other RNA
ccttgttgaagaggagaaaca

SEQ ID NO: 393 - RNA, 21 nt, synthetic construct
  misc_feature 1..21: note = Synthetic sequence
  source 1..21: mol_type = other RNA
gtttgagctactaagcaaata

SEQ ID NO: 394 - RNA, 21 nt, synthetic construct
  misc_feature 1..21: note = Synthetic sequence
  source 1..21: mol_type = other RNA
ccttctcctactacggcaagc

SEQ ID NO: 395 - RNA, 21 nt, synthetic construct
  misc_feature 1..21: note = Synthetic sequence
  source 1..21: mol_type = other RNA
gttcagccctgttacagaagt

SEQ ID NO: 396 - RNA, 21 nt, synthetic construct
  misc_feature 1..21: note = Synthetic sequence
  source 1..21: mol_type = other RNA
gcagggacagcatcatctatg

SEQ ID NO: 397 - RNA, 21 nt, synthetic construct
  misc_feature 1..21: note = Synthetic sequence
  source 1..21: mol_type = other RNA
ggttctctgtcatcgccgagg

SEQ ID NO: 398 - RNA, 21 nt, synthetic construct
  misc_feature 1..21: note = Synthetic sequence
  source 1..21: mol_type = other RNA
gctaaagtcccaaacggaagt

SEQ ID NO: 399 - RNA, 21 nt, synthetic construct
  misc_feature 1..21: note = Synthetic sequence
  source 1..21: mol_type = other RNA
gacgagaccttgctggataat

SEQ ID NO: 400 - RNA, 21 nt, synthetic construct
  misc_feature 1..21: note = Synthetic sequence
  source 1..21: mol_type = other RNA
ctaagcaaataccacactaac

SEQ ID NO: 401 - RNA, 21 nt, synthetic construct
  misc_feature 1..21: note = Synthetic sequence
  source 1..21: mol_type = other RNA
acgccgtggtgctgatcaagc

SEQ ID NO: 402 - RNA, 21 nt, synthetic construct
  misc_feature 1..21: note = Synthetic sequence
  source 1..21: mol_type = other RNA
gcagcatgagcgacctgtacg

SEQ ID NO: 403 - RNA, 21 nt, synthetic construct
  misc_feature 1..21: note = Synthetic sequence
  source 1..21: mol_type = other RNA
aggtcatcttcgaaggcgacc

SEQ ID NO: 404 - RNA, 21 nt, synthetic construct
  misc_feature 1..21: note = Synthetic sequence
  source 1..21: mol_type = other RNA
cagacgaggtacagtcggagc

SEQ ID NO: 405 - RNA, 21 nt, synthetic construct
  misc_feature 1..21: note = Synthetic sequence
  source 1..21: mol_type = other RNA
cagccctgttacagaagtact

SEQ ID NO: 406 - RNA, 21 nt, synthetic construct
  misc_feature 1..21: note = Synthetic sequence
  source 1..21: mol_type = other RNA
gagcagtactacagcctcacc

SEQ ID NO: 407 - RNA, 21 nt, synthetic construct
  misc_feature 1..21: note = Synthetic sequence
  source 1..21: mol_type = other RNA
gctggataatcacttgaatga

SEQ ID NO: 408 - RNA, 21 nt, synthetic construct
  misc_feature 1..21: note = Synthetic sequence
  source 1..21: mol_type = other RNA
acctgtacgacatgcagaagc

SEQ ID NO: 409 - RNA, 21 nt, synthetic construct
  misc_feature 1..21: note = Synthetic sequence
  source 1..21: mol_type = other RNA
gaatgaagctaaagtcccaaa

SEQ ID NO: 410 - RNA, 21 nt, synthetic construct
  misc_feature 1..21: note = Synthetic sequence
  source 1..21: mol_type = other RNA
gtgagtccactgaggtcaaat

SEQ ID NO: 411 - RNA, 21 nt, synthetic construct
  misc_feature 1..21: note = Synthetic sequence
  source 1..21: mol_type = other RNA
tcacttgaatgaagctaaagt

SEQ ID NO: 412 - RNA, 21 nt, synthetic construct
  misc_feature 1..21: note = Synthetic sequence
  source 1..21: mol_type = other RNA
agaccttgctggataatcact

SEQ ID NO: 413 - RNA, 21 nt, synthetic construct
  misc_feature 1..21: note = Synthetic sequence
  source 1..21: mol_type = other RNA
gtagggtaggtttgagctact

SEQ ID NO: 414 - RNA, 21 nt, synthetic construct
  misc_feature 1..21: note = Synthetic sequence
  source 1..21: mol_type = other RNA
gccctgttacagaagtacttg

SEQ ID NO: 415 - RNA, 21 nt, synthetic construct
  misc_feature 1..21: note = Synthetic sequence
  source 1..21: mol_type = other RNA
ccctgttacagaagtacttgg

SEQ ID NO: 416 - RNA, 21 nt, synthetic construct
  misc_feature 1..21: note = Synthetic sequence
  source 1..21: mol_type = other RNA
tgagcgacctgtacgacatgc

SEQ ID NO: 417 - RNA, 21 nt, synthetic construct
  misc_feature 1..21: note = Synthetic sequence
  source 1..21: mol_type = other RNA
agctgctgttcctgcacgaca

SEQ ID NO: 418 - RNA, 21 nt, synthetic construct
  misc_feature 1..21: note = Synthetic sequence
  source 1..21: mol_type = other RNA
acaccagggtggacacgaagc

SEQ ID NO: 419 - RNA, 21 nt, synthetic construct
  misc_feature 1..21: note = Synthetic sequence
  source 1..21: mol_type = other RNA
cgtccaaagacccttgtgagt

SEQ ID NO: 420 - RNA, 21 nt, synthetic construct
  misc_feature 1..21: note = Synthetic sequence
  source 1..21: mol_type = other RNA
gggctgtcccgtccaaagacc

SEQ ID NO: 421 - RNA, 21 nt, synthetic construct
  misc_feature 1..21: note = Synthetic sequence
  source 1..21: mol_type = other RNA
ggtcccactgctaccgctacc

SEQ ID NO: 422 - RNA, 21 nt, synthetic construct
  misc_feature 1..21: note = Synthetic sequence
  source 1..21: mol_type = other RNA
gtgttctacaccgacttcatg

SEQ ID NO: 423 - RNA, 21 nt, synthetic construct
  misc_feature 1..21: note = Synthetic sequence
  source 1..21: mol_type = other RNA
tgttcagcatcgacgaggagc

SEQ ID NO: 424 - RNA, 21 nt, synthetic construct
  misc_feature 1..21: note = Synthetic sequence
  source 1..21: mol_type = other RNA
tgttctacaccgacttcatgg

SEQ ID NO: 425 - RNA, 21 nt, synthetic construct
  misc_feature 1..21: note = Synthetic sequence
  source 1..21: mol_type = other RNA
tcaatggagtcttcagaaagg

SEQ ID NO: 426 - RNA, 21 nt, synthetic construct
  misc_feature 1..21: note = Synthetic sequence
  source 1..21: mol_type = other RNA
agcttgggtcccactgctacc

SEQ ID NO: 427 - RNA, 21 nt, synthetic construct
  misc_feature 1..21: note = Synthetic sequence
  source 1..21: mol_type = other RNA
aggccgtgttctacaccgact

SEQ ID NO: 428 - RNA, 21 nt, synthetic construct
  misc_feature 1..21: note = Synthetic sequence
  source 1..21: mol_type = other RNA
acgagaccttgctggataatc

SEQ ID NO: 429 - RNA, 21 nt, synthetic construct
  misc_feature 1..21: note = Synthetic sequence
  source 1..21: mol_type = other RNA
tgagcgtcagggtgatctacg

SEQ ID NO: 430 - RNA, 21 nt, synthetic construct
  misc_feature 1..21: note = Synthetic sequence
  source 1..21: mol_type = other RNA
ctgaggtcaaatcaatggagt

SEQ ID NO: 431 - RNA, 21 nt, synthetic construct
  misc_feature 1..21: note = Synthetic sequence
  source 1..21: mol_type = other RNA
ggagcagagacaccgcggaac

SEQ ID NO: 432 - RNA, 21 nt, synthetic construct
  misc_feature 1..21: note = Synthetic sequence
  source 1..21: mol_type = other RNA
ggttccggacccagaaccagg

SEQ ID NO: 433 - RNA, 21 nt, synthetic construct
  misc_feature 1..21: note = Synthetic sequence
  source 1..21: mol_type = other RNA
ttgcttagtagctcaaaccta

SEQ ID NO: 434 - RNA, 21 nt, synthetic construct
  misc_feature 1..21: note = Synthetic sequence
  source 1..21: mol_type = other RNA
tagttgtccaagtacttctgt

SEQ ID NO: 435 - RNA, 21 nt, synthetic construct
  misc_feature 1..21: note = Synthetic sequence
  source 1..21: mol_type = other RNA
ttagtagctcaaacctaccct

SEQ ID NO: 436 - RNA, 21 nt, synthetic construct
  misc_feature 1..21: note = Synthetic sequence
  source 1..21: mol_type = other RNA
tagtagctcaaacctacccta

SEQ ID NO: 437 - RNA, 21 nt, synthetic construct
  misc_feature 1..21: note = Synthetic sequence
  source 1..21: mol_type = other RNA
tactgctcctcgtcgatgctg

SEQ ID NO: 438 - RNA, 21 nt, synthetic construct
  misc_feature 1..21: note = Synthetic sequence
  source 1..21: mol_type = other RNA
ttcattcaagtgattatccag

SEQ ID NO: 439 - RNA, 21 nt, synthetic construct
  misc_feature 1..21: note = Synthetic sequence
  source 1..21: mol_type = other RNA
ttctgggtccggaaccagctg

SEQ ID NO: 440 - RNA, 21 nt, synthetic construct
  misc_feature 1..21: note = Synthetic sequence
  source 1..21: mol_type = other RNA
tcgtacaggtcgctcatgctg

SEQ ID NO: 441 - RNA, 21 nt, synthetic construct
  misc_feature 1..21: note = Synthetic sequence
  source 1..21: mol_type = other RNA
tagtaggagaaggtgctggag

SEQ ID NO: 442 - RNA, 21 nt, synthetic construct
  misc_feature 1..21: note = Synthetic sequence
  source 1..21: mol_type = other RNA
tttctcctcttcaacaaggag

SEQ ID NO: 443 - RNA, 21 nt, synthetic construct
  misc_feature 1..21: note = Synthetic sequence
  source 1..21: mol_type = other RNA
tttgcttagtagctcaaacct

SEQ ID NO: 444 - RNA, 21 nt, synthetic construct
  misc_feature 1..21: note = Synthetic sequence
  source 1..21: mol_type = other RNA
ttgccgtagtaggagaaggtg

SEQ ID NO: 445 - RNA, 21 nt, synthetic construct
  misc_feature 1..21: note = Synthetic sequence
  source 1..21: mol_type = other RNA
ttctgtaacagggctgaacaa

SEQ ID NO: 446 - RNA, 21 nt, synthetic construct
  misc_feature 1..21: note = Synthetic sequence
  source 1..21: mol_type = other RNA
tagatgatgctgtccctgccg

SEQ ID NO: 447 - RNA, 21 nt, synthetic construct
  misc_feature 1..21: note = Synthetic sequence
  source 1..21: mol_type = other RNA
tcggcgatgacagagaaccag

SEQ ID NO: 448 - RNA, 21 nt, synthetic construct
  misc_feature 1..21: note = Synthetic sequence
  source 1..21: mol_type = other RNA
ttccgtttgggactttagctt

SEQ ID NO: 449 - RNA, 21 nt, synthetic construct
  misc_feature 1..21: note = Synthetic sequence
  source 1..21: mol_type = other RNA
tatccagcaaggtctcgtcct

SEQ ID NO: 450 - RNA, 21 nt, synthetic construct
  misc_feature 1..21: note = Synthetic sequence
  source 1..21: mol_type = other RNA
tagtgtggtatttgcttagta

SEQ ID NO: 451 - RNA, 21 nt, synthetic construct
  misc_feature 1..21: note = Synthetic sequence
  source 1..21: mol_type = other RNA
ttgatcagcaccacggcgttg

SEQ ID NO: 452 - RNA, 21 nt, synthetic construct
  misc_feature 1..21: note = Synthetic sequence
  source 1..21: mol_type = other RNA
tacaggtcgctcatgctgcgc

SEQ ID NO: 453 - RNA, 21 nt, synthetic construct
  misc_feature 1..21: note = Synthetic sequence
  source 1..21: mol_type = other RNA
tcgccttcgaagatgacctgg

SEQ ID NO: 454 - RNA, 21 nt, synthetic construct
  misc_feature 1..21: note = Synthetic sequence
  source 1..21: mol_type = other RNA
tccgactgtacctcgtctggg

SEQ ID NO: 455 - RNA, 21 nt, synthetic construct
  misc_feature 1..21: note = Synthetic sequence
  source 1..21: mol_type = other RNA
tacttctgtaacagggctgaa

SEQ ID NO: 456 - RNA, 21 nt, synthetic construct
  misc_feature 1..21: note = Synthetic sequence
  source 1..21: mol_type = other RNA
tgaggctgtagtactgctccg

SEQ ID NO: 457 - RNA, 21 nt, synthetic construct
  misc_feature 1..21: note = Synthetic sequence
  source 1..21: mol_type = other RNA
attcaagtgattatccagcaa

SEQ ID NO: 458 - RNA, 21 nt, synthetic construct
  misc_feature 1..21: note = Synthetic sequence
  source 1..21: mol_type = other RNA
ttctgcatgtcgtacaggtcg

SEQ ID NO: 459 - RNA, 21 nt, synthetic construct
  misc_feature 1..21: note = Synthetic sequence
  source 1..21: mol_type = other RNA
tgggactttagcttcattcaa

SEQ ID NO: 460 - RNA, 21 nt, synthetic construct
  misc_feature 1..21: note = Synthetic sequence'
  source 1..21: mol_type = other RNA
ttgacctcagtggactcacaa

SEQ ID NO: 461 - RNA, 21 nt, synthetic construct
  misc_feature 1..21: note = Synthetic sequence
  source 1..21: mol_type = other RNA
tttagcttcattcaagtgatt

SEQ ID NO: 462 - RNA, 21 nt, synthetic construct
  misc_feature 1..21: note = Synthetic sequence
  source 1..21: mol_type = other RNA
tgattatccagcaaggtctcg

SEQ ID NO: 463 - RNA, 21 nt, synthetic construct
  misc_feature 1..21: note = Synthetic sequence
  source 1..21: mol_type = other RNA
tagctcaaacctaccctacga

SEQ ID NO: 464 - RNA, 21 nt, synthetic construct
  misc_feature 1..21: note = Synthetic sequence
  source 1..21: mol_type = other RNA
agtacttctgtaacagggctg

SEQ ID NO: 465 - RNA, 21 nt, synthetic construct
  misc_feature 1..21: note = Synthetic sequence
  source 1..21: mol_type = other RNA
aagtacttctgtaacagggct

SEQ ID NO: 466 - RNA, 21 nt, synthetic construct
  misc_feature 1..21: note = Synthetic sequence
  source 1..21: mol_type = other RNA
atgtcgtacaggtcgctcatg

SEQ ID NO: 467 - RNA, 21 nt, synthetic construct
  misc_feature 1..21: note = Synthetic sequence
  source 1..21: mol_type = other RNA
tcgtgcaggaacagcagctcg

SEQ ID NO: 468 - RNA, 21 nt, synthetic construct
  misc_feature 1..21: note = Synthetic sequence
  source 1..21: mol_type = other RNA
ttcgtgtccaccctggtgtag

SEQ ID NO: 469 - RNA, 21 nt, synthetic construct
  misc_feature 1..21: note = Synthetic sequence
  source 1..21: mol_type = other RNA
tcacaagggtctttggacggg

SEQ ID NO: 470 - RNA, 21 nt, synthetic construct
  misc_feature 1..21: note = Synthetic sequence
  source 1..21: mol_type = other RNA
tctttggacgggacagccctg

SEQ ID NO: 471 - RNA, 21 nt, synthetic construct
  misc_feature 1..21: note = Synthetic sequence
  source 1..21: mol_type = other RNA
tagcggtagcagtgggaccca

SEQ ID NO: 472 - RNA, 21 nt, synthetic construct
  misc_feature 1..21: note = Synthetic sequence
  source 1..21: mol_type = other RNA
tgaagtcggtgtagaacacgg

SEQ ID NO: 473 - RNA, 21 nt, synthetic construct
  misc_feature 1..21: note = Synthetic sequence
  source 1..21: mol_type = other RNA
tcctcgtcgatgctgaacagg

SEQ ID NO: 474 - RNA, 21 nt, synthetic construct
  misc_feature 1..21: note = Synthetic sequence
  source 1..21: mol_type = other RNA
atgaagtcggtgtagaacacg

SEQ ID NO: 475 - RNA, 21 nt, synthetic construct
  misc_feature 1..21: note = Synthetic sequence
  source 1..21: mol_type = other RNA
tttctgaagactccattgatt

SEQ ID NO: 476 - RNA, 21 nt, synthetic construct
  misc_feature 1..21: note = Synthetic sequence
  source 1..21: mol_type = other RNA
tagcagtgggacccaagcttg

SEQ ID NO: 477 - RNA, 21 nt, synthetic construct
  misc_feature 1..21: note = Synthetic sequence
  source 1..21: mol_type = other RNA
tcggtgtagaacacggcctcg

SEQ ID NO: 478 - RNA, 21 nt, synthetic construct
  misc_feature 1..21: note = Synthetic sequence
  source 1..21: mol_type = other RNA
ttatccagcaaggtctcgtcc

SEQ ID NO: 479 - RNA, 21 nt, synthetic construct
  misc_feature 1..21: note = Synthetic sequence
  source 1..21: mol_type = other RNA
tagatcaccctgacgctcagg

SEQ ID NO: 480 - RNA, 21 nt, synthetic construct
  misc_feature 1..21: note = Synthetic sequence
  source 1..21: mol_type = other RNA
tccattgatttgacctcagtg

SEQ ID NO: 481 - RNA, 21 nt, synthetic construct
  misc_feature 1..21: note = Synthetic sequence
  source 1..21: mol_type = other RNA
tccgcggtgtctctgctcccg

SEQ ID NO: 482 - RNA, 21 nt, synthetic construct
  misc_feature 1..21: note = Synthetic sequence
  source 1..21: mol_type = other RNA
tggttctgggtccggaaccag

SEQ ID NO: 483 - protein, 293 aa, Homo sapiens
MRVDSSADPTMSQEQGPGSSTPPSSPTLLDALLQNLYDFGGTEGETEQKKIIKKRENKKRDVMASAALAAEPSPLPGSLIRGQRKSASSFFKELREERHCAPSGTPTGPEILAAAVPPSSLKNNREQVEVVEFHSNKKRKLTPDHNKNTKQANPSVLERDVDTQEFNLEKARLEVHRFGITGYGKGKERILEQERAIMLGAKPPKKSYVNYKVLQEQIKEKKAAKEEEKRLAQETDIFKKKKRKGQEDRKSKKKSAPSILSNGRIGQVGKFKNGTLILSPVDIKKINSSRVAK

SEQ ID NO: 484 - protein, 292 aa, Homo sapiens
MRVDSSADPTMSQEQGPGSSTPPSSPTLLDALLQNLYDFGGTEGETEQKKIIKKRENKKRDVMASAALAAEPSPLPGSLIRGQRKSASSFFKELREERHCAPSGTPTGPEILAAAVPPSSLKNNREQVEVVEFHSNKKRKLTPDHNKNTKANPSVLERDVDTQEFNLEKARLEVHRFGITGYGKGKERILEQERAIMLGAKPPKKSYVNYKVLQEQIKEKKAAKEEEKRLAQETDIFKKKKRKGQEDRKSKKKSAPSILSNGRIGQVGKFKNGTLILSPVDIKKINSSRVAK

SEQ ID NO: 485 - protein, 808 aa, Homo sapiens
MKMAPQNADPESMQVQELSVPLPDPQKAGGAEAENCETISEGSIDRIPMRLWVMHGAVMFGREFCYAMETALVTPILLQIGLPEQYYSLTWFLSPILGLIFTPLIGSASDRCTLSWGRRRPFILALCVGVLFGVALFLNGSAIGLALGDVPNRQPIGIVLTVLGVVVLDFSADATEGPIRAYLLDVVDSEEQDMALNIHAFSAGLGGAIGYVLGGLDWTQTFLGSWFRTQNQVLFFFAAIIFTVSVALHLFSIDEEQYSPQQERSAEEPGALDGGEPHGVPAFPDEVQSEHELALDYPDVDIMRSKSDSALHVPDTALDLEPELLFLHDIEPSIFHDASYPATPRSTSQELAKTKLPRLATFLKEAAKEDETLLDNHLNEAKVPNGSGSPTKDALGGYTRVDTKPSATSSSMRRRRHAFRRQASSTFSYYGKLGSHCYRYRRANAVVLIKPSRSMSDLYDMQKRQRQHRHRNQSGATTSSGDTESEEGEGETTVRLLWLSMLKMPRELMRLCLCHLLTWFSVIAEAVFYTDFMGQVIFEGDPKAPSNSTAWQAYNAGVKMGCWGLVIYAATGAICSALLQKYLDNYDLSVRVIYVLGTLGFSVGTAVMAMFPNVYVAMVTISTMGIVSMSISYCPYALLGQYHDIKQYIHHSPGNSKRGFGIDCAILSCQVYISQILVASALGGVVDAVGTVRVIPMVASVGSFLGFLTATFLVIYPNVSEEAKEEQKGLSSPLAGEGRAGGNSEKPTVLKLTRKEGLQGPVETESVTPAGIDVCQISSHWLVPQLLESIFLYDYFRKKIFFSTMWFS

SEQ ID NO: 486 - protein, 798 aa, Homo sapiens
MGKASPASGLSRPKTLVSPLRSNQWSLQKGLPEQYYSLTWFLSPILGLIFTPLIGSASDRCTLSWGRRRPFILALCVGVLFGVALFLNGSAIGLALGDVPNRQPIGIVLTVLGVVVLDFSADATEGPIRAYLLDVVDSEEQDMALNIHAFSAGLGGAIGYVLGGLDWTQTFLGSWFRTQNQVLFFFAAIIFTVSVALHLFSIDEEQYSPQQERSAEEPGALDGGEPHGVPAFPDEVQSEHELALDYPDVDIMRSKSDSALHVPDTALDLEPELLFLHDIEPSIFHDASYPATPRSTSQELAKTKLPRLATFLKEAAKEDETLLDNHLNEAKVPNGSGSPTKDALGGYTRVDTKPSATSSSMRRRRHAFRRQASSTFSYYGKLGSHCYRYRRANAVVLIKPSRSMSDLYDMQKRQRQHRHRNQSGATTSSGDTESEEGEGETTVRLLWLSMLKMPRELMRLCLCHLLTWFSVIAEAVFYTDFMGQVIFEGDPKAPSNSTAWQAYNAGVKMGCWGLVIYAATGAICSALLQKYLDNYDLSVRVIYVLGTLGFSVGTAVMAMFPNVYVAMVTISTMGIVSMSISYCPYALLGQYHDIKQYIHHSPGNSKRGFGIDCAILSCQVYISQILVASALGGVVDAVGTVRVIPMVASVGSFLGFLTATFLVIYPNVSEEAKEEQKGLSSPLAGEGRAGGNSEKPTVLKLTRKEGLQGPVETERLQVLTSVRSRHIGWCRSCWRVFFFMIILEKKFSFPQCGSLRRMTYLLFLSELDTLCPGQPCPWAATAHQSWEEAGPGGLGRRQ

SEQ ID NO: 487 - protein, 717 aa, Homo sapiens
MGKASPASGLSRPKTLVSPLRSNQWSLQKGLPEQYYSLTWFLSPILGLIFTPLIGSASDRCTLSWGRRRPFILALCVGVLFGVALFLNGSAIGLALGDVPNRQPIGIVLTVLGVVVLDFSADATEGPIRAYLLDVVDSEEQDMALNIHAFSAGLGGAIGYVLGGLDWTQTFLGSWFRTQNQVLFFFAAIIFTVSVALHLFSIDEEQYSPQQERSAEEPGALDGGEPHGVPAFPDEVQSEHELALDYPDVDIMRSKSDSALHVPDTALDLEPELLFLHDIEPSIFHDASYPATPRSTSQELAKTKLPRLATFLKEAAKEDETLLDNHLNEAKVPNGSGSPTKDALGGYTRVDTKPSATSSSMRRRRHAFRRQASSTFSYYGKLGSHCYRYRRANAVVLIKPSRSMSDLYDMQKRQRQHRHRNQSGATTSSGDTESEEGEGETTVRLLWLSMLKMPRELMRLCLCHLLTWFSVIAEAVFYTDFMGQVIFEGDPKAPSNSTAWQAYNAGVKMGCWGLVIYAATGAICSALLQKYLDNYDLSVRVIYVLGTLGFSVGTAVMAMFPNVYVAMVTISTMGIVSMSISYCPYALLGQYHDIKQYIHHSPGNSKRGFGIDCAILSCQVYISQILVASALGGVVDAVGTVRVIPMVASVGSFLGFLTATFLVIYPNVSEEAKEEQKGLSSPLAGEGRAGGNSEKPTVLKLTRKEGLQGPVETESVV

SEQ ID NO: 488 - RNA, 1430 nt, Homo sapiens
  source 1..1430: mol_type = mRNA
gcggaacccggaagcgggggtgcagcgcggcagaatgagggttgattcctcggctgaccccacaatgtcgcaggagcaagggccggggtcctccacgcctcccagttctccgacacttcttgacgctctgctccagaacctttacgactttggaggtacagaaggtgaaacagaacagaagaagatcataaagaaaagggaaaacaagaagagagatgtgatggcttcagcggccttggcagcagagccatctcccctacctggttctctcataagaggccagaggaagagcgcttcgagcttcttcaaggaacttagagaagagcggcattgtgctccttctgggacccccacaggaccagagatccttgctgctgcagttcctccctcttccctaaagaacaatagggaacaagtagaagtggtagaatttcacagcaataaaaaaagaaaattgacgccagatcataacaagaacacaaagcaggctaatcctagtgttttggagagagatgtggatacacaagaatttaacctagaaaaagctcgtttagaagtgcaccggtttggtatcacgggttatggaaaaggaaaggagagaatcctggaacaggaacgtgccattatgctgggcgctaagcctcctaaaaagagttatgtgaattacaaggttttacaggagcaaattaaagaaaaaaaggcagcaaaggaagaagaaaagagactggcccaagaaacagatattttcaagaaaaagaagaggaaaggacaggaggacaggaaatccaaaaagaagtccgctcccagtattttgtcaaatggacggattggacaggttggaaaattcaaaaatggaacactgattctgagcccagttgatatcaagaaaataaattcttccagagtggccaaatgaagtactttgtcaaataaaacgaggatgggaacaactgagtcaactgcagcggccctggaccctgtgcgactctagatagatcgttcttaacacgcatttcacagactgctttttcattttagaaaaaaagtcaaacagaagtaacatttctagttgccgaagtgtctgcttcatggtgctgaacgtgtcaggcccagtgcagcatgaatgtgaagtgaacccactgaaaagtgatttttaatgttctccagcaaacttcactcctgtttcatgttcactggtatttggtgcatttgcttgtaatacagttttttataactttactgttttgtaaatgctagtagtttacattaaattgccttatatttttaatgatttaatgttatgatatcatgaaagaaccaagaatctagtaatttatcagttttgtggaaaagcagttttaatttttagcctaccatgtgtacaaaatctacttcataagaagttatataaataaaataggtttgtagta

SEQ ID NO: 489 - RNA, 1427 nt, Homo sapiens
  source 1..1427: mol_type = mRNA
gcggaacccggaagcgggggtgcagcgcggcagaatgagggttgattcctcggctgaccccacaatgtcgcaggagcaagggccggggtcctccacgcctcccagttctccgacacttcttgacgctctgctccagaacctttacgactttggaggtacagaaggtgaaacagaacagaagaagatcataaagaaaagggaaaacaagaagagagatgtgatggcttcagcggccttggcagcagagccatctcccctacctggttctctcataagaggccagaggaagagcgcttcgagcttcttcaaggaacttagagaagagcggcattgtgctccttctgggacccccacaggaccagagatccttgctgctgcagttcctccctcttccctaaagaacaatagggaacaagtagaagtggtagaatttcacagcaataaaaaaagaaaattgacgccagatcataacaagaacacaaaggctaatcctagtgttttggagagagatgtggatacacaagaatttaacctagaaaaagctcgtttagaagtgcaccggtttggtatcacgggttatggaaaaggaaaggagagaatcctggaacaggaacgtgccattatgctgggcgctaagcctcctaaaaagagttatgtgaattacaaggttttacaggagcaaattaaagaaaaaaaggcagcaaaggaagaagaaaagagactggcccaagaaacagatattttcaagaaaaagaagaggaaaggacaggaggacaggaaatccaaaaagaagtccgctcccagtattttgtcaaatggacggattggacaggttggaaaattcaaaaatggaacactgattctgagcccagttgatatcaagaaaataaattcttccagagtggccaaatgaagtactttgtcaaataaaacgaggatgggaacaactgagtcaactgcagcggccctggaccctgtgcgactctagatagatcgttcttaacacgcatttcacagactgctttttcattttagaaaaaaagtcaaacagaagtaacatttctagttgccgaagtgtctgcttcatggtgctgaacgtgtcaggcccagtgcagcatgaatgtgaagtgaacccactgaaaagtgatttttaatgttctccagcaaacttcactcctgtttcatgttcactggtatttggtgcatttgcttgtaatacagttttttataactttactgttttgtaaatgctagtagtttacattaaattgccttatatttttaatgatttaatgttatgatatcatgaaagaaccaagaatctagtaatttatcagttttgtggaaaagcagttttaatttttagcctaccatgtgtacaaaatctacttcataagaagttatataaataaaataggtttgtagta

SEQ ID NO: 490 - RNA, 7418 nt, Homo sapiens
  source 1..7418: mol_type = mRNA
gcagcctcgcagcgctctctccgcgcccgcgtcgctgactgaccgcccggccggccggccgagggagcagcccccgcccggacccgcagccgcctcccgaccccggtgcgcccggggctccgcgcccccgtagcccctgcccggcccggcccggccgccccgccgcccccgcggccccggccggaggagacagatcatccagtcactcgatggggattaccagcggaccacctgtttgtatcaccttcccacctctctgtagcaagaaaaatcacttcagcacctcttcagtaataatgatgaaactggtattaactgaatggcaattatggatttttaactctaattcacagtaaaccagcaagccatatgtttaaaatccaaccaaaaatcgttctgtgttgctgcatgtctcataccgttgtgagaagttgtgagatagccacttgtgcaaagaggaatgtaagtgttacctgcccgaggaacacagcagaagcagaaagagaagacgtcttaatttatcattacagatatatgaaagcattatttatatagatagaatatatagataaatacatatatttttggtggtaatgaaaatggctccgcagaatgccgacccggaatctatgcaagttcaagagttatccgtgcccctgccggacccgcagaaagccggaggcgcagaggccgagaactgcgagaccatcagcgaggggtccatagaccgaatccccatgcgcctgtgggtgatgcacggggcggtgatgtttggcagggagttctgttacgccatggaaaccgctctggtcacaccaatactgttgcagattggccttccggagcagtactacagcctcacctggttcctgagccccatccttggcctcatcttcacacctctcattgggtctgcgagtgaccggtgcaccctgagctggggccgccggcggcccttcatcctcgccctctgcgttggcgtcctctttggcgttgcacttttccttaacggctctgccatcggtctggccctcggcgatgtccccaaccggcagcccattggcatcgtgctcacggtgctgggagtggtggtcctggacttcagcgccgatgccaccgaggggcccatccgtgcctatctgctggacgtggtggacagcgaggagcaggacatggccctcaacatccacgccttctctgccggcctcggcggagccatcggctacgtgctgggtgggctggactggacccagaccttcctgggcagctggttccggacccagaaccaggtgctcttcttctttgccgccatcatcttcacggtgtccgtggccctgcacctgttcagcatcgacgaggagcagtacagcccgcagcaggagcgcagcgctgaggagcccggcgccctggatgggggcgagccgcacggcgtccctgccttcccagacgaggtacagtcggagcacgagctggccctggactacccggacgtggacatcatgcgcagcaaaagcgactcggcattgcacgtgccggacaccgcgctggacctggagcccgagctgctgttcctgcacgacatcgagccctccatcttccacgacgcctcctaccccgccaccccccgcagcaccagccaggagctcgccaagaccaagctgccccgcctggccaccttcctcaaggaagccgccaaggaggacgagaccttgctggataatcacttgaatgaagctaaagtcccaaacggaagtggctcccccacaaaagacgccctcggcggctacaccagggtggacacgaagccctcggccacgtcgagctccatgcggcggcggcggcacgcgttccgcaggcaggcctccagcaccttctcctactacggcaagcttgggtcccactgctaccgctaccggcgcgccaacgccgtggtgctgatcaagccgtcgcgcagcatgagcgacctgtacgacatgcagaagcggcagcggcagcaccggcaccggaaccagagcggggccaccacctccagcggggacaccgagagtgaggagggggagggcgagaccacggtgcgcctgctgtggctctccatgctgaagatgcccagggagctgatgcggctgtgcctctgccacctcctcacctggttctctgtcatcgccgaggccgtgttctacaccgacttcatgggccaggtcatcttcgaaggcgaccccaaggccccctcgaactcgaccgcctggcaagcctacaacgccggggtcaagatgggctgctggggcctggtcatttatgccgccactggtgctatttgttcagccctgttacagaagtacttggacaactacgacctgagcgtcagggtgatctacgtgctggggacgctgggcttctctgtcggcacagccgtgatggccatgtttcccaacgtctacgtcgccatggtcaccatcagcaccatgggcatcgtctccatgagcatctcctactgcccgtacgccctgctgggccagtaccatgacatcaagcagtacatccaccacagccccgggaactccaagcgagggtttggcatagattgtgccatcctgtcctgccaagtgtacatctcgcagatcctggtggcctctgcccttgggggcgtggtcgacgccgtggggactgtccgcgtcatccccatggtggcctctgtgggctctttcctgggcttcctgacggccacattcctggtgatctatcccaacgtgtcagaggaggccaaggaggagcagaaaggcctgtcttccccgttggccggcgaaggcagggccggtgggaacagcgaaaagcccaccgtgctgaagctcacgcggaaggagggcctgcagggaccggtggagacagagtccgtgacgcctgcaggtattgacgtctgtcagatctcgtcacattggctggtgccgcagctgttggagagtatttttctttatgattattttagaaaaaaaattttcttttccacaatgtggttctcttagaagaatgacgtatcttcttttcctcagcgagttggacacattgtgcccagggcagccctgtccttgggcagcgaccgcacaccaaagctgggaggaggctggtccggggggcctgggcagaagacagtgatttgcaggggtggctcccagacaccctgcccagggatgggctgggcaccacctgggggcggagcgtgagctccagacgagctcctgcgtgcgcgtgtgagtgtgtctgcgcccagccatgtgaccccgctcgtcccgtctgaaggactctcctaggaggccaggttgcccctccagaccgctcccaacgtcagggggaaggaaacgttgactttcactgcactttgattcgtctctaaaccatttgctggggattcctgagagcagagctcccagcgggccctgcctcccaagtcccgccgcaaggctacctcgggtgtgtggatgtgcgagggcctcccccgcttgcgaaggggacatgcgtgctggaacctgtcggaactccatgccttcctcgcctgctcacctgctcgacgctggaatcgggacaggtgcaaagggacgcagacgtctgggacagctaaggcccgtgtcaccggagggctccgcacagtcgttctggtttcaacgaataagcaaaactcgggcaagtactgcagctatttggaaatgttttccaaaccacagtctctttagaactaagcctatttgaaacggtcggtgtaggcttactgagatcaggagacagggaggccccgcacatcacacagataaagtcagacaattgtaattaatacttttgctgcctcaagttgttttttaaataaagtactttgaaatgcatgagaatcatgctgcaatatgatcattctagagcaaatatatatatacacgtatatatatttcaagatgaaactaaagcagtttttaaataaattacttgaattttctgtgtatttaaaggaacgactgtttaatgtacttgatgggcctctggtcttgccgtgtctcctgccgctggtggcactttgtagattgtgtgtttgtgtccgggtggcagttgggtacctgctcacgcacggtgtgtctgccaggccacggtgtcccaggatcgcagagggctgactttcaagacttcaagaacattttctggatgtgtggaaacttgagaatggccttgtgaatctcgtgcttggacagggcaagtccgactactgaaagtgctgccagctttgctgcgagccctccggccagcgggagccccgtgggctgggcactgtggcccttcttctctgggggacggcacccctggcttcctcacctcggccgggcgtccgtggcagctcactctatgcaacttgatcctctagcggctttaagactgtagatcccctctctgagacctggctgtacttgtcaggatctcgaggcgcagctcccgtcttagctggtttctccggcttctcgtcctgacgactataaaacagttggaggcaagaaagcagcggatgtggggtggcagtggcctgacccgaatcaagatccgacccaaaccacaccaaatgtgggttcatctgggggccaccccttgcctgaggcttcccaccctcatctgaaggcccaggggccggatccagggctcaccaaagccgattcctcgccagctgggagtgcagaagtctcagggcctggctgcgacttgatttttaggaggaagaggggcttcgcaacccccctctgaatagtgtgttaacccttgagatcccagcctcgactaatctgaagtaaggacaacaaaggccattcagtgccctccacatggccttgccacagtcactgtcagggtatgaacgtgccggaagccagtgccagccagggacaggcgtgactgttgtgtgctcctcggtgacaggagtcggtggctgcacactttgtagactcgtcaagctgtcagcacttcaggtgtttgcaagcaaagcccttcttagtgtgcaggtcagtgtgcagagcccaaatgaggggaccgcaggggctggggtccagggtcagtagagtcggtttctggagctgccttcctgggaggcaggtgtgggtgaccggggctctggcggtgcgtgtgggcccggcctggccacagcggggaccaggtcacgacatctttggcctcaacccctcccctgcactgagattatttccagattgcactcacttgaaaccgtccgtgtcgtcaccttgtgtcttaagggaagccgagaagaagggcagacgggcaggctgtcttgtctgcaaagcgccattgcgcccgcagcttgtgtgggtcaggctgcagctcgggtgtctgtggcttctaaccttgtacctcagacggatgcagtaacaaggcggggctggggacgccggtcagtgtcaaaggggaggtgctctggctgatagccttgcccgagagggacgaggaggccgtgcgggtgccccctggggaagctggccagccatccagtgctgaggacgcagctggagttgggctcgtggcacccttggggtgtgggctgtgcaggctggtggcctgggtgcctctgcacactgagtggagtgtcaggcagggtgtgtcggttggtcgagtcttgtgtaatgtgcgcagaccagttaccaaactaggataatgttggtctcatttgtggtggttttgttccctatacaagtcagctaagtaaagactcttttaacgagcttccccttaacacatggcagaagtttccaggtgcaggaatgcgagctggcgggaaggggcagaggccgtgagctctcagctgggccggcctgcctgtgtcccctttcctgggtctgtcggcagactggcatcatgacgttccctggtggctgaagagctagctttggagtgttgtttttctcactctcaggcaggggccttagctggaatcctccaacctgccactgaacacgtcagtgctgtgtgctgcctcttggacacctgcccttgaaagcctcaggcccctgggagaagcactctgtccagtcctgtccccgggggggaggcagggccactgagccctcctcagatggttagtggcttccaacagccatcaggagtgtttcttgaatgccccaggtgtggaggacttggtctgtgaccacctagaaccccagagctgaacaggaagccgtccctgcagcaacaagagggctggaagggggagctgcaggccaccctcggctctcccactgctggggcggtgatgttcgggtgacatgtttgaaaaatactcttaaagataccaactgttcccttatatggctaatggtttgtgcagccaccagcgatggcggcccctattagagaccaggtttgttaaaacaccaaatattgctgtccacactagacattaaccggcttcagaaaagatggacaccttttcccacgctgtttcgcttcttaactttggtccagctttagccaccacacagcgtgtgagggactgctgctgcggagtcagcctcgtttgtccctccgcctcccaccagcacgcgccgcttctgagagacaccagctccctgcctccaagcctggtgccacaggcctgtcgtgagggacccctgcttccgagagctcctgggggggttctgcccttcaccacctgggagaggtgtcagttcagttccgagttgaacaaggcccgtgcacacagcatgttgggggcccagcccaaagttcttgtcacctcctcatgcaaagccagccatcaccctccggccagagctcaaggtggccccttggccagcccctccttgggtcctccaggaggactgagcacccctcctagcggcatcccttgccctccacagtgctgccaggggcacgtcgctctgtgccgtggactgagaccatcccctggtgacagaatgacccgtttgttggaaatgcctcgttgccagagaaactccccaggcatctcggaacgaaactatttagttccattgtgaactggccacgggacagctttttatcaacttattaagttggagcactgtaatcgcgcttgctgagttagcagtggtggtaagcgtgtgttaaacacataatgttacgttttaggagagagaggtcgtaaggaagtgtcgtgtcgctcatgactctcttctattagttgggtaacagtggcctcatgtttgtgtctgtgtgtacacagagcccttaggttctgctctgtttctttgccaggtgaatgtttgtggcatgcgctgctgtccgcgcccctctgtcctgcgcagggttcagctgtgcggcgccctgatttcctccatgcacacagaacctccttgtgtctgtttctctgttcctctgtggctgactcaataaacttttccctctga

SEQ ID NO: 491 - RNA, 6973 nt, Homo sapiens
  source 1..6973: mol_type = mRNA
tgatagggcagagaggagcccatgctccagctgcggggagtgcctgcccgaggagctggccagtgaagacaggtgcctgccagtgaagacaggtgcccacaagctggacacagggtcggccctgagacaggacagcagctgcggaatgttcccgctggctttctggagcgccagcgtcctgcttgggtctggccaggggcctctagccaggctgttgaggcgcccctccttgttgaagaggagaaacagtggccccctgggagttgctgggaggtgcctggcacagggcggcagggacagcatcatctatggggaaagcaagcccagcctcagggctgtcccgtccaaagacccttgtgagtccactgaggtcaaatcaatggagtcttcagaaaggccttccggagcagtactacagcctcacctggttcctgagccccatccttggcctcatcttcacacctctcattgggtctgcgagtgaccggtgcaccctgagctggggccgccggcggcccttcatcctcgccctctgcgttggcgtcctctttggcgttgcacttttccttaacggctctgccatcggtctggccctcggcgatgtccccaaccggcagcccattggcatcgtgctcacggtgctgggagtggtggtcctggacttcagcgccgatgccaccgaggggcccatccgtgcctatctgctggacgtggtggacagcgaggagcaggacatggccctcaacatccacgccttctctgccggcctcggcggagccatcggctacgtgctgggtgggctggactggacccagaccttcctgggcagctggttccggacccagaaccaggtgctcttcttctttgccgccatcatcttcacggtgtccgtggccctgcacctgttcagcatcgacgaggagcagtacagcccgcagcaggagcgcagcgctgaggagcccggcgccctggatgggggcgagccgcacggcgtccctgccttcccagacgaggtacagtcggagcacgagctggccctggactacccggacgtggacatcatgcgcagcaaaagcgactcggcattgcacgtgccggacaccgcgctggacctggagcccgagctgctgttcctgcacgacatcgagccctccatcttccacgacgcctcctaccccgccaccccccgcagcaccagccaggagctcgccaagaccaagctgccccgcctggccaccttcctcaaggaagccgccaaggaggacgagaccttgctggataatcacttgaatgaagctaaagtcccaaacggaagtggctcccccacaaaagacgccctcggcggctacaccagggtggacacgaagccctcggccacgtcgagctccatgcggcggcggcggcacgcgttccgcaggcaggcctccagcaccttctcctactacggcaagcttgggtcccactgctaccgctaccggcgcgccaacgccgtggtgctgatcaagccgtcgcgcagcatgagcgacctgtacgacatgcagaagcggcagcggcagcaccggcaccggaaccagagcggggccaccacctccagcggggacaccgagagtgaggagggggagggcgagaccacggtgcgcctgctgtggctctccatgctgaagatgcccagggagctgatgcggctgtgcctctgccacctcctcacctggttctctgtcatcgccgaggccgtgttctacaccgacttcatgggccaggtcatcttcgaaggcgaccccaaggccccctcgaactcgaccgcctggcaagcctacaacgccggggtcaagatgggctgctggggcctggtcatttatgccgccactggtgctatttgttcagccctgttacagaagtacttggacaactacgacctgagcgtcagggtgatctacgtgctggggacgctgggcttctctgtcggcacagccgtgatggccatgtttcccaacgtctacgtcgccatggtcaccatcagcaccatgggcatcgtctccatgagcatctcctactgcccgtacgccctgctgggccagtaccatgacatcaagcagtacatccaccacagccccgggaactccaagcgagggtttggcatagattgtgccatcctgtcctgccaagtgtacatctcgcagatcctggtggcctctgcccttgggggcgtggtcgacgccgtggggactgtccgcgtcatccccatggtggcctctgtgggctctttcctgggcttcctgacggccacattcctggtgatctatcccaacgtgtcagaggaggccaaggaggagcagaaaggcctgtcttccccgttggccggcgaaggcagggccggtgggaacagcgaaaagcccaccgtgctgaagctcacgcggaaggagggcctgcagggaccggtggagacagaacgcctgcaggtattgacgtctgtcagatctcgtcacattggctggtgccgcagctgttggagagtatttttctttatgattattttagaaaaaaaattttcttttccacaatgtggttctcttagaagaatgacgtatcttcttttcctcagcgagttggacacattgtgcccagggcagccctgtccttgggcagcgaccgcacaccaaagctgggaggaggctggtccggggggcctgggcagaagacagtgatttgcaggggtggctcccagacaccctgcccagggatgggctgggcaccacctgggggcggagcgtgagctccagacgagctcctgcgtgcgcgtgtgagtgtgtctgcgcccagccatgtgaccccgctcgtcccgtctgaaggactctcctaggaggccaggttgcccctccagaccgctcccaacgtcagggggaaggaaacgttgactttcactgcactttgattcgtctctaaaccatttgctggggattcctgagagcagagctcccagcgggccctgcctcccaagtcccgccgcaaggctacctcgggtgtgtggatgtgcgagggcctcccccgcttgcgaaggggacatgcgtgctggaacctgtcggaactccatgccttcctcgcctgctcacctgctcgacgctggaatcgggacaggtgcaaagggacgcagacgtctgggacagctaaggcccgtgtcaccggagggctccgcacagtcgttctggtttcaacgaataagcaaaactcgggcaagtactgcagctatttggaaatgttttccaaaccacagtctctttagaactaagcctatttgaaacggtcggtgtaggcttactgagatcaggagacagggaggccccgcacatcacacagataaagtcagacaattgtaattaatacttttgctgcctcaagttgttttttaaataaagtactttgaaatgcatgagaatcatgctgcaatatgatcattctagagcaaatatatatatacacgtatatatatttcaagatgaaactaaagcagtttttaaataaattacttgaattttctgtgtatttaaaggaacgactgtttaatgtacttgatgggcctctggtcttgccgtgtctcctgccgctggtggcactttgtagattgtgtgtttgtgtccgggtggcagttgggtacctgctcacgcacggtgtgtctgccaggccacggtgtcccaggatcgcagagggctgactttcaagacttcaagaacattttctggatgtgtggaaacttgagaatggccttgtgaatctcgtgcttggacagggcaagtccgactactgaaagtgctgccagctttgctgcgagccctccggccagcgggagccccgtgggctgggcactgtggcccttcttctctgggggacggcacccctggcttcctcacctcggccgggcgtccgtggcagctcactctatgcaacttgatcctctagcggctttaagactgtagatcccctctctgagacctggctgtacttgtcaggatctcgaggcgcagctcccgtcttagctggtttctccggcttctcgtcctgacgactataaaacagttggaggcaagaaagcagcggatgtggggtggcagtggcctgacccgaatcaagatccgacccaaaccacaccaaatgtgggttcatctgggggccaccccttgcctgaggcttcccaccctcatctgaaggcccaggggccggatccagggctcaccaaagccgattcctcgccagctgggagtgcagaagtctcagggcctggctgcgacttgatttttaggaggaagaggggcttcgcaacccccctctgaatagtgtgttaacccttgagatcccagcctcgactaatctgaagtaaggacaacaaaggccattcagtgccctccacatggccttgccacagtcactgtcagggtatgaacgtgccggaagccagtgccagccagggacaggcgtgactgttgtgtgctcctcggtgacaggagtcggtggctgcacactttgtagactcgtcaagctgtcagcacttcaggtgtttgcaagcaaagcccttcttagtgtgcaggtcagtgtgcagagcccaaatgaggggaccgcaggggctggggtccagggtcagtagagtcggtttctggagctgccttcctgggaggcaggtgtgggtgaccggggctctggcggtgcgtgtgggcccggcctggccacagcggggaccaggtcacgacatctttggcctcaacccctcccctgcactgagattatttccagattgcactcacttgaaaccgtccgtgtcgtcaccttgtgtcttaagggaagccgagaagaagggcagacgggcaggctgtcttgtctgcaaagcgccattgcgcccgcagcttgtgtgggtcaggctgcagctcgggtgtctgtggcttctaaccttgtacctcagacggatgcagtaacaaggcggggctggggacgccggtcagtgtcaaaggggaggtgctctggctgatagccttgcccgagagggacgaggaggccgtgcgggtgccccctggggaagctggccagccatccagtgctgaggacgcagctggagttgggctcgtggcacccttggggtgtgggctgtgcaggctggtggcctgggtgcctctgcacactgagtggagtgtcaggcagggtgtgtcggttggtcgagtcttgtgtaatgtgcgcagaccagttaccaaactaggataatgttggtctcatttgtggtggttttgttccctatacaagtcagctaagtaaagactcttttaacgagcttccccttaacacatggcagaagtttccaggtgcaggaatgcgagctggcgggaaggggcagaggccgtgagctctcagctgggccggcctgcctgtgtcccctttcctgggtctgtcggcagactggcatcatgacgttccctggtggctgaagagctagctttggagtgttgtttttctcactctcaggcaggggccttagctggaatcctccaacctgccactgaacacgtcagtgctgtgtgctgcctcttggacacctgcccttgaaagcctcaggcccctgggagaagcactctgtccagtcctgtccccgggggggaggcagggccactgagccctcctcagatggttagtggcttccaacagccatcaggagtgtttcttgaatgccccaggtgtggaggacttggtctgtgaccacctagaaccccagagctgaacaggaagccgtccctgcagcaacaagagggctggaagggggagctgcaggccaccctcggctctcccactgctggggcggtgatgttcgggtgacatgtttgaaaaatactcttaaagataccaactgttcccttatatggctaatggtttgtgcagccaccagcgatggcggcccctattagagaccaggtttgttaaaacaccaaatattgctgtccacactagacattaaccggcttcagaaaagatggacaccttttcccacgctgtttcgcttcttaactttggtccagctttagccaccacacagcgtgtgagggactgctgctgcggagtcagcctcgtttgtccctccgcctcccaccagcacgcgccgcttctgagagacaccagctccctgcctccaagcctggtgccacaggcctgtcgtgagggacccctgcttccgagagctcctgggggggttctgcccttcaccacctgggagaggtgtcagttcagttccgagttgaacaaggcccgtgcacacagcatgttgggggcccagcccaaagttcttgtcacctcctcatgcaaagccagccatcaccctccggccagagctcaaggtggccccttggccagcccctccttgggtcctccaggaggactgagcacccctcctagcggcatcccttgccctccacagtgctgccaggggcacgtcgctctgtgccgtggactgagaccatcccctggtgacagaatgacccgtttgttggaaatgcctcgttgccagagaaactccccaggcatctcggaacgaaactatttagttccattgtgaactggccacgggacagctttttatcaacttattaagttggagcactgtaatcgcgcttgctgagttagcagtggtggtaagcgtgtgttaaacacataatgttacgttttaggagagagaggtcgtaaggaagtgtcgtgtcgctcatgactctcttctattagttgggtaacagtggcctcatgtttgtgtctgtgtgtacacagagcccttaggttctgctctgtttctttgccaggtgaatgtttgtggcatgcgctgctgtccgcgcccctctgtcctgcgcagggttcagctgtgcggcgccctgatttcctccatgcacacagaacctccttgtgtctgtttctctgttcctctgtggctgactcaataaacttttccctctga

SEQ ID NO: 492 - RNA, 2707 nt, Homo sapiens
  source 1..2707: mol_type = mRNA
tgatagggcagagaggagcccatgctccagctgcggggagtgcctgcccgaggagctggccagtgaagacaggtgcctgccagtgaagacaggtgcccacaagctggacacagggtcggccctgagacaggacagcagctgcggaatgttcccgctggctttctggagcgccagcgtcctgcttgggtctggccaggggcctctagccaggctgttgaggcgcccctccttgttgaagaggagaaacagtggccccctgggagttgctgggaggtgcctggcacagggcggcagggacagcatcatctatggggaaagcaagcccagcctcagggctgtcccgtccaaagacccttgtgagtccactgaggtcaaatcaatggagtcttcagaaaggccttccggagcagtactacagcctcacctggttcctgagccccatccttggcctcatcttcacacctctcattgggtctgcgagtgaccggtgcaccctgagctggggccgccggcggcccttcatcctcgccctctgcgttggcgtcctctttggcgttgcacttttccttaacggctctgccatcggtctggccctcggcgatgtccccaaccggcagcccattggcatcgtgctcacggtgctgggagtggtggtcctggacttcagcgccgatgccaccgaggggcccatccgtgcctatctgctggacgtggtggacagcgaggagcaggacatggccctcaacatccacgccttctctgccggcctcggcggagccatcggctacgtgctgggtgggctggactggacccagaccttcctgggcagctggttccggacccagaaccaggtgctcttcttctttgccgccatcatcttcacggtgtccgtggccctgcacctgttcagcatcgacgaggagcagtacagcccgcagcaggagcgcagcgctgaggagcccggcgccctggatgggggcgagccgcacggcgtccctgccttcccagacgaggtacagtcggagcacgagctggccctggactacccggacgtggacatcatgcgcagcaaaagcgactcggcattgcacgtgccggacaccgcgctggacctggagcccgagctgctgttcctgcacgacatcgagccctccatcttccacgacgcctcctaccccgccaccccccgcagcaccagccaggagctcgccaagaccaagctgccccgcctggccaccttcctcaaggaagccgccaaggaggacgagaccttgctggataatcacttgaatgaagctaaagtcccaaacggaagtggctcccccacaaaagacgccctcggcggctacaccagggtggacacgaagccctcggccacgtcgagctccatgcggcggcggcggcacgcgttccgcaggcaggcctccagcaccttctcctactacggcaagcttgggtcccactgctaccgctaccggcgcgccaacgccgtggtgctgatcaagccgtcgcgcagcatgagcgacctgtacgacatgcagaagcggcagcggcagcaccggcaccggaaccagagcggggccaccacctccagcggggacaccgagagtgaggagggggagggcgagaccacggtgcgcctgctgtggctctccatgctgaagatgcccagggagctgatgcggctgtgcctctgccacctcctcacctggttctctgtcatcgccgaggccgtgttctacaccgacttcatgggccaggtcatcttcgaaggcgaccccaaggccccctcgaactcgaccgcctggcaagcctacaacgccggggtcaagatgggctgctggggcctggtcatttatgccgccactggtgctatttgttcagccctgttacagaagtacttggacaactacgacctgagcgtcagggtgatctacgtgctggggacgctgggcttctctgtcggcacagccgtgatggccatgtttcccaacgtctacgtcgccatggtcaccatcagcaccatgggcatcgtctccatgagcatctcctactgcccgtacgccctgctgggccagtaccatgacatcaagcagtacatccaccacagccccgggaactccaagcgagggtttggcatagattgtgccatcctgtcctgccaagtgtacatctcgcagatcctggtggcctctgcccttgggggcgtggtcgacgccgtggggactgtccgcgtcatccccatggtggcctctgtgggctctttcctgggcttcctgacggccacattcctggtgatctatcccaacgtgtcagaggaggccaaggaggagcagaaaggcctgtcttccccgttggccggcgaaggcagggccggtgggaacagcgaaaagcccaccgtgctgaagctcacgcggaaggagggcctgcagggaccggtggagacagagtccgtggtctgagccgcactcccgtttacacacattccagtgggcgggtgggcgggcgggcgggcggcggggccaggccatgggcgggagcagagacaccgcggaaccctgcagatgctgtggccgacccggcagtgcgggccagagcccctccgcccccatagccacaattcagtagtcgtagggtaggtttgagctactaagcaaataccacactaaccactttttcgataattaaaagaatcatttgaaatatt